(19) United States
(12) Patent Application Publication
(10) Pub. No.: US 2022/0235051 A1
(43) Pub. Date: Jul. 28, 2022

(54) DI-FLUORO AZEPANES AS HBV CAPSID ASSEMBLY MODULATORS

(71) Applicant: Janssen Sciences Ireland Unlimited Company, Co Cork (IE)

(72) Inventors: Scott D. KUDUK, Raritan, NJ (US); Lindsey Graham DERATT, Raritan, NJ (US)

(21) Appl. No.: 17/595,612

(22) PCT Filed: May 27, 2020
(86) PCT No.: PCT/EP2020/064747

§ 371 Date: Nov. 19, 2021

Related U.S. Application Data
(60) Provisional application No. 62/853,554, filed on May 28, 2019.

(51) Int. Cl. C07D 471/14

(52) U.S. Cl. CPC C07D 471/14

(57) ABSTRACT

Disclosed are compounds, compositions and methods for treating of diseases, syndromes, conditions, and disorders that are affected by the modulation of CAM1. Such compounds are represented by Formula (I) as follows:

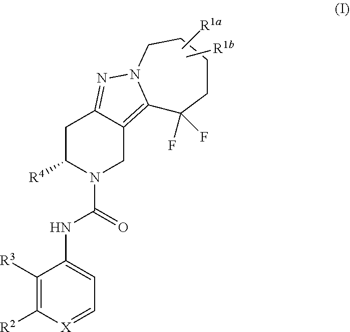

### FIELD OF THE PRESENT DISCLOSURE

The present disclosure is related to di-fluoro azepines compounds, pharmaceutical compositions comprising these compounds, chemical processes for preparing these compounds and their use in the treatment of diseases associated with HBV infection in animals, in particular humans.

### BACKGROUND

Chronic hepatitis B virus (HBV) infection is a significant global health problem, affecting over 5% of the world population (over 350 million people worldwide and 1.25 million individuals in the U.S.).

Despite the availability of a prophylactic HBV vaccine, the burden of chronic HBV infection continues to be a significant unmet worldwide medical problem, due to suboptimal treatment options and sustained rates of new infections in most parts of the developing world. Current treatments do not provide a cure and are limited to only two classes of agents (interferon alpha and nucleoside analogues/inhibitors of the viral polymerase); drug resistance, low efficacy, and tolerability issues limit their impact. The low cure rates of HBV are attributed at least in part to the fact that complete suppression of virus production is difficult to achieve with a single antiviral agent. However, persistent suppression of HBV DNA slows liver disease progression and helps to prevent hepatocellular carcinoma. Current therapy goals for HBV-infected patients are directed to reducing serum HBV DNA to low or undetectable levels, and to ultimately reducing or preventing the development of cirrhosis and hepatocellular carcinoma.

The HBV capsid protein plays essential functions during the viral life cycle. HBV capsid/core proteins form metastable viral particles or protein shells that protect the viral genome during intercellular passage, and also play a central role in viral replication processes, including genome encapsidation, genome replication, and virion morphogenesis and egress. Capsid structures also respond to environmental cues to allow un-coating after viral entry. Consistently, the appropriate timing of capsid assembly and dis-assembly, the appropriate capsid stability and the function of core protein have been found to be critical for viral infectivity.

The crucial function of HBV capsid proteins imposes stringent evolutionary constraints on the viral capsid protein sequence, leading to the observed low sequence variability and high conservation. Consistently, mutations in HBV capsid that disrupt its assembly are lethal, and mutations that perturb capsid stability severely attenuate viral replication. The high functional constraints on the multi-functional HBV core/capsid protein is consistent with a high sequence conservation, as many mutations are deleterious to function. Indeed, the core/capsid protein sequences are >90% identical across HBV genotypes and show only a small number of polymorphic residues. Resistance selection to HBV core/capsid protein binding compounds may therefore be difficult to select without large impacts on virus replication fitness.

Reports describing compounds that bind viral capsids and inhibit replication of HIV, rhinovirus and HBV provide strong pharmacological proof of concept for viral capsid proteins as antiviral drug targets.

There is a need in the art for therapeutic agents that can increase the suppression of virus production and that can treat, ameliorate, and/or prevent HBV infection. Administration of such therapeutic agents to an HBV infected patient, either as monotherapy or in combination with other HBV treatments or ancillary treatments, will lead to significantly reduced virus burden, improved prognosis, diminished progression of the disease and enhanced seroconversion rates.

In view of the clinical importance of HBV, the identification of compounds that can increase the suppression of virus production and that can treat, ameliorate, and/or prevent HBV infection represents an attractive avenue into the development of new therapeutic agents. Such compounds are provided herein.

### SUMMARY

The present disclosure is directed to the general and preferred embodiments defined, respectively, by the independent and dependent claims appended hereto, which are incorporated by reference herein. In particular, the present disclosure is directed to compounds of Formula (I):

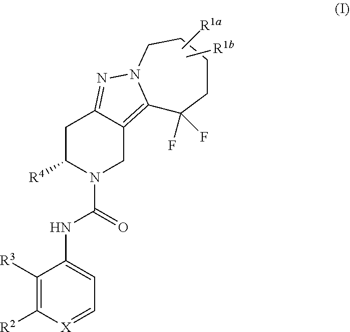

and pharmaceutically acceptable salts, stereoisomers, isotopic variants, N-oxides, or solvates of compounds of Formula (I);

wherein
R^{1a} is H, or OH;
R^{1b} is selected from the group consisting of: F, OH, C₁₋₄alkyl, C₂₋₄alkenyl, C₂₋₄ alkynyl, C₁₋₄haloalkyl, CH₂OH, C(CH₃)₂OH, CH₂CN, CH₂NH(C═O)CH₃, CH₂NH(C═O)OCH₃, CH₂OC₁₋₄haloalkyl, CH₂NH(C═O)CF₃, OC₁₋₄haloalkyl, or R^{1a} and R^{1b} come together to form ═CH₂;
R² is selected from the group consisting of: Br, CN, and C₁₋₄haloalkyl;
R³ is H, or F;
R⁴ is H or C₁₋₄alkyl; and
X is selected from the group consisting of: CH, CF, and N.

Further embodiments include pharmaceutically acceptable salts of compounds of Formula (I), pharmaceutically acceptable prodrugs of compounds of Formula (I), pharmaceutically active metabolites of compounds of Formula (I), and enantiomers and diastereomers of the compounds of Formula (I), as well as pharmaceutically acceptable salts thereof.

In embodiments, the compounds of Formula (I) are compounds selected from those species described or exemplified in the detailed description below.

The present disclosure is also directed to pharmaceutical compositions comprising one or more compounds of Formula (I), pharmaceutically acceptable salts of compounds of Formula (I), pharmaceutically acceptable prodrugs of compounds of Formula (I), and pharmaceutically active metabolites of Formula (I). Pharmaceutical compositions may further comprise one or more pharmaceutically acceptable excipients or one or more other agents or therapeutics.

The present disclosure is also directed to methods of using or uses of compounds of Formula (I). In embodiments, compounds of Formula (I) are used to treat or ameliorate hepatitis B viral (HBV) infection, increase the suppression of HBV production, interfere with HBV capsid assembly or other HBV viral replication steps or products thereof. The methods comprise administering to a subject in need of such method an effective amount of at least one compound of Formula (I), pharmaceutically acceptable salts of compounds of Formula (I), pharmaceutically acceptable prodrugs of compounds of Formula (I), and pharmaceutically active metabolites of compounds of Formula (I). Additional embodiments of methods of treatment are set forth in the detailed description.

An object of the present disclosure is to overcome or ameliorate at least one of the disadvantages of the conventional methodologies and/or prior art, or to provide a useful alternative thereto. Additional embodiments, features, and advantages of the present disclosure will be apparent from the following detailed description and through practice of the disclosed subject matter.

### DETAILED DESCRIPTION

Additional embodiments, features, and advantages of the subject matter of the present disclosure will be apparent from the following detailed description of such disclosure and through its practice. For the sake of brevity, the publications, including patents, cited in this specification are herein incorporated by reference.

Provided herein are compounds of Formula (I), including compounds of Formulae (IA) and (IB), and their pharmaceutically acceptable salts, pharmaceutically acceptable prodrugs, and pharmaceutically active metabolites of the disclosed compounds.

In one aspect, provided herein are compounds of Formula (I), and pharmaceutically acceptable salts, stereoisomers, isotopic variants, N-oxides, or solvates thereof,

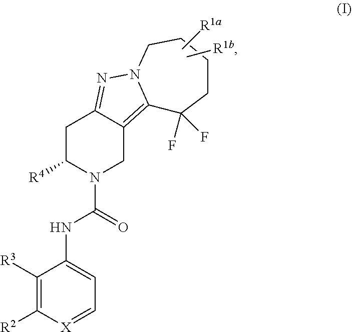

wherein
R^{1a} is H, or OH;
R^{1b} is selected from the group consisting of: F, OH, C₁₋₄alkyl, C₂₋₄alkenyl, C₂₋₄ alkynyl, C₁₋₄haloalkyl, CH₂OH, C(CH₃)₂OH, CH₂CN, CH₂NH(C═O)CH₃, CH₂NH(C═O)OCH₃, CH₂OC₁₋₄haloalkyl, CH₂NH(C═O)CF₃, OC₁₋₄haloalkyl, or R^{1a} and R^{1b} come together to form ═CH₂;
R² is selected from the group consisting of: Br, CN, and C₁₋₄haloalkyl;
R³ is H, or F;
R⁴ is H or C₁₋₄alkyl; and
X is selected from the group consisting of: CH, CF, and N.

In embodiments, the compound of Formula (I) is a compound wherein R^{1a} is H.

In embodiments, the compound of Formula (I) is a compound wherein R^{1a} is OH.

In embodiments, the compound of Formula (I) is a compound wherein R^{1a} is F.

In embodiments, the compound of Formula (I) is a compound wherein R^{1a} and R^{1b} come together to form ═CH₂.

In embodiments, the compound of Formula (I) is a compound wherein R^{1a} is H and R^{1b} is selected from the group consisting of: F, OH, CH₂OH, C(CH₃)₂OH, CH₂NH(C═O)CH₃, CH₂NH(C═O)CF₃, CH₂OCH₂CHF₂, and OCH₂CHF₂.

In embodiments, the compound of Formula (I) is a compound wherein R^{1a} is F and R^{1b} is CH₂OH.

In embodiments, the compound of Formula (I) is a compound wherein R^{1a} is OH and R^{1b} is selected from the group consisting of: CH₃, CH₂CH₃, CH═CH₂, C═CH, CH₂F, CH₂OH, CH₂CN, and CH₂OCH₂CHF₂.

In embodiments, the compound of Formula (I) is a compound wherein R² is Br, CN, CHF₂ or CF₃.

In embodiments, the compound of Formula (I) is a compound wherein R³ is H.

In embodiments, the compound of Formula (I) is a compound wherein R³ is F.

In embodiments, the compound of Formula (I) is a compound wherein R⁴ is H.

In embodiments, the compound of Formula (I) is a compound wherein R⁴ is CH₃.

In embodiments, the compound of Formula (I) is a compound wherein X is N.

In embodiments, the compound of Formula (I) is a compound wherein X is CF.

In embodiments, the compound of Formula (I) is a compound wherein X is CH.

In embodiments, the compound of Formula (I) is a compound wherein

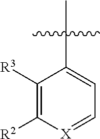

is 3-cyano-4-fluorophenyl, 4-fluoro-3-(trifluoromethyl)phenyl, 3-cyano-2,4-difluorophenyl, 3-bromo-2,4-difluorophenyl, 2-(difluoromethyl)-3-fluoropyridin-4-yl, or 2-bromo-3-fluoropyridin-4-yl.

An embodiment of the present disclosure is a compound of Formula (I) having the Formula (IA):

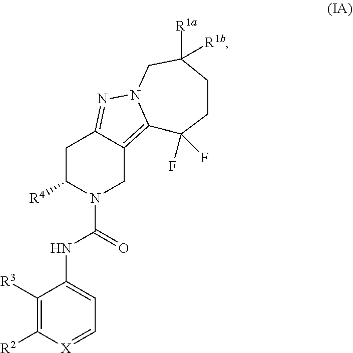

wherein
R^{1a} is H, or OH;
R^{1b} is selected from the group consisting of: F, OH, C₁₋₄alkyl, C₂₋₄alkenyl, C₂₋₄alkynyl, C₁₋₄haloalkyl, CH₂OH, C(CH₃)₂OH, CH₂CN, CH₂NH(C═O)CH₃, CH₂NH(C═O)OCH₃, CH₂OC₁₋₄haloalkyl, CH₂NH(C═O)CF₃, OC₁₋₄haloalkyl, or R^{1a} and R^{1b} come together to form ═CH₂;
R² is selected from the group consisting of: Br, CN, and C₁₋₄haloalkyl;
R³ is H, or F;
R⁴ is H or CH₃; and
X is selected from the group consisting of: CH, CF, and N;

and pharmaceutically acceptable salts, N-oxides or solvates of compounds of Formula (IA).

An embodiment of the of the present disclosure is a compound of Formula (I) having the Formula (IB):

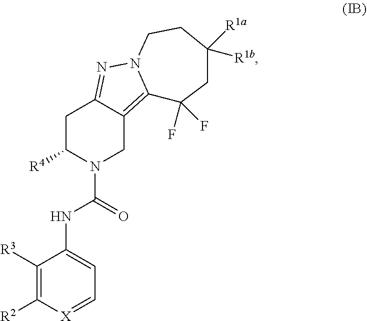

wherein,
R^{1a} is H, or OH;
R^{1b} is selected from the group consisting of: C₁₋₄haloalkyl, and CH₂OH;
R² is selected from the group consisting of: Br, CN, and C₁₋₄haloalkyl;
R³ is H, or F;
R⁴ is H or CH₃; and
X is selected from the group consisting of: CH, CF, and N;

and pharmaceutically acceptable salts, N-oxides or solvates of compounds of Formula (IB).

A further embodiment of the present disclosure is a compound as shown below in Table 1.

TABLE 1

| Ex # | Compound_Name |
|---|---|
| | N-(3-Cyano-4-fluorophenyl)-11,11-difluoro-8-methylene-3,4,8,9,10,11- |
| | hexahydro-1H-pyrido[4′,3′:3,4]pyrazolo[1,5-a]azepine-2(7H)-carboxamide; |
| 2 | 11,11-Difluoro-N-(4-fluoro-3-(trifluoromethyl)phenyl)-8-methylene- |
| | 3,4,8,9,10,11- hexahydro-1H-pyrido[4′,3′:3,4]pyrazolo[1,5-a]azepine-2(7H)- |
| | carboxamide; |
| 3 | (S)-N-(3-Cyano-4-fluorophenyl)-11,11-difluoro-8-hydroxy-3,4,8,9,10,11- |
| | hexahydro-1H-pyrido[4′,3′:3,4]pyrazolo[1,5-a]azepine-2(7H)-carboxamide; |
| 4 | (S)-11,11-Difluoro-N-(4-fluoro-3-(trifluoromethyl)phenyl)-8-hydroxy- |
| | 3,4,8,9,10,11-hexahydro-1H-pyrido[4′,3′:3,4]pyrazolo[1,5-a]azepine-2(7H)- |
| | carboxamide; |
| 5 | (S)-N-(2-Bromo-3-fluoropyridin-4-yl)-11,11-difluoro-8-hydroxy- |
| | 3,4,8,9,10,11-hexahydro-1H-pyrido[4′,3′:3,4]pyrazolo[1,5-a]azepine-2(7H)- |
| | carboxamide; |
| 6 | (S)-N-(3-Cyano-2,4-difluorophenyl)-11,11-difluoro-8-hydroxy- |
| | 3,4,8,9,10,11-hexahydro-1H-pyrido[4′,3′:3,4]pyrazolo[1,5-a]azepine-2(7H)- |
| | carboxamide; |
| 7 | (S)-N-(3-Bromo-2,4-difluorophenyl)-11,11-difluoro-8-hydroxy- |
| | 3,4,8,9,10,11-hexahydro-1H-pyrido[4′,3′:3,4]pyrazolo[1,5-a]azepine-2(7H)- |
| | carboxamide; |
| 8 | (R)-N-(3-Cyano-4-fluorophenyl)-11,11-difluoro-8-hydroxy-3,4,8,9,10,11- |
| | hexahydro-1H-pyrido[4′,3′:3,4]pyrazolo[1,5-a]azepine-2(7H)-carboxamide; |
| 9 | (R)-11,11-Difluoro-N-(4-fluoro-3-(trifluoromethyl)phenyl)-8-hydroxy- |
| | 3,4,8,9,10,11-hexahydro-1H-pyrido[4′,3′:3,4]pyrazolo[1,5-a]azepine-2(7H)- |
| | carboxamide; |
| 10 | (R)-N-(2-Bromo-3-fluoropyridin-4-yl)-11,11-difluoro-8-hydroxy- |
| | 3,4,8,9,10,11-hexahydro-1H-pyrido[4′,3′:3,4]pyrazolo[1,5-a]azepine-2(7H)- |
| | carboxamide; |
| 11 | (R)-N-(3-Cyano-2,4-difluorophenyl)-11,11-difluoro-8-hydroxy- |
| | 3,4,8,9,10,11-hexahydro-1H-pyrido[4′,3′:3,4]pyrazolo[1,5-a]azepine-2(7H)- |
| | carboxamide; |
| 12 | (R)-N-(3-Bromo-2,4-difluorophenyl)-11,11-difluoro-8-hydroxy- |
| | 3,4,8,9,10,11-hexahydro-1H-pyrido[4′,3′:3,4]pyrazolo[1,5-a]azepine-2(7H)- |
| | carboxamide; |
| 13 | (S)-N-(3-Cyano-4-fluorophenyl)-8-(2,2-difluoroethoxy)-11,11-difluoro- |
| | 3,4,8,9,10,11-hexahydro-1H-pyrido[4′,3′:3,4]pyrazolo[1,5-a]azepine-2(7H)- |
| | carboxamide; |
| 14 | (R)-N-(3-Cyano-4-fluorophenyl)-8-(2,2-difluoroethoxy)-11,11-difluoro- |
| | 3,4,8,9,10,11-hexahydro-1H-pyrido[4′,3′:3,4]pyrazolo[1,5-a]azepine-2(7H)- |
| | carboxamide; |
| 15 | (R)-N-(3-Cyano-4-fluorophenyl)-8,11,11-trifluoro-3,4,8,9,10,11-hexahydro- |
| | 1H-pyrido[4′,3′:3,4]pyrazolo[1,5-a]azepine-2(7H)-carboxamide; |
| 16 | (R)-8,11,11-Trifluoro-N-(4-fluoro-3-(trifluoromethyl)phenyl)-3,4,8,9,10,11- |
| | hexahydro-1H-pyrido[4′,3′:3,4]pyrazolo[1,5-a]azepine-2(7H)-carboxamide; |
| 17 | (S)-N-(3-Cyano-4-fluorophenyl)-8,11,11-trifluoro-3,4,8,9,10,11-hexahydro- |
| | 1H-pyrido[4′,3′:3,4]pyrazolo[1,5-a]azepine-2(7H)-carboxamide; |
| 18 | (S)-8,11,11-Trifluoro-N-(4-fluoro-3-(trifluoromethyl)phenyl)-3,4,8,9,10,11- |
| | hexahydro-1H-pyrido[4′,3′:3,4]pyrazolo[1,5-a]azepine-2(7H)-carboxamide; |
| 19 | (S*)-N-(3-Cyano-4-fluorophenyl)-11,11-difluoro-8-(hydroxymethyl)- |
| | 3,4,8,9,10,11-hexahydro-1H-pyrido[4′,3′:3,4]pyrazolo[1,5-a]azepine-2(7H)- |
| | carboxamide; |
| 20 | (S*)-11,11-Difluoro-N-(4-fluoro-3-(trifluoromethyl)phenyl)-8- |
| | (hydroxymethyl)-3,4,8,9,10,11-hexahydro-1H-pyrido[4′,3′:3,4]pyrazolo[1,5- |
| | a]azepine-2(7H)-carboxamide; |
| 21 | (S*)-N-(3-Cyano-2,4-difluorophenyl)-11,11-difluoro-8-(hydroxymethyl)- |
| | 3,4,8,9,10,11-hexahydro-1H-pyrido[4′,3′:3,4]pyrazolo[1,5-a]azepine-2(7H)- |
| | carboxamide; |
| 22 | (S*)-N-(2-Bromo-3-fluoropyridin-4-yl)-11,11-difluoro-8-(hydroxymethyl)- |
| | 3,4,8,9,10,11-hexahydro-1H-pyrido[4′,3′:3,4]pyrazolo[1,5-a]azepine-2(7H)- |
| | carboxamide; |
| 23 | (R*)-N-(3-Cyano-4-fluorophenyl)-11,11-difluoro-8-(hydroxymethyl)- |
| | 3,4,8,9,10,11-hexahydro-1H-pyrido[4′,3′:3,4]pyrazolo[1,5-a]azepine-2(7H)- |
| | carboxamide; |
| 24 | (R*)-11,11-Difluoro-N-(4-fluoro-3-(trifluoromethyl)phenyl)-8- |
| | (hydroxymethyl)-3,4,8,9,10,11-hexahydro-1H-pyrido[4′,3′:3,4]pyrazolo[1,5- |
| | a]azepine-2(7H)-carboxamide; |
| 25 | (R*)-N-(3-Cyano-2,4-difluorophenyl)-11,11-difluoro-8-(hydroxymethyl)- |
| | 3,4,8,9,10,11-hexahydro-1H-pyrido[4′,3′:3,4]pyrazolo[1,5-a]azepine-2(7H)- |
| | carboxamide; |
| 26 | (R*)-N-(2-Bromo-3-fluoropyridin-4-yl)-11,11-difluoro-8-(hydroxymethyl)- |
| | 3,4,8,9,10,11-hexahydro-1H-pyrido[4′,3′:3,4]pyrazolo[1,5-a]azepine-2(7H)- |
| | carboxamide; |
| 27 | (S*)-N-(3-Cyano-4-fluorophenyl)-8-((2,2-difluoroethoxy)methyl)-11,11- |
| | difluoro-3,4,8,9,10,11-hexahydro-1H-pyrido[4′,3′:3,4]pyrazolo[1,5- |
| | a]azepine-2(7H)-carboxamide; |
| 28 | (S*)-8-((2,2-Difluoroethoxy)methyl)-11,11-difluoro-N-(4-fluoro-3- |
| | (trifluoromethyl)phenyl)-3,4,8,9,10,11-hexahydro-1H- |
| | pyrido[4′,3′:3,4]pyrazolo[1,5-a]azepine-2(7H)-carboxamide; |
| 29 | (R*)-N-(3-Cyano-4-fluorophenyl)-8-((2,2-difluoroethoxy)methyl)-11,11- |
| | difluoro-3,4,8,9,10,11-hexahydro-1H-pyrido[4′,3′:3,4]pyrazolo[1,5- |
| | a]azepine-2(7H)-carboxamide; |
| 30 | (R*)-8-((2,2-Difluoroethoxy)methyl)-11,11-difluoro-N-(4-fluoro-3- |
| | (trifluoromethyl)phenyl)-3,4,8,9,10,11-hexahydro-1H- |
| | pyrido[4′,3′:3,4]pyrazolo[1,5-a]azepine-2(7H)-carboxamide; |
| 31 | (R*)-N-(3-Cyano-4-fluorophenyl)-8-((2,2-difluoroethoxy)methyl)-11,11- |
| | difluoro-8-hydroxy-3,4,8,9,10,11-hexahydro-1H- |
| | pyrido[4′,3′:3,4]pyrazolo[1,5-a]azepine-2(7H)-carboxamide; |
| 32 | (R*)-N-(2-Bromo-3-fluoropyridin-4-yl)-8-((2,2-difluoroethoxy)methyl)- |
| | 11,11-difluoro-8-hydroxy-3,4,8,9,10,11-hexahydro-1H- |
| | pyrido[4′,3′:3,4]pyrazolo[1,5-a]azepine-2(7H)-carboxamide; |
| 33 | (R*)-8-((2,2-Difluoroethoxy)methyl)-N-(2-(difluoromethyl)-3- |
| | fluoropyridin-4-yl)-11,11-difluoro-8-hydroxy-3,4,8,9,10,11-hexahydro-1H- |
| | pyrido[4′,3′:3,4]pyrazolo[1,5-a]azepine-2(7H)-carboxamide; |
| 34 | (S*)-N-(3-Cyano-4-fluorophenyl)-8-((2,2-difluoroethoxy)methyl)-11,11- |
| | difluoro-8-hydroxy-3,4,8,9,10,11-hexahydro-1H- |
| | pyrido[4′,3′:3,4]pyrazolo[1,5-a]azepine-2(7H)-carboxamide; |
| 35 | (S*)-N-(2-Bromo-3-fluoropyridin-4-yl)-8-((2,2-difluoroethoxy)methyl)- |
| | 11,11-difluoro-8-hydroxy-3,4,8,9,10,11-hexahydro-1H- |
| | pyrido[4′,3′:3,4]pyrazolo[1,5-a]azepine-2(7H)-carboxamide; |
| 36 | (S*)-8-((2,2-Difluoroethoxy)methyl)-N-(2-(difluoromethyl)-3- |
| | fluoropyridin-4-yl)-11,11-difluoro-8-hydroxy-3,4,8,9,10,11-hexahydro-1H- |
| | pyrido[4′,3′:3,4]pyrazolo[1,5-a]azepine-2(7H)-carboxamide; |
| 37 | N-(3-Cyano-4-fluorophenyl)-11,11-difluoro-8-(fluoromethyl)-8-hydroxy- |
| | 3,4,8,9,10,11-hexahydro-1H-pyrido[4′,3′:3,4]pyrazolo[1,5-a]azepine-2(7H)- |
| | carboxamide; |
| 38 | (S*)-N-(2-Bromo-3-fluoropyridin-4-yl)-11,11-difluoro-8-(fluoromethyl)-8- |
| | hydroxy-3,4,8,9,10,11-hexahydro-1H-pyrido[4′,3′:3,4]pyrazolo[1,5- |
| | a]azepine-2(7H)-carboxamide; |
| 39 | (R*)-N-(2-Bromo-3-fluoropyridin-4-yl)-11,11-difluoro-8-(fluoromethyl)-8- |
| | hydroxy-3,4,8,9,10,11-hexahydro-1H-pyrido[4′,3′:3,4]pyrazolo[1,5- |
| | a]azepine-2(7H)-carboxamide; |
| 40 | N-(3-Cyano-4-fluorophenyl)-8-(cyanomethyl)-11,11-difluoro-8-hydroxy- |
| | 3,4,8,9,10,11-hexahydro-1H-pyrido[4′,3′:3,4]pyrazolo[1,5-a]azepine-2(7H)- |
| | carboxamide; |
| 41 | (R*)-N-(2-Bromo-3-fluoropyridin-4-yl)-8-(cyanomethyl)-11,11-difluoro-8- |
| | hydroxy-3,4,8,9,10,11-hexahydro-1H-pyrido[4′,3′:3,4]pyrazolo[1,5- |
| | a]azepine-2(7H)-carboxamide; |
| 42 | (R*)-8-(Cyanomethyl)-N-(2-(difluoromethyl)-3-fluoropyridin-4-yl)-11,11- |
| | difluoro-8-hydroxy-3,4,8,9,10,11-hexahydro-1H- |
| | pyrido[4′,3′:3,4]pyrazolo[1,5-a]azepine-2(7H)-carboxamide; |
| 43 | (S*)-N-(2-Bromo-3-fluoropyridin-4-yl)-8-(cyanomethyl)-11,11-difluoro-8- |
| | hydroxy-3,4,8,9,10,11-hexahydro-1H-pyrido[4′,3′:3,4]pyrazolo[1,5- |
| | a]azepine-2(7H)-carboxamide; |
| 44 | (S*)-8-(Cyanomethyl)-N-(2-(difluoromethyl)-3-fluoropyridin-4-yl)-11,11- |
| | difluoro-8-hydroxy-3,4,8,9,10,11-hexahydro-1H- |
| | pyrido[4′,3′:3,4]pyrazolo[1,5-a]azepine-2(7H)-carboxamide; |
| 45 | N-(3-Cyano-4-fluorophenyl)-8,11,11-trifluoro-8-(hydroxymethyl)- |
| | 3,4,8,9,10,11-hexahydro-1H-pyrido[4′,3′:3,4]pyrazolo[1,5-a]azepine-2(7H)- |
| | carboxamide; |
| 46 | N-(2-Bromo-3-fluoropyridin-4-yl)-8,11,11-trifluoro-8-(hydroxymethyl)- |
| | 3,4,8,9,10,11-hexahydro-1H-pyrido[4′,3′:3,4]pyrazolo[1,5-a]azepine-2(7H)- |
| | carboxamide; |
| 47 | (S*)-N-(2-Bromo-3-fluoropyridin-4-yl)-8,11,11-trifluoro-8- |
| | (hydroxymethyl)-3,4,8,9,10,11-hexahydro-1H-pyrido[4′,3′:3,4]pyrazolo[1,5- |
| | a]azepine-2(7H)-carboxamide; |
| 48 | (R*)-N-(2-Bromo-3-fluoropyridin-4-yl)-8,11,11-trifluoro-8- |
| | (hydroxymethyl)-3,4,8,9,10,11-hexahydro-1H-pyrido[4′,3′:3,4]pyrazolo[1,5- |
| | a]azepine-2(7H)-carboxamide; |
| 49 | 8-Acetamidomethyl)-N-(3-cyano-4-fluorophenyl)-11,11-difluoro- |
| | 3,4,8,9,10,11-hexahydro-1H-pyrido[4′,3′:3,4]pyrazolo[1,5-a]azepine-2(7H)- |
| | carboxamide; |
| 50 | 8-(Acetamidomethyl)-N-(2-bromo-3-fluoropyridin-4-yl)-11,11-difluoro- |
| | 3,4,8,9,10,11-hexahydro-1H-pyrido[4′,3′:3,4]pyrazolo[1,5-a]azepine-2(7H)- |
| | carboxamide; |
| 51 | N-(3-Cyano-4-fluorophenyl)-11,11-difluoro-8-((2,2,2-trifluoroacetamido) |
| | methyl)-3,4,8,9,10,11-hexahydro-1H-pyrido[4′,3′:3,4]pyrazolo[1,5- |
| | a]azepine-2(7H)-carboxamide; |
| 52 | N-(2-Bromo-3-fluoropyridin-4-yl)-11,11-difluoro-8-((2,2,2- |
| | trifluoroacetamido)methyl)-3,4,8,9,10,11-hexahydro-1H- |
| | pyrido[4′,3′:3,4]pyrazolo[1,5-a]azepine-2(7H)-carboxamide; |
| 53 | Methyl ((2-((3-cyano-4-fluorophenyl)carbamoyl)-11,11-difluoro- |
| | 2,3,4,7,8,9,10,11-octahydro-1H-pyrido[4′,3′:3,4]pyrazolo[1,5-a]azepin-8- |
| | yl)methyl)carbamate; |
| 54 | Methyl ((2-((2-bromo-3-fluoropyridin-4-yl)carbamoyl)-11,11-difluoro- |
| | 2,3,4,7,8,9,10,11-octahydro-1H-pyrido[4′,3′:3,4]pyrazolo[1,5-a]azepin-8- |
| | yl)methyl)carbamate; |
| 55 | N-(3-Cyano-4-fluorophenyl)-11,11-difluoro-8-hydroxy-8-methyl- |
| | 3,4,8,9,10,11-hexahydro-1H-pyrido[4′,3′:3,4]pyrazolo[1,5-a]azepine-2(7H)- |
| | carboxamide; |
| 56 | (R*)-N-(3-Cyano-4-fluorophenyl)-11,11-difluoro-8-hydroxy-8-vinyl- |
| | 3,4,8,9,10,11-hexahydro-1H-pyrido[4′,3′:3,4]pyrazolo[1,5-a]azepine-2(7H)- |
| | carboxamide; |
| 57 | (S*)-N-(3-Cyano-4-fluorophenyl)-11,11-difluoro-8-hydroxy-8-vinyl- |
| | 3,4,8,9,10,11-hexahydro-1H-pyrido[4′,3′:3,4]pyrazolo[1,5-a]azepine-2(7H)- |
| | carboxamide; |
| 58 | N-(3-Cyano-4-fluorophenyl)-8-ethynyl-11,11-difluoro-8-hydroxy- |
| | 3,4,8,9,10,11-hexahydro-1H-pyrido[4′,3′:3,4]pyrazolo[1,5-a]azepine-2(7H)- |
| | carboxamide; |
| 59 | (R*)-N-(3-Cyano-4-fluorophenyl)-8-ethynyl-11,11-difluoro-8-hydroxy- |
| | 3,4,8,9,10,11-hexahydro-1H-pyrido[4′,3′:3,4]pyrazolo[1,5-a]azepine-2(7H)- |
| | carboxamide; |
| 60 | (S*)-N-(3-Cyano-4-fluorophenyl)-8-ethynyl-11,11-difluoro-8-hydroxy- |
| | 3,4,8,9,10,11-hexahydro-1H-pyrido[4′,3′:3,4]pyrazolo[1,5-a]azepine-2(7H)- |
| | carboxamide; |
| 61 | (S*)-N-(3-Cyano-4-fluorophenyl)-8-ethyl-11,11-difluoro-8-hydroxy- |
| | 3,4,8,9,10,11-hexahydro-1H-pyrido[4′,3′:3,4]pyrazolo[1,5-a]azepine-2(7H)- |
| | carboxamide; |
| 62 | (R*)-N-(3-Cyano-4-fluorophenyl)-8-ethyl-11,11-difluoro-8-hydroxy- |
| | 3,4,8,9,10,11-hexahydro-1H-pyrido[4′,3′:3,4]pyrazolo[1,5-a]azepine-2(7H)- |
| | carboxamide; |
| 63 | (R*)-N-(2-Bromo-3-fluoropyridin-4-yl)-8-ethynyl-11,11-difluoro-8- |
| | hydroxy-3,4,8,9,10,11-hexahydro-1H-pyrido[4′,3′:3,4]pyrazolo[1,5- |
| | a]azepine-2(7H)-carboxamide; |
| 64 | (R*)-N-(2-(Difluoromethyl)-3-fluoropyridin-4-yl)-8-ethynyl-11,11- |
| | difluoro-8-hydroxy-3,4,8,9,10,11-hexahydro-1H- |
| | pyrido[4′,3′:3,4]pyrazolo[1,5-a]azepine-2(7H)-carboxamide; |
| 65 | (S*)-N-(2-Bromo-3-fluoropyridin-4-yl)-8-ethynyl-11,11-difluoro-8- |
| | hydroxy-3,4,8,9,10,11-hexahydro-1H-pyrido[4′,3′:3,4]pyrazolo[1,5- |
| | a]azepine-2(7H)-carboxamide; |
| 66 | (S*)-N-(2-(Difluoromethyl)-3-fluoropyridin-4-yl)-8-ethynyl-11,11- |
| | difluoro-8-hydroxy-3,4,8,9,10,11-hexahydro-1H- |
| | pyrido[4′,3′:3,4]pyrazolo[1,5-a]azepine-2(7H)-carboxamide; |
| 67 | (3R,8R)-N-(3-cyano-4-fluorophenyl)-11,11-difluoro-8-hydroxy-3-methyl- |
| | 3,4,8,9,10,11-hexahydro-1H-pyrido[4′,3′:3,4]pyrazolo[1,5-a]azepine-2(7H)- |
| | carboxamide; |
| 68 | (3R,8R)-N-(2-Bromo-3-fluoropyridin-4-yl)-11,11-difluoro-8-hydroxy-3- |
| | methyl-3,4,8,9,10,11-hexahydro-1H-pyrido[4′,3′:3,4]pyrazolo[1,5-a]azepine- |
| | 2(7H)-carboxamide; |
| 69 | (3R,8R)-11,11-Difluoro-N-(4-fluoro-3-(trifluoromethyl)phenyl)-8-hydroxy- |
| | 3-methyl-3,4,8,9,10,11-hexahydro-1H-pyrido[4′,3′:3,4]pyrazolo[1,5- |
| | a]azepine-2(7H)-carboxamide; |
| 70 | (3R,8R)-N-(2-(Difluoromethyl)-3-fluoropyridin-4-yl)-11,11-difluoro-8- |
| | hydroxy-3-methyl-3,4,8,9,10,11-hexahydro-1H- |
| | pyrido[4′,3′:3,4]pyrazolo[1,5-a]azepine-2(7H)-carboxamide; |
| 71 | (3R,8S)-N-(3-Cyano-4-fluorophenyl)-11,11-difluoro-8-hydroxy-3-methyl- |
| | 3,4,8,9,10,11-hexahydro-1H-pyrido[4′,3′:3,4]pyrazolo[1,5-a]azepine-2(7H)- |
| | carboxamide; |
| 72 | (3R,8S)-N-(2-Bromo-3-fluoropyridin-4-yl)-11,11-difluoro-8-hydroxy-3- |
| | methyl-3,4,8,9,10,11-hexahydro-1H-pyrido[4′,3′:3,4]pyrazolo[1,5-a]azepine- |
| | 2(7H)-carboxamide; |
| 73 | (3R,8S)-11,11-Difluoro-N-(4-fluoro-3-(trifluoromethyl)phenyl)-8-hydroxy- |
| | 3-methyl-3,4,8,9,10,11-hexahydro-1H-pyrido[4′,3′:3,4]pyrazolo[1,5- |
| | a]azepine-2(7H)-carboxamide; |
| 74 | (3R,8S)-N-(2-(Difluoromethyl)-3-fluoropyridin-4-yl)-11,11-difluoro-8- |
| | hydroxy-3-methyl-3,4,8,9,10,11-hexahydro-1H- |
| | pyrido[4′,3′:3,4]pyrazolo[1,5-a]azepine-2(7H)-carboxamide; |
| 75 | (3R,8S*)-N-(3-Cyano-4-fluorophenyl)-11,11-difluoro-8-(hydroxymethyl)- |
| | 3-methyl-3,4,8,9,10,11-hexahydro-1H-pyrido[4′,3′:3,4]pyrazolo[1,5- |
| | a]azepine-2(7H)-carboxamide; |
| 76 | (3R,8S*)-11,11-Difluoro-N-(4-fluoro-3-(trifluoromethyl)phenyl)-8- |
| | (hydroxymethyl)-3-methyl-3,4,8,9,10,11-hexahydro-1H- |
| | pyrido[4′,3′:3,4]pyrazolo[1,5-a]azepine-2(7H)-carboxamide; |
| 77 | (3R,8S*)-N-(2-Bromo-3-fluoropyridin-4-yl)-11,11-difluoro-8- |
| | (hydroxymethyl)-3-methyl-3,4,8,9,10,11-hexahydro-1H- |
| | pyrido[4′,3′:3,4]pyrazolo[1,5-a]azepine-2(7H)-carboxamide; |
| 78 | (3R,8S*)-N-(2-(Difluoromethyl)-3-fluoropyridin-4-yl)-11,11-difluoro-8- |
| | (hydroxymethyl)-3-methyl-3,4,8,9,10,11-hexahydro-1H- |
| | pyrido[4′,3′:3,4]pyrazolo[1,5-a]azepine-2(7H)-carboxamide; |
| 79 | (3R,8R*)-N-(3-Cyano-4-fluorophenyl)-11,11-difluoro-8-(hydroxymethyl)- |
| | 3-methyl-3,4,8,9,10,11-hexahydro-1H-pyrido[4′,3′:3,4]pyrazolo[1,5- |
| | a]azepine-2(7H)-carboxamide; |
| 80 | (3R,8R*)-11,11-Difluoro-N-(4-fluoro-3-(trifluoromethyl)phenyl)-8- |
| | (hydroxymethyl)-3-methyl-3,4,8,9,10,11-hexahydro-1H- |
| | pyrido[4′,3′:3,4]pyrazolo[1,5-a]azepine-2(7H)-carboxamide; |
| 81 | (3R,8R*)-N-(2-Bromo-3-fluoropyridin-4-yl)-11,11-difluoro-8- |
| | (hydroxymethyl)-3-methyl-3,4,8,9,10,11-hexahydro-1H- |
| | pyrido[4′,3′:3,4]pyrazolo[1,5-a]azepine-2(7H)-carboxamide; |
| 82 | (3R,8R*)-N-(2-(Difluoromethyl)-3-fluoropyridin-4-yl)-11,11-difluoro-8- |
| | (hydroxymethyl)-3-methyl-3,4,8,9,10,11-hexahydro-1H- |
| | pyrido[4′,3′:3,4]pyrazolo[1,5-a]azepine-2(7H)-carboxamide; |
| 83 | (3R,8R*)-N-(3-Cyano-4-fluorophenyl)-11,11-difluoro-8-(fluoromethyl)-8- |
| | hydroxy-3-methyl-3,4,8,9,10,11-hexahydro-1H- |
| | pyrido[4′,3′:3,4]pyrazolo[1,5-a]azepine-2(7H)-carboxamide; |
| 84 | (3R,8R*)-N-(2-Bromo-3-fluoropyridin-4-yl)-11,11-difluoro-8- |
| | (fluoromethyl)-8-hydroxy-3-methyl-3,4,8,9,10,11-hexahydro-1H- |
| | pyrido[4′,3′:3,4]pyrazolo[1,5-a]azepine-2(7H)-carboxamide; |
| 85 | (3R,8R*)-N-(2-(Difluoromethyl)-3-fluoropyridin-4-yl)-11,11-difluoro-8- |
| | (fluoromethyl)-8-hydroxy-3-methyl-3,4,8,9,10,11-hexahydro-1H- |
| | pyrido[4′,3′:3,4]pyrazolo[1,5-a]azepine-2(7H)-carboxamide; |
| 86 | (3R,8S*)-N-(3-Cyano-4-fluorophenyl)-11,11-difluoro-8-(fluoromethyl)-8- |
| | hydroxy-3-methyl-3,4,8,9,10,11-hexahydro-1H- |
| | pyrido[4′,3′:3,4]pyrazolo[1,5-a]azepine-2(7H)-carboxamide; |
| 87 | (3R,8S*)-N-(2-Bromo-3-fluoropyridin-4-yl)-11,11-difluoro-8- |
| | (fluoromethyl)-8-hydroxy-3-methyl-3,4,8,9,10,11-hexahydro-1H- |
| | pyrido[4′,3′:3,4]pyrazolo[1,5-a]azepine-2(7H)-carboxamide; |
| 88 | (3R,8S*)-N-(2-(Difluoromethyl)-3-fluoropyridin-4-yl)-11,11-difluoro-8- |
| | (fluoromethyl)-8-hydroxy-3-methyl-3,4,8,9,10,11-hexahydro-1H- |
| | pyrido[4′,3′:3,4]pyrazolo[1,5-a]azepine-2(7H)-carboxamide; |
| 89 | (3R,8S*)-N-(2-Bromo-3-fluoropyridin-4-yl)-8-(cyanomethyl)-11,11- |
| | difluoro-8-hydroxy-3-methyl-3,4,8,9,10,11-hexahydro-1H- |
| | pyrido[4′,3′:3,4]pyrazolo[1,5-a]azepine-2(7H)-carboxamide; |
| 90 | (3R,8S*)-8-(Cyanomethyl)-N-(2-(difluoromethyl)-3-fluoropyridin-4-yl)- |
| | 11,11-difluoro-8-hydroxy-3-methyl-3,4,8,9,10,11-hexahydro-1H- |
| | pyrido[4′,3′:3,4]pyrazolo[1,5-a]azepine-2(7H)-carboxamide; |
| 91 | (3R,8R*)-N-(2-Bromo-3-fluoropyridin-4-yl)-8-(cyanomethyl)-11,11- |
| | difluoro-8-hydroxy-3-methyl-3,4,8,9,10,11-hexahydro-1H- |
| | pyrido[4′,3′:3,4]pyrazolo[1,5-a]azepine-2(7H)-carboxamide; |
| 92 | (3R,8R*)-8-(Cyanomethyl)-N-(2-(difluoromethyl)-3-fluoropyridin-4-yl)- |
| | 11,11-difluoro-8-hydroxy-3-methyl-3,4,8,9,10,11-hexahydro-1H- |
| | pyrido[4′,3′:3,4]pyrazolo[1,5-a]azepine-2(7H)-carboxamide; |
| 93 | (3R,8R*)-N-(3-Cyano-4-fluorophenyl)-8-((2,2-difluoroethoxy)methyl)- |
| | 11,11-difluoro-8-hydroxy-3-methyl-3,4,8,9,10,11-hexahydro-1H- |
| | pyrido[4′,3′:3,4]pyrazolo[1,5-a]azepine-2(7H)-carboxamide; |
| 94 | (3R,8S*)-N-(3-Cyano-4-fluorophenyl)-8-((2,2-difluoroethoxy)methyl)- |
| | 11,11-difluoro-8-hydroxy-3-methyl-3,4,8,9,10,11-hexahydro-1H- |
| | pyrido[4′,3′:3,4]pyrazolo[1,5-a]azepine-2(7H)-carboxamide; |
| 95 | (3R,8R*)-N-(3-Cyano-4-fluorophenyl)-8-ethynyl-11,11-difluoro-8- |
| | hydroxy-3-methyl-3,4,8,9,10,11-hexahydro-1H- |
| | pyrido[4′,3′:3,4]pyrazolo[1,5-a]azepine-2(7H)-carboxamide; |
| 96 | (3R,8R*)-N-(2-Bromo-3-fluoropyridin-4-yl)-8-ethynyl-11,11-difluoro-8- |
| | hydroxy-3-methyl-3,4,8,9,10,11-hexahydro-1H- |
| | pyrido[4′,3′:3,4]pyrazolo[1,5-a]azepine-2(7H)-carboxamide; |
| 97 | (3R,8R*)-N-(2-(Difluoromethyl)-3-fluoropyridin-4-yl)-8-ethynyl-11,11- |
| | difluoro-8-hydroxy-3-methyl-3,4,8,9,10,11-hexahydro-1H- |
| | pyrido[4′,3′:3,4]pyrazolo[1,5-a]azepine-2(7H)-carboxamide; |
| 98 | (3R,8S*)-N-(3-Cyano-4-fluorophenyl)-8-ethynyl-11,11-difluoro-8- |
| | hydroxy-3-methyl-3,4,8,9,10,11-hexahydro-1H- |
| | pyrido[4′,3′:3,4]pyrazolo[1,5-a]azepine-2(7H)-carboxamide; |
| 99 | (3R,8S*)-N-(2-Bromo-3-fluoropyridin-4-yl)-8-ethynyl-11,11-difluoro-8- |
| | hydroxy-3-methyl-3,4,8,9,10,11-hexahydro-1H- |
| | pyrido[4′,3′:3,4]pyrazolo[1,5-a]azepine-2(7H)-carboxamide; |
| 100 | (3R,8S*)-N-(2-Difluoromethyl)-3-fluoropyridin-4-yl)-8-ethynyl-11,11- |
| | difluoro-8-hydroxy-3-methyl-3,4,8,9,10,11-hexahydro-1H- |
| | pyrido[4′,3′:3,4]pyrazolo[1,5-a]azepine-2(7H)-carboxamide; |
| 101 | (3R,8S*)-N-(3-Cyano-4-fluorophenyl)-11,11-difluoro-8-(2-hydroxypropan- |
| | 2-yl)-3-methyl-3,4,8,9,10,11-hexahydro-1H-pyrido[4′,3′:3,4]pyrazolo[1,5- |
| | a]azepine-2(7H)-carboxamide; |
| 102 | (3R,8S*)-N-(2-Bromo-3-fluoropyridin-4-yl)-11,11-difluoro-8-(2- |
| | hydroxypropan-2-yl)-3-methyl-3,4,8,9,10,11-hexahydro-1H- |
| | pyrido[4′,3′:3,4]pyrazolo[1,5-a]azepine-2(7H)-carboxamide; |
| 103 | (3R,8R*)-N-(3-Cyano-4-fluorophenyl)-11,11-difluoro-8-(2- |
| | hydroxypropan-2-yl)-3-methyl-3,4,8,9,10,11-hexahydro-1H- |
| | pyrido[4′,3′:3,4]pyrazolo[1,5-a]azepine-2(7H)-carboxamide; |
| 104 | (3R,8R*)-N-(2-Bromo-3-fluoropyridin-4-yl)-11,11-difluoro-8-(2- |
| | hydroxypropan-2-yl)-3-methyl-3,4,8,9,10,11-hexahydro-1H- |
| | pyrido[4′,3′:3,4]pyrazolo[1,5-a]azepine-2(7H)-carboxamide; |
| 105 | methyl (((3R,8R*)-2-((3-cyano-4-fluorophenyl)carbamoyl)-11,11-difluoro- |
| | 3-methyl-2,3,4,7,8,9,10,11-octahydro-1H-pyrido[4′,3′:3,4]pyrazolo[1,5- |
| | a]azepin-8-yl)methyl)carbamate; |
| 106 | Methyl (((3R,8R*)-2-((2-bromo-3-fluoropyridin-4-yl)carbamoyl)-11,11- |
| | difluoro-3-methyl-2,3,4,7,8,9,10,11-octahydro-1H- |
| | pyrido[4′,3′:3,4]pyrazolo[1,5-a]azepin-8-yl)methyl)carbamate; |
| 107 | Methyl (((3R,8R*)-2-((2-(difluoromethyl)-3-fluoropyridin-4-yl) |
| | carbamoyl)-11,11-difluoro-3-methyl-2,3,4,7,8,9,10,11-octahydro-1H- |
| | pyrido[4′,3′:3,4]pyrazolo[1,5-a]azepin-8-yl)methyl)carbamate; |
| 108 | Methyl (((3R,8S*)-2-((3-cyano-4-fluorophenyl)carbamoyl)-11,11-difluoro- |
| | 3-methyl-2,3,4,7,8,9,10,11-octahydro-1H-pyrido[4′,3′:3,4]pyrazolo[1,5- |
| | a]azepin-8-yl)methyl)carbamate; |
| 109 | Methyl (((3R,8S*)-2-((2-bromo-3-fluoropyridin-4-yl)carbamoyl)-11,11- |
| | difluoro-3-methyl-2,3,4,7,8,9,10,11-octahydro-1H- |
| | pyrido[4′,3′:3,4]pyrazolo[1,5-a]azepin-8-yl)methyl)carbamate; |
| 110 | Methyl (((3R,8S*)-2-((2-(difluoromethyl)-3-fluoropyridin-4-yl)carbamoyl)- |
| | 11,11-difluoro-3-methyl-2,3,4,7,8,9,10,11-octahydro-1H- |
| | pyrido[4′,3′:3,4]pyrazolo[1,5-a]azepin-8-yl)methyl)carbamate; |
| 111 | (3R,8S*)-N-(3-Cyano-4-fluorophenyl)-8,11,11-trifluoro-8- |
| | (hydroxymethyl)-3-methyl-3,4,8,9,10,11-hexahydro-1H- |
| | pyrido[4′,3′:3,4]pyrazolo[1,5-a]azepine-2(7H)-carboxamide; |
| 112 | (3R,8S*)-N-(2-Bromo-3-fluoropyridin-4-yl)-8,11,11-trifluoro-8- |
| | (hydroxymethyl)-3-methyl-3,4,8,9,10,11-hexahydro-1H- |
| | pyrido[4′,3′:3,4]pyrazolo[1,5-a]azepine-2(7H)-carboxamide; |
| 113 | (3R,8S*)-N-(2-(Difluoromethyl)-3-fluoropyridin-4-yl)-8,11,11-trifluoro-8- |
| | (hydroxymethyl)-3-methyl-3,4,8,9,10,11-hexahydro-1H- |
| | pyrido[4′,3′:3,4]pyrazolo[1,5-a]azepine-2(7H)-carboxamide; |
| 114 | (3R,8R*)-N-(3-Cyano-4-fluorophenyl)-8,11,11-trifluoro-8-(hydroxyl |
| | methyl)-3-methyl-3,4,8,9,10,11-hexahydro-1H-pyrido[4′,3′:3,4]pyrazolo[1,5- |
| | a]azepine-2(7H)-carboxamide; |
| 115 | (3R,8R*)-N-(2-Bromo-3-fluoropyridin-4-yl)-8,11,11-trifluoro-8- |
| | (hydroxylmethyl)-3-methyl-3,4,8,9,10,11-hexahydro-1H- |
| | pyrido[4′,3′:3,4]pyrazolo[1,5-a]azepine-2(7H)-carboxamide; |
| 116 | (3R,8R*)-N-(2-(Difluoromethyl)-3-fluoropyridin-4-yl)-8,11,11-trifluoro-8- |
| | (hydroxymethyl)-3-methyl-3,4,8,9,10,11-hexahydro-1H- |
| | pyrido[4′,3′:3,4]pyrazolo[1,5-a]azepine-2(7H)-carboxamide; |
| 117 | (3R,9S*)-N-(2-Bromo-3-fluoropyridin-4-yl)-11,11-difluoro-9-hydroxyl-3- |
| | methyl-3,4,8,9,10,11-hexahydro-1H-pyrido[4′,3′:3,4]pyrazolo[1,5-a]azepine- |
| | 2(7H)-carboxamide; |
| 118 | (3R,9R*)-N-(2-Bromo-3-fluoropyridin-4-yl)-11,11-difluoro-9-hydroxyl-3- |
| | methyl-3,4,8,9,10,11-hexahydro-1H-pyrido[4′,3′:3,4]pyrazolo[1,5-a]azepine- |
| | 2(7H)-carboxamide; |
| 119 | (3R,9S*)-N-(3-Cyano-4-fluorophenyl)-11,11-difluoro-9-hydroxyl-3-methyl- |
| | 3,4,8,9,10,11-hexahydro-1H-pyrido[4′,3′:3,4]pyrazolo[1,5-a]azepine-2(7H)- |
| | carboxamide; |
| 120 | (3R,9R*)-N-(3-Cyano-4-fluorophenyl)-11,11-difluoro-9-hydroxy-3-methyl- |
| | 3,4,8,9,10,11-hexahydro-1H-pyrido[4′,3′:3,4]pyrazolo[1,5-a]azepine-2(7H)- |
| | carboxamide; |
| 121 | (3R,9S*)-N-(2-(Difluoromethyl)-3-fluoropyridin-4-yl)-11,11-difluoro-9- |
| | hydroxy-3-methyl-3,4,8,9,10,11-hexahydro-1H- |
| | pyrido[4′,3′:3,4]pyrazolo[1,5-a]azepine-2(7H)-carboxamide; |
| 122 | (3R,9R*)-N-(2-(Difluoromethyl)-3-fluoropyridin-4-yl)-11,11-difluoro-9- |
| | hydroxy-3-methyl-3,4,8,9,10,11-hexahydro-1H- |
| | pyrido[4′,3′:3,4]pyrazolo[1,5-a]azepine-2(7H)-carboxamide; |
| 123 | (3R,9R*)-N-(2-Bromo-3-fluoropyridin-4-yl)-11-fluoro-9-hydroxy-3- |
| | methyl-3,4,8,9-tetrahydro-1H-pyrido[4′,3′:3,4]pyrazolo[1,5-a]azepine-2(7H)- |
| | carboxamide; |
| 124 | (3R,9S*)-N-(2-Bromo-3-fluoropyridin-4-yl)-11-fluoro-9-hydroxy-3-methyl- |
| | 3,4,8,9-tetrahydro-1H-pyrido[4′,3′:3,4]pyrazolo[1,5-a]azepine-2(7H)- |
| | carboxamide; |
| 125 | (3R,9R*)-N-(3-Cyano-4-fluorophenyl)-11,11-difluoro-9-hydroxy-9- |
| | (hydroxymethyl)-3-methyl-3,4,8,9,10,11-hexahydro-1H- |
| | pyrido[4′,3′:3,4]pyrazolo[1,5-a]azepine-2(7H)-carboxamide; |
| 126 | (3R,9S*)-N-(3-Cyano-4-fluorophenyl)-11,11-difluoro-9-hydroxy-9- |
| | (hydroxymethyl)-3-methyl-3,4,8,9,10,11-hexahydro-1H- |
| | pyrido[4′,3′:3,4]pyrazolo[1,5-a]azepine-2(7H)-carboxamide; |
| 127 | (3R,9R*)-N-(2-Bromo-3-fluoropyridin-4-yl)-11,11-difluoro-9- |
| | (fluoromethyl)-9-hydroxy-3-methyl-3,4,8,9,10,11-hexahydro-1H- |
| | pyrido[4′,3′:3,4]pyrazolo[1,5-a]azepine-2(7H)-carboxamide; |
| 128 | (3R,9R*)-N-(3-Cyano-4-fluorophenyl)-11,11-difluoro-9-(fluoromethyl)-9- |
| | hydroxy-3-methyl-3,4,8,9,10,11-hexahydro-1H- |
| | pyrido[4′,3′:3,4]pyrazolo[1,5-a]azepine-2(7H)-carboxamide; |
| 129 | (3R,9S*)-N-(2-Bromo-3-fluoropyridin-4-yl)-11,11-difluoro-9- |
| | (fluoromethyl)-9-hydroxy-3-methyl-3,4,8,9,10,11-hexahydro-1H- |
| | pyrido[4′,3′:3,4]pyrazolo[1,5-a]azepine-2(7H)-carboxamide; |
| 130 | (3R,9S*)-N-(3-Cyano-4-fluorophenyl)-11,11-difluoro-9-(fluoromethyl)-9- |
| | hydroxy-3-methyl-3,4,8,9,10,11-hexahydro-1H- |
| | pyrido[4′,3′:3,4]pyrazolo[1,5-a]azepine-2(7H)-carboxamide; |
| 131 | (3R,9R)-N-(3-Cyano-4-fluorophenyl)-11,11-difluoro-9-(fluoromethyl)-3- |
| | methyl-3,4,8,9,10,11-hexahydro-1H-pyrido[4′,3′:3,4]pyrazolo[1,5-a]azepine- |
| | 2(7H)-carboxamide; |
| 132 | (3R,9S*)-N-(3-Cyano-4-fluorophenyl)-11,11-difluoro-9-(fluoromethyl)-3- |
| | methyl-3,4,8,9,10,11-hexahydro-1H-pyrido[4′,3′:3,4]pyrazolo[1,5-a]azepine- |
| | 2(7H)-carboxamide; |
| 133 | (S*)-N-(2-Bromo-3-fluoropyridin-4-yl)-11,11-difluoro-9-(hydroxymethyl)- |
| | 3,4,8,9,10,11-hexahydro-1H-pyrido[4′,3′:3,4]pyrazolo[1,5-a]azepine-2(7H)- |
| | carboxamide; |
| 134 | (S*)-N-(3-Cyano-4-fluorophenyl)-11,11-difluoro-9-(hydroxymethyl)- |
| | 3,4,8,9,10,11-hexahydro-1H-pyrido[4′,3′:3,4]pyrazolo[1,5-a]azepine-2(7H)- |
| | carboxamide; |
| 135 | (R*)-N-(2-Bromo-3-fluoropyridin-4-yl)-11,11-difluoro-9-(hydroxymethyl)- |
| | 3,4,8,9,10,11-hexahydro-1H-pyrido[4′,3′:3,4]pyrazolo[1,5-a]azepine-2(7H)- |
| | carboxamide; |
| 136 | (R*)-N-(3-Cyano-4-fluorophenyl)-11,11-difluoro-9-(hydroxymethyl)- |
| | 3,4,8,9,10,11-hexahydro-1H-pyrido[4′,3′:3,4]pyrazolo[1,5-a]azepine-2(7H)- |
| | carboxamide; |
| 137 | (R*)-N-(2-Bromo-3-fluoropyridin-4-yl)-11,11-difluoro-9-hydroxy- |
| | 3,4,8,9,10,11-hexahydro-1H-pyrido[4′,3′:3,4]pyrazolo[1,5-a]azepine-2(7H)- |
| | carboxamide; |
| 138 | (R*)-N-(3-Cyano-4-fluorophenyl)-11,11-difluoro-9-hydroxy-3,4,8,9,10,11- |
| | hexahydro-1H-pyrido[4′,3′:3,4]pyrazolo[1,5-a]azepine-2(7H)-carboxamide; |
| 139 | (R*)-N-(2-(Difluoromethyl)-3-fluoropyridin-4-yl)-11,11-difluoro-9- |
| | hydroxy-3,4,8,9,10,11-hexahydro-1H-pyrido[4′,3′:3,4]pyrazolo[1,5- |
| | a]azepine-2(7H)-carboxamide; |
| 140 | (S*)-N-(2-Bromo-3-fluoropyridin-4-yl)-11,11-difluoro-9-hydroxy- |
| | 3,4,8,9,10,11-hexahydro-1H-pyrido[4′,3′:3,4]pyrazolo[1,5-a]azepine-2(7H)- |
| | carboxamide; |
| 141 | (S*)-N-(3-Cyano-4-fluorophenyl)-11,11-difluoro-9-hydroxy-3,4,8,9,10,11- |
| | hexahydro-1H-pyrido[4′,3′:3,4]pyrazolo[1,5-a]azepine-2(7H)-carboxamide; |
| 142 | (S*)-N-(2-(Difluoromethyl)-3-fluoropyridin-4-yl)-11,11-difluoro-9- |
| | hydroxy-3,4,8,9,10,11-hexahydro-1H-pyrido[4′,3′:3,4]pyrazolo[1,5- |
| | a]azepine-2(7H)-carboxamide; |
| 143 | (3R)-N-(3-Cyano-4-fluorophenyl)-11,11-difluoro-10-hydroxy-3-methyl- |
| | 1,3,4,7,8,9,10,11-octahydro-2H-pyrido[4′,3′:3,4]pyrazolo[1,5-a]azepine-2- |
| | carboxamide; |

and pharmaceutically acceptable salts, N-oxides, or solvates thereof.

### Pharmaceutical Compositions

Also disclosed herein are pharmaceutical compositions comprising

(A) at least one compound of Formula (I):

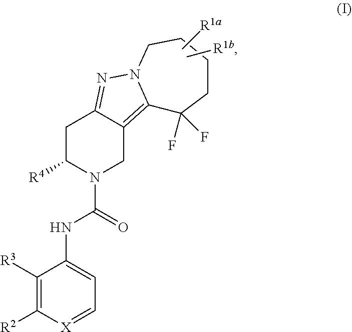

wherein
R^{1a} is H, or OH;
R^{1b} is selected from the group consisting of: F, OH, C₁₋₄alkyl, C₂₋₄alkenyl, C₂₋₄alkynyl, C₁₋₄haloalkyl, CH₂OH, C(CH₃)₂OH, CH₂CN, CH₂NH(C═O)CH₃, CH₂NH(C═O)OCH₃, CH₂OC₁₋₄haloalkyl, CH₂NH(C═O)CF₃, OC₁₋₄haloalkyl, or R^{1a} and R^{1b} come together to form ═CH₂;
R² is selected from the group consisting of: Br, CN, and C₁₋₄haloalkyl;
R³ is H, or F;
R⁴ is H or C₁₋₄alkyl; and
X is selected from the group consisting of: CH, CF, and N;
   and pharmaceutically acceptable salts, stereoisomers, isotopic variants, N-oxides or solvates of compounds of Formula (I); and

(B) at least one pharmaceutically acceptable excipient.

An embodiment of the present disclosure is a pharmaceutical composition comprising at least one pharmaceutically acceptable excipient and at least one compound listed in Table 1, as well as any pharmaceutically acceptable salt, N-oxide or solvate of such compound, or any pharmaceutically acceptable prodrugs of such compound, or any pharmaceutically active metabolite of such compound.

In embodiments, the pharmaceutical composition comprises at least one additional active or therapeutic agent. Additional active therapeutic agents may include, for example, an anti-HBV agent such as an HBV polymerase inhibitor, interferon, viral entry inhibitor, viral maturation inhibitor, capsid assembly modulator, reverse transcriptase inhibitor, immunomodulatory agent such as a TLR-agonist, or any other agents that affects the HBV life cycle and/or the consequences of HBV infection. The active agents of the present disclosure are used, alone or in combination with one or more additional active agents, to formulate pharmaceutical compositions of the present disclosure.

As used herein, the term “composition” or “pharmaceutical composition” refers to a mixture of at least one compound useful within the present disclosure with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the present disclosure within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the present disclosure, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of the compound useful within the present disclosure, and are physiologically acceptable to the patient. Supplementary active compounds may also be incorporated into the compositions. The “pharmaceutically acceptable carrier” may further include a pharmaceutically acceptable salt of the compound useful within the present disclosure. Other additional ingredients that may be included in the pharmaceutical compositions used in the practice of the present disclosure are known in the art and described, for example in Remington's Pharmaceutical Sciences (Genaro, Ed., Mack Publishing Co., 1985, Easton, Pa.), which is incorporated herein by reference.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients include calcium carbonate, calcium phosphate, various sugars and types of starch, cellulose derivatives, gelatin, vegetable oils, and polyethylene glycols.

Delivery forms of the pharmaceutical compositions containing one or more dosage units of the active agents may be prepared using suitable pharmaceutical excipients and compounding techniques known or that become available to those skilled in the art. The compositions may be administered in the inventive methods by a suitable route of delivery, e.g., oral, parenteral, rectal, topical, or ocular routes, or by inhalation.

The preparation may be in the form of tablets, capsules, sachets, dragees, powders, granules, lozenges, powders for reconstitution, liquid preparations, or suppositories. Preferably, the compositions are formulated for intravenous infusion, topical administration, or oral administration.

For oral administration, the compounds of the present disclosure can be provided in the form of tablets or capsules, or as a solution, emulsion, or suspension. To prepare the oral compositions, the compounds may be formulated to yield a dosage of, e.g., from about 0.05 to about 100 mg/kg daily, or from about 0.05 to about 35 mg/kg daily, or from about 0.1 to about 10 mg/kg daily. For example, a total daily dosage of about 5 mg to 5 g daily may be accomplished by dosing once, twice, three, or four times per day.

Oral tablets may include a compound according to the present disclosure mixed with pharmaceutically acceptable excipients such as inert diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are suitable disintegrating agents. Binding agents may include starch and gelatin. The lubricating agent, if present, may be magnesium stearate, stearic acid or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, compounds of the present disclosure may be mixed with a solid, semi-solid, or liquid diluent. Soft gelatin capsules may be prepared by mixing the compound of the present disclosure with water, an oil such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions or syrups or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The active agents of this present disclosure may also be administered by non-oral routes. For example, the compositions may be formulated for rectal administration as a suppository. For parenteral use, including intravenous, intramuscular, intraperitoneal, or subcutaneous routes, the compounds of the present disclosure may be provided in sterile aqueous solutions or suspensions, buffered to an appropriate pH and isotonicity or in parenterally acceptable oil. Suitable aqueous vehicles include Ringer's solution and isotonic sodium chloride. Such forms will be presented in unit-dose form such as ampules or disposable injection devices, in multi-dose forms such as vials from which the appropriate dose may be withdrawn, or in a solid form or pre-concentrate that can be used to prepare an injectable formulation. Illustrative infusion doses may range from about 1 to 1000 μg/kg/minute of compound, admixed with a pharmaceutical carrier over a period ranging from several minutes to several days.

For topical administration, the compounds may be mixed with a pharmaceutical carrier at a concentration of about 0.1% to about 10% of drug to vehicle. Another mode of administering the compounds of the present disclosure may utilize a patch formulation to affect transdermal delivery.

Compounds of the present disclosure may alternatively be administered in methods of this present disclosure by inhalation, via the nasal or oral routes, e.g., in a spray formulation also containing a suitable carrier.

### Methods of Use

The disclosed compounds are useful in the treatment and prevention of HBV infection in a subject such as a human subject.

In a non-limiting aspect, these compounds may (i) modulate or disrupt HBV assembly and other HBV core protein functions necessary for HBV replication or the generation of infectious particles, (ii) inhibit the production of infectious virus particles or infection, or (iii) interact with HBV capsid to effect defective viral particles with reduced infectivity or replication capacity acting as capsid assembly modulators. In particular, and without being bound to any particular mechanism of action, it is believed that the disclosed compounds are useful in HBV treatment by disrupting, accelerating, reducing, delaying and/or inhibiting normal viral capsid assembly and/or disassembly of immature or mature particles, thereby inducing aberrant capsid morphology leading to antiviral effects such as disruption of virion assembly and/or disassembly, virion maturation, virus egress and/or infection of target cells. The disclosed compounds may act as a disruptor of capsid assembly interacting with mature or immature viral capsid to perturb the stability of the capsid, thus affecting its assembly and/or disassembly. The disclosed compounds may perturb protein folding and/or salt bridges required for stability, function and/or normal morphology of the viral capsid, thereby disrupting and/or accelerating capsid assembly and/or disassembly. The disclosed compounds may bind capsid and alter metabolism of cellular polyproteins and precursors, leading to abnormal accumulation of protein monomers and/or oligomers and/or abnormal particles, which causes cellular toxicity and death of infected cells. The disclosed compounds may cause failure of the formation of capsids of optimal stability, affecting efficient uncoating and/or disassembly of viruses (e.g., during infectivity). The disclosed compounds may disrupt and/or accelerate capsid assembly and/or disassembly when the capsid protein is immature. The disclosed compounds may disrupt and/or accelerate capsid assembly and/or disassembly when the capsid protein is mature. The disclosed compounds may disrupt and/or accelerate capsid assembly and/or disassembly during viral infectivity which may further attenuate HBV viral infectivity and/or reduce viral load. The disruption, acceleration, inhibition, delay and/or reduction of capsid assembly and/or disassembly by the disclosed compounds may eradicate the virus from the host organism. Eradication of HBV from a subject by the disclosed compounds advantageously obviates the need for chronic long-term therapy and/or reduces the duration of long-term therapy.

An additional embodiment of the present disclosure is a method of treating a subject suffering from an HBV infection, comprising administering to a subject in need of such treatment an effective amount of at least one compound of Formula (I).

In another aspect, provided herein is a method of reducing the viral load associated with an HBV infection in an individual in need thereof, comprising administering to the individual a therapeutically effective amount of a compound of Formula (I), or a pharmaceutically acceptable salt thereof.

In another aspect, provided herein is a method of reducing reoccurrence of an HBV infection in an individual in need thereof, comprising administering to the individual a therapeutically effective amount of a compound of Formula (I), or a pharmaceutically acceptable salt thereof.

In another aspect, provided herein is a method of inhibiting or reducing the formation or presence of HBV DNA-containing particles or HBV RNA-containing particles in an individual in need thereof, comprising administering to the individual a therapeutically effective amount of a compound of Formula (I), or a pharmaceutically acceptable salt thereof.

In another aspect, provided herein is a method of reducing an adverse physiological impact of an HBV infection in an individual in need thereof, comprising administering to the individual a therapeutically effective amount of a compound of Formula (I), or a pharmaceutically acceptable salt thereof.

In another aspect, provided herein is a method of inducing remission of hepatic injury from an HBV infection in an individual in need thereof, comprising administering to the individual a therapeutically effective amount of a compound of Formula (I), or a pharmaceutically acceptable salt thereof.

In another aspect, provided herein is a method of reducing the physiological impact of long-term antiviral therapy for HBV infection in an individual in need thereof, comprising administering to the individual a therapeutically effective amount of a compound of Formula (I), or a pharmaceutically acceptable salt thereof.

In another aspect, provided herein is a method of prophylactically treating an HBV infection in an individual in need thereof, wherein the individual is afflicted with a latent HBV infection, comprising administering to the individual a therapeutically effective amount of a compound of Formula (I), or a pharmaceutically acceptable salt thereof.

In embodiments, the disclosed compounds are suitable for monotherapy. In embodiments, the disclosed compounds are effective against natural or native HBV strains. In embodiments, the disclosed compounds are effective against HBV strains resistant to currently known drugs.

In another embodiment, the compounds provided herein can be used in methods of modulating (e.g., inhibiting or disrupting) the activity, stability, function, and viral replication properties of HBV cccDNA.

In yet another embodiment, the compounds of the present disclosure can be used in methods of diminishing or preventing the formation of HBV cccDNA.

In another embodiment, the compounds provided herein can be used in methods of modulating (e.g., inhibiting or disrupting) the activity of HBV cccDNA.

In yet another embodiment, the compounds of the present disclosure can be used in methods of diminishing the formation of HBV cccDNA.

In another embodiment, the disclosed compounds can be used in methods of modulating, inhibiting, or disrupting the generation or release of HBV RNA particles from within the infected cell.

In a further embodiment, the total burden (or concentration) of HBV RNA particles is modulated. In a preferred embodiment, the total burden of HBV RNA is diminished.

In another embodiment, the methods provided herein reduce the viral load in the individual to a greater extent or at a faster rate compared to the administering of a compound selected from the group consisting of an HBV polymerase inhibitor, interferon, viral entry inhibitor, viral maturation inhibitor, distinct capsid assembly modulator, antiviral compounds of distinct or unknown mechanism, and any combination thereof.

In another embodiment, the methods provided herein cause a lower incidence of viral mutation and/or viral resistance than the administering of a compound selected from the group consisting of an HBV polymerase inhibitor, interferon, viral entry inhibitor, viral maturation inhibitor, distinct capsid assembly modulator, antiviral compounds of distinct or unknown mechanism, and combination thereof.

In another embodiment, the methods provided herein further comprise administering to the individual at least one HBV vaccine, a nucleoside HBV inhibitor, an interferon or any combination thereof.

In an aspect, provided herein is a method of treating an HBV infection in an individual in need thereof, comprising reducing the HBV viral load by administering to the individual a therapeutically effective amount of a compound of Formula (I) (as well as Formula (IA) or Formula (IB)), or a pharmaceutically acceptable salt thereof, alone or in combination with a reverse transcriptase inhibitor; and further administering to the individual a therapeutically effective amount of HBV vaccine.

An additional embodiment of the present disclosure is a method of treating a subject suffering from an HBV infection, comprising administering to a subject in need of such treatment an effective amount of at least one compound of Formula (I).

In another aspect, provided herein is a method of reducing the viral load associated with an HBV infection in an individual in need thereof, comprising administering to the individual a therapeutically effective amount of a compound of Formula (I), or a pharmaceutically acceptable salt thereof.

In another aspect, provided herein is a method of reducing reoccurrence of an HBV infection in an individual in need thereof, comprising administering to the individual a therapeutically effective amount of a compound of Formula (I), or a pharmaceutically acceptable salt thereof.

In another aspect, provided herein is a method of inhibiting or reducing the formation or presence of HBV DNA-containing particles or HBV RNA-containing particles in an individual in need thereof, comprising administering to the individual a therapeutically effective amount of a compound of Formula (I), or a pharmaceutically acceptable salt thereof.

In another aspect, provided herein is a method of reducing an adverse physiological impact of an HBV infection in an individual in need thereof, comprising administering to the individual a therapeutically effective amount of a compound of Formula (I), or a pharmaceutically acceptable salt thereof.

In another aspect, provided herein is a method of inducing remission of hepatic injury from an HBV infection in an individual in need thereof, comprising administering to the individual a therapeutically effective amount of a compound of Formula (I), or a pharmaceutically acceptable salt thereof.

In another aspect, provided herein is a method of reducing the physiological impact of long-term antiviral therapy for HBV infection in an individual in need thereof, comprising administering to the individual a therapeutically effective amount of a compound of Formula (I), or a pharmaceutically acceptable salt thereof.

In another aspect, provided herein is a method of prophylactically treating an HBV infection in an individual in need thereof, wherein the individual is afflicted with a latent HBV infection, comprising administering to the individual a therapeutically effective amount of a compound of Formula (I), or a pharmaceutically acceptable salt thereof.

In an embodiment, the methods provided herein further comprise monitoring the HBV viral load of the subject, wherein the method is carried out for a period of time such that the HBV virus is undetectable.

### Combinations

Provided herein are combinations of one or more of the disclosed compounds with at least one additional therapeutic agent. In embodiments, the methods provided herein can further comprise administering to the individual at least one additional therapeutic agent. In embodiments, the disclosed compounds are suitable for use in combination therapy. The compounds of the present disclosure may be useful in combination with one or more additional compounds useful for treating HBV infection. These additional compounds may comprise compounds of the present disclosure or compounds known to treat, prevent, or reduce the symptoms or effects of HBV infection.

In an exemplary embodiment, additional active ingredients are those that are known or discovered to be effective in the treatment of conditions or disorders involved in HBV infection, such as another HBV capsid assembly modulator or a compound active against another target associated with the particular condition or disorder involved in HBV infection, or the HBV infection itself. The combination may serve to increase efficacy (e.g., by including in the combination a compound potentiating the potency or effectiveness of an active agent according to the present disclosure), decrease one or more side effects, or decrease the required dose of the active agent according to the present disclosure. In a further embodiment, the methods provided herein allow for administering of the at least one additional therapeutic agent at a lower dose or frequency as compared to the administering of the at least one additional therapeutic agent alone that is required to achieve similar results in prophylactically treating an HBV infection in an individual in need thereof.

Such compounds include but are not limited to HBV combination drugs, HBV vaccines, HBV DNA polymerase inhibitors, immunomodulatory agents, toll-like receptor (TLR) modulators, interferon alpha receptor ligands, hyaluronidase inhibitors, hepatitis b surface antigen (HBsAg) inhibitors, cytotoxic T-lymphocyte-associated protein 4 (ipi4) inhibitors, cyclophilin inhibitors, HBV viral entry inhibitors, antisense oligonucleotide targeting viral mRNA, short interfering RNAs (siRNA) and ddRNAi endonuclease modulators, ribonucleotide reductase inhibitors, HBV E antigen inhibitors, covalently closed circular DNA (cccDNA) inhibitors, famesoid X receptor agonists, HBV antibodies, CCR2 chemokine antagonists, thymosin agonists, cytokines, nucleoprotein modulators, retinoic acid-inducible gene 1 simulators, NOD2 stimulators, phosphatidylinositol 3-kinase (PI3K) inhibitors, indoleamine-2, 3-dioxygenase (IDO) pathway inhibitors, PD-1 inhibitors, PD-Ll inhibitors, recombinant thymosin alpha-1, bruton's tyrosine kinase (BTK) inhibitors, KDM inhibitors, HBV replication inhibitors, arginase inhibitors, and any other agent that affects the HBV life cycle and/or affect the consequences of HBV infection or combinations thereof.

In embodiments, the compounds of the present disclosure may be used in combination with an HBV polymerase inhibitor, immunomodulatory agents, interferon such as pegylated interferon, viral entry inhibitor, viral maturation inhibitor, capsid assembly modulator, reverse transcriptase inhibitor, a cyclophilin/TNF inhibitor, immunomodulatory agent such as a TLR-agonist, an HBV vaccine, and any other agent that affects the HBV life cycle and/or affect the consequences of HBV infection or combinations thereof.

In particular, the compounds of the present disclosure may be used in combination with one or more agents (or a salt thereof) selected from the group consisting of

HBV reverse transcriptase inhibitors, and DNA and RNA polymerase inhibitors, including but not limited to: lamivudine (3TC, Zeffix, Heptovir, Epivir, and Epivir-HBV), entecavir (Baraclude, Entavir), adefovir dipivoxil (Hepsara, Preveon, bis-POM PMEA), tenofovir disoproxil fumarate (Viread, TDF or PMPA);

interferons, including but not limited to interferon alpha (IFN-α), interferon beta (IFN-β), interferon lambda (IFN-λ) and interferon gamma (IFN-γ);

viral entry inhibitors;

viral maturation inhibitors;

literature-described capsid assembly modulators, such as, but not limited to BAY 41-4109;

reverse transcriptase inhibitor;

an immunomodulatory agent such as a TLR-agonist; and

agents of distinct or unknown mechanism, such as but not limited to AT-61 ((E)-N-(1-chloro-3-oxo-1-phenyl-3-(piperidin-1-yl)prop-1-en-2-yl)benzamide), AT-130 ((E)-N-(1-bromo-1-(2-methoxy phenyl)-3-oxo-3-(piperidin-1-yl)prop-1-en-2-yl)-4-nitrobenzamide), and similar analogs.

In embodiments, the additional therapeutic agent is an interferon. The term “interferon” or “IFN” refers to any member the family of highly homologous species-specific proteins that inhibit viral replication and cellular proliferation, and modulate immune response. Human interferons are grouped into three classes; Type I, which include interferon-alpha (IFN-α), interferon-beta (IFN-β), and interferon-omega (IFN-ω), Type II, which includes interferon-gamma (IFN-γ), and Type III, which includes interferon-lambda (IFN-4 Recombinant forms of interferons that have been developed and are commercially available are encompassed by the term “interferon” as used herein. Subtypes of interferons, such as chemically modified or mutated interferons, are also encompassed by the term “interferon” as used herein. Chemically modified interferons include pegylated interferons and glycosylated interferons. Examples of interferons also include, but are not limited to, interferon-alpha-2a, interferon-alpha-2b, interferon-alpha-n1, interferon-beta-1a, interferon-beta-1b, interferon-lamda-1, interferon-lamda-2, and interferon-lamda-3. Examples of pegylated interferons include pegylated interferon-alpha-2a and pegylated interferson alpha-2b.

Accordingly, in one embodiment, the compounds of Formula I (including IA and IB) can be administered in combination with an interferon selected from the group consisting of interferon alpha (IFN-α), interferon beta (IFN-β), interferon lambda (IFN-λ), and interferon gamma (IFN-γ). In one specific embodiment, the interferon is interferon-alpha-2a, interferon-alpha-2b, or interferon-alpha-n1. In another specific embodiment, the interferon-alpha-2a or interferon-alpha-2b is pegylated. In a preferred embodiment, the interferon-alpha-2a is pegylated interferon-alpha-2a (PEGASYS).

In another embodiment, the additional therapeutic agent is selected from immune modulator or immune stimulator therapies, which includes biological agents belonging to the interferon class.

Further, the additional therapeutic agent may be an agent that disrupts the function of other essential viral protein(s) or host proteins required for HBV replication or persistence.

In another embodiment, the additional therapeutic agent is an antiviral agent that blocks viral entry or maturation or targets the HBV polymerase such as nucleoside or nucleotide or non-nucleos(t)ide polymerase inhibitors. In a further embodiment of the combination therapy, the reverse transcriptase inhibitor and/or DNA and/or RNA polymerase inhibitor is Zidovudine, Didanosine, Zalcitabine, ddA, Stavudine, Lamivudine, Abacavir, Emtricitabine, Entecavir, Apricitabine, Atevirapine, ribavirin, acyclovir, famciclovir, valacyclovir, ganciclovir, valganciclovir, Tenofovir, Adefovir, PMPA, cidofovir, Efavirenz, Nevirapine, Delavirdine, or Etravirine.

In an embodiment, the additional therapeutic agent is an immunomodulatory agent that induces a natural, limited immune response leading to induction of immune responses against unrelated viruses. In other words, the immunomodulatory agent can effect maturation of antigen presenting cells, proliferation of T-cells and cytokine release (e.g., IL-12, IL-18, IFN-alpha, -beta, and gamma and TNF-alpha among others).

In a further embodiment, the additional therapeutic agent is a TLR modulator or a TLR agonist, such as a TLR-7 agonist or TLR-9 agonist. In further embodiment of the combination therapy, the TLR-7 agonist is selected from the group consisting of SM360320 (9-benzyl-8-hydroxy-2-(2-methoxy-ethoxy)adenine) and AZD 8848 (methyl [3-({[3-(6-amino-2-butoxy-8-oxo-7,8-dihydro-9H-purin-9-yl)propyl][3-(4-morpholinyl)propyl]amino}methyl)phenyl]acetate).

In any of the methods provided herein, the method may further comprise administering to the individual at least one HBV vaccine, a nucleoside HBV inhibitor, an interferon or any combination thereof. In an embodiment, the HBV vaccine is at least one of RECOMBIVAX HB, ENGERIX-B, ELOVAC B, GENEVAC-B, or SHANVAC B.

In another aspect, provided herein is method of treating an HBV infection in an individual in need thereof, comprising reducing the HBV viral load by administering to the individual a therapeutically effective amount of a compound of the present disclosure alone or in combination with a reverse transcriptase inhibitor; and further administering to the individual a therapeutically effective amount of HBV vaccine. The reverse transcriptase inhibitor may be one of Zidovudine, Didanosine, Zalcitabine, ddA, Stavudine, Lamivudine, Abacavir, Emtricitabine, Entecavir, Apricitabine, Atevirapine, ribavirin, acyclovir, famciclovir, valacyclovir, ganciclovir, valganciclovir, Tenofovir, Adefovir, PMPA, cidofovir, Efavirenz, Nevirapine, Delavirdine, or Etravirine.

For any combination therapy described herein, synergistic effect may be calculated, for example, using suitable methods such as the Sigmoid-Eₘₐₓ equation (Holford & Scheiner, 19981, Clin. Pharmacokinet. 6: 429-453), the equation of Loewe additivity (Loewe & Muischnek, 1926, Arch. Exp. Pathol Pharmacol. 114: 313-326) and the median-effect equation (Chou & Talalay, 1984, Adv. Enzyme Regul. 22: 27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Definitions

Listed below are definitions of various terms used to describe this present disclosure. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the applicable art. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e. to at least one) of the grammatical object of the article. By way of example, “an element” means one element or more than one element. Furthermore, use of the term “including” as well as other forms, such as “include,” “includes,” and “included,” is not limiting.

As used in the specification and in the claims, the term “comprising” can include the embodiments “consisting of” and “consisting essentially of” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated compounds, which allows the presence of only the named compounds, along with any pharmaceutically acceptable carriers, and excludes other compounds.

All ranges disclosed herein are inclusive of the recited endpoint and independently combinable (for example, the range of “from 50 mg to 300 mg” is inclusive of the endpoints, 50 mg and 300 mg, and all the intermediate values). The endpoints of the ranges and any values disclosed herein are not limited to the precise range or value; they are sufficiently imprecise to include values approximating these ranges and/or values.

As used herein, approximating language can be applied to modify any quantitative representation that can vary without resulting in a change in the basic function to which it is related. Accordingly, a value modified by a term or terms, such as “substantially,” cannot be limited to the precise value specified, in some cases. In at least some instances, the approximating language can correspond to the precision of an instrument for measuring the value.

The term “alkyl” refers to a straight- or branched-chain alkyl group having from 1 to 12 carbon atoms in the chain. Examples of alkyl groups include methyl (Me, which also may be structurally depicted by the symbol, “/”), ethyl (Et), n-propyl, isopropyl, butyl, isobutyl, sec-butyl, tert-butyl (tBu), pentyl, isopentyl, tert-pentyl, hexyl, isohexyl, and groups that in light of the ordinary skill in the art and the teachings provided herein would be considered equivalent to any one of the foregoing examples. The term C₁₋₄alkyl as used here refers to a straight- or branched-chain alkyl group having from 1 to 4 carbon atoms in the chain. The term C₁₋₆alkyl as used here refers to a straight- or branched-chain alkyl group having from 1 to 6 carbon atoms in the chain.

The term “alkenyl” refers to an alkyl group that contains in the straight or branched hydrocarbon chain one or more double bonds. An alkenyl group may be unsubstituted or substituted.

The term “alkynyl” refers to an alkyl group that contains in the straight or branched hydrocarbon chain one or more triple bonds. An alkynyl group may be unsubstituted or substituted.

The term “heteroaryl” refers to a monocyclic or fused bicyclic heterocycle (ring structure having ring atoms selected from carbon atoms and up to four heteroatoms selected from nitrogen, oxygen, and sulfur) having from 3 to 9 ring atoms per heterocycle. Illustrative examples of heteroaryl groups include the following entities, in the form of properly bonded moieties:

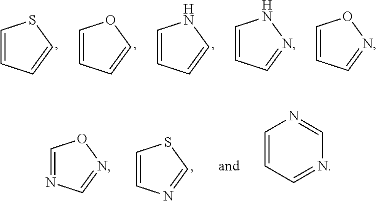

Those skilled in the art will recognize that the species of heteroaryl groups listed or illustrated above are not exhaustive, and that additional species within the scope of these defined terms may also be selected.

The term “cyano” refers to the group —CN.

The term “halo” represents chloro, fluoro, bromo or iodo.

The term “perhaloalkyl” or “haloalkyl” refers to a straight- or branched-chain alkyl group having from 1 to 6 carbon atoms in the chain optionally substituting hydrogens with halogens. The term “C₁₋₄haloalkyl” as used here refers to a straight- or branched-chain alkyl group having from 1 to 4 carbon atoms in the chain, optionally substituting hydrogens with halogens. The term “C₁₋₆ haloalkyl” as used here refers to a straight- or branched-chain alkyl group having from 1 to 6 carbon atoms in the chain, optionally substituting hydrogens with halogens. Examples of “perhaloalkyl”, “haloalkyl” groups include trifluoromethyl (CF₃), difluoromethyl (CF₂H), monofluoromethyl (CH₂F), pentafluoroethyl (CF₂CF₃), tetrafluoroethyl (CHFCF₃),monofluoroethyl (CH₂CH₂F), trifluoroethyl (CH₂CF₃), tetrafluorotrifluoromethylethyl (—CF(CF₃)₂), and groups that in light of the ordinary skill in the art and the teachings provided herein would be considered equivalent to any one of the foregoing examples.

The term “substituted” means that the specified group or moiety bears one or more substituents. The term “unsubstituted” means that the specified group bears no substituents. The term “optionally substituted” means that the specified group is unsubstituted or substituted by one or more substituents. Where the term “substituted” is used to describe a structural system, the substitution is meant to occur at any valency-allowed position on the system. In cases where a specified moiety or group is not expressly noted as being optionally substituted or substituted with any specified substituent, it is understood that such a moiety or group is intended to be unsubstituted.

The terms “para”, “meta”, and “ortho” have the meanings as understood in the art. Thus, for example, a fully substituted phenyl group has substituents at both “ortho”(o) positions adjacent to the point of attachment of the phenyl ring, both “meta” (m) positions, and the one “para” (p) position across from the point of attachment. To further clarify the position of substituents on the phenyl ring, the 2 different ortho positions will be designated as ortho and ortho′ and the 2 different meta positions as meta and meta′ as illustrated below.

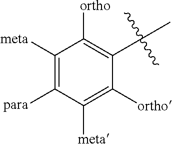

When referring to substituents on a pyridyl group, the terms “para”, “meta”, and “ortho” refer to the placement of a substituent relative to the point of attachment of the pyridyl ring. For example, the structure below is described as 3-pyridyl with the X¹ substituent in the ortho position, the X² substituent in the meta position, and X³ substituent in the para position:

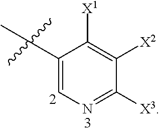

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with respect to the maximum amount of the same entity that could be obtained under the particular stoichiometric conditions. Concentrations that are given as percentages refer to mass ratios, unless indicated differently.

The terms “buffered” solution or “buffer” solution are used herein interchangeably according to their standard meaning. Buffered solutions are used to control the pH of a medium, and their choice, use, and function is known to those of ordinary skill in the art. See, for example, G. D. Considine, ed., Van Nostrand's Encyclopedia of Chemistry, p. 261, 5^{th} ed. (2005), describing, inter alia, buffer solutions and how the concentrations of the buffer constituents relate to the pH of the buffer. For example, a buffered solution is obtained by adding MgSO₄ and NaHCO₃ to a solution in a 10:1 w/w ratio to maintain the pH of the solution at about 7.5.

Any formula given herein is intended to represent compounds having structures depicted by the structural formula as well as certain variations or forms. In particular, compounds of any formula given herein may have asymmetric centers and therefore exist in different enantiomeric forms. All optical isomers of the compounds of the general formula, and mixtures thereof, are considered within the scope of the formula. Thus, any formula given herein is intended to represent a racemate, one or more enantiomeric forms, one or more diastereomeric forms, one or more atropisomeric forms, and mixtures thereof. Furthermore, certain structures may exist as geometric isomers (i.e., cis and trans isomers), as tautomers, or as atropisomers.

It is also to be understood that compounds that have the same molecular formula but differ in the nature or sequence of bonding of their atoms or the arrangement of their atoms in space are termed “isomers.”

Stereoisomers that are not mirror images of one another are termed “diastereomers” and those that are non-superimposable mirror images of each other are termed “enantiomers.” When a compound has an asymmetric center, for example, it is bonded to four different groups, and a pair of enantiomers is possible. An enantiomer can be characterized by the absolute configuration of its asymmetric center and is described by the R-and S-sequencing rules of Cahn and Prelog, or by the manner in which the molecule rotates the plane of polarized light and designated as dextrorotatory or levorotatory (i.e., as (+)- or (−)-isomers respectively). A chiral compound can exist as either an individual enantiomer or as a mixture thereof. A mixture containing equal proportions of the enantiomers is called a “racemic mixture.”

“Tautomers” refer to compounds that are interchangeable forms of a particular compound structure, and that vary in the displacement of hydrogen atoms and electrons. Thus, two structures may be in equilibrium through the movement of n electrons and an atom (usually H). For example, enols and ketones are tautomers because they are rapidly interconverted by treatment with either acid or base. Another example of tautomerism is the aci-and nitro-forms of phenyl nitromethane, that are likewise formed by treatment with acid or base.

Tautomeric forms may be relevant to the attainment of the optimal chemical reactivity and biological activity of a compound of interest.

The compounds of this present disclosure may possess one or more asymmetric centers; such compounds can therefore be produced as individual (R)- or (S)-stereoisomers or as mixtures thereof.

Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

Certain examples contain chemical structures that are depicted as an absolute enantiomer but are intended to indicate enatiopure material that is of unknown configuration. In these cases (R*) or (S*) is used in the name to indicate that the absolute stereochemistry of the corresponding stereocenter is unknown. Thus, a compound designated as (R*) refers to an enantiopure compound with an absolute configuration of either (R) or (S). In cases where the absolute stereochemistry has been confirmed, the structures are named using (R) and (S).

The symbols [symbol] and [symbol] are used as meaning the same spatial arrangement in chemical structures shown herein. Analogously, the symbols [symbol] and [symbol] are used as meaning the same spatial arrangement in chemical structures shown herein.

Additionally, any formula given herein is intended to refer also to hydrates, solvates, and polymorphs of such compounds, and mixtures thereof, even if such forms are not listed explicitly. Certain compounds of Formula (I), or pharmaceutically acceptable salts of compounds of Formula (I), may be obtained as solvates. Solvates include those formed from the interaction or complexation of compounds of the present disclosure with one or more solvents, either in solution or as a solid or crystalline form. In some embodiments, the solvent is water and the solvates are hydrates. In addition, certain crystalline forms of compounds of Formula (I), or pharmaceutically acceptable salts of compounds of Formula (I) may be obtained as co-crystals. In certain embodiments of the present disclosure, compounds of Formula (I) were obtained in a crystalline form. In other embodiments, crystalline forms of compounds of Formula (I) were cubic in nature. In other embodiments, pharmaceutically acceptable salts of compounds of Formula (I) were obtained in a crystalline form. In still other embodiments, compounds of Formula (I) were obtained in one of several polymorphic forms, as a mixture of crystalline forms, as a polymorphic form, or as an amorphous form. In other embodiments, compounds of Formula (I) convert in solution between one or more crystalline forms and/or polymorphic forms.

Reference to a compound herein stands for a reference to any one of: (a) the actually recited form of such compound, and (b) any of the forms of such compound in the medium in which the compound is being considered when named. For example, reference herein to a compound such as R—COOH, encompasses reference to any one of, for example, R—COOH₍ₛ₎, R—COOH₍ₛₒₗ₎, and R—COO⁻₍ₛₒₗ₎. In this example, R—COOH₍ₛ₎ refers to the solid compound, as it could be for example in a tablet or some other solid pharmaceutical composition or preparation; R—COOH₍ₛₒₗ₎ refers to the undissociated form of the compound in a solvent; and R—COO⁻₍ₛₒₗ₎ refers to the dissociated form of the compound in a solvent, such as the dissociated form of the compound in an aqueous environment, whether such dissociated form derives from R—COOH, from a salt thereof, or from any other entity that yields R—COO⁻ upon dissociation in the medium being considered. In another example, an expression such as “exposing an entity to compound of formula R—COOH” refers to the exposure of such entity to the form, or forms, of the compound R—COOH that exists, or exist, in the medium in which such exposure takes place. In still another example, an expression such as “reacting an entity with a compound of formula R—COOH” refers to the reacting of (a) such entity in the chemically relevant form, or forms, of such entity that exists, or exist, in the medium in which such reacting takes place, with (b) the chemically relevant form, or forms, of the compound R—COOH that exists, or exist, in the medium in which such reacting takes place. In this regard, if such entity is for example in an aqueous environment, it is understood that the compound R—COOH is in such same medium, and therefore the entity is being exposed to species such as R—COOH_{(aq)} and/or R—COO⁻_{(aq)}, where the subscript “(aq)” stands for “aqueous” according to its conventional meaning in chemistry and biochemistry. A carboxylic acid functional group has been chosen in these nomenclature examples; this choice is not intended, however, as a limitation but it is merely an illustration. It is understood that analogous examples can be provided in terms of other functional groups, including but not limited to hydroxyl, basic nitrogen members, such as those in amines, and any other group that interacts or transforms according to known manners in the medium that contains the compound. Such interactions and transformations include, but are not limited to, dissociation, association, tautomerism, solvolysis, including hydrolysis, solvation, including hydration, protonation, and deprotonation. No further examples in this regard are provided herein because these interactions and transformations in a given medium are known by any one of ordinary skill in the art.

In another example, a zwitterionic compound is encompassed herein by referring to a compound that is known to form a zwitterion, even if it is not explicitly named in its zwitterionic form. Terms such as zwitterion, zwitterions, and their synonyms zwitterionic compound(s) are standard IUPAC-endorsed names that are well known and part of standard sets of defined scientific names. In this regard, the name zwitterion is assigned the name identification CHEBI:27369 by the Chemical Entities of Biological Interest (ChEBI) dictionary of molecular entities. As generally well known, a zwitterion or zwitterionic compound is a neutral compound that has formal unit charges of opposite sign. Sometimes these compounds are referred to by the term “inner salts”. Other sources refer to these compounds as “dipolar ions”, although the latter term is regarded by still other sources as a misnomer. As a specific example, aminoethanoic acid (the amino acid glycine) has the formula H₂NCH₂COOH, and it exists in some media (in this case in neutral media) in the form of the zwitterion ⁺H₃NCH₂COO⁻. Zwitterions, zwitterionic compounds, inner salts and dipolar ions in the known and well established meanings of these terms are within the scope of this present disclosure, as would in any case be so appreciated by those of ordinary skill in the art. Because there is no need to name each and every embodiment that would be recognized by those of ordinary skill in the art, no structures of the zwitterionic compounds that are associated with the compounds of this present disclosure are given explicitly herein. They are, however, part of the embodiments of this present disclosure. No further examples in this regard are provided herein because the interactions and transformations in a given medium that lead to the various forms of a given compound are known by any one of ordinary skill in the art.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the present disclosure include isotopes of hydrogen, carbon, nitrogen, oxygen, phosphorus, sulfur, fluorine, chlorine, and iodine such as ²H, ³H, ¹¹C, ¹³C, ¹⁴C, ¹⁵N, ¹⁸O, ¹⁷O, ³¹P, ³²P, ³⁵S, ¹⁸F, ³⁶Cl, ¹²⁵I, respectively. Such isotopically labeled compounds are useful in metabolic studies (preferably with ¹⁴C), reaction kinetic studies (with, for example deuterium (i.e., D or ²H); or tritium (i.e., T or ³H)), detection or imaging techniques such as positron emission tomography (PET) or single-photon emission computed tomography (SPECT) including drug or substrate tissue distribution assays, or in radioactive treatment of patients. In particular, an ¹⁸F or ¹¹C labeled compound may be particularly preferred for PET or SPECT studies. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this present disclosure and prodrugs thereof can generally be prepared by carrying out the procedures disclosed in the schemes or in the examples and preparations described below by substituting a readily available isotopically labeled reagent for a non-isotopically labeled reagent.

When referring to any formula given herein, the selection of a particular moiety from a list of possible species for a specified variable is not intended to define the same choice of the species for the variable appearing elsewhere. In other words, where a variable appears more than once, the choice of the species from a specified list is independent of the choice of the species for the same variable elsewhere in the formula, unless stated otherwise.

According to the foregoing interpretive considerations on assignments and nomenclature, it is understood that explicit reference herein to a set implies, where chemically meaningful and unless indicated otherwise, independent reference to embodiments of such set, and reference to each and every one of the possible embodiments of subsets of the set referred to explicitly.

By way of a first example on substituent terminology, if substituent S¹ₑₓₐₘₚₗₑ is one of S₁ and S₂, and substituent S²ₑₓₐₘₚₗₑ is one of S₃ and S₄, then these assignments refer to embodiments of this present disclosure given according to the choices S¹ₑₓₐₘₚₗₑ is S₁ and S²ₑₓₐₘₚₗₑ is S₃; S¹ₑₓₐₘₚₗₑ is S₁ and S²ₑₓₐₘₚₗₑ is S₄; S¹ₑₓₐₘₚₗₑ is S₂ and S²ₑₓₐₘₚₗₑ is S₃; S¹ₑₓₐₘₚₗₑ is S₂ and S²ₑₓₐₘₚₗₑ is S₄; and equivalents of each one of such choices. The shorter terminology “S¹ₑₓₐₘₚₗₑ is one of S₁ and S₂, and S²ₑₓₐₘₚₗₑ is one of S₃ and S₄” is accordingly used herein for the sake of brevity, but not by way of limitation. The foregoing first example on substituent terminology, which is stated in generic terms, is meant to illustrate the various substituent assignments described herein. The foregoing convention given herein for substituents extends, when applicable, to members such as R^{a}, R^{1a}, R^{1b}, R², R³, R⁴, PG, PG¹, PG², m, n, and X, and any other generic substituent symbol used herein.

Furthermore, when more than one assignment is given for any member or substituent, embodiments of this present disclosure comprise the various groupings that can be made from the listed assignments, taken independently, and equivalents thereof. By way of a second example on substituent terminology, if it is herein described that substituent Sₑₓₐₘₚₗₑ is one of S₁, S₂, and S₃, this listing refers to embodiments of this present disclosure for which Sₑₓₐₘₚₗₑ is S₁; Sₑₓₐₘₚₗₑ is S₂; Sₑₓₐₘₚₗₑ is S₃; Sₑₓₐₘₚₗₑ is one of S₁ and S₂; Sₑₓₐₘₚₗₑ is one of S₁ and S₃; Sₑₓₐₘₚₗₑ is one of S₂ and S₃; Sₑₓₐₘₚₗₑ is one of S₁, S₂ and S₃; and Sₑₓₐₘₚₗₑ is any equivalent of each one of these choices. The shorter terminology “Sₑₓₐₘₚₗₑ is one of S₁, S₂, and S₃” is accordingly used herein for the sake of brevity, but not by way of limitation. The foregoing second example on substituent terminology, which is stated in generic terms, is meant to illustrate the various substituent assignments described herein. The foregoing convention given herein for substituents extends, when applicable, to members such as R^{a}, R^{1a}, R^{1b}, R², R³, R⁴, PG, PG¹, PG², m, n, and X, and any other generic substituent symbol used herein.

The nomenclature “Cᵢ₋ⱼ” with j>i, when applied herein to a class of substituents, is meant to refer to embodiments of this present disclosure for which each and every one of the number of carbon members, from i to j including i and j, is independently realized. By way of example, the term C₁₋₄ refers independently to embodiments that have one carbon member (C₁), embodiments that have two carbon members (C₂), embodiments that have three carbon members (C₃), and embodiments that have four carbon members (C₄).

The term Cₙ₋ₘalkyl refers to an aliphatic chain, whether straight or branched, with a total number N of carbon members in the chain that satisfies n≤N≤m, with m>n. Any disubstituent referred to herein is meant to encompass the various attachment possibilities when more than one of such possibilities are allowed. For example, reference to disubstituent-A-B-, where A≠B, refers herein to such disubstituent with A attached to a first substituted member and B attached to a second substituted member, and it also refers to such disubstituent with A attached to the second substituted member and B attached to the first substituted member.

The present disclosure includes also pharmaceutically acceptable salts of the compounds of Formula (I) (as well as Formula (IA) and Formula (IB), preferably of those described above and of the specific compounds exemplified herein, and methods of treatment using such salts.

The term “pharmaceutically acceptable” means approved or approvable by a regulatory agency of Federal or a state government or the corresponding agency in countries other than the United States, or that is listed in the U.S. Pharmcopoeia or other generally recognized pharmacopoeia for use in animals, and more particularly, in humans.

A “pharmaceutically acceptable salt” is intended to mean a salt of a free acid or base of compounds represented by Formula (I) that are non-toxic, biologically tolerable, or otherwise biologically suitable for administration to the subject. It should possess the desired pharmacological activity of the parent compound. See, generally, G. S. Paulekuhn, et al., “Trends in Active Pharmaceutical Ingredient Salt Selection based on Analysis of the Orange Book Database”, J. Med. Chem., 2007, 50:6665-72, S. M. Berge, et al., “Pharmaceutical Salts”, J Pharm Sci., 1977, 66:1-19, and Handbook of Pharmaceutical Salts, Properties, Selection, and Use, Stahl and Wermuth, Eds., Wiley-VCH and VHCA, Zurich, 2002. Examples of pharmaceutically acceptable salts are those that are pharmacologically effective and suitable for contact with the tissues of patients without undue toxicity, irritation, or allergic response. A compound of Formula (I) may possess a sufficiently acidic group, a sufficiently basic group, or both types of functional groups, and accordingly react with a number of inorganic or organic bases, and inorganic and organic acids, to form a pharmaceutically acceptable salt.

The present disclosure also relates to pharmaceutically acceptable prodrugs of the compounds of Formula (I), and treatment methods employing such pharmaceutically acceptable prodrugs. The term “prodrug” means a precursor of a designated compound that, following administration to a subject, yields the compound in vivo via a chemical or physiological process such as solvolysis or enzymatic cleavage, or under physiological conditions (e.g., a prodrug on being brought to physiological pH is converted to the compound of Formula (I). A “pharmaceutically acceptable prodrug” is a prodrug that is non-toxic, biologically tolerable, and otherwise biologically suitable for administration to the subject. Illustrative procedures for the selection and preparation of suitable prodrug derivatives are described, for example, in “Design of Prodrugs”, ed. H. Bundgaard, Elsevier, 1985.

The present disclosure also relates to pharmaceutically active metabolites of the compounds of Formula (I), which may also be used in the methods of the present disclosure. A “pharmaceutically active metabolite” means a pharmacologically active product of metabolism in the body of a compound of Formula (I) or salt thereof. Prodrugs and active metabolites of a compound may be determined using routine techniques known or available in the art. See, e.g., Bertolini, et al., J Med Chem. 1997, 40, 2011-2016; Shan, et al., J Pharm Sci. 1997, 86 (7), 765-767; Bagshawe, Drug Dev Res. 1995, 34, 220-230; Bodor, Adv Drug Res. 1984, 13, 224-331; Bundgaard, Design of Prodrugs (Elsevier Press, 1985); and Larsen, Design and Application of Prodrugs, Drug Design and Development (Krogsgaard-Larsen, et al., eds., Harwood Academic Publishers, 1991).

As used herein, the term “composition” or “pharmaceutical composition” refers to a mixture of at least one compound provided herein with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound provided herein within or to the patient such that it can perform its intended function. Typically, such constructs are carried or transported from one organ, or portion of the body, to another organ, or portion of the body. Each carrier must be “acceptable” in the sense of being compatible with the other ingredients of the formulation, including the compound provided herein, and not injurious to the patient. Some examples of materials that can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound provided herein, and are physiologically acceptable to the patient. Supplementary active compounds can also be incorporated into the compositions. The “pharmaceutically acceptable carrier” can further include a pharmaceutically acceptable salt of the compound provided herein. Other additional ingredients that can be included in the pharmaceutical compositions provided herein are known in the art and described, for example in Remington's Pharmaceutical Sciences (Genaro, Ed., Mack Publishing Co., 1985, Easton, Pa.), which is incorporated herein by reference.

The term “stabilizer,” as used herein, refers to polymers capable of chemically inhibiting or preventing degradation of a compound of Formula I. Stabilizers are added to formulations of compounds to improve chemical and physical stability of the compound.

The term “tablet,” as used herein, denotes an orally administrable, single-dose, solid dosage form that can be produced by compressing a drug substance or a pharmaceutically acceptable salt thereof, with suitable excipients (e.g., fillers, disintegrants, lubricants, glidants, and/or surfactants) by conventional tableting processes. The tablet can be produced using conventional granulation methods, for example, wet or dry granulation, with optional comminution of the granules with subsequent compression and optional coating. The tablet can also be produced by spray-drying.

As used herein, the term “capsule” refers to a solid dosage form in which the drug is enclosed within either a hard or soft soluble container or “shell.” The container or shell can be formed from gelatin, starch and/or other suitable substances.

As used herein, the terms “effective amount,” “pharmaceutically effective amount,” and “therapeutically effective amount” refer to a nontoxic but sufficient amount of an agent to provide the desired biological result. That result may be reduction or alleviation of the signs, symptoms, or causes of a disease, or any other desired alteration of a biological system. An appropriate therapeutic amount in any individual case may be determined by one of ordinary skill in the art using routine experimentation.

The term “combination,” “therapeutic combination,” “pharmaceutical combination,” or “combination product” as used herein refer to a non-fixed combination or a kit of parts for the combined administration where two or more therapeutic agents can be administered independently, at the same time or separately within time intervals, especially where these time intervals allow that the combination partners show a cooperative, e.g., synergistic, effect.

The term “modulators” include both inhibitors and activators, where “inhibitors” refer to compounds that decrease, prevent, inactivate, desensitize, or down-regulate HBV assembly and other HBV core protein functions necessary for HBV replication or the generation of infectious particles.

As used herein, the term “capsid assembly modulator” refers to a compound that disrupts or accelerates or inhibits or hinders or delays or reduces or modifies normal capsid assembly (e.g., during maturation) or normal capsid disassembly (e.g., during infectivity) or perturbs capsid stability, thereby inducing aberrant capsid morphology and function. In one embodiment, a capsid assembly modulator accelerates capsid assembly or disassembly, thereby inducing aberrant capsid morphology. In another embodiment, a capsid assembly modulator interacts (e.g. binds at an active site, binds at an allosteric site, modifies and/or hinders folding and the like) with the major capsid assembly protein (CA), thereby disrupting capsid assembly or disassembly. In yet another embodiment, a capsid assembly modulator causes a perturbation in structure or function of CA (e.g., ability of CA to assemble, disassemble, bind to a substrate, fold into a suitable conformation, or the like), which attenuates viral infectivity and/or is lethal to the virus.

As used herein, the term “treatment” or “treating,” is defined as the application or administration of a therapeutic agent, i.e., a compound of the present disclosure (alone or in combination with another pharmaceutical agent), to a patient, or application or administration of a therapeutic agent to an isolated tissue or cell line from a patient (e.g., for diagnosis or ex vivo applications), who has an HBV infection, a symptom of HBV infection or the potential to develop an HBV infection, with the purpose to cure, heal, alleviate, relieve, alter, remedy, ameliorate, improve or affect the HBV infection, the symptoms of HBV infection or the potential to develop an HBV infection. Such treatments may be specifically tailored or modified, based on knowledge obtained from the field of pharmacogenomics.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “patient,” “individual” or “subject” refers to a human or a non-human mammal. Non-human mammals include, for example, livestock and pets, such as ovine, bovine, porcine, canine, feline and murine mammals. Preferably, the patient, subject or individual is human.

In treatment methods according to the present disclosure, an effective amount of a pharmaceutical agent according to the present disclosure is administered to a subject suffering from or diagnosed as having such a disease, disorder, or condition. An “effective amount” means an amount or dose sufficient to generally bring about the desired therapeutic or prophylactic benefit in patients in need of such treatment for the designated disease, disorder, or condition. Effective amounts or doses of the compounds of the present disclosure may be ascertained by routine methods such as modeling, dose escalation studies or clinical trials, and by taking into consideration routine factors, e.g., the mode or route of administration or drug delivery, the pharmacokinetics of the compound, the severity and course of the disease, disorder, or condition, the subject's previous or ongoing therapy, the subject's health status and response to drugs, and the judgment of the treating physician. An example of a dose is in the range of from about 0.001 to about 200 mg of compound per kg of subject's body weight per day, preferably about 0.05 to 100 mg/kg/day, or about 1 to 35 mg/kg/day, in single or divided dosage units (e.g., BID, TID, QID). For a 70-kg human, an illustrative range for a suitable dosage amount is from about 0.05 to about 7 g/day, or about 0.2 to about 2.5 g/day.

An example of a dose of a compound is from about 1 mg to about 2,500 mg. In some embodiments, a dose of a compound of the present disclosure used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound (i.e., another drug for HBV treatment) as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

Once improvement of the patient's disease, disorder, or condition has occurred, the dose may be adjusted for preventative or maintenance treatment. For example, the dosage or the frequency of administration, or both, may be reduced as a function of the symptoms, to a level at which the desired therapeutic or prophylactic effect is maintained. Of course, if symptoms have been alleviated to an appropriate level, treatment may cease. Patients may, however, require intermittent treatment on a long-term basis upon any recurrence of symptoms.

HBV infections that may be treated according to the disclosed methods include HBV genotype A, B, C, and/or D infections. However, in an embodiment, the methods disclosed may treat any HBV genotype (“pan-genotypic treatment”). HBV genotyping may be performed using methods known in the art, for example, INNO-LIPA® HBV Genotyping, Innogenetics N.V., Ghent, Belgium).

### EXAMPLES

Exemplary compounds useful in methods of the present disclosure will now be described by reference to the illustrative synthetic schemes for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Unless otherwise specified, the variables are as defined above in reference to Formula (I). Reactions may be performed between the melting point and the reflux temperature of the solvent, and preferably between 0° C. and the reflux temperature of the solvent. Reactions may be heated employing conventional heating or microwave heating. Reactions may also be conducted in sealed pressure vessels above the normal reflux temperature of the solvent.

Abbreviations and acronyms used herein include the following set forth in Table 2:

TABLE 2

| Term | Acronym |
|---|---|
| Aqueous | aq |
| Atmosphere | atm |
| tert-butyloxycarbonyl | Boc, BOC |
| Broad | br |
| Capsid assembly | CA |
| Doublet of doublets | dd |
| Dimethylsulfoxide | DMSO |
| Deoxyribonucleic Acid | DNA |
| Diethyl ether | Ether, Et₂O |
| Diisopropylethylamine | DIEA |
| Ethyl Acetate | EtOAc, or EA |
| Ethanol | EtOH |
| Electrospray ionization | ESI |
| Normal-phase silica gel chromatography | FCC |
| Grams | g |
| Hours | h or hr |
| Hepatitis B Virus | HBV |
| Acetic acid | HOAc |
| High-pressure liquid chromatography | HPLC |
| Hertz | Hz |
| Isopropyl alcohol | iPrOH, IPA |
| Potassium tert-butoxide | KOtBu |
| Lithium aluminum hydride | LAH |
| Liquid chromatography and mass spectrometry | LCMS |
| Lithium bis(trimethylsilyl)amide | LHMDS, |
| | LiHMDS |
| Molar | M |
| multiplet | m |
| Mass to charge ratio | m/z |
| Methanol | MeOH |
| Milligrams | mg |
| Megahertz | MHz |
| Minute | min |
| Milliliter | mL |
| Microliter | μL |
| Millimole | mmol |
| Micromole | μmol |
| Mass spectrometry | MS |
| Mesityl chloride | MsCl |
| Normal | N |
| Nuclear magnetic resonance | NMR |
| Polymerase chain reaction | PCR |
| Petroleum ether | PE |
| 9-(2-Phosphonyl-methoxypropyly)adenine | PMPA |
| Parts per million | ppm |
| Precipitate | ppt |
| Retention time | Rₜ |
| Reverse Phase | RP |
| Ribonucleic Acid | RNA |
| Room temperature | rt |
| singlet | s |
| Saturated | sat |
| Supercritical Fluid Chromatography | SFC |
| Temperature | T |
| triplet | t |
| Triethylamine | TEA |
| Trifluoroacetic acid | TFA |
| Tetrahydrofuran | THF |
| Thin layer chromatography | TLC |
| Toll-like receptor | TLR |
| Tumor necrosis factor | TNF |
| Volume in milliliters of solvent per gram of | V, or volumes |
| substrate | |

### Synthesis

Exemplary compounds useful in methods of the present disclosure will now be described by reference to the illustrative synthetic schemes for their general preparation below and the specific examples to follow.

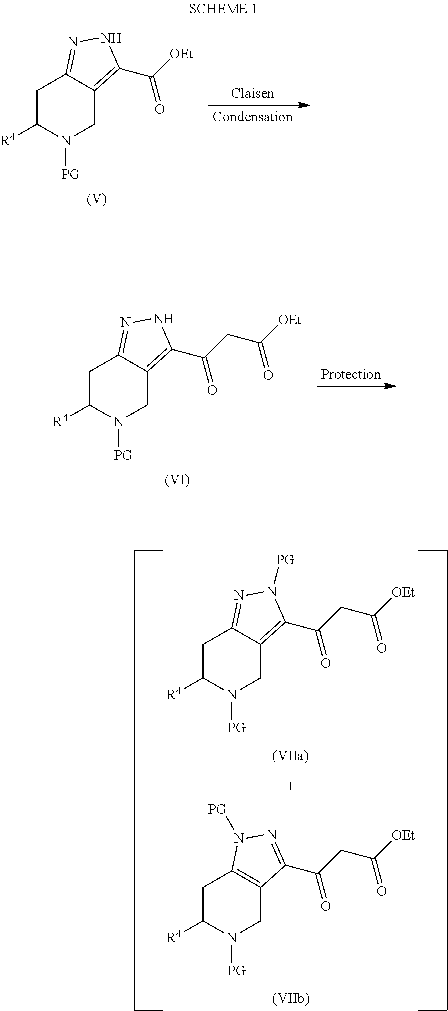

According to SCHEME 1, a compound of formula (V), where R⁴ is H or C₁₋₄alkyl, and PG is BOC, undergoes a Claisen-type reaction or acylation with ethyl acetate; in the presence of a suitable base such as sodium hydride, potassium hydride, lithium diisopropylamide (LDA), lithium hexamethyldisilylamide (LHMDS), sodium bis(trimethylsilyl)amide (NaHMDS), potassium butoxide, and the like; preferably sodium bis(trimethylsilyl)amide (NaHMDS); in a suitable solvent such as tetrahydrofuran (THF), dioxane, dimeth-oxyethane, toluene, xylenes, acetonitrile (ACN), dimethysulfoxide, dimethylformamide (DMF), dimethylacetamide (DMA), N-methylpyrrolidone, and the like; preferably THF; at a temperature ranging from −70 to 100° C., preferably −65 to 40° C.; for a period of 2 h to 24 h. A compound of formula (VI) is protected employing established methodologies, such as those described in T. W. Greene and P. G. M. Wuts, “Protective Groups in Organic Synthesis,” 3 ed., John Wiley & Sons, 1999, to provide a mixture of compounds of formula (VIIa) and formula (VIIb), where R⁴ is H or C₁₋₄alkyl, and PG is BOC.

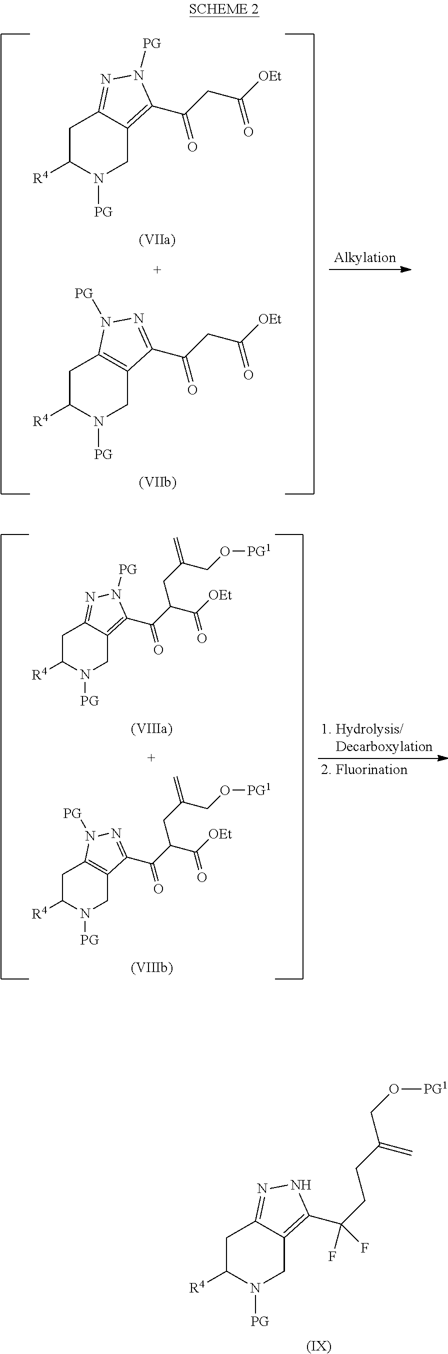

According to SCHEME 2, alkylation of β-ketoester compounds of formula (VIIa) and (VIIb), where R⁴ is H or C₁₋₄alkyl, and PG is BOC, is achieved employing an alkyl halide such as ((2-(bromomethyl)allyl)oxy)(tert-butyl)diphenylsilane, a base such as K₂CO₃; NaI; in a suitable solvent such as acetone, and the like; to provide a mixture of compounds of formulas (VIIIa) and (VIIIb). Hydrolysis/decarboxylation of a mixture of compounds of formula (VIIIa) and (VIIIb) is achieved using a base such as with potassium hydroxide, and the like; in a suitable solvent such a as MeOH, H₂O, or a mixture thereof. Subsequent fluorination is achieved employing conditions known to one skilled in the art, to provide a compound of formula (IX), where R⁴ is H or C₁₋₄alkyl, PG is BOC, and PG¹ is TBDPS. For example, treatment with a deoxo-fluorinating agent dialkylaminosulfotrifluoride (DAST), and the like, in a suitable solvent such as dichloromethane, and the like, provides a compound of formula (IX).

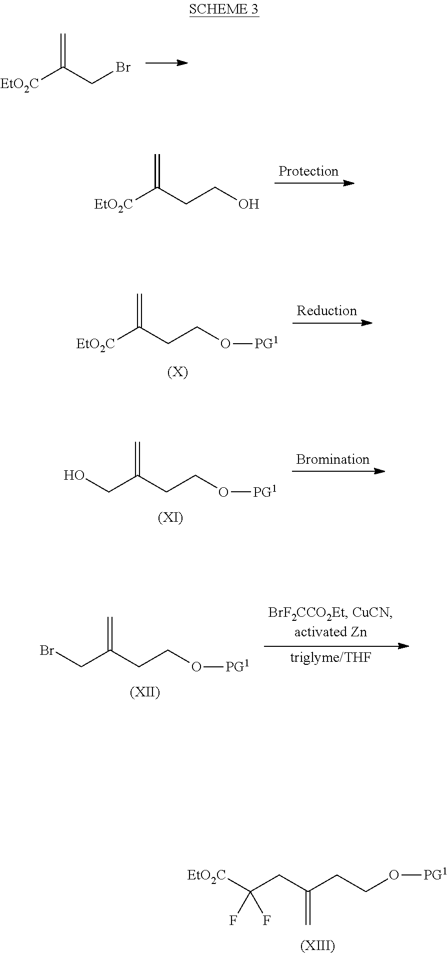

According to SCHEME 3, commercially available or synthetically accessible ethyl 4-hydroxy-2-methylenebutanoate is protected with a silyl protecting group such as t-butyldiphenylsilyl ether (TBDPS), trimethylsilyl (TMS), tert-butyldimethylsilyl (TBDMS), and triisopropylsilyl (TIPS) ethers, preferably TBDPS. For example reaction of ethyl 4-hydroxy-2-methylenebutanoate with an alkyldiarylsilyl halide such as t-butyldiphenylsilyl chloride; imidazole; with or without the presence of a catalyst such as 4-(dimethylamino)pyridine (DMAP); in a suitable solvent such as dimethylformamide (DMF), tetrahydrofuran (THF), and the like; for a period of about 10 hours; at room temperature; to provide a compound of formula (X). Reduction of a compound of formula (X) with a reducing agent such as diisobutylaluminium hydride (DIBAL-H), in a suitable solvent such as THF, at temperatures ranging from −70 to 100° C., preferably −65 to 40° C.; for a period of 2 h to 24 h.

An alcohol compound of formula (XI) is brominated under Appel halogenation conditions known to one skilled in the art. For example, a compound of formula (XI) is reacted triphenylphosphine, a tetrahalomethanes such as CBr₄, in a suitable solvent such as DCM, and the like, to provide a bromo compound of formula (XII).

Coupling of a compound of formula (XII) with ethyl bromodifluoroacetate in the presence of activated zinc powder and CuCN, in a suitable solvent such as triglyme/THF, at temperatures ranging from 10 to 40° C., for a period of 16 to 24 h, provides a compound of formula (XIII), where PG¹ is TBDPS.

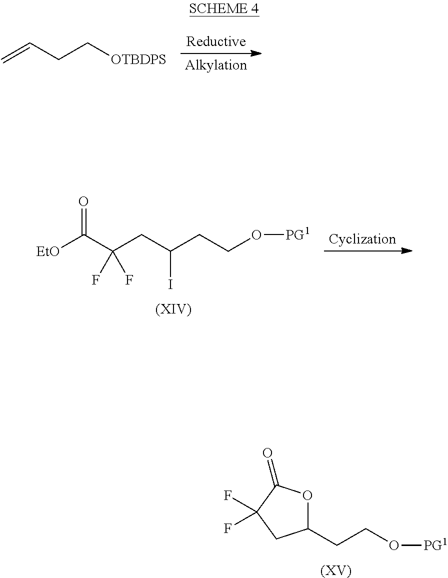

According to SCHEME 4, commercially available or synthetically accessible (but-3-en-1-yloxy)(tert-butyl)diphenylsilane undergoes reductive alkylation employing an alkali metal dithionite, such as sodium dithionite as an initiator; sodium hydrogen carbonate as the base; and ethyl 2,2-difluoro-2-iodoacetate; in a suitable solvent such as a mixture of acetonitrile and water; to provide a compound of formula (XIV), where PG¹ is TBDPS. A lactone compound of formula (XV), where PG¹ is TBDPS, is prepared from a compound of formula (XIV) in an intramolecular cyclization employing aq. Na₂CO₃, at elevated temperature, for a period of 5-8 h.

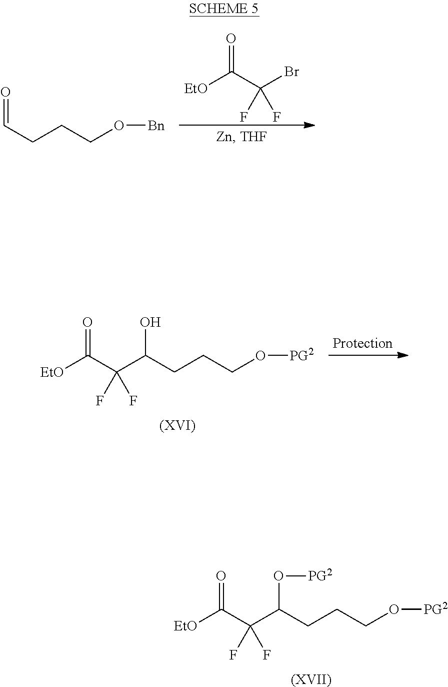

According to SCHEME 5, 4-(benzyloxy)butanal is reacted under Reformatsky conditions with an a-halo ester such as ethyl 2-bromo-2,2-difluoroacetate, metallic zinc, in a solvent such as THF, to provide an β-hydroxy-ester compound of formula (XVI), where PG² is benzyl. Subsequent benzylation of the β-hydroxy-ester compound of formula (XVI), with benzyl bromide and sodium hydride affords a compound of formula (XVII).

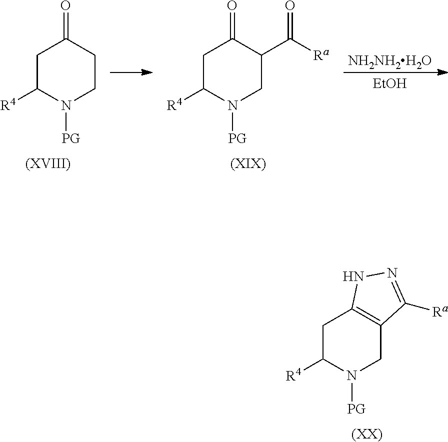

According to SCHEME 6, a compound of formula (XVIII), where R⁴ is H or C₁₋₄alkyl, and PG is BOC or CBz, is reacted with a base such as lithium bis(trimethylsilyl)amide (LiHMDS) and the like; followed by condensation with a suitable acylating agent such as diethyloxalate, a compound of formula (XII), formula (XV), or formula (XVII); in a suitable solvent such as THF, and the like; at temperatures ranging from −78 to 60° C.; to provide a compound of formula (XIX), where R^{a} is CO₂Et, CF₂CH₂C(═CH₂)(CH₂CH₂OTBDPS), CF₂CH₂CH(OH)(CH₂CH₂OTBDPS), or CF₂CH(OBn)(CH₂CH₂CH₂OBn); and R⁴ is H or C₁₋₄alkyl. Condensation of formula (XIX), where R^{a} is CO₂Et, CF₂CH₂C(═CH₂)(CH₂CH₂OTBDPS), CF₂CH₂CH(OH)(CH₂CH₂OTBDPS), or CF₂CH(OBn)(CH₂CH₂CH₂OBn); and R⁴ is H or C₁₋₄alkyl; with hydrazine, in EtOH, provides a compound of formula (XX).

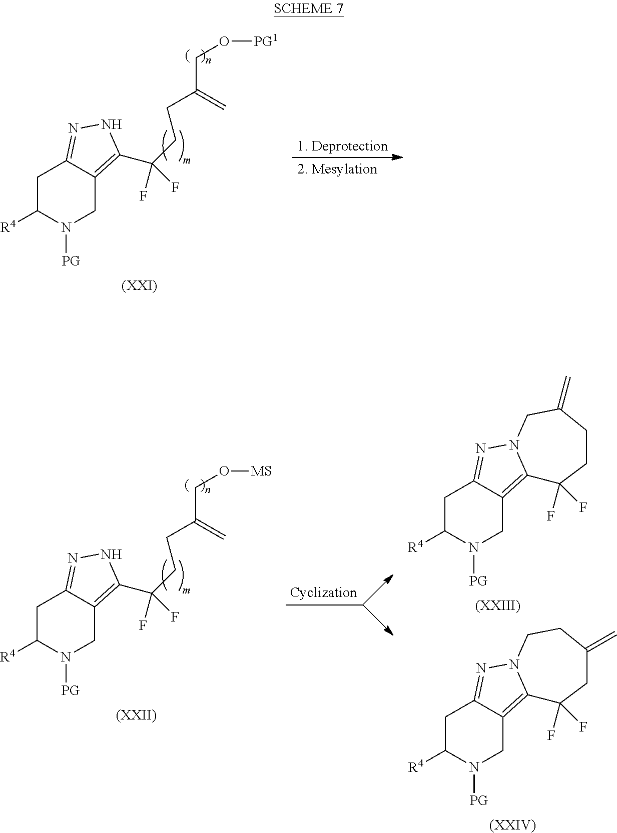

According to SCHEME 7, a compound of formula (XXI), where R⁴ is H or C₁₋₄alkyl, PG is BOC, PG¹ is TBDSP, and m is 1 and n is 2, or m is 2 and n is 1; is de-silylated with tetra-n-butylammonium fluoride (TBAF), in a suitable solvent such as THF and the like. Subsequent mesylation of the hydroxy employing methanesulfonyl chloride (mesyl chloride), a suitable base such as triethylamine (TEA), in a suitable solvent such as DCM, and the like, provides a compound of formula (XXII). Intramolecular cyclization employing a base such as DBU, in a suitable solvent such as THF, and the like, provides compounds of formula (XXIII) and formula (XXIV).

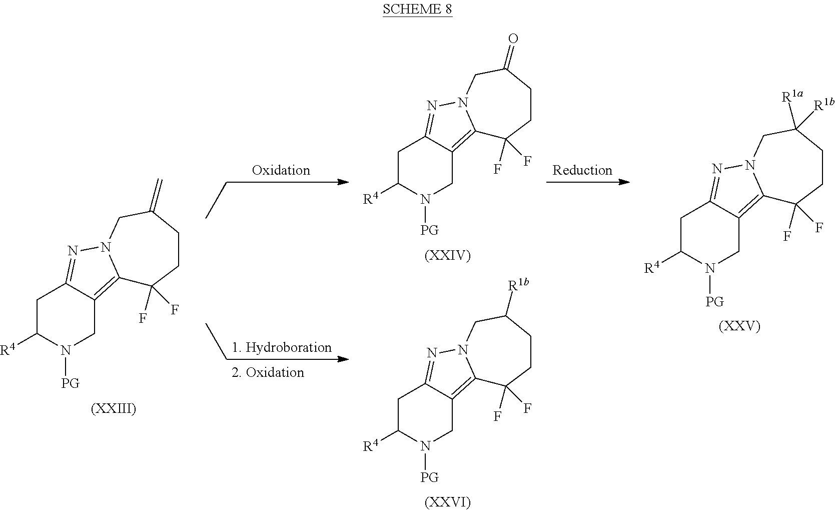

According to SCHEME 8, an olefin compound of formula (XXIII) (also a compound of XXIV can be used in the synthetic schemes as described for compounds of formula (XXIII)), is oxidized employing conditions such as NaIO₄, and OsO₄, to provide a compound of formula (XXV). Reduction of a carbonyl compound of formula (XXV) where R⁴ is H or C₁₋₄alkyl, and PG is BOC, is achieved employing a reducing agent such as NaBH₄, and the like; in a suitable solvent such as DMF, THF, and the like; to provide a compound of formula (XXVI), where R^{1a} is H and R^{1b} is OH.

Alternately, hydroboration of an olefin of compound of formula (XXIII) is achieved employing a hydroborating agent such as 9-borabicyclo[3.3.1]nonane (9-BBN), dicyclohexyl borane, diisoamyl borane and borinane (preferably 9-BBN); in a suitable solvent such as THF, and the like; at a temperature of about 0° C. Subsequent oxidation, employing hydrogen peroxide; at a temperature ranging from −30° C. to room temperature; affords a racemic mixture of hydroxymethyl compounds of formula (XXVI), where R^{1b} is CH₂OH, and PG is BOC. In some embodiments, the oxidation takes place in an alkaline environment created by the addition of a base, for example, sodium hydroxide or potassium hydroxide.

Conversion of a hydroxy compound of formula (XXV), where R^{1a} is H and R^{1b} is OH, to the corresponding fluoro derivative of formula (XXV), where R^{1a} is H and R^{1b} is F, is achieved using the deoxo-fluorinating agent dialkylaminosulfotrifluoride (DAST), in a suitable solvent such as dichloromethane, and the like.

Alkylation of a hydroxy compound of formula (XXV), where R^{1a} is H and R^{1b} is OH, or a compound of formula (XXVI) where R^{1b} is CH₂OH, employing an alkyl halide, an alkyl sulfonate, or a haloalkyl sulfonate; a base such as NaH, K₂CO₃, CsCO₃, and the like; in a suitable solvent such as THF, ACN, and the like; at temperatures ranging from −40° C. to 0° C.; for a period of 1-5 h; provides compounds formula (XXV), where R^{1a} is H and R^{1b} is OC₁₋₄alkyl or OC₁₋₄haloalkyl, or compounds of formula (XXVI) where R^{1b} is CH₂OC₁₋₄alkyl or CH₂OC₁₋₄haloalkyl respectively.

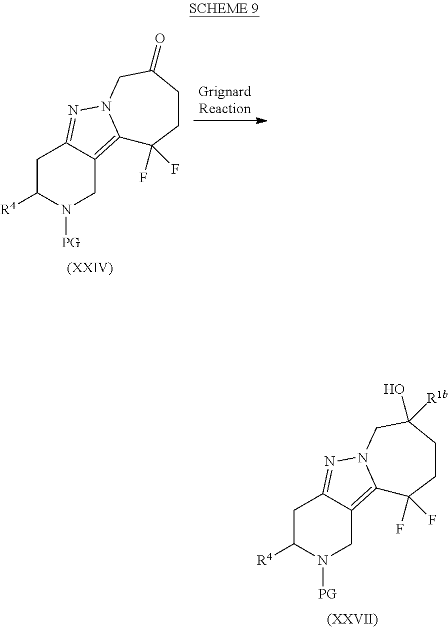

According to SCHEME 9, reaction of a carbonyl compound of formula (XXIV), where R⁴ is H or C₁₋₄alkyl, and PG is BOC; with a Grignard reagent of formula R^{1b}MgY, where Y is halogen, and R^{1b} is C₁₋₄alkyl, C₂₋₄alkenyl, or C₂₋₄alkynyl; with or without the addition of NaCl, LiBr, or LiCl; affords an alcohol compound of formula (XXVII). For example, reaction of a compound of formula (XXIV) with a Grignard reagent, such as ethynyl magnesium bromide, vinyl magnesium bromide, methyl magnesium bromide, and the like; in a suitable solvent such as DCM, THF, and the like; affords a compound of formula (XXVII), where R^{1b} is C₁₋₄alkyl, C₂₋₄alkenyl, or C₂₋₄alkynyl.

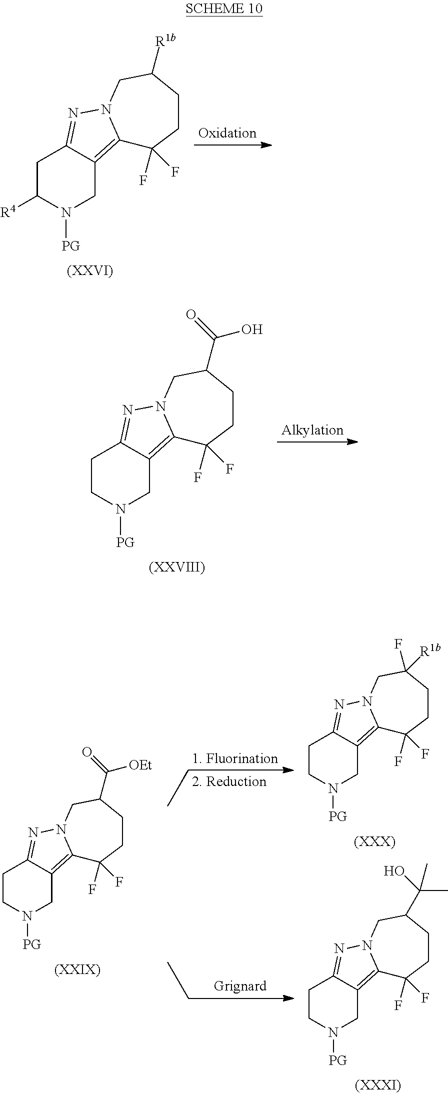

According to SCHEME 10, oxidation of an alcohol compound of formula (XXVI), where R^{1b} is CH₂OH, R⁴ is H or C₁₋₄alkyl, and PG is BOC, is achieved employing conditions known to one skilled in the art, to provide a carboxylic acid compound of formula (XXVIII). For example, reaction of an alcohol compound of formula (XXVI), where R^{1b} is CH₂OH, with the oxidation catalyst tetrapropylammonium perruthenate (TPAP); and N-methylmorpholine N-oxide (NMO) as the co-oxidant; in a suitable solvent such as ACN, DCM, DMF, and the like; provides a carboxylic acid compound of formula (XXVIII).

Alkylation of a carboxylic acid compound of formula (XXVIII), employing an alkyl halide such as EtI, MeI, and the like; a base such as K₂CO₃, CsCO₃, and the like; in a suitable solvent such as THF, ACN, and the like; at temperatures ranging from 0° C. to 20° C.; for a period of 10-20 h; provides an ester compound of formula (XXIX).

An ester compound of formula (XXIX) is deprotonated with lithium diisopropylamide (LDA) followed by treatment with a fluorinating agent such as N-fluorobenzenedisulfonimide (NFSI), 1-chloromethyl-4-fluoro-1,4-diazoniabicyclo[2.2.2]octane bis(tetrafluoroborate) (Selectfluor®), and the like; in a suitable solvent such as THF, DMF, or a mixture thereof. Reduction of the ester, employing a reducing agent such as LiBH₄, and the like; in a suitable solvent such as THF, and the like; affords a compound of formula (XXX), where R^{1b} is CH₂OH.

An ester compound of formula (XXIX), where R^{1b} is CH₂OH, R⁴ is C₁₋₄alkyl, and PG is BOC is reacted with MeMgBr; in a solvent such as THF, and the like; at temperatures ranging from −40° C. to 0° C.; for a period of 1-5 h; provides a compound of formula (XXXI).

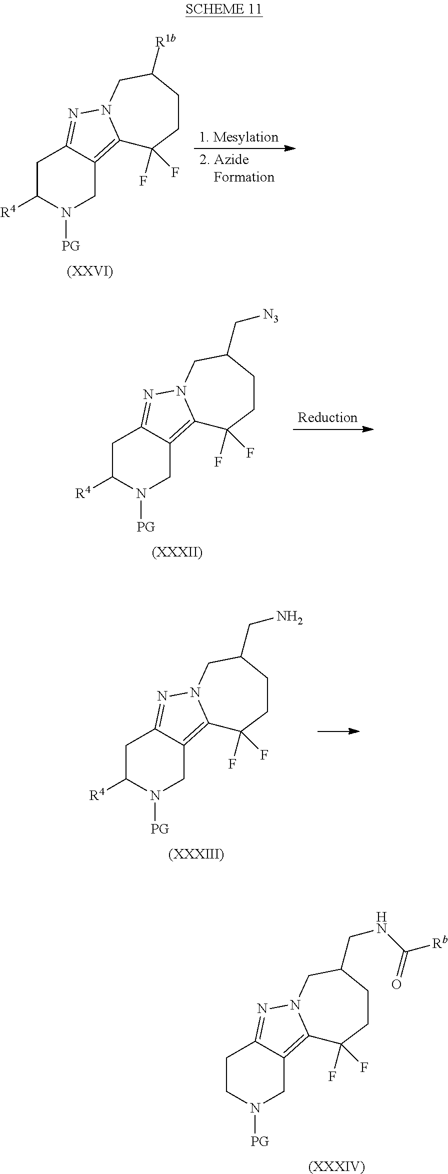

According to SCHEME 11, an alcohol compound of formula (XXVI), where R^{1b} is CH₂OH is converted to a mesylate leaving group employing conditions known to one skilled in the art. One of skill in the art will appreciate that many possible leaving groups may be used. Particular examples include, but are not limited to triflate, mesylate, paratoluene sulfonate, nosylate, and brosylate. The leaving group is then displaced using an azide, such as DPPA or NaN₃. For example, displacement of the sulfonate ester leaving group with sodium azide, in a suitable solvent which does not adversely affect the reaction (e.g. chloroform, dichloromethane, tetrahydrofuran, pyridine, dimethylsulfoxide, N,N-dimethylformamide, hexamethylphosphoramide, etc. or a mixture thereof); at temperatures ranging from room temperature to 120° C.; provides an azide compound of formula (XXXII). An azide compound of formula (XXXII) is reduced to the corresponding amine, employing reduction conditions known to one skilled in the art (for examples, see Larock, R. C. in Comprehensive Organic Transformations, Wiley-VCH Publishers, 1999). In a preferred method, the azide is reduced with zinc and ammonium chloride.

Acylation of a compound of formula (XXXIII), employing an acylating reagent selected from an acyl derivative, an acyl halide such as acetyl chloride, and the like; an acid anhydride such as acetic anhydride, trifluoroacetic anhydride, and the like; or a chloroformate such as methyl chloroformate; a base such as triethylamine (TEA), and the like; in a suitable solvent such as DMF, DCM, and the like; affords a compound of formula (XXXIV).

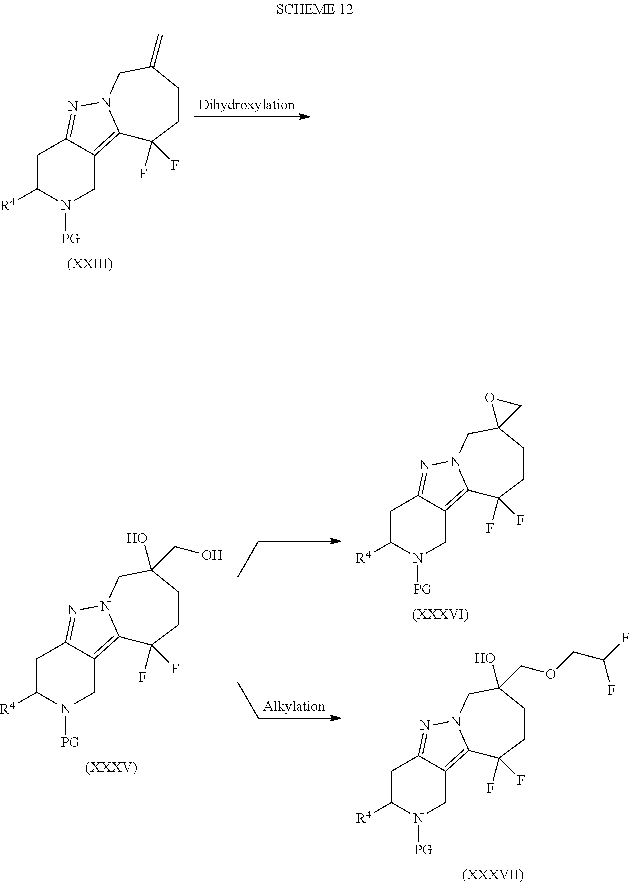

According to SCHEME 12, a compound of formula (XXIII), where R⁴ is H or C₁₋₄alkyl, undergoes an osmium-catalyzed dihydroxylation, employing conditions known to one skilled in the art, to provide a compound of formula (XXXV). For example, a compound of formula (XXIII), where R⁴ is H or C₁₋₄alkyl; is reacted with an oxidant such as an osmium-containing compound like OsO₄ (or OsO₄ can also be prepared in situ by the oxidation of K₂OsO₂(OH)₄ with NMO); an amine oxide co-oxidant such as NMO, and the like; in a suitable solvent such as THF, acetone, H₂O, or a mixture thereof; to provide a compound of formula (XXXV). A diol compound of formula (XXXV) is converted to an epoxide compound of formula (XXXVI) employing n-perfluorobutanesulfonyl fluoride with 1,8-diazabicyclo[5.4.0]undec-7-ene (DBU), a suitable solvent such as THF, at temperatures ranging from 0° C. to 20° C., for a period of 4-7 h.

Alkylation of a hydroxy compound of formula (XXXV), employing an haloalkyl sulfonate such as 2,2-difluoroethyl trifluoromethanesulfonate; a base such as NaH, NaHMDS, and the like; in a suitable solvent such as THF, ACN, DMF, or a mixture thereof; at temperatures ranging from −78° C. to 0° C., provides a compound of formula (XXXVII), where R⁴ is H or C₁₋₄alkyl.

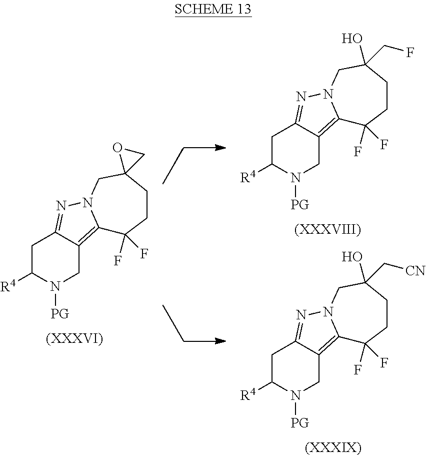

According to SCHEME 13, an epoxide compound of formula (XXXVI) is opened by anhydrous acids to form a corresponding fluoroalcohol compound of formula (XXXVIII). For example, an epoxide compound of formula (XXXVI) is reacted with an amineHF such as Et₃N.3HF, at a temperature of about 100° C., employing conventional or microwave heating, for a period of about 3-7 h, to provide a fluoroalcohol compound of formula (XXXVIII).

Cyanide-induced ring opening of an epoxide compound of formula (XXXVI) is achieved employing a cyanide source such as KCN, TMSCN, and the like; a Lewis Acid such as LiClO₄, and the like; in a suitable solvent such as THF, ACN, and the like; to provide beta-hydroxy nitrile compound of formula (XXXIX).

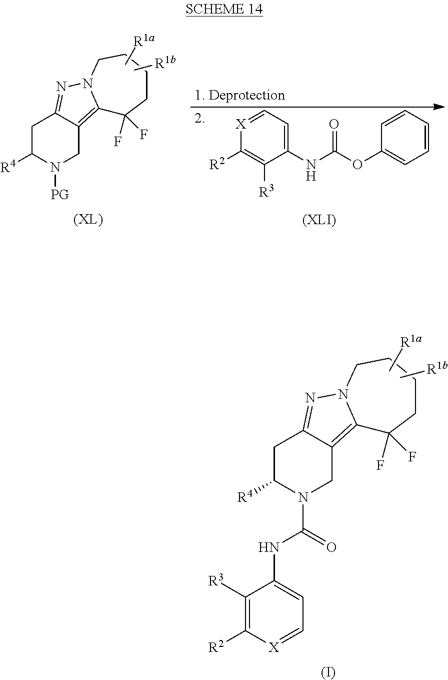

According to SCHEME 14, a compound of formula (XL) (which encompasses compounds of formulas (XXIII), (XXIV), (XXV), (XXVI), (XXVII), (XXX), (XXXI), (XXXIV), (XXXV) (XXXVII), (XXXVIII), and (XXXIX)), wherein R^{1a}, R^{1b}, and R⁴ are as defined in claim 1, and PG is BOC or Cbz; is deprotected employing conditions known to one skilled in the art (wherein when PG is Cbz, deprotection of the CBz group is achieved employing Pd/C; under an Hz, in the presence of (Boc)O, in a suitable solvent such as EtOH, and the like to provide a compound of formula (XL) where PG is BOC). Subsequent reaction with a commercially available or synthetically accessible compound of formula (XLI), where X, R² and R³ are as defined in claim 1; a suitable base such as TEA, and the like; in a suitable solvent such as DCM, and the like; provides a compound of Formula (I).

A compound of Formula (I), where R^{1a} is OH, and R^{1b} is C₂₋₄alkynyl, is reduced employing hydrogenation conditions known to one skilled in the art, for example reaction with Pd/C under H₂, in a suitable solvent such as THF, to provide a compound of Formula (I), where R^{1a} is OH, and R^{1b} is C₂₋₄alkyl.

Compounds of Formula (I) may be converted to their corresponding salts using methods known to one of ordinary skill in the art. For example, an amine of Formula (I) is treated with trifluoroacetic acid, HCl, or citric acid in a solvent such as Et₂O, CH₂Cl₂, THF, MeOH, chloroform, or isopropanol to provide the corresponding salt form. Alternately, trifluoroacetic acid or formic acid salts are obtained as a result of reverse phase HPLC purification conditions. Crystalline forms of pharmaceutically acceptable salts of compounds of Formula (I) may be obtained in crystalline form by recrystallization from polar solvents (including mixtures of polar solvents and aqueous mixtures of polar solvents) or from non-polar solvents (including mixtures of non-polar solvents).

Where the compounds according to this present disclosure have at least one chiral center, they may accordingly exist as enantiomers. Where the compounds possess two or more chiral centers, they may additionally exist as diastereomers. It is to be understood that all such isomers and mixtures thereof are encompassed within the scope of the present disclosure.

Compounds of formulas represented in the SCHEMES above represented as “stereomeric mixture” (means a mixture of two or more stereoisomers and includes enantiomers, diastereomers and combinations thereof) are separated by SFC resolution.

Compounds prepared according to the schemes described above may be obtained as single forms, such as single enantiomers, by form-specific synthesis, or by resolution. Compounds prepared according to the schemes above may alternately be obtained as mixtures of various forms, such as racemic (1:1) or non-racemic (not 1:1) mixtures. Where racemic and non-racemic mixtures of enantiomers are obtained, single enantiomers may be isolated using conventional separation methods known to one of ordinary skill in the art, such as chiral chromatography, recrystallization, diastereomeric salt formation, derivatization into diastereomeric adducts, biotransformation, or enzymatic transformation. Where regioisomeric or diastereomeric mixtures are obtained, as applicable, single isomers may be separated using conventional methods such as chromatography or crystallization.

### General Information

The following specific examples are provided to further illustrate the present disclosure and various preferred embodiments.

In obtaining the compounds described in the examples below and the corresponding analytical data, the following experimental and analytical protocols were followed unless otherwise indicated.

Unless otherwise stated, reaction mixtures were magnetically stirred at room temperature (rt) under a nitrogen atmosphere. Where solutions were “dried,” they were generally dried over a drying agent such as Na₂SO₄ or MgSO₄. Where mixtures, solutions, and extracts were “concentrated”, they were typically concentrated on a rotary evaporator under reduced pressure.

Normal-phase silica gel chromatography (FCC) was performed on silica gel (SiO₂) using prepacked cartridges.

Preparative reverse-phase high performance liquid chromatography (RP HPLC) was performed on either:
METHOD A. A Gilson GX-281 semi-prep-HPLC with Phenomenex Synergi C18(10 μm, 150×25 mm), or Boston Green ODS C18(5 μm, 150×30 mm), and mobile phase of 5-99% ACN in water (with 0.225% FA) over 10 min and then hold at 100% ACN for 2 min, at a flow rate of 25 mL/min. or
METHOD B. A Gilson GX-281 semi-prep-HPLC with Phenomenex Synergi C18(10 μm, 150×25 mm), or Boston Green ODS C18(5 μm, 150×30 mm), and mobile phase of 5-99% ACN in water (0.1% TFA) over 10 min and then hold at 100% ACN for 2 min, at a flow rate of 25 mL/min. or
METHOD C. A Gilson GX-281 semi-prep-HPLC with Phenomenex Synergi C18(10 μm, 150×25 mm), or Boston Green ODS C18(5 μm, 150×30 mm), and mobile phase of 5-99% ACN in water(0.05% HCl) over 10 min and then hold at 100% ACN for 2 min, at a flow rate of 25 mL/min. or
METHOD D. a Gilson GX-281 semi-prep-HPLC with Phenomenex Gemini C18 (10 μm, 150×25 mm), AD(10 μm, 250 mm×30 mm), or Waters)(Bridge C18 column (5 μm, 150×30 mm), mobile phase of 0-99% ACN in water (with 0.05% ammonia hydroxide v/v) over 10 min and then hold at 100% ACN for 2 min, at a flow rate of 25 mL/min. or
METHOD E. a Gilson GX-281 semi-prep-HPLC with Phenomenex Gemini C18 (10 μm, 150×25 mm), or Waters XBridge C18 column (5 μm, 150×30 mm), mobile phase of 5-99% ACN in water(10 mM NH4HCO3) over 10 min and then hold at 100% ACN for 2 min, at a flow rate of 25 mL/min.
   Preparative supercritical fluid high performance liquid chromatography (SFC) was performed either on a Thar 80 Prep-SFC system, or Waters 80Q Prep-SFC system from Waters. The ABPR was set to 100 bar to keep the CO₂ in SF conditions, and the flow rate may verify according to the compound characteristics, with a flow rate ranging from 50 g/min to 70 g/min. The column temperature was ambient temperature

Mass spectra (MS) were obtained on a SHIMADZU LCMS-2020 MSD or Agilent 1200\G6110A MSD using electrospray ionization (ESI) in positive mode unless otherwise indicated. Calculated (calcd.) mass corresponds to the exact mass.

Nuclear magnetic resonance (NMR) spectra were obtained on Bruker model AVIII 400 spectrometers. Definitions for multiplicity are as follows: s=singlet, d=doublet, t=triplet, q=quartet, m=multiplet, br=broad. It will be understood that for compounds comprising an exchangeable proton, said proton may or may not be visible on an NMR spectrum depending on the choice of solvent used for running the NMR spectrum and the concentration of the compound in the solution.

Chemical names were generated using ChemDraw Ultra 12.0, ChemDraw Ultra 14.0 (CambridgeSoft Corp., Cambridge, Mass.) or ACD/Name Version 10.01 (Advanced Chemistry).

Compounds designated as R* or S* are enantiopure compounds where the absolute configuration was not determined.

### Intermediate 1: tert-Butyl 11,11-difluoro-8-methylene-3,4,8,9,10,11-hexahydro-1H-pyrido[4′,3′:3,4]pyrazolo[1,5-a]azepine-2(7H)-carboxylate

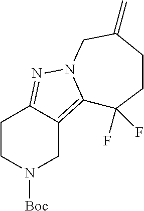 [SMILES: C=C1CCC(F)(F)c2c3c(nn2C1)CCN(C(=O)OC(C)(C)C)C3]

Step A. tert-Butyl 3-(3-ethoxy-3-oxopropanoyl)-6,7-dihydro-2H-pyrazolo[4,3-c]pyridine-5(4H)-carboxylate. To a solution of ethyl acetate (20.88 g, 237.02 mmol, 23.20 mL) in THF (120 mL) was added NaHMDS (1 M, 474.04 mL) at −65° C. under N₂, followed by a solution of 5-tert-butyl 3-ethyl 6,7-dihydro-2H-pyrazolo[4,3-c]pyridine-3,5(4H)-dicarboxylate (28 g, 94.81 mmol) in THF (200 mL) over 1 h at −65° C. The mixture was stirred at 45° C. for 10 h. The mixture was quenched with HCl (1 N, 1.5 L) and diluted in ethyl acetate (1500 mL). The organic phase was dried over Na₂SO₄, filtered and concentrated in vacuo. The residue was purified by column chromatography (SiO₂, Petroleum ether/Ethyl acetate=10/1 to 1/1) to give the title compound (28.4 g, 84.18 mmol, 88.79% yield) as yellow solid. MS (ESI): mass calcd. for C₁₆H₂₃N₃O₅, 337.2; m/z found, 360.1 [M+Na]+.

Step B. Mixture of di-tert-butyl 3-(3-ethoxy-3-oxopropanoyl)-6,7-dihydro-2H-pyrazolo[4,3-c]pyridine-2,5(4H)-dicarboxylate and di-tert-butyl 3-(3-ethoxy-3-oxopropanoyl)-6,7-dihydro-1H-pyrazolo[4,3-c]pyridine-1,5(4H)-dicarboxylate. To a solution of tert-butyl 3-(3-ethoxy-3-oxopropanoyl)-6,7-dihydro-2H-pyrazolo[4,3-c]pyridine-5(4H)-carboxylate (18 g, 53.35 mmol), TEA (16.20 g, 160.06 mmol, 22.28 mL) and DMAP (651.82 mg, 5.34 mmol) in dichloromethane (DCM) (200 mL) was added Boc₂O (11.64 g, 53.35 mmol, 12.26 mL). The mixture was stirred at 15° C. for 2 h. The mixture was poured into HCl (1 N, 250 mL) and extracted with ethyl acetate (200 mL×2). The combined organic phase was washed with brine (200 mL), dried over anhydrous Na₂SO₄, filtered and concentrated under vacuum. The residue was purified by Silica Flash Column (Eluent of 0-20% Ethyl acetate/Petroleum) to give a mixture of di-tert-butyl 3-(3-ethoxy-3-oxopropanoyl)-6,7-dihydro-2H-pyrazolo[4,3-c]pyridine-2,5(4H)-dicarboxylate and di-tert-butyl 3-(3-ethoxy-3-oxopropanoyl)-6,7-dihydro-1H-pyrazolo[4,3-c]pyridine-1,5(4H)-dicarboxylate (20 g, 22.86 mmol, 42.84% yield, 100% purity) colorless oil. MS (ESI): mass calcd. for C₂₁H₃₁N₃O₇, 437.2; m/z found, 460.1 [M+Na]+.

Step C. Mixture of di-tert-butyl3-(4-(((tert-butyldiphenylsilyl)oxy)methyl) -2-(ethoxycarbonyl)pent-4-enoyl)-6,7-dihydro-2H-pyrazolo[4,3-c]pyridine-2,5(4H)-dicarboxylate and di-tert-butyl3-(4-(((tert-butyldiphenylsilyl)oxy)methyl)-2-(ethoxycarbonyl)pent-4-enoyl)-6,7-dihydro-1H-pyrazolo[4,3-c]pyridine-1,5(4H)-dicarboxylate. To a mixture of di-tert-butyl 3-(3-ethoxy-3-oxopropanoyl)-6,7-dihydro-2H-pyrazolo[4,3-c]pyridine-2,5(4H)-dicarboxylate and di-tert-butyl 3-(3-ethoxy-3-oxopropanoyl)-6,7-dihydro-1H-pyrazolo[4,3-c]pyridine-1,5(4H)-dicarboxylate (14.00 g, 32.04 mmol) in acetone (150 mL) were added K₂CO₃ (6.64g, 48.05 mmol), NaI (960.39 mg, 6.41 mmol) and ((2-(bromomethyl)allyl)oxy)(tert-butyl)diphenylsilane (14.97 g, 38.44 mmol). The mixture was stirred at 55° C. for 4 h. The mixture was poured into HCl (1 N, 400 mL) at 0° C. and extracted with ethyl acetate (300 mL×3). The combined organic phase was washed with brine (500 mL), dried over Na₂SO₄, filtered and concentrated in vacuo. The residue was purified by column chromatography (SiO₂, petroleum ether/ethyl acetate=30/1 to 20/1) to give a mixture of di-tert-butyl3-(4-(((tert-butyldiphenylsilyl)oxy)methyl)-2-(ethoxycarbonyl)pent-4-enoyl)-6,7-dihydro-2H-pyrazolo[4,3-c]pyridine-2,5(4H)-dicarboxylate and di-tert-butyl3-(4-(((tert-butyldiphenylsilyl)oxy)methyl)-2-(ethoxycarbonyl)pent-4-enoyl)-6,7-dihydro-1H-pyrazolo[4,3-c]pyridine-1,5(4H)-dicarboxylate (13.5g, 16.83 mmol, 52.53% yield, 93% purity) as yellow oil. MS (ESI): mass calcd. for C₄₁H₅₅N₃O₈Si, 745.4; m/z found, 768.5 [M+Na]+.

Step D. tert-Butyl 3-(4-(((tert-butyldiphenylsilyl)oxy)methyl)pent-4-enoyl)-6,7-dihydro-2H-pyrazolo[4,3-c]pyridine-5(4H)-carboxylate. To a mixture of di-tert-butyl 3-(4-(((tert-butyldiphenylsilyl)oxy)methyl)-2-(ethoxycarbonyl)pent-4-enoyl)-6,7-dihydro-2H-pyrazolo[4,3-c]pyridine-2,5(4H)-dicarboxylate and di-tert-butyl 3-(4-(((tert-butyldiphenylsilyl)oxy)methyl)-2-(ethoxycarbonyl)pent-4-enoyl)-6,7-dihydro-1H-pyrazolo[4,3-c]pyridine-1,5(4H)-dicarboxylate (13.5 g, 16.83 mmol) in MeOH (50 mL) was added a solution of KOH (1.89 g, 33.66 mmol) in H₂O (10 mL), the mixture was stirred at 65° C. for 3 h. The mixture was poured into HCl (1 N, 300 mL) and extracted with ethyl acetate (200 mL×3). The combined organic phase was washed with brine (200 mL), dried over anhydrous Na₂SO₄, filtered and concentrated in vacuo. The residue was purified by silica gel chromatography (SiO₂, petroleum ether/ethyl acetate=1/0 to 3/1) to give the title compound (8.9 g, 15.51 mmol, 92.15% yield) as yellow oil.

Step E. tert-Butyl 3-(4-(((tert-butyldiphenylsilyl)oxy)methyl)-1,1-difluoropent-4-en-1-yl)-6,7-dihydro-2H-pyrazolo[4,3-c]pyridine-5(4H)-carboxylate. To a solution of tert-butyl 3-(4-(((tert-butyldiphenylsilyl)oxy)methyl)pent-4-enoyl)-6,7-dihydro-2H-pyrazolo[4,3-c]pyridine-5(4H)-carboxylate (28 g, 48.80 mmol) in dichloromethane (300 mL) were added DAST (47.19 g, 292.79 mmol, 38.68 mL) and EtOH (449.61 mg, 9.76 mmol, 570.57 μL) at 0° C. under N₂. The mixture was stirred at 0° C. for 2 h. The reaction mixture was added dropwise into NaHCO₃ (sat. aq. 300 mL) at 0° C., then extracted with DCM (150 mL×2). The combined organic phase was washed with brine (200 mL), dried over Na₂SO₄, filtered and concentrated in vacuo. The residue was purified by silica gel chromatography (diameter: 100 mm, 100-200 mesh silica gel, petroleum ether/ethyl acetate=20/1 to 5/1) to give the title compound (18.8 g, 31.55 mmol, 64.66% yield) as yellow oil. MS (ESI): mass calcd. for C₃₃H₄₃F₂N₃O₃Si, 595.3; m/z found, 596.3 [M+H]+. ¹H NMR (400 MHz, CDCl₃) δ=7.68 (dd, J=1.5, 7.9 Hz, 4H), 7.47-7.34 (m, 6H), 5.19 (s, 1H), 4.91 (s, 1H), 4.52 (br s, 2H), 4.20-4.12 (m, 2H), 3.70 (br s, 2H), 2.73 (t, J=5.4 Hz, 2H), 2.51-2.35 (m, 2H), 2.31-2.17 (m, 2H), 1.48 (s, 9H), 1.06 (s, 9H).

Step F. tert-Butyl3-(1,1-difluoro-4-(hydroxymethyl)pent-4-en-1-yl)-6,7-dihydro-2H-pyrazolo[4,3-c]pyridine-5(4H)-carboxylate. To a solution of tert-butyl 3-(4-(((tert-butyldiphenylsilyl)oxy)methyl)-1,1-difluoropent-4-en-1-yl)-6,7-dihydro-2H-pyrazolo[4,3-c]pyridine-5(4H)-carboxylate (17 g, 28.53 mmol) in THF (200 mL) was added TBAF (1 M, 37.09 mL). The mixture was stirred at 15° C. for 4 h. The residue was poured into water (200 mL) and extracted with ethyl acetate (80 mL×3). The combined organic phase was washed with brine (80 mL), dried over anhydrous Na₂SO₄, filtered and concentrated in vacuo. The residue was purified by column chromatography (SiO₂, petroleum ether/ethyl acetate=5/1 to 1/1) to give the title compound (8.2 g, 22.26 mmol, 78.00% yield, 97% purity) as yellow oil. MS (ESI): mass calcd. for C₁₇H₂₅F₂N₃O₃, 357.2; m/z found, 358.3 [M+H]+. ¹H NMR (400 MHz, CDCl₃) δ=5.03 (s, 1H), 4.90 (s, 1H), 4.54 (s, 2H), 4.18-4.14 (m, 2H), 3.71 (s, 2H), 2.75 (t, J=5.2 Hz, 2H), 2.57-2.42 (m, 2H), 2.35 (br s, 2H), 1.49 (s, 9H).

Step G. tert-Butyl 3-(1,1-difluoro-4-(((methylsulfonyl)oxy)methyl)pent-4-en-1-yl) -6,7-dihydro-2H-pyrazolo[4,3-c]pyridine-5(4H)-carboxylate. To a solution of tert-butyl 3-(1,1-difluoro-4-(hydroxymethyl)pent-4-en-1-yl)-6,7-dihydro-2H-pyrazolo[4,3-c]pyridine-5(4H)-carboxylate (8.2 g, 22.94 mmol) in DCM (100 mL) were added TEA (6.54 g, 64.66 mmol, 9 mL) and MsCl (3.15 g, 27.53 mmol, 2.13 mL) at 0° C. under N₂. The mixture was stirred at 0° C. for 1 h. The mixture was poured into water (100 mL) and extracted with ethyl acetate (100 mL×2). The combined organic phase was washed with brine (100 mL), dried over Na₂SO₄, filtered and concentrated in vacuo to give the title compound (10 g, crude) as yellow oil which used in the next step without further purification. MS (ESI): mass calcd. for C₁₈H₂₇F₂N₃O₅S, 435.2; m/z found, 436.0 [M+H]+.

Step H. tert-Butyl 11,11-difluoro-8-methylene-3,4,8,9,10,11-hexahydro-1H-pyrido [4′,3′:3,4]pyrazolo[1,5-a]azepine-2(7H)-carboxylate. To a solution of tert-butyl 3-(1,1-difluoro-4-(((methylsulfonyl)oxy)methyl)pent-4-en-1-yl)-6,7-dihydro-2H-pyrazolo[4,3-c]pyridine-5(4H)-carboxylate (10 g, crude) in MeCN (100 mL) was added DBU (6.42 g, 42.17 mmol, 6.36 mL). The mixture was stirred at 10° C. for 1 h. The mixture was poured into ice-water (150 mL) and extracted with ethyl acetate (100 mL×3). The combined organic phase was washed with brine (100 mL), dried over Na₂SO₄, filtered and concentrated in vacuo. The residue was purified by silica gel chromatography (silica gel, petroleum ether/ethyl acetate=20/1 to 5/1) to give the title compound (5.8 g, 17.09 mmol) as colorless oil. MS (ESI): mass calcd. for C₁₇H₂₃F₂N₃O₂, 339.2; m/z found, 340.2 [M+H]+.

### Intermediate 2: (5)-tert-Butyl 11,11-difluoro-8-hydroxy-3,4,8,9,10,11-hexahydro-1H-pyrido[4′,3′:3,4]pyrazolo[1,5-a]azepine-2(7H)-carboxylate

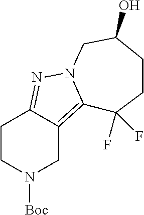 [SMILES: CC(C)(C)OC(=O)N1CCc2nn3c(c2C1)C(F)(F)CC[C@H](O)C3]

Step A. tert-Butyl 11,11-difluoro-8-oxo-3,4,8,9,10,11-hexahydro-1H-pyrido[4′,3′:3,4] pyrazolo[1,5-a]azepine-2(7H)-carboxylate. To a solution of tert-butyl 11,11-difluoro-8-methylene-3,4,8,9,10,11-hexahydro-1H -pyrido[4′,3′:3,4]pyrazolo[1,5-a]azepine-2(7H)-carboxylate (400 mg, 1.10 mmol) in THF (4 mL) and H₂O (2 mL) was added NaIO₄ (937.80 mg, 4.38 mmol, 242.95 4) and OsO₄ (27.87 mg, 109.61 μmol, 5.69 μL) at 0° C. The mixture was stirred at 20° C. for 16 h. Two same batches preformed in parallel were combined. The combined mixture was poured into H₂O (50 mL) at 0° C. and extracted with ethyl acetate (50 mL×4). The combined organic phase was washed with brine (50 mL), dried over Na₂SO₄, filtered and concentrated in vacuo. The residue was purified by silica gel chromatography (100-200 mesh silica gel, petroleum ether/ethyl acetate=3/1 to 2/1) to give the title compound (670 mg, 93% purity) as white solid. MS (ESI): mass calcd. for C₁₆H₂₁F₂N₃O₃, 341.2; m/z found, 360.3 [M+H₂O+H]+.

Step B. tert-Butyl 11,11-difluoro-8-hydroxy-3,4,8,9,10,11-hexahydro-1H-pyrido [4′,3′:3,4]pyrazolo[1,5-a]azepine-2(7H)-carboxylate. To a solution of tert-butyl 11,11-difluoro-8-oxo-3,4,8,9,10,11-hexahydro-1H-pyrido[4′,3′:3,4]pyrazolo[1,5-a]azepine-2(7H)-carboxylate (670 mg, 1.83 mmol) in THF (10 mL) was added NaBH₄ (138.12 mg, 3.65 mmol) at 0° C. The mixture was stirred at 0° C. for 0.5 h. The mixture was poured into water (30 mL) and extracted with ethyl acetate (30 mL×4). The combined organic phase was washed with brine (50 mL), dried over anhydrous Na₂SO₄, filtered and concentrated in vacuo. The residue was purified by silica gel chromatography (100-200 mesh silica gel, petroleum ether/ethyl acetate=3/1 to 2/1) to give the title compound (430 mg, 1.18 mmol, 64.49% yield, 94% purity) as white solid. MS (ESI): mass calcd. for C₁₆H₂₃F₂N₃O₃, 343.2; m/z found, 344.3 [M+H]+.

Step C. (5)-tert-Butyl 11,11-difluoro-8-hydroxy-3,4,8,9,10,11-hexahydro-1H-pyrido [4′,3′:3,4]pyrazolo[1,5-a]azepine-2(7H)-carboxylate. Tert-butyl-11,11-difluoro-8-hydroxy-3,4,8,9,10,11-hexahydro-1H-pyrido[4′,3′:3,4]pyrazolo[1,5-a]azepine-2(7H)-carboxylate was resolved by SFC (condition: column: AD(250 mm*30 mm,10 um);mobile phase: [0.1% NH₃.H₂O MeOH]; B %: 35%-35%, 2.5 min; 60 min) to give (5)-tert-butyl 11,11-difluoro-8-hydroxy-3,4,8,9,10,11-hexahydro-1H-pyrido[4′,3′:3,4]pyrazolo[1,5-a]azepine-2(7H)-carboxylate (Peak 1 on SFC (AD-3S_3_5_40_3ML Column: Chiralpak AD-3 100×4.6 mm I.D., 3 um Mobile phase: methanol (0.05% DEA) in CO₂ from 5% to 40%; Flow rate: 3 mL/min Wavelength: 220 nm), retention time=1.388 min, 240 mg, 643.04 μmol, 45.17% yield, 92% purity) as white solid and (R)-tert-butyl 11,11-difluoro-8-hydroxy-3,4,8,9,10,11-hexahydro-1H-pyrido[4′,3′:3,4]pyrazolo[1,5-a]azepine-2(7H)-carboxylate (Peak 2 on SFC, retention time=1.968 min, 220 mg, 634.30 μmol, 44.56% yield, 99% purity) as white solid.

### Intermediate 3: (R)-tert-Butyl11,11-difluoro-8-hydroxy-3,4,8,9,10,11-hexahydro-1H-pyrido[4′,3′:3,4]pyrazolo[1,5-a]azepine-2(7H)-carboxylate

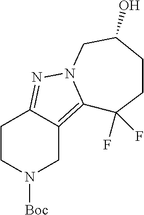 [SMILES: CC(C)(C)OC(=O)N1CCc2nn3c(c2C1)C(F)(F)CC[C@@H](O)C3]

The title compound was separated by SFC from tert-butyl-11,11-difluoro-8-hydroxy-3,4,8,9,10,11-hexahydro-1H-pyrido[4′,3′:3,4]pyrazolo[1,5-a]azepine-2(7H)-carboxylate in a manner analogous to Intermediate 2, Step C: (Peak 2 on SFC, retention time=1.968 min, 220 mg, 634.30 μmol, 44.56% yield, 99% purity) as white solid.

### Intermediate 4: (5)-tert-Butyl 8-(2,2-difluoroethoxy)-11,11-difluoro-3,4,8,9,10,11-hexahydro-1H-pyrido[4′,3′:3,4]pyrazolo[1,5-a]azepine-2(7H)-carboxylate

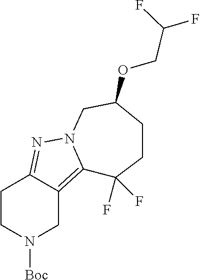 [SMILES: CC(C)(C)OC(=O)N1CCc2nn3c(c2C1)C(F)(F)CC[C@H](OCC(F)F)C3]

To a solution of (5)-tert-butyl 11,11-difluoro-8-hydroxy-3,4,8,9,10,11-hexahydro-1H-pyrido[4′,3′:3,4]pyrazolo[1,5-a]azepine-2(7H)-carboxylate (Intermediate 2, 100 mg, 267.93 μmol) in THF (1 mL) was added NaH (23.3 mg, 582.50 μmol, 60% purity). The mixture was stirred at 0° C. for 0.5 h, and then 2,2-difluoroethyl trifluoromethanesulfonate (172.10 mg, 803.80 μmol) was added into the mixture. The mixture was stirred at 0° C. for 4 h. LCMS showed the starting material was consumed completely and a main peak with desired mass was detected. The mixture was poured into ice-water (20 mL) and extracted with ethyl acetate (20 mL×3). The combined organic phase was washed with brine (30 mL), dried over Na₂SO₄, filtered and concentrated in vacuo to give the title compound (100 mg, crude) as colorless oil. MS (ESI): mass calcd. for C₁₈H₂₅N₃O₃F₄, 407.2; m/z found, 408.3 [M+H]+.

### Intermediate 5: (R)-tert-butyl 8-(2,2-difluoroethoxy)-11,11-difluoro-3,4,8,9,10,11-hexahydro-1H-pyrido[4′,3′:3,4]pyrazolo[1,5-a]azepine-2(7H)-carboxylate

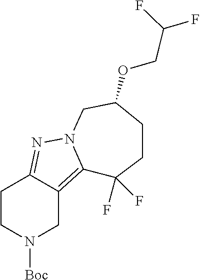 [SMILES: CC(C)(C)OC(=O)N1CCc2nn3c(c2C1)C(F)(F)CC[C@@H](OCC(F)F)C3]

The title compound was prepared in a manner analogous to Intermediate 4, however using (R)-tert-butyl 11,11-difluoro-8-hydroxy-3,4,8,9,10,11-hexahydro-1H-pyrido[4′,3′:3,4]pyrazolo[1,5-a]azepine-2(7H)-carboxylate (Intermediate 3) instead of (S)-tert-butyl 11,11-difluoro-8-hydroxy-3,4,8,9,10,11-hexahydro-1H-pyrido[4′,3′:3,4] pyrazolo[1,5-a]azepine-2(7H)-carboxylate (Intermediate 2). MS (ESI): mass calcd. for C₁₈H₂₅N₃O₃F₄, 407.2; m/z found, 408.3 [M+H]+.

### Intermediate 6: (R)-tert-Butyl 8,11,11-trifluoro-3,4,8,9,10,11-hexahydro-1H-pyrido [4′,3′:3,4]pyrazolo[1,5-a]azepine-2(7H)-carboxylate

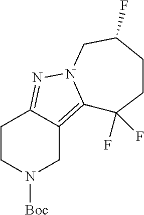 [SMILES: CC(C)(C)OC(=O)N1CCc2nn3c(c2C1)C(F)(F)CC[C@@H](F)C3]

To a solution of (5)-tert-butyl 11,11-difluoro-8-hydroxy-3,4,8,9,10,11-hexahydro-1H-pyrido[4′,3′:3,4]pyrazolo[1,5-a]azepine-2(7H)-carboxylate (Intermediate 2, 240 mg, 677.99 μmol) in DCM (10 mL) was added DAST (437.14 mg, 2.71 mmol, 358.31 μL) at −40° C. The mixture was stirred at 20° C. for 1 h. The reaction was poured into NaHCO₃ (sat.aq, 30 mL) and extracted with dichloromethane (30 mL×3). The combined organic phase was washed with brine (30 mL), dried over anhydrous Na₂SO₄, filtered and concentrated in vacuo. The residue was purified by silica gel chromatography (100-200 mesh silica gel, Petroleum ether/Ethyl acetate=30/1 to 10/1) to give the title compound (170 mg, 80% purity) as colorless oil. MS (ESI): mass calcd. for C₁₆H₂₂F₃N₃O₂, 345.2; m/z found, 346.2 [M+H]+.

### Intermediate 7: (S)-tert-Butyl 8,11,11-trifluoro-3,4,8,9,10,11-hexahydro-1H-pyrido [4′,3′:3,4]pyrazolo[1,5-a]azepine-2(7H)-carboxylate

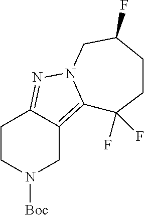 [SMILES: CC(C)(C)OC(=O)N1CCc2nn3c(c2C1)C(F)(F)CC[C@H](F)C3]

The title compound was prepared in a manner analogous to Intermediate 6, however using (R)-tert-butyl 11,11-difluoro-8-hydroxy-3,4,8,9,10,11-hexahydro-1H-pyrido[4′,3′:3,4]pyrazolo[1,5-a]azepine-2(7H)-carboxylate (Intermediate 3) instead of (S)-tert-butyl 11,11-difluoro-8-hydroxy-3,4,8,9,10,11-hexahydro-1H-pyrido[4′,3′:3,4] pyrazolo[1,5-a]azepine-2(7H)-carboxylate (Intermediate 2). MS (ESI): mass calcd. for C₁₆H₂₂F₃N₃O₂, 345.2; m/z found, 346.0 [M+H]+.

### Intermediate 8: (S*)-tert-Butyl 11,11-difluoro-8-(hydroxymethyl)-3,4,8,9,10,11-hexahydro-1H-pyrido[4′,3′:3,4]pyrazolo[1,5-a]azepine-2(7H)-carboxylate

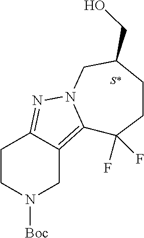 [SMILES: CC(C)(C)OC(=O)N1CCc2nn3c(c2C1)C(F)(F)CC[C@H](CO)C3]

### Step A. tert-butyl 11,11-difluoro-8-(hydroxymethyl)-3,4,8,9,10,11-hexahydro-1H-pyrido[4′,3′:3,4]pyrazolo[1,5-a]azepine-2(7H)-carboxylate

To a solution of tert-butyl 11,11-difluoro-8-methylene-3,4,8,9,10,11-hexahydro-1H-pyrido[4′,3′:3,4]pyrazolo[1,5-a]azepine-2(7H)-carboxylate (400 mg, 1.18 mmol) in THF (5 mL) was added 9-BBN (0.5 M, 23.57 mL) at 0° C., the mixture was stirred at 0° C. for 2 h. A solution of NaOH (471.41 mg, 11.79 mmol) in H₂O (0.5 mL) was added at −30° C. followed by H₂O₂ (1.60 g, 14.14 mmol, 1.36 mL, 30% purity), the reaction mixture was stirred at 25° C. for 1 h. The mixture was diluted with H₂O (80 mL) and extracted with EtOAc (70 mL×2), the combined organic layer was dried over Na₂SO₄, filtered and concentrated in vacuo. The residue was purified by column chromatography (SiO₂, Petroleum ether/Ethyl acetate=100/1 to 1/1) to give the title compound (530 mg, 1.48 mmol, 66.20% yield) as white solid. MS (ESI): mass calcd. for C₁₇H₂₅F₂N₃O₃, 357.2; m/z found, 358.1 [M+H]+.

Step B. (S*)-tert-butyl 11,11-difluoro-8-(hydroxymethyl)-3,4,8,9,10,11-hexahydro-1H-pyrido[4′,3′:3,4]pyrazolo[1,5-a]azepine-2(7H)-carboxylate

Tert-butyl 11,11-difluoro-8-(hydroxymethyl)-3,4,8,9,10,11-hexahydro-1H-pyrido [4′,3′:3,4]pyrazolo[1,5-a]azepine-2(7H)-carboxylate was resolved by SFC (condition: column: AD (250 mm*30 mm, 5 um); mobile phase: [0.1% NH₃.H₂O MeOH]; B %: 20%-20%,1.5 min; 250 min) to give (S*)-tert-butyl 11,11-difluoro-8-(hydroxymethyl)-3,4,8,9,10,11-hexahydro-1H-pyrido[4′,3′:3,4]pyrazolo[1,5-a]azepine-2(7H)-carboxylate (Peak 1 on SFC (Amycoat_MeOH (DEA)_5_40_3mL-35T Column: Amycoat 50×4.6 mm I.D., 3 um Mobile phase: methanol (0.05% DEA) in CO₂ from 5% to 40% Flow rate: 3 mL/min Wavelength: 220 nm), retention time=0.872 min, 165 mg, 98% purity) as white solid, and (R*)-tert-butyl 11,11-difluoro-8-(hydroxymethyl)-3,4,8,9,10,11-hexahydro-1H-pyrido[4′,3′:3,4]pyrazolo[1,5-a]azepine-2(7H)-carboxylate (Peak 2 on SFC, retention time=0.932 min, 205 mg, 97% purity) as white solid and the racemate (116 mg, 324.57 μmol) as white solid.

### Intermediate 9: (R*)-tert-Butyl 11,11-difluoro-8-(hydroxymethyl)-3,4,8,9,10,11-hexahydro-1H-pyrido[4′,3′:3,4]pyrazolo[1,5-a]azepine-2(7H)-carboxylate

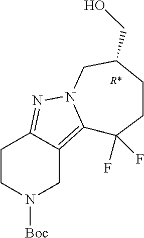 [SMILES: CC(C)(C)OC(=O)N1CCc2nn3c(c2C1)C(F)(F)CC[C@@H](CO)C3]

The title compound was separated from tert-butyl 11,11-difluoro-8-(hydroxymethyl)-3,4,8,9,10,11-hexahydro-1H-pyrido[4′,3′:3,4]pyrazolo[1,5-a]azepine-2(7H)-carboxylate in a manner analogous to Intermediate 8, Step B by SFC(Peak 2 on SFC, retention time=0.932 min, 205 mg, 97% purity) as white solid.

### Intermediate 10: (S*)-tert-Butyl 8-((2,2-difluoroethoxy)methyl)-11,11-difluoro-3,4,8,9,10,11-hexahydro-1H-pyrido[4′,3′:3,4]pyrazolo[1,5-a]azepine-2(7H)-carboxylate

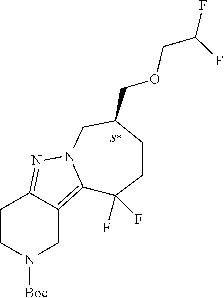 [SMILES: CC(C)(C)OC(=O)N1CCc2nn3c(c2C1)C(F)(F)CC[C@H](COCC(F)F)C3]

To the solution of (S*)-tert-butyl 11,11-difluoro-8-(hydroxymethyl)-3,4,8,9,10,11-hexahydro-1H-pyrido[4′,3′:3,4]pyrazolo[1,5-a]azepine-2(7H)-carboxylate (120 mg, 335.76 μmol) in DMF (2 mL) was added NaH (80.58 mg, 2.01 mmol, 60% purity) at −40° C. under N₂, and then the mixture was stirred at −40° C. for 0.5 h. 2,2-difluoroethyl trifluoromethanesulfonate (215.67 mg, 1.01 mmol) was added to the mixture, the mixture was stirred at −40° C. for 2 h under N₂. The mixture was poured into ice-water (10 mL) and stirred for 1 min. The aqueous phase was extracted with ethyl acetate (5 mL×2). The combined organic phase was washed with brine (10 mL), dried over anhydrous Na₂SO₄, filtered and concentrated under vacuum. The residue was purified by column chromatography (SiO₂, Petroleum ether/Ethyl acetate=100/1 to 1/1) to give the title compound (144 mg, crude) as yellow oil, which was used directly for next step. MS (ESI): mass calcd. for C₁₉H₂₇F₄N₃O₃ 421.2; m/z found, 422.1 [M+H]+.

### Intermediate 11: (R*)-tert-butyl 8-((2,2-difluoroethoxy)methyl)-11,11-difluoro-3,4,8,9,10,11-hexahydro-1H-pyrido[4′,3′:3,4]pyrazolo[1,5-a]azepine-2(7H)-carboxylate

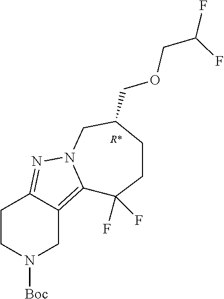 [SMILES: CC(C)(C)OC(=O)N1CCc2nn3c(c2C1)C(F)(F)CC[C@@H](COCC(F)F)C3]

The title compound was prepared in a manner analogous to Intermediate 10, however using (R*)-tert-butyl 11,11-difluoro-8-(hydroxymethyl)-3,4,8,9,10,11-hexahydro-1H-pyrido[4′,3′:3,4]pyrazolo[1,5-a]azepine-2(7H)-carboxylate (Intermediate 9) for (S*)-tert-butyl 11,11-difluoro-8-(hydroxymethyl)-3,4,8,9,10,11-hexahydro-1H-pyrido[4′,3′:3,4]pyrazolo[1,5-a]azepine-2(7H)-carboxylate (Intermediate 8). MS (ESI): mass calcd. for C₁₉H₂₇F₄N₃O₃ 421.2; m/z found, 422.1 [M+H]+.

### Intermediate 12: (R*)-tert-Butyl 8-((2,2-difluoroethoxy)methyl)-11,11-difluoro-8-hydroxy-3,4,8,9,10,11-hexahydro-1H-pyrido[4′,3′:3,4]pyrazolo[1,5-a]azepine-2(7H)-carboxylate

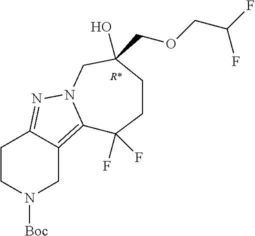 [SMILES: CC(C)(C)OC(=O)N1CCc2nn3c(c2C1)C(F)(F)CC[C@](O)(COCC(F)F)C3]

### Step A. tert-Butyl 11,11-difluoro-8-hydroxy-8-(hydroxymethyl)-3,4,8,9,10,11-hexahydro-1H-pyrido[4′,3′:3,4]pyrazolo[1,5-a]azepine-2(7H)-carboxylate

To a solution of tert-butyl 11,11-difluoro-8-methylene-3,4,8,9,10,11-hexahydro-1H-pyrido[4′,3′:3,4]pyrazolo[1,5-a]azepine-2(7H)-carboxylate (1 g, 2.68 mmol) in THF (10 mL) and H₂O (5 mL) were added K₂OsO₄.2H₂O (98.80 mg, 268.14 μmol) and NMO (471.18 mg, 4.02 mmol, 424.49 μL). The mixture was stirred at 25° C. for 16 h. The mixture was diluted in H₂O (60 mL) and extracted with EtOAc (80 mL×3), the combined organic layer was washed Na₂SO₃ (sat.aq, 50 mL×3), dried over Na₂SO₄, filtered and concentrated in vacuo. The residue was diluted with EtOAc (10 mL) and filtered, the solid was collected to give the title compound (800 mg, 2.14 mmol, 79.90% yield) as white solid. MS (ESI): mass calcd. for C₁₇H₂₅F₂N₃O₄ 373.2; m/z found, 374.1 [M+H]+.

### Step B. tert-Butyl 8-((2,2-difluoroethoxy)methyl)-11,11-difluoro-8-hydroxy-3,4,8,9,10,11-hexahydro-1H-pyrido[4′,3′:3,4]pyrazolo[1,5-a]azepine-2(7H)-carboxylate

To a solution of tert-butyl 11,11-difluoro-8-hydroxy-8-(hydroxymethyl)-3,4,8,9,10,11-hexahydro-1H-pyrido[4′,3′:3,4]pyrazolo[1,5-a]azepine-2(7H)-carboxylate (643 mg, 1.63 mmol) in THF (7 mL) and DMF (4 mL) was added NaHMDS (1 M, 2.12 mL) at −78° C., the mixture was stirred at −78° C. for 30 min, then a solution of 2,2-difluoroethyl trifluoromethanesulfonate (524.30 mg, 2.45 mmol) in THF (1 mL) was added at −78° C., the mixture was stirred at −78° C. for 0.5 h. The mixture was quenched with NH₄Cl (sat. aq, 40 mL) and extracted with EtOAc (60 mL×2), the combined organic layer was washed with H₂O (60 mL×2), dried over Na₂SO₄, filtered and concentrated in vacuo. The residue was diluted with EtOAc (10 mL) and filtered. The filtrate was concentrated in vacuo. The residue was purified with the other two batches of tert-butyl 11,11-difluoro-8-hydroxy-8-(hydroxymethyl)-3,4,8,9,10,11-hexahydro-1H-pyrido[4′,3′:3,4]pyrazolo[1,5-a]azepine-2(7H)-carboxylate 200 mg scale and 1.1 g scale) by column chromatography (SiO₂, petroleum ether/ethyl acetate=2/3 to 1/9) to give the title compound (800 mg) as colorless oil. MS (ESI): mass calcd. for C₁₉H₂₇F₄N₃O₄ 437.2; m/z found, 438.1 [M+H]+.

### Step C. (R*)-tert-Butyl 8-((2,2-difluoroethoxy)methyl)-11,11-difluoro-8-hydroxy-3,4,8,9,10,11-hexahydro-1H-pyrido[4′,3′:3,4]pyrazolo[1,5-a]azepine-2(7H)-carboxylate

Tert-butyl 8-((2,2-difluoroethoxy)methyl)-11,11-difluoro-8-hydroxy-3,4,8,9,10,11-hexahydro-1H-pyrido[4′,3′:3,4]pyrazolo[1,5-a]azepine-2(7H)-carboxylate (800 mg) was resolved by SFC (condition: column: DAICEL CHIRALCEL OD-H(250 mm*30 mm,5 um);mobile phase: [0.1% NH₃.H₂O IPA]; B %: 15%-15%, 2.3 min; 900 min) to give (R*)-tert-butyl 8-((2,2-difluoroethoxy)methyl)-11,11-difluoro-8-hydroxy-3,4,8,9,10,11-hexahydro-1H-pyrido[4′,3′:3,4]pyrazolo[1,5-a]azepine-2(7H)-carboxylate (Peak 1 on SFC (Cellucoat_IPA(DEA)_5_40_3mL-35T Column: Cellucoat 50×4.6 mm I.D., 3 um Mobile phase: iso-propanol (0.05% DEA) in CO₂ from 5% to 40% Flow rate: 3 mL/min Wavelength: 220 nm), retention time=0.900 min, 0.253 g, 93.2% purity) as white solid and (S*)-tert-butyl 8-((2,2-difluoroethoxy) methyl)-11,11-difluoro-8-hydroxy-3,4,8,9,10,11-hexahydro-1H-pyrido[4′,3′:3,4]pyrazolo[1,5-a]azepine-2(7H)-carboxylate (Peak 2 on SFC (Cellucoat_IPA(DEA)_5_40_3mL-35T Column: Cellucoat 50×4.6 mm I.D., 3 um Mobile phase: iso-propanol (0.05% DEA) in CO₂ from 5% to 40% Flow rate: 3 mL/min Wavelength: 220 nm), retention time=0.942 min, 0.431 g, 96.4% purity) as yellow oil.

### Intermediate 13: (S*)-tert-Butyl 8-((2,2-difluoroethoxy)methyl)-11,11-difluoro-8-hydroxy-3,4,8,9,10,11-hexahydro-1H-pyrido[4′,3′:3,4]pyrazolo[1,5-a]azepine-2(7H)-carboxylate

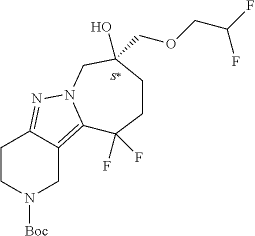 [SMILES: CC(C)(C)OC(=O)N1CCc2nn3c(c2C1)C(F)(F)CC[C@@](O)(COCC(F)F)C3]

The title compound was separated by SFC from tert-butyl 8-((2,2-difluoroethoxy)methyl)-11,11-difluoro-8-hydroxy-3,4,8,9,10,11-hexahydro-1H-pyrido[4′,3′:3,4]pyrazolo[1,5-a]azepine-2(7H)-carboxylate (product from Intermediate 12, Step B) in a manner analogous to Intermediate 12, Step C: (Peak 2 on SFC, retention time=0.942 min, 0.431 g, 96.4% purity) as yellow oil.

### Intermediate 14: tert-Butyl11,11-difluoro-8-(fluoromethyl)-8-hydroxy-3,4,8,9,10,11-hexahydro-1H-pyrido[4′,3′:3,4]pyrazolo[1,5-a]azepine-2(7H)-carboxylate

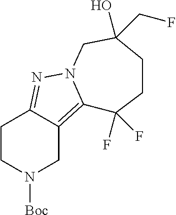 [SMILES: CC(C)(C)OC(=O)N1CCc2nn3c(c2C1)C(F)(F)CCC(O)(CF)C3]

Step A. tert-Butyl 11′,11′-difluoro-3′,4′,7′,9′,10′,11′-hexahydrospiro[oxirane-2,8′-pyrido[4′,3′:3,4]pyrazolo[1,5-a]azepine]-2′(1′H)-carboxylate. To a solution of tert-butyl 11,11-difluoro-8-hydroxy-8-(hydroxymethyl)-3,4,8,9,10,11-hexahydro-1H-pyrido[4′,3′:3,4]pyrazolo[1,5-a]azepine-2(7H)-carboxylate (500 mg, 1.34 mmol) in THF (5 mL) was added DBU (407.71 mg, 2.68 mmol, 403.67 μL). The resulting solution was cooled to 0° C., then 1,1,2,2,3,3,4,4,4-nonafluorobutane-1-sulfonyl fluoride (728.14 mg, 2.41 mmol, 423.34 4) was added dropwise. The reaction was stirred for 1 h at 15° C. LCMS showed a main peak with desired mass was detected. The mixture was concentrated in vacuo. The residue was purified by column chromatography (SiO₂, petroleum ether/ethyl acetate=1/0 to 1/1) to give the title compound (460 mg, 1.22 mmol, 91.15% yield, 94.3% purity) as white solid. MS (ESI): mass calcd. for C₁₇H₂₃F₂N₃O₃ 355.2; m/z found, 356.3 [M+H]+.

Step B. tert-Butyl11,11-difluoro-8-(fluoromethyl)-8-hydroxy-3,4,8,9,10,11-hexahydro-1H-pyrido[4′,3′:3,4]pyrazolo[1,5-a]azepine-2(7H)-carboxylate. The solution of tert-butyl 11′,11′-difluoro-3′,4′,7′,9′,10′,11′-hexahydrospiro[oxirane-2,8′-pyrido[4′,3′:3,4]pyrazolo[1,5-a]azepine]-2′(1′H)-carboxylate (200 mg, 530.70 μmol) in Et₃N₃HF(9.89 g, 61.35 mmol, 10 mL) was heated to 100° C. for 5 h. The mixture was combined with another batch of tert-butyl 11′,11′-difluoro-3′,4′,7′,9′,10′,11′-hexahydrospiro[oxirane-2,8′-pyrido[4′,3′:3,4]pyrazolo[1,5-a]azepine]-2′(1′H)-carboxylate (100 mg scale). The combined solution was diluted in H₂O (25 mL) and extracted with EtOAc (30 mL×3), the combined organic layer was dried over Na₂SO₄ and filtered. The filtrate was concentrated in vacuo. The residue was purified by column chromatography (SiO₂, petroleum ether/ethyl acetate=10/1 to 1/1) to give the title compound (180 mg) as white solid.

### Intermediate 15: (S*)-tert-Butyl 11,11-difluoro-8-(fluoromethyl)-8-hydroxy-3,4,8,9,10,11-hexahydro-1H-pyrido[4′,3′:3,4]pyrazolo[1,5-a]azepine-2(7H)-carboxylate

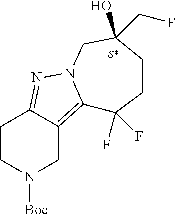 [SMILES: CC(C)(C)OC(=O)N1CCc2nn3c(c2C1)C(F)(F)CC[C@@](O)(CF)C3]

Tert-butyl 11,11-difluoro-8-(fluoromethyl)-8-hydroxy-3,4,8,9,10,11-hexahydro-1H-pyrido[4′,3′:3,4]pyrazolo[1,5-a]azepine-2(7H)-carboxylate (180 mg) was resolved by SFC (condition: column: DAICEL CHIRALPAK AD(250 mm*30 mm,10 um); mobile phase: [0.1% NH₃.H₂O EtOH]; B %: 30%-30%, 4 min:50 min) to give (S*)-tert-butyl 11,11-difluoro-8-(fluoromethyl)-8-hydroxy-3,4,8,9,10,11-hexahydro-1H-pyrido[4′,3′:3,4]pyrazolo[1,5-a]azepine-2(7H)-carboxylate (Peak 1 on SFC (Amycoat_EtOH(DEA)_5_40_3mL-35T Column:Amycoat 50×4.6 mm I.D., 3 um Mobile phase: ethanol (0.05% DEA) in CO₂ from 5% to 40% Flow rate: 3 mL/min Wavelength: 220 nm), retention time=0.856 min, 71 mg, 93% purity) as yellow solid and (R*)-tert-butyl 11,11-difluoro-8-(fluoromethyl)-8-hydroxy-3,4,8,9,10,11-hexahydro-1H-pyrido[4′,3′:3,4]pyrazolo[1,5-a]azepine-2(7H)-carboxylate (Peak 2 on SFC (Amycoat_EtOH(DEA)_5_40_3mL-35T Column:Amycoat 50×4.6 mm I.D., 3 um Mobile phase: ethanol (0.05% DEA) in CO₂ from 5% to 40% Flow rate: 3 mL/min Wavelength: 220 nm), retention time=1.235 min, 77 mg, 97% purity) as yellow solid.

### Intermediate 16: (R*)-tert-Butyl 11,11-difluoro-8-(fluoromethyl)-8-hydroxy-3,4,8,9,10,11-hexahydro-1H-pyrido[4′,3′:3,4]pyrazolo[1,5-a]azepine-2(7H)-carboxylate

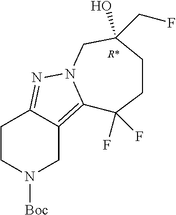 [SMILES: CC(C)(C)OC(=O)N1CCc2nn3c(c2C1)C(F)(F)CC[C@](O)(CF)C3]

The title compound was resolved from tert-butyl 11,11-difluoro-8-(fluoromethyl)-8-hydroxy-3,4,8,9,10,11-hexahydro-1H-pyrido[4′,3′:3,4]pyrazolo[1,5-a]azepine-2(7H)-carboxylate (product from Intermediate 14, Step B) in a manner analogous to Intermediate 15 by SFC: (Peak 2 on SFC, retention time=1.235 min, 77 mg, 97% purity) as yellow solid.

### Intermediate 17: tert-Butyl 8-(cyanomethyl)-11,11-difluoro-8-hydroxy-3,4,8,9,10,11-hexahydro-1H-pyrido[4′,3′:3,4]pyrazolo[1,5-a]azepine-2(7H)-carboxylate

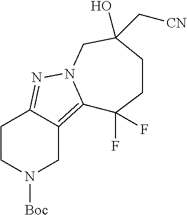 [SMILES: CC(C)(C)OC(=O)N1CCc2nn3c(c2C1)C(F)(F)CCC(O)(CC#N)C3]

A mixture of tert-butyl 11′,11′-difluoro-3′,4′,7′,9′,10′,11′-hexahydrospiro[oxirane-2,8′-pyrido[4′,3′:3,4]pyrazolo[1,5-a]azepine]-2′(1′H)-carboxylate (150 mg, 388.32 μmol), KCN (41.23 mg, 633.18 μmol, 27.13 μL) and LiClO₄ (67.36 mg, 633.13 μmol, 27.83 μL) in MeCN (3 mL) was degassed and purged with N₂ 3 times, and then the mixture was stirred at 60° C. for 16 h under N₂ atmosphere. LCMS showed the starting material was consumed completely. The mixture was poured into ice-water (10 mL) and stirred for 1 min. The aqueous phase was extracted with ethyl acetate (5 mL×2). The combined organic phase was washed with brine (10 mL), dried over Na₂SO₄, filtered and concentrated under vacuum. The aqueous phase was quenched with NaClO (50 mL). The residue was purified by column chromatography (SiO₂, Petroleum ether/Ethyl acetate=100/1 to 1/1) to give the title compound (126 mg, 321.92 μmol, 82.90% yield, 97.7% purity) as white solid.

### Intermediate 18: (R*)-tert-Butyl 8-(cyanomethyl)-11,11-difluoro-8-hydroxy-3,4,8,9,10,11-hexahydro-1H-pyrido[4′,3′:3,4]pyrazolo[1,5-a]azepine-2(7H)-carboxylate

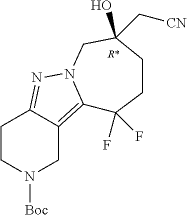 [SMILES: CC(C)(C)OC(=O)N1CCc2nn3c(c2C1)C(F)(F)CC[C@@](O)(CC#N)C3]

Tert-butyl 8-(cyanomethyl)-11,11-difluoro-8-hydroxy-3,4,8,9,10,11-hexahydro-1H-pyrido[4′,3′:3,4]pyrazolo[1,5-a]azepine-2(7H)-carboxylate (400 mg) was resolved by SFC (condition: column: DAICEL CHIRALPAK AD(250 mm*30 mm,10 um); mobile phase: [0.1% NH₃.H₂O EtOH]; B %: 20%-20%,3.0 min; 60 min) to give (R*)-tert-butyl 8-(cyanomethyl)-11,11-difluoro-8-hydroxy-3,4,8,9,10,11-hexahydro-1H-pyrido[4′,3′:3,4]pyrazolo[1,5-a]azepine-2(7H)-carboxylate (Peak 1 on SFC (Amycoat_EtOH(DEA)_5_40_3mL-35T Column:Amycoat 50×4.6 mm I.D., 3 um Mobile phase: ethanol (0.05% DEA) in CO₂ from 5% to 40% Flow rate: 3 mL/min Wavelength: 220 nm), retention time=0.993 min, 170 mg, 98.63% purity) as yellow solid and (S*)-tert-butyl 8-(cyanomethyl)-11,11-difluoro-8-hydroxy-3,4,8,9,10,11-hexahydro-1H-pyrido[4′,3′:3,4]pyrazolo[1,5-a]azepine-2(7H)-carboxylate (Peak 2 on SFC, retention time=1.188 min, 203 mg, 98.72% purity) as yellow solid.

### Intermediate 19: (S*)-tert-Butyl 8-(cyanomethyl)-11,11-difluoro-8-hydroxy-3,4,8,9,10,11-hexahydro-1H-pyrido[4′,3′:3,4]pyrazolo[1,5-a]azepine-2(7H)-carboxylate

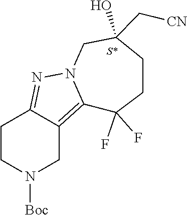 [SMILES: CC(C)(C)OC(=O)N1CCc2nn3c(c2C1)C(F)(F)CC[C@](O)(CC#N)C3]

The title compound was separated by SFC from tert-butyl 8-(cyanomethyl) -11,11-difluoro-8-hydroxy-3,4,8,9,10,11-hexahydro-1H-pyrido[4′,3′:3,4]pyrazolo[1,5-a]azepine-2(7H)-carboxylate (product from Intermediate 17) in a manner analogous to Intermediate 18: (Peak 2 on SFC, retention time=1.188 min, 203 mg, 98.72% purity) as yellow solid.

### Intermediate 20: tert-Butyl 8,11,11-trifluoro-8-(hydroxymethyl)-3,4,8,9,10,11-hexahydro-1H-pyrido[4′,3′:3,4]pyrazolo[1,5-a]azepine-2(7H)-carboxylate

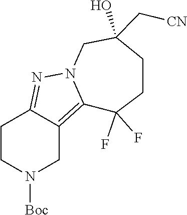 [SMILES: CC(C)(C)OC(=O)N1CCc2nn3c(c2C1)C(F)(F)CC[C@](O)(CC#N)C3]

Step A. 2-(tert-Butoxycarbonyl)-11,11-difluoro-2,3,4,7,8,9,10,11-octahydro-1H-pyrido[4′,3′:3,4]pyrazolo[1,5-a]azepine-8-carboxylic acid. To a solution of tert-butyl 11,11-difluoro-8-(hydroxymethyl)-3,4,8,9,10,11-hexahydro-1H-pyrido[4′,3′:3,4]pyrazolo[1,5-a]azepine-2(7H)-carboxylate (800 mg, 2.24 mmol) in MeCN (8 mL) were added TPAP (196.66 mg, 559.61 μmol) and NMO (1.31 g, 11.19 mmol). The mixture was stirred at 20° C. for 0.5 h. The mixture was concentrated in vacuo to give the title compound (2 g, crude) as black oil which was used in the next step directly. MS (ESI): mass calcd. for C₁₇H₂₃F₂N₃O₄, 371.2; m/z found, 372.3 [M+H]+.

Step B. 2-tert-Butyl 8-ethyl 11,11-difluoro-3,4,8,9,10,11-hexahydro-1H-pyrido [4′,3′:3,4]pyrazolo[1,5-a]azepine-2,8(7H)-dicarboxylate. To a solution of 2-(tert-butoxycarbonyl)-11,11-difluoro-2,3,4,7,8,9,10,11-octahydro-1H-pyrido[4′,3′:3,4]pyrazolo[1,5-a]azepine-8-carboxylic acid (2 g, crude) in MeCN (20 mL) was added Cs₂CO₃ (1.75 g, 5.37 mmol) followed by EtI (839.95 mg, 5.39 mmol, 430.74 μL). The mixture was stirred at 20° C. for 16 h. The mixture was diluted with H₂O (150 mL) and extracted with ethyl acetate (150 mL×3), the combined organic layers were dried over Na₂SO₄, filtered and concentrated in vacuo. The residue was combined with another batch of 2-tert-butoxycarbonyl-11,11-difluoro-3,4,7,8,9,10-hexahydro-1H-pyrido[2,3]pyrazolo[2,4-a]azepine-8-carboxylic acid (500 mg scale) to purify by column chromatography (SiO₂, Petroleum ether/Ethyl acetate=1/0 to 1/4) to give the title compound (530 mg) as yellow oil. MS (ESI): mass calcd. for C₁₉H₂₇F₂N₃O₄, 399.2; m/z found, 400.3 [M+H]+.

Step C. 2-tert-Butyl 8-ethyl 8,11,11-trifluoro-3,4,8,9,10,11-hexahydro-1H-pyrido [4′,3′:3,4]pyrazolo[1,5-a]azepine-2,8(7H)-dicarboxylate. To a solution of lithium diisopropylamide (LDA) (1 M, 1.88 mL) in THF (0.5 mL) was added a solution of 2-tert-butyl 8-ethyl 11,11-difluoro-3,4,8,9,10,11-hexahydro-1H-pyrido[4′,3′:3,4]pyrazolo[1,5-a]azepine-2,8(7H)-dicarboxylate (250 mg, 625.89 μmol) in THF (1.5 mL) at −78° C. The mixture was stirred at −78° C. for 30 min. Then a solution of Selectfluor® (288.25 mg, 813.66 μmol) in DMF (0.5 mL) was added at −78° C. The mixture was stirred at −70° C. for 1 h. The reaction mixture was quenched with NH₄Cl (sat.aq, 20 mL) and extracted with ethyl acetate (20 mL×2), the combined organic layer was dried over Na₂SO₄, filtered and concentrated in vacuo. The residue was purified by column chromatography (SiO₂, Petroleum ether/Ethyl acetate=15/1 to 6/1) to give the title compound (70 mg, 155.12 mol, 24.78% yield, 92.5% purity) as colorless oil. MS (ESI): mass calcd. for C₁₉H₂₆F₃N₃O₄, 417.2; m/z found, 418.4 [M+H]+.

Step D. tert-Butyl 8,11,11-trifluoro-8-(hydroxymethyl)-3,4,8,9,10,11-hexahydro-1H-pyrido[4′,3′:3,4]pyrazolo[1,5-a]azepine-2(7H)-carboxylate. To a solution of 2-tert-butyl 8-ethyl 8,11,11-trifluoro-3,4,8,9,10,11-hexahydro-1H-pyrido[4′,3′:3,4]pyrazolo[1,5-a]azepine-2,8(7H)-dicarboxylate (60 mg, 132.96 μmol) in THF (2 mL) was added LiBH₄ (6.26 mg, 287.37 μmol) at 0° C. The mixture was stirred at 15° C. for 1 h. The mixture was quenched with NH₄Cl (sat.aq, 5 mL) and diluted in H₂O (10 mL), extracted with ethyl acetate (15 mL×3). The combined organic layer was dried over Na₂SO₄, filtered and concentrated in vacuo to give the title compound (78 mg) as white solid which was used in the next step directly. MS (ESI): mass calcd. for C₁₇H₂₄F₃N₃O₃, 375.2; m/z found, 376.1 [M+H]+.

### Intermediate 21: (S*)-tert-Butyl 8,11,11-trifluoro-8-(hydroxymethyl)-3,4,8,9,10,11-hexahydro-1H-pyrido[4′,3′:3,4]pyrazolo[1,5-a]azepine-2(7H)-carboxylate

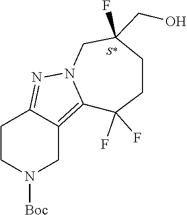 [SMILES: CC(C)(C)OC(=O)N1CCc2nn3c(c2C1)C(F)(F)CC[C@@](F)(CO)C3]

Tert-butyl 8,11,11-trifluoro-8-(hydroxymethyl)-3,4,8,9,10,11-hexahydro-1H-pyrido[4′,3′:3,4]pyrazolo[1,5-a]azepine-2(7H)-carboxylate (140 mg) was resolved by SFC (condition: column: DAICEL CHIRALPAK AD(250 mm×30 mm, 10 um);mobile phase: [0.1% NH₃.H₂O MeOH]; B %: 20%-20%,2.5 min; 50 min) to give (S*)-tert-butyl 8,11,11-trifluoro-8-(hydroxymethyl)-3,4,8,9,10,11-hexahydro-1H-pyrido[4′,3′:3,4]pyrazolo[1,5-a]azepine-2(7H)-carboxylate (Peak 1 on SFC (AD-3_5CM_MeOH(DEA)_5_40_3ML_T35 Column: Chiralpak AD-3 50×4.6 mm I.D., 3 um Mobile phase: methanol (0.05% DEA) in CO₂ from 5% to 40% Flow rate: 3 mL/min Wavelength: 220 nm), retention time=0.868 min, 53 mg, 94% purity) as white solid and (R*)-tert-butyl 8,11,11-trifluoro-8-(hydroxymethyl)-3,4,8,9,10,11-hexahydro-1H-pyrido[4′,3′:3,4]pyrazolo[1,5-a]azepine-2(7H)-carboxylate (Peak 2 on SFC, retention time=0.952 min, 52 mg, 98% purity) as white solid.

### Intermediate 22: (R*)-tert-Butyl 8,11,11-trifluoro-8-(hydroxymethyl)-3,4,8,9,10,11-hexahydro-1H-pyrido[4′,3′:3,4]pyrazolo[1,5-a]azepine-2(7H)-carboxylate

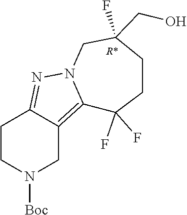 [SMILES: CC(C)(C)OC(=O)N1CCc2nn3c(c2C1)C(F)(F)CC[C@](F)(CO)C3]

The title compound was separated by SFC from tert-butyl 8,11,11-trifluoro-8-(hydroxymethyl)-3,4,8,9,10,11-hexahydro-1H-pyrido[4′,3′:3,4]pyrazolo[1,5-a]azepine-2(7H)-carboxylate (product from Intermediate 20, Step D) in a manner analogous to Intermediate 21: (Peak 2 on SFC, retention time=0.952 min, 52 mg, 98% purity) as white solid.

### Intermediate 23: tert-Butyl 8-(acetamidomethyl)-11,11-difluoro-3,4,8,9,10,11-hexahydro-1H-pyrido[4′,3′:3,4]pyrazolo[1,5-a]azepine-2(7H)-carboxylate

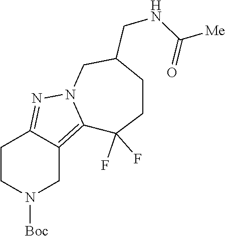 [SMILES: CC(=O)NCC1CCC(F)(F)c2c3c(nn2C1)CCN(C(=O)OC(C)(C)C)C3]

Step A. tert-Butyl 11,11-difluoro-8-(((methylsulfonyl)oxy)methyl)-3,4,8,9,10,11-hexahydro-1H-pyrido[4′,3′:3,4]pyrazolo[1,5-a]azepine-2(7H)-carboxylate. To a solution of tert-butyl 11,11-difluoro-8-(hydroxymethyl)-3,4,8,9,10,11-hexahydro-1H-pyrido[4′,3′:3,4]pyrazolo[1,5-a]azepine-2(7H)-carboxylate (0.5 g, 1.40 mmol) in DCM (4 mL) were added MsCl (192.31 mg, 1.68 mmol, 129.944) and TEA (424.70 mg, 4.20 mmol, 584.184) at 0° C. The mixture was stirred at 0° C. for 1 h. The mixture was poured onto ice-water (50 mL) and extracted with dichloromethane (50 mL×2). The combined organic phase was washed with brine (60 mL), dried over Na₂SO₄, filtered and concentrated in vacuo to give the title compound (0.7 g, crude) as colorless oil. MS (ESI): mass calcd. for C₁₈H₂₇F₂N₃O₅S, 435.2; m/z found, 436.1 [M+H]+.

Step B. tert-Butyl 8-(azidomethyl)-11,11-difluoro-3,4,8,9,10,11-hexahydro-1H-pyrido[4′,3′:3,4]pyrazolo[1,5-a]azepine-2(7H)-carboxylate.

To a solution of tert-butyl 11,11-difluoro-8-(((methylsulfonyl)oxy)methyl)-3,4,8,9,10,11-hexahydro-1H-pyrido[4′,3′:3,4]pyrazolo[1,5-a]azepine-2(7H)-carboxylate (0.7 g, crude) in DMF (4 mL) was added NaN₃ (417.99 mg, 6.43 mmol) at 0° C. under N₂. The mixture was stirred at 50° C. for 12 h. The mixture was diluted with ethyl acetate (40 mL) and washed with brine (20 mL×3). The organic phase was dried over Na₂SO₄, filtered and concentrated in vacuo to give the title compound (0.65 g, crude) as yellow oil. MS (ESI): mass calcd. for C₁₇H₂₄F₂N₆O₂, 382.2; m/z found, 383.4 [M+1]⁺.

Step C. tert-Butyl 8-(aminomethyl)-11,11-difluoro-3,4,8,9,10,11-hexahydro-1H-pyrido[4′,3′:3,4]pyrazolo[1,5-a]azepine-2(7H)-carboxylate. To a solution of tert-butyl 8-(azidomethyl)-11,11-difluoro-3,4,8,9,10,11-hexahydro-1H-pyrido[4′,3′:3,4]pyrazolo[1,5-a]azepine-2(7H)-carboxylate (0.65 g, crude) in EtOH (2 mL) and H₂O (0.2 mL) were added Zn (222.29 mg, 3.40 mmol) and NH₄Cl (272.77 mg, 5.10 mmol, 178.28 4). The mixture was stirred at 15° C. for 24 h. The reaction mixture was filtered and concentrated in vacuo to give the title compound (452 mg, crude) as white solid. MS (ESI): mass calcd. for C₁₇H₂₆F₂N₄O₂, 356.2; m/z found, 357.3 [M+H]+.

Step D. tert-Butyl 8-(acetamidomethyl)-11,11-difluoro-3,4,8,9,10,11-hexahydro-1H-pyrido[4′,3′:3,4]pyrazolo[1,5-a]azepine-2(7H)-carboxylate. To a solution of tert-butyl 8-(aminomethyl)-11,11-difluoro-3,4,8,9,10,11-hexahydro-1H-pyrido[4′,3′:3,4]pyrazolo[1,5-a]azepine-2(7H)-carboxylate (50 mg, crude) and TEA (70.98 mg, 701.44 μmol, 97.63 μL) in DCM (3 mL) was added acetyl acetate (57.29 mg, 561.15 mol, 52.564) at 0° C. under N₂. The mixture was stirred at 10° C. for 1 h. The mixture was diluted with water (30 mL) and extracted with DCM (30 mL×2). The combined organic phase was washed with brine (60 mL), dried over anhydrous Na₂SO₄, filtered and concentrated in vacuo. The residue was purified by column chromatography (SiO₂, Petroleum ether/Ethyl acetate=3/1 to 0/1) to give the title compound (37 mg, 92.86 mol, 66.19% yield) as colorless oil. MS (ESI): mass calcd. for C₁₉H₂₈F₂N₄O₃, 398.2; m/z found, 399.0 [M+H]+.

### Intermediate 24: tert-Butyl 11,11-difluoro-8-((2,2,2-trifluoroacetamido)methyl)-3,4,8,9,10,11-hexahydro-1H-pyrido[4′,3′:3,4]pyrazolo[1,5-a]azepine-2(7H)-carboxylate

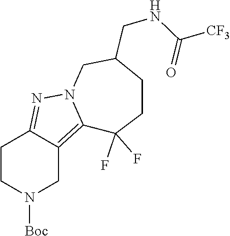 [SMILES: CC(C)(C)OC(=O)N1CCc2nn3c(c2C1)C(F)(F)CCC(CNC(=O)C(F)(F)F)C3]

To a solution of tert-butyl 8-(aminomethyl)-11,11-difluoro-3,4,8,9,10,11-hexahydro-1H-pyrido[4′,3′:3,4]pyrazolo[1,5-a]azepine-2(7H)-carboxylate (100 mg) and TEA (141.96 mg, 1.40 mmol, 195.264) in DCM (3 mL) was added 2,2,2-trifluoroacetic anhydride (235.72 mg, 1.12 mmol, 156.11 μL) at 0° C. The mixture was stirred at 15° C. for 1 h. The mixture was diluted with water (20 mL). The resultant solution was extracted with DCM (20 mL×2). The combined organic phase was washed with brine (30 mL), dried over anhydrous Na₂SO₄, filtered and concentrated in vacuo. The residue was purified by column chromatography (SiO₂, Petroleum ether/Ethyl acetate=5/1 to 2/1) to give the title compound (90 mg, 198.93 μμmol) as yellow oil. MS (ESI): mass calcd. for C₁₉H₂₅N₄F₅O₃, 452.2; m/z found, 453.1 [M+H]+. ¹H NMR (400 MHz, CDCl₃) δ=6.66 (br s, 1H), 4.57 (br s, 2H), 4.46-4.20 (m, 2H), 3.71 (br s, 2H), 3.55-3.43 (m, 1H), 3.07-2.94 (m, 1H), 2.73 (br s, 2H), 2.43-2.19 (m, 3H), 2.18-2.09 (m, 1H), 1.90-1.76 (m, 1H), 1.51 (s, 9H).

### Intermediate 25: tert-Butyl 11,11-difluoro-8-(((methoxycarbonyl)amino)methyl)-3,4,8,9,10,11-hexahydro-1H-pyrido[4′,3′:3,4]pyrazolo[1,5-a]azepine-2(7H)-carboxylate

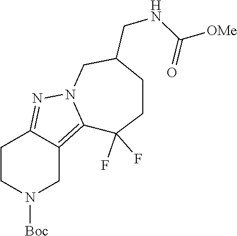 [SMILES: COC(=O)NCC1CCC(F)(F)c2c3c(nn2C1)CCN(C(=O)OC(C)(C)C)C3]

To a solution of tert-butyl 8-(aminomethyl)-11,11-difluoro-3,4,8,9,10,11-hexahydro-1H-pyrido[4′,3′:3,4]pyrazolo[1,5-a]azepine-2(7H)-carboxylate (0.15 g) and TEA (212.94 mg, 2.10 mmol, 292.90 μL) in DCM (3 mL) was added methyl carbonochloridate (159.08 mg, 1.68 mmol, 130.39 μL) at 0° C. The mixture was stirred at 10° C. for 1 h. The mixture was diluted with water (30 mL). The resultant solution was extracted with dichloromethane (30 mL×2). The combined organic phase was washed with brine (60 mL), dried over Na₂SO₄, filtered and concentrated in vacuo. The residue was purified by column chromatography (SiO₂, Petroleum ether/Ethyl acetate=5/1 to 1/1) to give the title compound (114 mg, 275.07 μmol, 65.36% yield) as white solid. MS (ESI): mass calcd. for C₁₉H₂₈F₂N₄O₄, 414.2; m/z found, 415.3 [M+H]+. ¹H NMR (400 MHz, CDCl₃) δ=4.80 (br s, 1H), 4.46 (s, 2H), 4.31 (d, J=14.5 Hz, 1H), 4.11-4.02 (m, 1H), 3.60 (s, 5H), 3.19-3.06 (m, 1H), 2.90-2.75 (m, 1H), 2.63 (t, J=5.5 Hz, 2H), 2.39-2.23 (m, 1H), 2.22-2.07 (m, 1H), 2.05-1.85 (m, 1H), 1.86-1.67 (m, 1H), 1.41 (s, 9H).

### Intermediate 26: tert-Butyl11,11-difluoro-8-hydroxy-8-methyl-3,4,8,9,10,11-hexahydro-1H-pyrido[4′,3′:3,4]pyrazolo[1,5-a]azepine-2(7H)-carboxylate

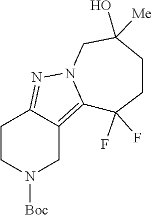 [SMILES: CC1(O)CCC(F)(F)c2c3c(nn2C1)CCN(C(=O)OC(C)(C)C)C3]

To a solution of MeMgBr (3 M, 648.40 μL) in THF (1 mL) was added a solution of tert-butyl 11,11-difluoro-8-oxo-3,4,8,9,10,11-hexahydro-1H-pyrido[4′,3′:3,4]pyrazolo[1,5-a]azepine-2(7H)-carboxylate (0.2 g, 486.30 μmol) in THF (2 mL) at −30° C. under N₂. The mixture was stirred at 0° C. for 4 h then warmed to 25° C. and stirred for 2 h. The reaction mixture was quenched by NH₄Cl (sat.aq, 20 mL) at 0° C., then extracted with ethyl acetate (20 mL×2). The combined organic layer was washed with brine (30 mL), dried over Na₂SO₄, filtered and concentrated under reduced pressure. The residue was purified by RP HPLC (condition E) to give the title compound (37 mg, 103.53 μmol, 21.29% yield) as yellow oil. MS (ESI): mass calcd. for C₁₇H₂₅F₂N₃O₃, 357.2; m/z found, 358.2 [M+H]+. ¹H NMR (400 MHz, CDCl₃) δ=4.60-4.51 (br m, 2H), 4.25 (s, 2H), 3.73-3.66 (br m, 2H), 2.74-2.71 (m, 2H), 2.70-2.47 (m, 2H), 2.10-2.00 (m, 2H), 1.53 (s, 9H), 1.32 (s, 3H).

### Intermediate 27: tert-butyl11,11-difluoro-8-hydroxy-8-vinyl-3,4,8,9,10,11-hexahydro pyrido[4′,3′:3,4]pyrazolo[1,5-a]azepine-2(7H)-carboxylate

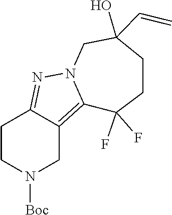 [SMILES: C=CC1(O)CCC(F)(F)c2c3c(nn2C1)CCN(C(=O)OC(C)(C)C)C3]

To a solution of tert-butyl 11,11-difluoro-8-oxo-3,4,8,9,10,11-hexahydro-1H-pyrido [4′,3′:3,4]pyrazolo[1,5-a]azepine-2(7H)-carboxylate (0.15 g, 421.85 μmol) in DCM (3 mL) was added vinylMgBr (1 M, 843.70 μL) at −40° C. under N₂. The mixture was stirred at −40° C. under N₂ for 6 h. The mixture was quenched with NH₄Cl (sat.aq, 30 mL). The resultant solution was extracted with ethyl acetate (30 mL). The organic phase was dried over Na₂SO₄, filtered and concentrated in vacuo. The residue was purified by RP HPLC (condition D) to give the title compound (0.041 g, 110.99 μmol, 26.31% yield) as colorless oil. MS (ESI): mass calcd. for C₁₈H₂₅F₂N₃O₃, 369.2; m/z found, 370.2 [M+H]+.

### Intermediate 28: tert-Butyl 8-ethynyl-11,11-difluoro-8-hydroxy-3,4,8,9,10,11-hexahydro-1H-pyrido[4′,3′:3,4]pyrazolo[1,5-a]azepine-2(7H)-carboxylate

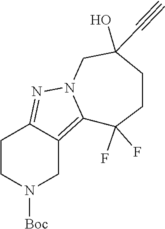 [SMILES: C#CC1(O)CCC(F)(F)c2c3c(nn2C1)CCN(C(=O)OC(C)(C)C)C3]

To a solution of tert-butyl 11,11-difluoro-8-oxo-3,4,8,9,10,11-hexahydro-1H-pyrido [4′,3′:3,4]pyrazolo[1,5-a]azepine-2(7H)-carboxylate (400 mg, 1.15 mmol) and LiCl (97.36 mg, 2.30 mmol, 47.03 μL) in DCM (5 mL) was added ethynyl magnesium bromide (0.5 M, 11.48 mL) at 0° C., the mixture was stirred at 15° C. for 14 h. The reaction was quenched by NH₄Cl (sat.aq, 100 mL) and then extracted with EtOAc (100 mL×2). The combined organic phase was washed with brine (80 mL), dried over Na₂SO₄, filtered and concentrated in vacuo. The crude product was purified with other three batches (400 mg scale, 200 mg scale and 800 mg scale) by RP HPLC(condition D) to give total 720 mg of the title compound as yellow solid. MS (ESI): mass calcd. for C₁₈H₂₃F₂N₃O₃, 367.2; m/z found, 368.2 [M+H]+. ¹H NMR (400 MHz, CDCl₃) δ=4.54-4.40 (m, 4H), 3.70-3.59 (m, 2H), 2.69-2.48 (m, 2H), 2.37-2.30 (m, 2H), 2.27-2.22 (m, 3H), 1.45 (s, 9H).

### Intermediate 29: (R*)-tert-Butyl 8-ethynyl-11,11-difluoro-8-hydroxy-3,4,8,9,10,11-hexahydro-1H-pyrido[4′,3′:3,4]pyrazolo[1,5-a]azepine-2(7H)-carboxylate

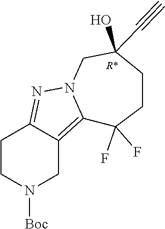 [SMILES: C#C[C@]1(O)CCC(F)(F)c2c3c(nn2C1)CCN(C(=O)OC(C)(C)C)C3]

Tert-butyl 8-ethynyl-11,11-difluoro-8-hydroxy-3,4,8,9,10,11-hexahydro-1H-pyrido[4′,3′:3,4]pyrazolo[1,5-a]azepine-2(7H)-carboxylate (700 mg) was resolved by SFC (condition: column: DAICEL CHIRALCEL OJ-H(250 mm*30 mm,5 um); mobile phase: [0.1% NH₃.H₂O IPA]; B %: 15%-15%, 5 min:180 min) to give (R*)-tert-butyl 8-ethynyl-11,11-difluoro-8-hydroxy-3,4,8,9,10,11-hexahydro-1H-pyrido[4′,3′:3,4]pyrazolo[1,5-a]azepine-2(7H)-carboxylate (Peak 1 on SFC (IC-3_5CM_MeOH(DEA)_5_40_3ML_T35 Column: Chiralpak IC-3 50×4.6 mm I.D., 3 um Mobile phase: methanol (0.05% DEA) in CO₂ from 5% to 40% Flow rate: 3 mL/min Wavelength: 220 nm), retention time=0.651 min, 110 mg, 290.43 μmol) as colorless oil, and (S*)-tert-butyl 8-ethynyl-11,11-difluoro-8-hydroxy-3,4,8,9,10,11-hexahydro-1H-pyrido[4′,3′:3,4]pyrazolo[1,5-a]azepine-2(7H)-carboxylate (Peak 2 on SFC, retention time=0.747 min, 200 mg, 517.16 μmol) as colorless oil.

### Intermediate 30: (S*)-tert-Butyl 8-ethynyl-11,11-difluoro-8-hydroxy-3,4,8,9,10,11-hexahydro-1H-pyrido[4′,3′:3,4]pyrazolo[1,5-a]azepine-2(7H)-carboxylate

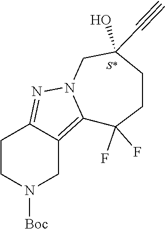 [SMILES: C#C[C@@]1(O)CCC(F)(F)c2c3c(nn2C1)CCN(C(=O)OC(C)(C)C)C3]

The title compound was separated by SFC from tert-butyl 8-ethynyl-11,11-difluoro-8-hydroxy-3,4,8,9,10,11-hexahydro-1H-pyrido[4′,3′:3,4]pyrazolo[1,5-a]azepine-2(7H)-carboxylate (Intermediate 28) in a manner analogous to Intermediate 29: (Peak 2 on SFC (IC-3_5CM_MeOH(DEA)_5_40_3ML_T35 Column: Chiralpak IC-3 50×4.6 mm I.D., 3 um Mobile phase: methanol (0.05% DEA) in CO₂ from 5% to 40% Flow rate: 3 mL/min Wavelength: 220 nm), retention time=0.747 min, 200 mg, 517.16 μmol, [a]²⁵_{D}=+2.4 (c=0.52, MeOH)) as colorless oil.

### Intermediate 31: (3R,8R)-tert-Butyl 11,11-difluoro-8-hydroxy-3-methyl-3,4,8,9,10,11-hexahydro-1H-pyrido[4′,3′:3,4]pyrazolo[1,5-a]azepine-2(7H)-carboxylate

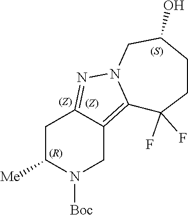

Step A. (2R)-tert-Butyl 5-(2-ethoxy-2-oxoacetyl)-2-methyl-4-oxopiperidine-1-carboxylate. The three-necked round bottom flask was cooled to −78° C. and LiHMDS (1 M, 304.77 mL) was added, then a solution of (R)-tert-butyl 2-methyl-4-oxopiperidine-1-carboxylate (50 g, 234.44 mmol) in THF (500 mL) was added dropwise and the reaction mixture was stirred at −78° C. for 30 minutes under N₂. To the mixture was added diethyl oxalate (44.54 g, 304.77 mmol, 41.63 mL) dropwise. After addition the reaction mixture was warmed to 25° C. over 30 minutes and stirred at 25° C. for another 2 h. The reaction solution was quenched with HCl (1N) until pH=2˜3. The resultant solution was extracted with EtOAc (500 mL×3), the combined organic phase was dried over anhydrous Na₂SO₄, filtered and concentrated under vacuum. The residue was purified by column chromatography (SiO₂, petroleum ether/ethyl acetate=1/1 to 1/1) to give the title compound (73 g, crude) as yellow oil and used directly for next step.

Step B. (R)-5-tert-Butyl 3-ethyl 6-methyl-6,7-dihydro-1H-pyrazolo[4,3-c]pyridine-3,5(4H)-dicarboxylate. To a mixture of (2R)-tert-butyl 5-(2-ethoxy-2-oxoacetyl)-2-methyl-4-oxopiperidine-1-carboxylate (73 g, crude) in EtOH (600 mL) was added NH₂NH₂H₂O (11.08 g, 221.33 mmol, 10.76 mL). The mixture was stirred at 25° C. for 4 h. The mixture was concentrated and the residue was purified by flash column chromatography (SiO₂, petroleum ether/ethyl acetate=10/1 to 1/1) to give the title compound (50. 5 g, 161.62 mmol) as yellow solid. MS (ESI): mass calcd. for C₁₅H₂₃N₃O₄, 309.2; m/z found, 310.1 [M+H]+.

Step C. (R)-tert-Butyl 3-(3-ethoxy-3-oxopropanoyl)-6-methyl-6,7-dihydro-2H-pyrazolo[4,3-c]pyridine-5(4H)-carboxylate. To a solution of ethyl acetate (22.78 g, 258.60 mmol, 25.32 mL) in THF (400 mL) was added NaHMDS (1 M, 646.50 mL) at −65° C. under N₂, followed by a solution of (R)-5-tert-butyl 3-ethyl 6-methyl-6,7-dihydro-1H-pyrazolo[4,3-c]pyridine-3,5(4H)-dicarboxylate (40 g, 129.30 mmol) in THF (400 mL) after 0.5 h. The mixture was stirred at 45° C. for 16 h. The reaction mixture was quenched by HCl (1N) until pH˜6 at 0° C. The resultant solution was extracted with EtOAc (800 mL×2) and the combined organic layer was washed with brine (1 L), dried over Na₂SO₄, filtered and concentrated under reduced pressure. The residue was purified by column chromatography (SiO₂, petroleum ether/ethyl acetate=3/1, Rf=0.4) to give the title compound (40 g, 113.83 mmol, 88.04% yield, 100% purity) as yellow oil. MS (ESI): mass calcd. for C₁₇H₂₅N₃O₅, 351.2; m/z found, 352.3 [M+H]+.

Step D. Mixture of (R)-di-tert-butyl 3-(3-ethoxy-3-oxopropanoyl)-6-methyl-6,7-dihydro-2H-pyrazolo[4,3-c]pyridine-2,5(4H)-dicarboxylate and (R)-di-tert-butyl 3-(3-ethoxy-3-oxopropanoyl)-6-methyl-6,7-dihydro-1H-pyrazolo[4,3-c]pyridine-1,5(4H)-dicarboxylate. To a solution of (R)-tert-butyl 3-(3-ethoxy-3-oxopropanoyl)-6-methyl-6,7-dihydro-2H-pyrazolo[4,3-c]pyridine-5(4H)-carboxylate (40 g, 113.83 mmol), TEA (33.17 g, 327.80 mmol, 45.63 mL) and DMAP (1.39 g, 11.38 mmol) in DCM (400 mL) was added Boc₂O (22.86 g, 104.73 mmol, 24.06 mL), and then the mixture was stirred at 20° C. for 16 h. The reaction mixture was quenched with HCl (1 N, 1 L) at 0° C. and the resultant solution was extracted with DCM (500 mL×3). The combined organic layer was washed with brine (500 mL), dried over Na₂SO₄, filtered and concentrated under reduced pressure. The residue was purified by silica gel chromatography (100-200 mesh silica gel, petroleum ether/ethyl acetate=5/1 to 2/1, Rf=0.6) to give the title compound (44.2 g, 45.52 mmol, 39.99% yield, 93% purity) as colorless oil. MS (ESI): mass calcd. for C₂₂H₃₃N₃O₇, 451.2; m/z found, 452.3 [M+H]+.

Step E. Mixture of (6R)-di-tert-butyl 3-(4-(((tert-butyldiphenylsilyl)oxy)methyl)-2-(ethoxycarbonyl)pent-4-enoyl)-6-methyl-6,7-dihydro-2H-pyrazolo[4,3-c]pyridine-2,5(4H)-dicarboxylate and (6R)-di-tert-butyl 3-(4-(((tert-butyldiphenylsilyl)oxy)methyl) -2-(ethoxycarbonyl)pent-4-enoyl)-6-methyl-6,7-dihydro-1H-pyrazolo[4,3-c]pyridine-1,5(4H)-dicarboxylate. To a solution of the mixture of (R)-di-tert-butyl 3-(3-ethoxy-3-oxopropanoyl)-6-methyl-6,7-dihydro-2H-pyrazolo[4,3-c]pyridine-2,5(4H)-dicarboxylate and (R)-di-tert -butyl3-(3-ethoxy-3-oxopropanoyl)-6-methyl-6,7-dihydro-1H-pyrazolo[4,3-c]pyridine -1,5(4H)-dicarboxylate (44.20 g, 90.94 mmol) in acetone (500 mL) were added K₂CO₃ (18.85 g, 136.41 mmol), NaI (2.73 g, 18.19 mmol) and ((2-(bromomethyl)allyl)oxy)(tert-butyl)diphenylsilane (40.72 g, 104.58 mmol). The mixture was stirred at 55° C. under N₂ for 4 h. The reaction mixture was added dropwise into IN HCl (1 L) at 0° C. and extracted with EtOAc (800 mL×2). The combined organic phase was washed with brine (500 mL), dried over anhydrous Na₂SO₄, filtered and concentrated in vacuo. The residue was purified by silica gel chromatography (100-200 mesh silica gel, petroleum ether/ethyl acetate=50/1 to 3/1) to give the title compound (34 g, 42.05 mmol, 46.24% yield, 94% purity) as colorless oil. MS (ESI): mass calcd. for C₄₂H₅₇N₃O₈Si, 759.4; m/z found, 760.5 [M+H]+.

Step F. (R)-tert-Butyl 3-(4-(((tert-butyldiphenylsilyl)oxy)methyl)pent-4-enoyl)-6-methyl-6,7-dihydro-2H-pyrazolo[4,3-c]pyridine-5(4H)-carboxylate. To a solution of the mixture of (6R)-di-tert-butyl 3-(4-(((tert-butyldiphenylsilyl)oxy) methyl)-2-(ethoxycarbonyl)pent-4-enoyl)-6-methyl-6,7-dihydro-2H-pyrazolo[4,3-c]pyridine-2,5(4H)-dicarboxylate and (6R)-di-tert-butyl 3-(4-(((tert-butyldiphenylsilyl)oxy)methyl)-2-(ethoxycarbonyl)pent-4-enoyl)-6-methyl-6,7-dihydro-1H-pyrazolo[4,3-c]pyridine-1,5(4H)-dicarboxylate (34.00 g, 42.05 mmol) in MeOH (300 mL) was added a solution of KOH (4.72 g, 84.11 mmol) in H₂O (50 mL) at 20° C., the mixture was stirred at 65° C. for 8 h. The reaction mixture was added dropwise into HCl (1N, 1 L) at 0° C., and then extracted with EtOAc (1 L×2). The combined organic phase was washed with brine (800 mL), dried over anhydrous Na₂SO₄, filtered and concentrated in vacuo. The residue was purified by silica gel chromatography (100-200 mesh silica gel, petroleum ether/ethyl acetate=30/1 to 5/1) to give the title compound (21.2 g, 33.54 mmol, 79.76% yield, 93% purity) as colorless oil. MS (ESI): mass calcd. for C₃₄H₄₅N₃O₄Si, 587.3; m/z found, 588.5 [M+H]⁺.

Step G. (R)-tert-Butyl 3-(4-(((tert-butyldiphenylsilyl)oxy)methyl)-1,1-difluoropent-4-en-1-yl)-6-methyl-6,7-dihydro-2H-pyrazolo[4,3-c]pyridine-5(4H)-carboxylate. To a solution of (R)-tert-butyl 3-(4-(((tert-butyldiphenylsilyl)oxy)methyl)pent-4-enoyl)-6-methyl-6,7-dihydro-2H-pyrazolo[4,3-c]pyridine-5(4H)-carboxylate (11 g, 18.71 mmol) in DCM (150 mL) were added DAST (18.10 g, 112.28 mmol, 14.83 mL) and EtOH (172.42 mg, 3.74 mmol, 218.81 4) at 0° C. The mixture was stirred at 0° C. for 2 h. The reaction mixture was added dropwise into NaHCO₃ (sat.aq, 700 mL) at 0° C. then extracted with DCM (400 mL×2). The combined organic phase was washed with brine (400 mL), dried over Na₂SO₄, filtered and concentrated in vacuo. The residue was purified by silica gel chromatography (100-200 mesh silica gel, petroleum ether/ethyl acetate=20/1 to 5/1) to give the title compound (6.9 g, 10.86 mmol, 58.05% yield, 96% purity) as yellow oil. MS (ESI): mass calcd. for C₃₄H₄₅F₂N₃O₃Si, 609.3; m/z found, 610.5 [M+H]⁺.

Step H. (R)-tert-Butyl 3-(1,1-difluoro-4-(hydroxymethyl)pent-4-en-1-yl)-6-methyl-6,7-dihydro-2H-pyrazolo[4,3-c]pyridine-5(4H)-carboxylate. To a solution of (R)-tert-butyl 3-(4-(((tert-butyldiphenylsilyl)oxy)methyl)-1,1-difluoropent-4-en-1-yl)-6-methyl-6,7-dihydro-2H-pyrazolo[4,3-c]pyridine-5(4H)-carboxylate (7.05 g, 11.56 mmol) in THF (70 mL) was added TBAF (1 M in THF, 13.3 mL). The mixture was stirred at 17° C. for 3 h. The reaction mixture was poured into water (200 mL) and extracted with ethyl acetate (60 mL×3). The combined organic phase was washed with brine (80 mL), dried over anhydrous Na₂SO₄, filtered and concentrated in vacuo. The residue was purified by silica gel chromatography (100-200 mesh silica gel, petroleum ether/ethyl acetate=10/1 to 1/1) to give the title compound (3.8 g, 10.03 mmol, 86.73% yield, 98% purity) as yellow oil. MS (ESI): mass calcd. for C₁₈H₂₇F₂N₃O₃, 371.2; m/z found, 372.3 [M+H]+.

Step I. (R)-tert-Butyl 3-(1,1-difluoro-4-(((methylsulfonyl)oxy)methyl)pent-4-en-1-yl) -6-methyl-6,7-dihydro-2H-pyrazolo[4,3-c]pyridine-5(4H)-carboxylate. To a solution of (R)-tert-butyl 3-(1,1-difluoro-4-(hydroxymethyl)pent-4-en-1-yl)-6-methyl-6,7-dihydro-2H-pyrazolo[4,3-c]pyridine-5(4H)-carboxylate (3.8 g, 10.03 mmol) and TEA (3.04 g, 30.08 mmol, 4.19 mL) in DCM (40 mL) was added a solution of MsCl (1.49 g, 13.03 mmol, 1.01 mL) in DCM (3 mL) at 0° C. The mixture was stirred at 0° C. for 1.5 h. The mixture was poured into water (100 mL) and extracted with ethyl acetate (50 mL×2). The combined organic phase was washed with brine (50 mL), dried over anhydrous Na₂SO₄, filtered and concentrated in vacuo to give the title compound (4.7 g, crude) as yellow oil. MS (ESI): mass calcd. for C₁₉H₂₉F₂N₃O₅S, 449.2; m/z found, 450.4 [M+H]+.

Step J. (R)-tert-Butyl 11,11-difluoro-3-methyl-8-methylene-3,4,8,9,10,11-hexahydro-1H-pyrido[4′,3′:3,4]pyrazolo[1,5-a]azepine-2(7H)-carboxylate. To a solution of (R)-tert-butyl 3-(1,1-difluoro-4-(((methylsulfonyl)oxy)methyl)pent-4-en-1-yl)-6-methyl-6,7-dihydro-2H-pyrazolo[4,3-c]pyridine-5(4H)-carboxylate (4.7 g, crude) in THF (50 mL) was added DBU (2.39 g, 15.70 mmol, 2.37 mL). The mixture was stirred at 15° C. for 14 h. The mixture was poured into ice-water (150 mL) and extracted with ethyl acetate (100 mL×3). The combined organic phase was washed with brine (100 mL), dried over anhydrous Na₂SO₄, filtered and concentrated in vacuo. The residue was purified by silica gel chromatography (100-200 mesh silica gel, petroleum ether/ethyl acetate=20/1 to 10/1) to give the title compound (2.3 g, 6.31 mmol, 97% purity) as colorless oil. MS (ESI): mass calcd. for C₁₅H₂₅F₂N₃O₂, 353.2; m/z found, 354.3 [M+H]⁺.

Step K. (R)-tert-Butyl 11,11-difluoro-3-methyl-8-oxo-3,4,8,9,10,11-hexahydro-1H-pyrido[4′,3′:3,4]pyrazolo[1,5-a]azepine-2(7H)-carboxylate. To a solution of (R)-tert-butyl 11,11-difluoro-3-methyl-8-methylene-3,4,8,9,10,11-hexahydro-1H-pyrido[4′,3′:3,4]pyrazolo[1,5-a]azepine-2(7H)-carboxylate (1 g, 2.83 mmol) in THF (20 mL) and H₂O (5 mL) were added NaIO₄ (2.42 g, 11.32 mmol, 627.184) and OsO₄ (71.94 mg, 282.96 μmol, 14.68 μL) at 0° C. The mixture was stirred at 15° C. for 16 h. The mixture was poured into Na₂SO₃ (sat.aq, 100 mL) at 0° C. and extracted with ethyl acetate (50 mL×2). The combined organic phase was washed with brine (100 mL), dried over anhydrous Na₂SO₄, filtered and concentrated in vacuo to give the title compound (1 g, 2.81 mmol, 99.44% yield) as white solid.

Step L. (3R)-tert-Butyl 11,11-difluoro-8-hydroxy-3-methyl-3,4,8,9,10,11-hexahydro-1H-pyrido[4′,3′:3,4]pyrazolo[1,5-a]azepine-2(7H)-carboxylate. A solution of (R)-tert-butyl 11,11-difluoro-3-methyl-8-oxo-3,4,8,9,10,11-hexahydro-1H-pyrido[4′,3′:3,4]pyrazolo[1,5-a]azepine-2(7H)-carboxylate (800 mg, 2.25 mmol) in EtOH (15 mL) was added NaBH₄ (127.75 mg, 3.38 mmol) at 0° C., and then the mixture was stirred at 15° C. for 2 h under N₂ atmosphere. The mixture was poured into ice-water (10 mL), then removed EtOH under vacuum. The mixture was extracted with EtOAc (5 mL×2), and the combined organic phase was washed with brine (10 mL), dried over anhydrous Na₂SO₄, filtered and concentrated under vacuum to give the title compound (800 mg, 2.17 mmol, 96.45% yield, 97% purity) as white solid. MS (ESI): mass calcd. for C₁₇H₂₅F₂N₃O₃, 357.2; m/z found, 358.3 [M+H]+.

Step M. (3R,8R)-tert-Butyl 11,11-difluoro-8-hydroxy-3-methyl-3,4,8,9,10,11-hexahydro-1H-pyrido[4′,3′:3,4]pyrazolo[1,5-a]azepine-2(7H)-carboxylate. (3R)-tert-Butyl-11,11-difluoro-8-hydroxy-3-methyl-3,4,8,9,10,11-hexahydro-1H-pyrido[4′,3′:3,4]pyrazolo[1,5-a]azepine-2(7H)-carboxylate (900 mg) was resolved by SFC (condition: column: DAICEL CHIRALPAK IC(250 mm*50 mm,10 um); mobile phase: [0.1% NH₃.H₂O MeOH]; B %: 20%-20%,1.9 min:400 min) to give (3R,8R)-tert-butyl 11,11-difluoro-8-hydroxy-3-methyl-3,4,8,9,10,11-hexahydro-1H-pyrido[4′,3′:3,4]pyrazolo[1,5-a]azepine-2(7H)-carboxylate (Peak 1 on SFC (IC-3_5CM_MeOH(DEA)_5_40_3ML_T35 Column: Chiralpak IC-3 50×4.6 mm I.D., 3 um Mobile phase: methanol (0.05% DEA) in CO₂ from 5% to 40% Flow rate: 3 mL/min Wavelength: 220 nm), retention time=0.848 min, 369 mg, 1.03 mmol, 40.88% yield, 99.7% purity) as white solid. [a]²⁵_{D}=+48.119 (c=0.43, in DCM)).

### Intermediate 32: (3R,8S)-tert-Butyl 11,11-difluoro-8-hydroxy-3-methyl-3,4,8,9,10,11-hexahydro-1H-pyrido[4′,3′:3,4]pyrazolo[1,5-a]azepine-2(7H)-carboxylate

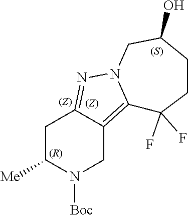

The title compound was separated by SFC from (3R)-tert-butyl 11,11-difluoro-8-hydroxy-3-methyl-3,4,8,9,10,11-hexahydro-1H-pyrido[4′,3′:3,4]pyrazolo[1,5-a]azepine-2(7H)-carboxylate (product from Intermediate 31, Step L) in a manner analogous to Intermediate 31, Step M: (Peak 2 on SFC (“IC-3_5CM_MeOH(DEA)_5_40_3ML_T35 Column: Chiralpak IC-3 50×4.6 mm I.D., 3 um Mobile phase: methanol (0.05% DEA) in CO₂ from 5% to 40% Flow rate: 3 mL/min Wavelength: 220 nm”) , retention time=0.940 min, 311 mg, 838.86 μmol, 33.31% yield, 96.4% purity) as white solid.

### Intermediate 33: (3R,8S*)-tert-Butyl 11,11-difluoro-8-(hydroxymethyl)-3-methyl-3,4,8,9,10,11-hexahydro-1H-pyrido[4′,3′:3,4]pyrazolo[1,5-a]azepine-2(7H)-carboxylate

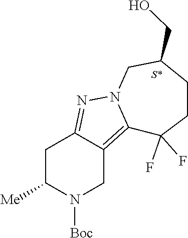 [SMILES: C[C@@H]1Cc2nn3c(c2CN1C(=O)OC(C)(C)C)C(F)(F)CC[C@H](CO)C3]

### Step A. (3R)-tert-Butyl 11,11-difluoro-8-(hydroxymethyl)-3-methyl-3,4,8,9,10,11-hexahydro-1H-pyrido[4′,3′:3,4]pyrazolo[1,5-a]azepine-2(7H)-carboxylate

To a solution of (R)-tert-butyl 11,11-difluoro-3-methyl-8-methylene-3,4,8,9,10,11-hexahydro-1H-pyrido[4′,3′:3,4]pyrazolo[1,5-a]azepine-2(7H)-carboxylate (1.4 g, 3.84 mmol) in THF (15 mL) was added 9-BBN (0.5 M in THF, 192.13 mL) at 0° C., the mixture was stirred at 0° C. for 2 h. A solution of NaOH (1.54 g, 38.43 mmol) in H₂O (2 mL) was added at −30° C., then H₂O₂ (5.23 g, 46.11 mmol, 4.43 mL, 30% purity) was added and the reaction mixture was stirred at 10° C. for 12 h The mixture was quenched with NaHSO₃ (sat.aq, 400 mL) and extracted with ethyl acetate (200 mL×3). The combined organic layer was washed with H₂O (200 mL×2), brine (200 mL×2), dried over Na₂SO₄, filtered and concentrated in vacuo. The residue was purified by silica gel chromatography (100-200 mesh silica gel, petroleum ether/ethyl acetate=5/1 to 2/1) to give the title compound (1.05 g, 2.74 mmol, 71.36% yield, 97% purity) as white solid. MS (ESI): mass calcd. for C₁₈H₂₇F₂N₃O₃, 371.2; m/z found, 372.3 [M+H]+.

### Step B. (3R,8S*)-tert-Butyl11,11-difluoro-8-(hydroxymethyl)-3-methyl-3,4,8,9,10,11-hexahydro-1H-pyrido[4′,3′:3,4]pyrazolo[1,5-a]azepine-2(7H)-carboxylate

(3R)-tert-butyl-11,11-difluoro-8-(hydroxymethyl)-3-methyl-3,4,8,9,10,11-hexahydro-1H-pyrido[4′,3′:3,4]pyrazolo[1,5-a]azepine-2(7H)-carboxylate (1 g, 2.69 mmol) was resolved by SFC (condition: DAICEL CHIRALPAK IC(250 mm*30 mm,5 um); mobile phase: [0.1% NH₃.H₂O MeOH]; B %: 25%-25%, 1.9 min:180 min) to give (3R,8S*)-tert-butyl-11,11-difluoro-8-(hydroxymethyl)-3-methyl-3,4,8,9,10,11-hexahydro-1H-pyrido[4′,3′:3,4]pyrazolo[1,5-a]azepine-2(7H)-carboxylate (Peak 1 on SFC (AD-3_5CM_MeOH(DEA)_5_40_3ML_T35 Column: Chiralpak AD-3 50×4.6 mm I.D., 3 um Mobile phase: methanol (0.05% DEA) in CO₂ from 5% to 40% Flow rate: 3 mL/min Wavelength: 220 nm) , retention time=0.734 min, 320 mg, 792.63 μmol, 29.44% yield, 92% purity. [a]²⁵_{D}=+35.125 (c=0.98, in MeOH)) as colorless oil.

### Intermediate 34: (3R,8R*)-tert-Butyl 11,11-difluoro-8-(hydroxymethyl)-3-methyl-3,4,8,9,10,11-hexahydro-1H-pyrido[4′,3′:3,4]pyrazolo[1,5-a]azepine-2(7H)-carboxylate

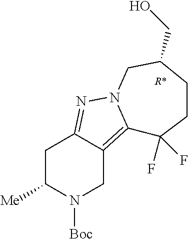 [SMILES: C[C@@H]1Cc2nn3c(c2CN1C(=O)OC(C)(C)C)C(F)(F)CC[C@@H](CO)C3]

The title compound was separated by SFC from (3R)-tert-butyl 11,11-difluoro-8-(hydroxymethyl)-3-methyl-3,4,8,9,10,11-hexahydro-1H-pyrido[4′,3′:3,4]pyrazolo[1,5-a]azepine-2(7H)-carboxylate (product from Intermediate 33, Step A) in a manner analogous to Intermediate 33, Step B: (Peak 2 on SFC (AD-3_5CM_MeOH(DEA)_5_40_3ML_T35 Column: Chiralpak AD-3 50×4.6 mm I.D., 3 um Mobile phase: methanol (0.05% DEA) in CO₂ from 5% to 40% Flow rate: 3 mL/min Wavelength: 220 nm), retention time=0.842 min, 240 mg, 594.47 μmol, 22.08% yield, 92% purity) as colorless oil.

### Intermediate 35: (3R,8R*)-tert-Butyl 11,11-difluoro-8-(fluoromethyl)-8-hydroxy-3-methyl-3,4,8,9,10,11-hexahydro-1H-pyrido[4′,3′:3,4]pyrazolo[1,5-a]azepine-2(7H)-carboxylate

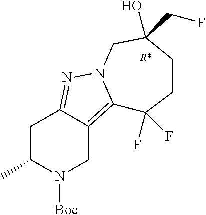 [SMILES: C[C@@H]1Cc2nn3c(c2CN1C(=O)OC(C)(C)C)C(F)(F)CC[C@](O)(CF)C3]

### Step A. (3R)-tert-Butyl 11,11-difluoro-8-hydroxy-8-(hydroxymethyl)-3-methyl-3,4, 8,9,10,11-hexahydro-1H-pyrido[4′,3′:3,4]pyrazolo[1,5-a]azepine-2(7H)-carboxylate

To a solution of (R)-tert-butyl 11,11-difluoro-3-methyl-8-methylene-3,4,8,9,10,11-hexahydro-1H-pyrido[4′,3′:3,4]pyrazolo[1,5-a]azepine-2(7H)-carboxylate (2.5 g, 7.07 mmol) in THF (200 mL) and H₂O (100 mL) were added K₂OsO₄.2H₂O (245 mg, 664.93 μmol) and NMO (1.17 g, 9.99 mmol, 1.05 mL). The mixture was stirred at 25° C. for 32 h. The mixture was diluted with H₂O (200 mL) and extracted with EtOAc (320 mL×3), the combined organic layer was washed with Na₂SO₃ (sat.aq, 150 mL×3), dried over Na₂SO₄, filtered and concentrated in vacuo. The residue was triturated with petroleum ether/EtOAc (10/1, 33 mL) and filtered to give the title compound (2.42 g, 6.25 mmol, 88.30% yield) as white solid. MS (ESI): mass calcd. for C₁₈H₂₇F₂N₃O₄, 387.2; m/z found, 388.0 [M+H]+.

### Step B. (3′R)-tert-Butyl 11′,11′-difluoro-3′-methyl-3′,4′,7′,9′,10′,11′-hexahydrospiro [oxirane-2,8′-pyrido[4′,3′:3,4]pyrazolo[1,5-a]azepine]-2′(1′H)-carboxylate

To a solution of (3R)-tert-butyl 11,11-difluoro-8-hydroxy-8-(hydroxymethyl)-3-methyl-3,4,8,9,10,11-hexahydro-1H-pyrido[4′,3′:3,4]pyrazolo[1,5-a]azepine-2(7H)-carboxylate (1.8 g, 4.65 mmol) in THF (50 mL) was added DBU (1.41 g, 9.29 mmol, 1.40 mL). The resulting solution was cooled to 0° C., and 1,1,2,2,3,3,4,4,4-nonafluorobutane-1-sulfonyl fluoride (2.53 g, 8.36 mmol, 1.47 mL) was added dropwise. The reaction was stirred at 15° C. for 3 h. The solution was concentrated and purified by column chromatography (SiO₂, Petroleum ether/Ethyl acetate=20/1 to 1/1) to give the title compound (2.6 g, impure) as light yellow oil.

### Step C. (3R)-tert-Butyl 11,11-difluoro-8-(fluoromethyl)-8-hydroxy-3-methyl-3,4,8,9,10,11-hexahydro-1H-pyrido[4′,3′:3,4]pyrazolo[1,5-a]azepine-2(7H)-carboxylate

A solution of (3′R)-tert-butyl 11′,11′-difluoro-3′-methyl-3′,4′,7′,9′,10′,11′-hexahydrospiro[oxirane-2,8′-pyrido[4′,3′:3,4]pyrazolo[1,5-a]azepine]-2′(1′H)-carboxylate (1.3 g) in Et₃N₃HF (6.46 g, 40.07 mmol, 6.5 mL) was stirred at 100° C. for 5 h. The mixture was diluted in H₂O (50 mL) and extracted with EtOAc (30 mL×3). The combined organic layer was washed with brine (50 mL), dried over Na₂SO₄, filtered and concentrated. The residue was purified by column chromatography (SiO₂, petroleum ether/ethyl acetate=20/1 to 2/1) twice to give the title compound (1 g, 2.33 mmol, 90.9% purity) as colorless oil. MS (ESI): mass calcd. for C₁₈H₂₆F₃N₃O₃, 389.2; m/z found, 390.2 [M+H]+.

### Step D. (3R,8R*)-tert-Butyl 11,11-difluoro-8-(fluoromethyl)-8-hydroxy-3-methyl-3,4,8,9,10,11-hexahydro-1H-pyrido[4′,3′:3,4]pyrazolo[1,5-a]azepine-2(7H)-carboxylate

(3R)-tert-butyl11,11-difluoro-8-(fluoromethyl)-8-hydroxy-3-methyl-3,4,8,9,10,11-hexahydro-1H-pyrido[4′,3′:3,4]pyrazolo[1,5-a]azepine-2(7H)-carboxylate (1 g, 90% purity) was resolved by SFC (condition: column: DAICEL CHIRALPAK AD-H(250 mm*30 mm,5 um); mobile phase: [0.1% NH₃.H₂O IPA]; B %: 15%-15%,1.7 min; 160 min) to give (3R,8R*)-tert-butyl11,11-difluoro-8-(fluoromethyl) -8-hydroxy-3-methyl-3,4,8,9,10,11-hexahydro-1H -pyrido[4′,3′:3,4]pyrazolo[1,5-a]azepine-2(7H)-carboxylate (Peak 1 on SFC (Amycoat_IPA(DEA)_5_40_3mL-35T Column: Amycoat 50×4.6 mm I.D., 3 um Mobile phase: iso-propanol (0.05% DEA) in CO₂ from 5% to 40% Flow rate: 3 mL/min Wavelength: 220 nm), retention time=0.855 min, 190 mg, 469.85 μmol, 20.33% yield, 96.298% purity. [a]²⁵_{D}=+66.332 (c=0.72, MeOH)) as white solid.

### Intermediate 36: (3R,8S*)-tert-Butyl 11,11-difluoro-8-(fluoromethyl)-8-hydroxy-3-methyl-3,4,8,9,10,11-hexahydro-1H-pyrido[4′,3′:3,4]pyrazolo[1,5-a]azepine-2(7H)-carboxylate

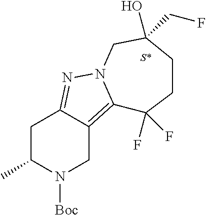 [SMILES: C[C@@H]1Cc2nn3c(c2CN1C(=O)OC(C)(C)C)C(F)(F)CC[C@@](O)(CF)C3]

The title compound was separated by SFC from (3R)-tert-butyl 11,11-difluoro-8-(fluoromethyl)-8-hydroxy-3-methyl-3,4,8,9,10,11-hexahydro-1H-pyrido[4′,3′:3,4]pyrazolo[1,5-a]azepine-2(7H)-carboxylate (product from Intermediate 35, Step C) in a manner analogous to Intermediate 35, Step D: (Peak 2 on SFC (Amycoat_IPA(DEA)_5_40_3mL-35T Column: Amycoat 50×4.6 mm I.D., 3 um Mobile phase: iso-propanol (0.05% DEA) in CO₂ from 5% to 40% Flow rate: 3 mL/min Wavelength: 220 nm), retention time=0.909 min, 200 mg, 507.95 mol, 21.98% yield, 98.9% purity. [a]²⁵_{D}=+25.3 (c=0.51, MeOH)) as white solid.

### Intermediate 37: (3R,8S*)-tert-Butyl 8-(cyanomethyl)-11,11-difluoro-8-hydroxy-3-methyl-3,4,8,9,10,11-hexahydro-1H-pyrido[4′,3′:3,4]pyrazolo[1,5-a]azepine-2(7H)-carboxylate

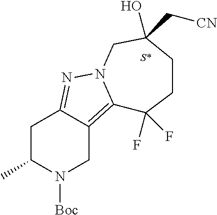 [SMILES: C[C@@H]1Cc2nn3c(c2CN1C(=O)OC(C)(C)C)C(F)(F)CC[C@](O)(CC#N)C3]

### Step A. (3R)-tert-Butyl 8-(cyanomethyl)-11,11-difluoro-8-hydroxy-3-methyl-3,4,8,9,10,11-hexahydro-1H-pyrido[4′,3′:3,4]pyrazolo[1,5-a]azepine-2(7H)-carboxylate

A mixture of (3′R)-tert-butyl 11′,11′-difluoro-3′-methyl-3′,4′,7′,9′,10′,11′-hexahydrospiro[oxirane-2,8′-pyrido[4′,3′:3,4]pyrazolo[1,5-a]azepine]-2′(1′H)-carboxylate (1 g, crude), LiClO₄ (432.01 mg, 4.06 mmol, 178.514) and KCN (264.41 mg, 4.06 mmol, 173.95 μL) in MeCN (3 mL) was degassed and purged with N₂ 3 times. The mixture was stirred at 60° C. for 16 h under N₂ atmosphere. The mixture was poured into ice-water (30 mL) and stirred for 1 min. The aqueous phase was extracted with ethyl acetate (25 mL×2). The combined organic phase was washed with brine (40 mL), dried over anhydrous Na₂SO₄, filtered and concentrated under vacuum. The aqueous phase was quenched by pouring into NaClO (80 mL). The residue was purified by column chromatography (SiO₂, Petroleum ether/Ethyl acetate=5/1 to 2/1) to give the title compound (714 mg, 1.76 mmol, 65.08% yield, 97.82% purity) as white solid. MS (ESI): mass calcd. for C₁₉H₂₆F₂N₄O₃, 396.2; m/z found, 397.3 [M+H]+.

### Step B. (3R,8S*)-tert-Butyl 8-(cyanomethyl)-11,11-difluoro-8-hydroxy-3-methyl-3,4,8,9,10,11-hexahydro-1H-pyrido[4′,3′:3,4]pyrazolo[1,5-a]azepine-2(7H)-carboxylate

(3R)-tert-butyl 8-(cyanomethyl)-11,11-difluoro-8-hydroxy-3-methyl-3,4,8,9,10,11-hexahydro-1H-pyrido[4′,3′:3,4]pyrazolo[1,5-a]azepine-2(7H)-carboxylate (714 mg) was resolved by SFC (condition: column: REGIS (s,s) WHELK-O1 (250 mm*50 mm, 10 um);mobile phase: [0.1% NH₃.H₂O MeOH]; B %: 20%-20%,5.5 min; 400 min) to give (3R,8S*)-tert-butyl-8-(cyanomethyl)-11,11-difluoro-8-hydroxy-3-methyl-3,4,8,9,10,11-hexahydro-1H-pyrido[4′,3′:3,4]pyrazolo[1,5-a]azepine-2(7H)-carboxylate (Peak 1 on SFC (Whelk-o1-3_10CM_MeOH(DEA)_5_40_3ML_T35 Column: Chiralcel Whelk-o1-3 100×4.6 mm I.D., 3 um Mobile phase: methanol (0.05% DEA) in CO₂ from 5% to 40% Flow rate: 3 mL/min Wavelength: 220 nm), retention time=1.819 min, 351 mg, 876.55 μmol, 49.75% yield, 99% purity, [a]²⁵_{D}=+22.78 (c=+0.66, MeOH)) as colorless oil.

### Intermediate 38: (3R,8R*)-tert-Butyl 8-(cyanomethyl)-11,11-difluoro-8-hydroxy-3-methyl-3,4,8,9,10,11-hexahydro-1H-pyrido[4′,3′:3,4]pyrazolo[1,5-a]azepine-2(7H)-carboxylate

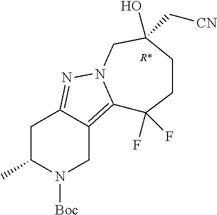 [SMILES: C[C@@H]1Cc2nn3c(c2CN1C(=O)OC(C)(C)C)C(F)(F)CC[C@@](O)(CC#N)C3]

The title compound was separated by SFC from (3R)-tert-butyl 8-(cyanomethyl)-11,11-difluoro-8-hydroxy-3-methyl-3,4,8,9,10,11-hexahydro-1H-pyrido[4′,3′:3,4]pyrazolo[1,5-a]azepine-2(7H)-carboxylate (product from Intermediate 37, Step A) in a manner analogous to Intermediate 37, Step B: (Peak 2 on SFC, retention time=1.889 min, 340 mg, 797.62 μmol, 45.27% yield, 93% purity, [a]²⁵_{D}=+46.36 (c=0.48, MeOH)) as colorless oil.

### Intermediate 39: (3R,8R*)-tert-Butyl 8-((2,2-difluoroethoxy)methyl)-11,11-difluoro-8-hydroxy-3-methyl-3,4,8,9,10,11-hexahydro-1H-pyrido[4′,3′:3,4]pyrazolo[1,5-a]azepine-2(7H)-carboxylate

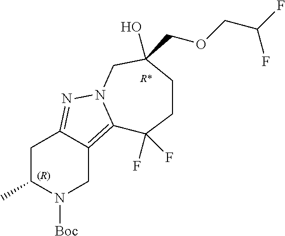

### Step A. (3R)-tert-Butyl 8-((2,2-difluoroethoxy)methyl)-11,11-difluoro-8-hydroxy-3-methyl-3,4,8,9,10,11-hexahydro-1H-pyrido[4′,3′:3,4]pyrazolo[1,5-a]azepine-2(7H) -carboxylate

To a solution of (3R)-tert-butyl 11,11-difluoro-8-hydroxy-8-(hydroxymethyl)-3-methyl-3,4,8,9,10,11-hexahydro-1H-pyrido[4′,3′:3,4]pyrazolo[1,5-a]azepine-2(7H)-carboxylate (1 g, 2.58 mmol) in THF (10 mL) and DMF (5 mL) was added NaHMDS (1 M, 3.18 mL) at −78° C. The mixture was stirred at −78° C. for 0.5 h, then a solution of 2,2-difluoroethyl trifluoromethanesulfonate (785.88 mg, 3.67 mmol) in THF (2 mL) was added at −78° C., and stirred at −78° C. for 0.5 h. The mixture was quenched with NH₄Cl (sat.aq, 100 mL) and extracted with EtOAc (100 mL×2), the combined organic layer was dried over Na₂SO₄, filtered. The filtrate was concentrated in vacuo. The residue was purified by column chromatography (SiO₂, Petroleum/EtOAc=10/1 to 2/1EtOAc/MeOH=10/1) twice to give the title compound (430 mg, 922.00 μmol, 35.72% yield, 96.8% purity) as colorless oil. MS (ESI): mass calcd. for C₂₀H₂₉F₄N₃O₄, 451.2; m/z found, 452.1 [M+H]+.

### Step B. (3R,8R*)-tert-Butyl 8-((2,2-difluoroethoxy)methyl)-11,11-difluoro-8-hydroxy-3-methyl-3,4,8,9,10,11-hexahydro-1H-pyrido[4′,3′:3,4]pyrazolo[1,5-a]azepine-2(7H)-carboxylate

(3R)-tert-butyl 8-((2,2-difluoroethoxy)methyl)-11,11-difluoro-8-hydroxy-3-methyl-3,4,8,9,10,11-hexahydro-1H-pyrido[4′,3′:3,4]pyrazolo[1,5-a]azepine-2(7H)-carboxylate (430 mg) was resolved by SFC (condition: column: DAICEL CHIRALPAK AD-H(250 mm*30 mm, 5 um); mobile phase: [0.1% NH₃.H₂O EtOH]; B %: 15%-15%,1.8 min; 90 min) to give (3R,8R*)-tert-butyl 8-((2,2-difluoroethoxy)methyl) -11,11-difluoro-8-hydroxy-3-methyl-3,4,8,9,10,11-hexahydro-1H-pyrido[4′,3′:3,4]pyrazolo[1,5-a]azepine-2(7H)-carboxylate (Peak 1 on SFC (Amycoat_EtOH(DEA)_5_40_3mL-35T Column:Amycoat 50×4.6 mm I.D., 3 um Mobile phase: ethanol (0.05% DEA) in CO₂ from 5% to 40% Flow rate: 3 mL/min Wavelength: 220 nm), retention time=0.638 min, 150 mg, 322.29 μmol, 33.84% yield, 97% purity, [a]²⁵_{D}=+61.2 (c=0.5, MeOH)) as white solid.

### Intermediate 40: (3R,8S*)-tert-Butyl 8-((2,2-difluoroethoxy)methyl)-11,11-difluoro-8-hydroxy-3-methyl-3,4,8,9,10,11-hexahydro-1H-pyrido[4′,3′:3,4]pyrazolo[1,5-a]azepine-2(7H)-carboxylate

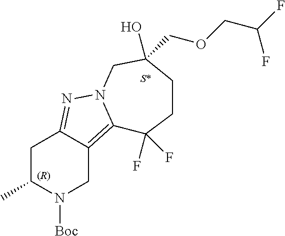

The title compound was resolved by SFC from (3R)-tert-butyl 8-((2,2-difluoroethoxy)methyl)-11,11-difluoro-8-hydroxy-3-methyl-3,4,8,9,10,11-hexahydro-1H-pyrido[4′,3′:3,4]pyrazolo[1,5-a]azepine-2(7H)-carboxylate (product from Intermediate 39, Step A) in a manner analogous to Intermediate 39, Step B: (Peak 2 on SFC (Amycoat_EtOH(DEA)_5_40_3mL-35T Column:Amycoat 50×4.6 mm I.D., 3 um Mobile phase: ethanol (0.05% DEA) in CO₂ from 5% to 40% Flow rate: 3 mL/min Wavelength: 220 nm), retention time=0.744 min, 170 mg, 365.26 μmol, 38.35% yield, 97% purity) as white solid.

### Intermediate 41: (3R,8R*)-tert-Butyl 8-ethynyl-11,11-difluoro-8-hydroxy-3-methyl-3,4,8,9,10,11-hexahydro-1H-pyrido[4′,3′:3,4]pyrazolo[1,5-a]azepine-2(7H)-carboxylate

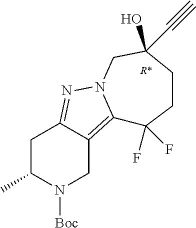 [SMILES: C#C[C@]1(O)CCC(F)(F)c2c3c(nn2C1)C[C@@H](C)N(C(=O)OC(C)(C)C)C3]

### Step A. (3R)-tert-Butyl 8-ethynyl-11,11-difluoro-8-hydroxy-3-methyl-3,4,8,9,10,11-hexahydro-1H-pyrido[4′,3′:3,4]pyrazolo[1,5-a]azepine-2(7H)-carboxylate

To a solution of (R)-tert-butyl 11,11-difluoro-3-methyl-8-oxo-3,4,8,9,10,11-hexahydro-1H-pyrido[4′,3′:3,4]pyrazolo[1,5-a]azepine-2(7H)-carboxylate (350 mg, 955.32 μmol) and LiCl (80.99 mg, 1.91 mmol, 39.13 μL) in DCM (10 mL) was added bromo(ethynyl)magnesium (0.5 M, 9.55 mL) at 0° C. The mixture was stirred at 15° C. for 14 h. The mixture was quenched by NH₄Cl (sat.aq, 100 mL) and then extracted with EtOAc (100 mL×2). The combined organic phase was washed with brine (100 mL), dried over anhydrous Na₂SO₄, filtered and concentrated in vacuo. The residue was purified by RP HPLC(condition D) to give the title compound (690 mg, 1.77 mmol, 61.86% yield, 98% purity) as yellow solid. MS (ESI): mass calcd. for C₁₉H₂₅F₂N₃O₃, 381.2; m/z found, 382.4 [M+H]+.

### Step B. (3R,8R*)-tert-Butyl 8-ethynyl-11,11-difluoro-8-hydroxy-3-methyl-3,4,8,9,10,11-hexahydro-1H-pyrido[4′,3′:3,4]pyrazolo[1,5-a]azepine-2(7H)-carboxylate

(3R)-tert-butyl 8-ethynyl-11,11-difluoro-8-hydroxy-3-methyl-3,4,8,9,10,11-hexahydro-1H-pyrido[4′,3′:3,4]pyrazolo[1,5-a]azepine-2(7H)-carboxylate (690 mg) was resolved by SFC (condition: column: DAICEL CHIRALPAK AD-H(250 mm*30 mm,5 um); mobile phase: [0.1% NH₃.H₂O IPA]; B %: 20%-20%, 1.3 min: 110 min) to give (3R,8R*)-tert-butyl 8-ethynyl-11,11-difluoro-8-hydroxy-3-methyl-3,4,8,9,10,11-hexahydro-1H-pyrido [4′,3′:3,4]pyrazolo[1,5-a]azepine-2(7H)-carboxylate (Peak 1 on SFC (AD-3_5CM_MeOH(DEA)_5_40_3ML_T35 Column: Chiralpak AD-3 50×4.6 mm I.D., 3 um Mobile phase: methanol (0.05% DEA) in CO₂ from 5% to 40% Flow rate: 3 mL/min Wavelength: 220 nm), retention time=0.842 min, 240 mg, 616.65 wok 34.78% yield, 98% purity) as white solid.

### Intermediate 42: (3R,8S*)-tert-Butyl 8-ethynyl-11,11-difluoro-8-hydroxy-3-methyl-3,4,8,9,10,11-hexahydro-1H-pyrido[4′,3′:3,4]pyrazolo[1,5-a]azepine-2(7H)-carboxylate

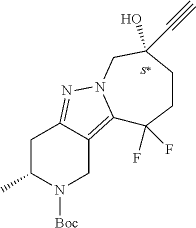 [SMILES: C#C[C@@]1(O)CCC(F)(F)c2c3c(nn2C1)C[C@@H](C)N(C(=O)OC(C)(C)C)C3]

The title compound was separated by SFC from (3R)-tert-butyl 8-ethynyl-11,11-difluoro-8-hydroxy-3-methyl-3,4,8,9,10,11-hexahydro-1H-pyrido[4′,3′:3,4]pyrazolo[1,5-a]azepine-2(7H)-carboxylate (product from Intermediate 41, Step A) in a manner analogous to Intermediate 41, Step B: (Peak 2 on SFC (AD-3_5CM_MeOH(DEA)_5_40_3ML_T35 Column: Chiralpak AD-3 50×4.6 mm I.D., 3 um Mobile phase: methanol (0.05% DEA) in CO₂ from 5% to 40% Flow rate: 3 mL/min Wavelength: 220 nm), retention time=0.937 min, 350 mg, 899.28 μmol, 50.72% yield, 98% purity, [a]²⁵_{D}=+31.4 (c=0.47, CH₃Cl)) as white solid.

### Intermediate 43: (3R,8S*)-tert-Butyl 11,11-difluoro-8-(2-hydroxypropan-2-yl)-3-methyl-3,4,8,9,10,11-hexahydro-1H-pyrido[4′,3′:3,4]pyrazolo[1,5-a]azepine-2(7H)-carboxylate

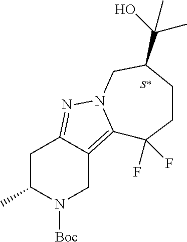 [SMILES: C[C@@H]1Cc2nn3c(c2CN1C(=O)OC(C)(C)C)C(F)(F)CC[C@H](C(C)(C)O)C3]

### Step A. (3R)-2-(tert-Butoxycarbonyl)-11,11-difluoro-3-methyl-2,3,4,7,8,9,10,11-octahydro-1H-pyrido[4′,3′:3,4]pyrazolo[1,5-a]azepine-8-carboxylic acid

To a solution of (3R)-tert-butyl 11,11-difluoro-8-(hydroxymethyl)-3-methyl-3,4,8,9,10,11-hexahydro-1H-pyrido[4′,3′:3,4]pyrazolo[1,5-a]azepine-2(7H)-carboxylate (250 mg, 673.09 μμmol) in MeCN (4 mL) was added TPAP (59.14 mg, 168.27 μmol) and NMO (394.26 mg, 3.37 mmol, 355.19 μL). The mixture was stirred at 20° C. for 3 h. The mixture was concentrated in vacuo to give the title compound (800 mg, crude) as black oil, MS (ESI): mass calcd. for C₁₈H₂₅F₂N₃O₄ 385.2; m/z found, 386.3 [M+H]+.

### Step B. (3R)-2-tert-Butyl 8-ethyl 11,11-difluoro-3-methyl-3,4,8,9,10,11-hexahydro-1H-pyrido[4′,3′:3,4]pyrazolo[1,5-a]azepine-2,8(7H)-dicarboxylate

To a solution of (3R)-2-(tert-butoxycarbonyl)-11,11-difluoro-3-methyl-2,3,4,7, 8,9,10,11-octahydro-1H-pyrido[4′,3′:3,4]pyrazolo[1,5-a]azepine-8-carboxylic acid (800 mg, crude) in MeCN (5 mL) was added Cs₂CO₃ (676.32 mg, 2.08 mmol) followed by EtI (323.75 mg, 2.08 mmol, 166.03 μL). The mixture was stirred at 20° C. for 16 h. The mixture was diluted with MeCN (50 mL) and filtered. The filtrate was concentrated in vacuo. The residue was purified by column chromatography (SiO₂, Petroleum ether/EtOAc=1/01/9) to give the title compound (150 mg, 362.79 μmol) as colorless oil. MS (ESI): mass calcd. for C₂₀H₂₉F₂N₃O₄ 413.2; m/z found, 414.4 [M+H]+.

### Step C. (3R)-tert-Butyl 11,11-difluoro-8-(2-hydroxypropan-2-yl)-3-methyl-3,4,8,9,10,11-hexahydro-1H-pyrido[4′,3′:3,4]pyrazolo[1,5-a]azepine-2(7H)-carboxylate

To a solution of (3R)-2-tert-butyl 8-ethyl 11,11-difluoro-3-methyl-3,4,8,9,10,11-hexahydro-1H-pyrido[4′,3′:3,4]pyrazolo[1,5-a]azepine-2,8(7H)-dicarboxylate (220 mg, 532.10 μmol) in THF (3 mL) was added MeMgBr (3 M, 1 mL) at −40° C. The mixture was stirred at 0° C. for 2 h. The reaction was quenched with NH₄Cl (sat.aq, 10 mL) and extracted with EtOAc (20 mL×3), the combined organic layer was dried over Na₂SO₄, filtered and concentrated in vacuo. The residue was purified by column chromatography (SiO₂, Petroleum ether/EtOAc=10/1 to 1/1) to give the title compound (180 mg, 450.59 μmol, 84.68% yield) as colorless oil.

### Step D. (3R,8S*)-tert-Butyl 11,11-difluoro-8-(2-hydroxypropan-2-yl)-3-methyl-3,4,8,9,10,11-hexahydro-1H-pyrido[4′,3′:3,4]pyrazolo[1,5-a]azepine-2(7H)-carboxylate

(3R)-tert-butyl11,11-difluoro-8-(2-hydroxypropan-2-yl)-3-methyl-3,4,8,9,10,11-hexahydro-1H-pyrido[4′,3′:3,4]pyrazolo[1,5-a]azepine-2(7H)-carboxylate (160 mg) was separated by SFC (condition: column: DAICEL CHIRALPAK AD(250 mm*30 mm,10 um); mobile phase: [0.1% NH₃.H₂O EtOH]; B %: 30%-30%, 1.8 min: 60 min) to give (3R,8S*)-tert-butyl-11,11-difluoro-8-(2-hydroxypropan-2-yl)-3-methyl-3,4,8,9,10,11-hexahydro-1H-pyrido[4′,3′:3,4]pyrazolo[1,5-a]azepine-2(7H)-carboxylate (Peak 1 on SFC (Amycoat_EtOH(DEA)_5_40_3mL-35T Column:Amycoat 50×4.6 mm I.D., 3 um Mobile phase: ethanol (0.05% DEA) in CO₂ from 5% to 40% Flow rate: 3 mL/min Wavelength: 220 nm.), retention time=0.814 min, 52 mg, 93% purity) as yellow solid.

### Intermediate 44: (3R,8R*)-tert-Butyl 11,11-difluoro-8-(2-hydroxypropan-2-yl)-3-methyl-3,4,8,9,10,11-hexahydro-1H-pyrido[4′,3′:3,4]pyrazolo[1,5-a]azepine-2(7H)-carboxylate

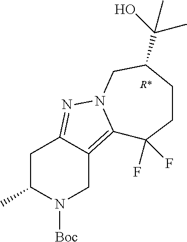 [SMILES: C[C@@H]1Cc2nn3c(c2CN1C(=O)OC(C)(C)C)C(F)(F)CC[C@@H](C(C)(C)O)C3]

The title compound was separated by SFC from (3R)-tert-butyl 11,11-difluoro-8-(2-hydroxypropan-2-yl)-3-methyl-3,4,8,9,10,11-hexahydro-1H-pyrido[4′,3′:3,4]pyrazolo[1,5-a]azepine-2(7H)-carboxylate (product from Intermediate 43, step C) in a manner analogous to Intermediate 43, Step D: (Peak 2 on SFC (Amycoat_EtOH(DEA)_5_40_3mL-35T Column:Amycoat 50×4.6 mm I.D., 3 um Mobile phase: ethanol (0.05% DEA) in CO₂ from 5% to 40% Flow rate: 3 mL/min Wavelength: 220 nm.), retention time=0.934 min, 45 mg, 98% purity) was obtained as yellow solid.

### Intermediate 45: (3R,8R*)-tert-Butyl 11,11-difluoro-8-(((methoxycarbonyl)amino) methyl)-3-methyl-3,4,8,9,10,11-hexahydro-1H-pyrido[4′,3′:3,4]pyrazolo[1,5-a]azepine-2(7H)-carboxylate

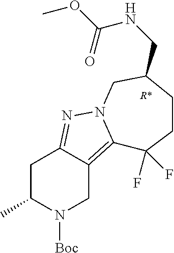 [SMILES: COC(=O)NC[C@H]1CCC(F)(F)c2c3c(nn2C1)C[C@@H](C)N(C(=O)OC(C)(C)C)C3]

### Step A. (3R)-tert-Butyl 11,11-difluoro-3-methyl-8-(((methylsulfonyl)oxy)methyl)-3,4,8,9,10,11-hexahydro-1H-pyrido[4′,3′:3,4]pyrazolo[1,5-a]azepine-2(7H)-carboxylate

To a solution of (3R)-tert-butyl 11,11-difluoro-8-(hydroxymethyl)-3-methyl-3,4,8,9,10,11-hexahydro-1H-pyrido[4′,3′:3,4]pyrazolo[1,5-a]azepine-2(7H)-carboxylate (600 mg, 1.62 mmol) in DCM (10 mL) was added MsCl (222.06 mg, 1.94 mmol, 150.04 4) and TEA (490.39 mg, 4.85 mmol, 674.54 μL) at 0° C. The mixture was stirred at 0° C. for 1 h. The mixture was poured into ice-water (20 mL). The aqueous phase was extracted with DCM (10 mL). The organic phase was dried over Na₂SO₄, filtered and concentrated in vacuum to give the title compound (735 mg, crude) as a colorless oil,

### Step B. (3R)-tert-Butyl 8-(azidomethyl)-11,11-difluoro-3-methyl-3,4,8,9,10,11-hexahydro-1H-pyrido[4′,3′:3,4]pyrazolo[1,5-a]azepine-2(7H)-carboxylate

To a solution of (3R)-tert-butyl 11,11-difluoro-3-methyl-8-(((methylsulfonyl)oxy)methyl)-3,4,8,9,10,11-hexahydro-1H-pyrido[4′,3′:3,4]pyrazolo[1,5-a]azepine-2(7H)-carboxylate (726 mg, crude) in DMF (10 mL) was added NaN₃ (419.99 mg, 6.46 mmol) at 0° C. under N₂. The mixture was stirred at 50° C. for 12 h. The mixture was diluted with EtOAc (40 mL) and washed with brine (20 mL×3). The organic phase was dried over Na₂SO₄, filtered and concentrated in vacuo to give the title compound (652 mg, crude) as a colorless oil. MS (ESI): mass calcd. for C₁₈H₂₆F₂N₆O₂ 396.2; m/z found, 397.4 [M+H]+.

### Step C. (3R)-tert-Butyl 8-(aminomethyl)-11,11-difluoro-3-methyl-3,4,8,9,10,11-hexahydro-1H-pyrido[4′,3′:3,4]pyrazolo[1,5-a]azepine-2(7H)-carboxylate

A mixture of (3R)-tert-butyl 8-(azidomethyl)-11,11-difluoro-3-methyl-3,4,8,9,10,11-hexahydro-1H-pyrido[4′,3′:3,4]pyrazolo[1,5-a]azepine-2(7H)-carboxylate (640 mg, crude), NH₄Cl (259.07 mg, 4.84 mmol), Zn (211.13 mg, 3.23 mmol) in EtOH (8 mL) and H₂O (0.8 mL) was degassed and purged with N₂ 3 times. The mixture was stirred at 15° C. for 16 h under N₂ atmosphere. The mixture was filtered and concentrated in vacuum to give the title compound (602 mg, crude) as a white solid. MS (ESI): mass calcd. for C₁₈H₂₈F₂N₄O₂ 370.2; m/z found, 371.4 [M+H]+.

### Step D. (3R)-tert-Butyl 11,11-difluoro-8-(((methoxycarbonyl)amino)methyl)-3-methyl-3,4,8,9,10,11-hexahydro-1H-pyrido[4′,3′:3,4]pyrazolo[1,5-a]azepine-2(7H)-carboxylate

A mixture of (3R)-tert-butyl 8-(aminomethyl)-11,11-difluoro-3-methyl-3,4,8,9,10,11-hexahydro-1H-pyrido[4′,3′:3,4]pyrazolo[1,5-a]azepine-2(7H)-carboxylate (598 mg, crude), TEA (816.76 mg, 8.07 mmol) in DCM (10 mL) was added methyl carbonochloridate (610.19 mg, 6.46 mmol) at 0° C. under N₂. The mixture was stirred at 10° C. for 1 h under N₂ atmosphere. The mixture was poured into ice-water (20 mL). The aqueous phase was extracted with DCM (10 mL). The organic phase was dried over Na₂SO₄, filtered and concentrated in vacuo. The residue was purified by column chromatography (SiO₂, Petroleum ether/Ethyl acetate=100/1 to 1:1) to give the title compound (390 mg, 910.21 μmol) as a white solid. MS (ESI): mass calcd. for C₂₀H₃₀F₂N₄O₄ 428.2; m/z found, 429.4 [M+H]+.

### Step E. (3R,8R*)-tert-Butyl 11,11-difluoro-8-(((methoxycarbonyl)amino)methyl)-3-methyl-3,4,8,9,10,11-hexahydro-1H-pyrido[4′,3′:3,4]pyrazolo[1,5-a]azepine-2(7H)-carboxylate

(3R)-tert-butyl 11,11-difluoro-8-(((methoxycarbonyl)amino)methyl)-3-methyl-3,4,8,9,10,11-hexahydro-1H-pyrido[4′,3′:3,4]pyrazolo[1,5-a]azepine-2(7H)-carboxylate (535 mg) was resolved by SFC(condition: column: DAICEL CHIRALPAK AD-H(250 mm*30 mm,5 um); mobile phase: [0.1% NH₃.H₂O IPA]; B %: 20%-20%, 2.3 min; 150 min) to give (3R,8R*)-tert-butyl 11,11-difluoro-8-(((methoxycarbonyl) amino)methyl)-3-methyl-3,4,8,9,10,11-hexahydro-1H-pyrido[4′,3′:3,4]pyrazolo[1,5-a]azepine-2(7H)-carboxylate (peak 1 on SFC(“Amycoat_IPA(DEA)_5_40_3mL-35T Column: Amycoat 50×4.6 mm I.D., 3 um Mobile phase: iso-propanol (0.05% DEA) in CO₂ from 5% to 40% Flow rate: 3 mL/min Wavelength: 220 nm”), retention time=1.145 min, 182 mg, 399.28 wok 31.98% yield, 94% purity, [a]²⁵_{D}=+26.45 (c=0.51, MeOH)) as colorless oil.

### Intermediate 46: (3R,8S*)-tert-Butyl 11,11-difluoro-8-(((methoxy carbonyl)amino) methyl)-3-methyl-3,4,8,9,10,11-hexahydro-1H-pyrido[4′,3′:3,4]pyrazolo[1,5-a]azepine-2(7H)-carboxylate

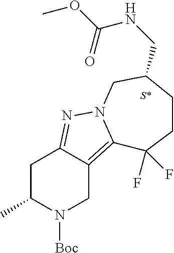 [SMILES: COC(=O)NC[C@@H]1CCC(F)(F)c2c3c(nn2C1)C[C@@H](C)N(C(=O)OC(C)(C)C)C3]

The title compound was resolved by SFC from (3R)-tert-butyl 11,11-difluoro-8-(((methoxycarbonyl)amino)methyl)-3-methyl-3,4,8,9,10,11-hexahydro-1H-pyrido[4′,3′:3,4]pyrazolo[1,5-a]azepine-2(7H)-carboxylate in a manner analogous to Intermediate 45, Step E: (Peak 2 on SFC (“Amycoat_IPA(DEA)_5_40_3mL-35T Column: Amycoat 50×4.6 mm I.D., 3 um Mobile phase: iso-propanol (0.05% DEA) in CO₂ from 5% to 40% Flow rate: 3 mL/min Wavelength: 220 nm”), retention time=1.249 min, 288 mg, 651.99 wok 52.22% yield, 97% purity, [a]²⁵_{D}=+59.53 (c=0.51, MeOH)) as white solid.

### Intermediate 47: (3R,8S*)-tert-Butyl 8,11,11-trifluoro-8-(hydroxymethyl)-3-methyl-3,4,8,9,10,11-hexahydro-1H-pyrido[4′,3′:3,4]pyrazolo[1,5-a]azepine-2(7H)-carboxylate

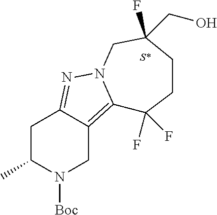 [SMILES: C[C@@H]1Cc2nn3c(c2CN1C(=O)OC(C)(C)C)C(F)(F)CC[C@@](F)(CO)C3]

### Step A. (3R)-2-tert-Butyl 8-ethyl 8,11,11-trifluoro-3-methyl-3,4,8,9,10,11-hexahydro-1H-pyrido[4′,3′:3,4]pyrazolo[1,5-a]azepine-2,8(7H)-dicarboxylate

To a solution of (3R)-2-tert-butyl 8-ethyl 11,11-difluoro-3-methyl-3,4,8,9,10,11-hexahydro-1H-pyrido[4′,3′:3,4]pyrazolo[1,5-a]azepine-2,8(7H)-dicarboxylate (300 mg, 725.59 μmol) and NFSI (549.14 mg, 1.74 mmol) in THF (5 mL) was added LiHMDS (1 M, 1.74 mL) dropwise at −70° C. The mixture was stirred at −70° C. for 1 h. The mixture was poured into NH₄Cl (sat.aq,20 mL) at 0° C. and extracted with EtOAc (30 mL×2), the combined organic layer was dried over Na₂SO₄, filtered and concentrated in vacuo. The residue was purified by column chromatography (SiO₂, Petroleum ether/Ethyl acetate=10% to 30%) to give the title compound (250 mg, 557.43 μmol, 76.82% yield, 96.2% purity) as yellow oil. MS (ESI): mass calcd. for C₂₀H₂₈F₃N₃O₄ 431.2; m/z found, 432.3 [M+H]+.

### Step B. (3R)-tert-Butyl 8,11,11-trifluoro-8-(hydroxymethyl)-3-methyl-3,4,8,9,10,11-hexahydro-1H-pyrido[4′,3′:3,4]pyrazolo[1,5-a]azepine-2(7H)-carboxylate

To a suspension of LiBH₄ (89.37 mg, 4.10 mmol) in THF (1 mL) was added a solution of (3R)-2-tert-butyl 8-ethyl 8,11,11-trifluoro-3-methyl-3,4,8,9,10,11-hexahydro-1H-pyrido[4′,3′:3,4]pyrazolo[1,5-a]azepine-2,8(7H)-dicarboxylate (590 mg, 1.37 mmol) in THF (1 mL) at 0° C. The mixture was stirred at 15° C. for 1 h. The mixture was poured into NH₄Cl (sat.aq, 30 mL) at 0° C. and the resulting mixture was stand for 16° C. The resulting mixture was extracted with EtOAc(80 mL×3). The combined organic layer was dried over Na₂SO₄, filtered and concentrated in vacuo. The residue was purified by column chromatography (SiO₂, Petroleum ether/Ethyl acetate=20% to 30%) to give the title compound (450 mg, 1.13 mmol, 82.98% yield, 98.2% purity) as colorless oil. MS (ESI): mass calcd. for C₁₈H₂₆F₃N₃O₃ 389.2; m/z found, 390.4 [M+H]+.

### Step C. (3R,8S*)-tert-Butyl 8,11,11-trifluoro-8-(hydroxymethyl)-3-methyl-3,4,8,9, 10,11-hexahydro-1H-pyrido[4′,3′:3,4]pyrazolo[1,5-a]azepine-2(7H)-carboxylate

(3R)-tert-butyl 8,11,11-trifluoro-8-(hydroxymethyl)-3-methyl-3,4,8,9,10,11-hexahydro-1H-pyrido[4′,3′:3,4]pyrazolo[1,5-a]azepine-2(7H)-carboxylate was resolved by SFC(column: DAICEL CHIRALPAK AD-H(250 mm*30 mm,5 um); mobile phase: [0.1% NH₃H₂O MEOH]; B %: 25%-25%, 3.2 min; 55 min) to give (3R,8S*)-tert-butyl 8,11,11-trifluoro-8-(hydroxymethyl)-3-methyl-3,4,8,9,10,11-hexahydro-1H-pyrido[4′,3′:3,4]pyrazolo[1,5-a]azepine-2(7H)-carboxylate(peak 1 on SFC “Amycoat_MeOH(DEA)_5_40_3mL-35T Column: Amycoat 50×4.6 mm I.D., 3 um Mobile phase: methanol (0.05% DEA) in CO₂ from 5% to 40% Flow rate: 3 mL/min Wavelength: 220 nm”, Retention time=0.733, 330 mg, 832.18 mol, 58.92% yield, 98.2% purity, [a]²⁵_{D}=+67.3 (c=0.53, MeOH)) as colorless oil.

### Intermediate 48: (3R,8R*)-tert-Butyl 8,11,11-trifluoro-8-(hydroxymethyl)-3-methyl-3,4,8,9,10,11-hexahydro-1H-pyrido[4′,3′:3,4]pyrazolo[1,5-a]azepine-2(7H)-carboxylate

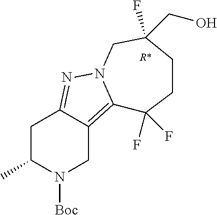 [SMILES: C[C@@H]1Cc2nn3c(c2CN1C(=O)OC(C)(C)C)C(F)(F)CC[C@](F)(CO)C3]

The title compound was resolved by SFC from (3R)-tert-butyl 8,11,11-trifluoro-8-(hydroxymethyl)-3-methyl-3,4,8,9,10,11-hexahydro-1H-pyrido[4′,3′:3,4]pyrazolo[1,5-a]azepine-2(7H)-carboxylate in a manner analogous to intermediate 47, Step C (peak 2 on SFC“Amycoat_MeOH(DEA)_5_40_3mL-35T Column: Amycoat 50×4.6 mm I.D., 3 um Mobile phase: methanol (0.05% DEA) in CO₂ from 5% to 40% Flow rate: 3 mL/min Wavelength: 220 nm”, retention time=0.947, 130 mg, 332.50 mol, 23.54% yield, 99.6% purity, [a]²⁵_{D}=+30.2 (c=0.4, MeOH)) as white solid.

### Intermediate 49: (R)-tert-Butyl 11,11-difluoro-3-methyl-9-methylene-3,4,8,9,10,11-hexahydro-1H-pyrido[4′,3′:3,4]pyrazolo[1,5-a]azepine-2(7H)-carboxylate

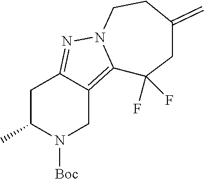

Step A. Ethyl 4-hydroxy-2-methylenebutanoate. To a solution of ethyl 2-(bromomethyl)prop-2-enoate (42 g, 217.57 mmol) in EtOH (400 mL) and H₂O (200 mL) were added indium (27.48 g, 239.33 mmol) and formaldehyde (31.78 g, 391.63 mmol, 29.16 mL, 37% purity). The mixture was stirred at 15° C. for 16 hr. The solution was poured into aqueous 1 N HCl (600 mL). The mixture was extracted with ethyl acetate (400 mL×2). The combined organic phase was washed with sat. aq NaHCO₃(300 mL) and brine (300 mL), dried over anhydrous Na₂SO₄, filtered and concentrated. The residue was purified by column chromatography (SiO₂, Petroleum ether/Ethyl acetate=2/1) to give the title compound (28 g, 194.22 mmol, 89.27% yield) as colorless oil. ¹H NMR (400 MHz, CDCl₃) δ=6.24 (s, 1H), 5.66 (s, 1H), 4.28-4.16 (m, 2H), 3.76 (t, J=6.0 Hz, 2H), 2.58 (t, J=6.4 Hz, 2H), 1.35-1.26 (m, 3H).

Step B. Ethyl 4-((tert-butyldiphenylsilyl)oxy)-2-methylenebutanoate. To a solution of ethyl 4-hydroxy-2-methylene-butanoate (28 g, 194.22 mmol) and TBDPSCl (53.38 g, 194.22 mmol) in DCM (300 mL) was added imidazole (15.87 g, 233.06 mmol) at 15° C. under N₂. The mixture was stirred at 15° C. for 2 h. The reaction mixture was quenched with 1 N HCl (500 mL) at 0° C., and then extracted with dichloromethane (400 mL×2). The combined organic layer was washed with brine (800 mL), dried over Na₂SO₄, filtered and concentrated under reduced pressure. The residue was purified by column chromatography (SiO₂, Petroleum ether/Ethyl acetate=20/1) to give the title compound (50 g, 130.70 mmol, 67.29% yield) as colorless oil. ¹H NMR (400 MHz, CDCl₃) δ=7.80-7.62 (m, 4H), 7.46-7.30 (m, 6H), 6.23 (s, 1H), 5.22 (s, 1H), 4.22-4.09 (m, 2H), 3.80 (t, J=6.4 Hz, 2H), 2.63-2.55 (m, 2H), 1.32-1.23 (m, 3H), 1.05 (s, 9H).

Step C. 4-((tert-Butyldiphenylsilyl)oxy)-2-methylenebutan-1-ol. To a solution of ethyl 4-((tert-butyldiphenylsilyl)oxy)-2-methylenebutanoate (50 g, 130.70 mmol) in THF (500 mL) was added DIBAL-H (1 M, 261.39 mL) at −60° C. under N₂. The solution was stirred at −40° C. for 2 h. The solution was poured into sat. L (+)-tartaric acid potassium Sodium salt (Seignette salt/Rochelle salt) (800 mL), then ethyl acetate (300 mL) was added. The mixture was stirred for 16 h. The mixture was extracted with ethyl acetate (500 mL). The combined organic phase was washed with brine (800 mL), dried with anhydrous Na₂SO₄, filtered and concentrated. The residue was purified by column chromatography (SiO₂, Petroleum ether/Ethyl acetate=1/0-10/1) to give the title compound (40 g, 117.46 mmol, 89.88% yield) as colorless oil. ¹H NMR (400 MHz, CDCl₃) δ=7.71-7.63 (m, 4H), 7.48-7.34 (m, 6H), 5.08 (s, 1H), 4.90 (s, 1H), 4.09 (s, 2H), 3.83-3.73 (m, 2H), 2.36 (t, J=6.0 Hz, 2H), 1.06 (s, 9H).

Step D. ((3-(Bromomethyl)but-3-en-1-yl)oxy)(tert-butyl)diphenylsilane. To a solution of 4-((tert-butyldiphenylsilyl)oxy)-2-methylenebutan-1-ol (40 g, 117.46 mmol) in DCM (500 mL) was added CBr₄ (58.43 g, 176.20 mmol) and PPh₃ (46.21 g, 176.20 mmol) under N₂. The solution was stirred at 15° C. for 2 h. The reaction mixture was concentrated under reduced pressure and triturated in Petroleum ether/Ethyl acetate=10/1 (200 mL). the filtrate was concentrated under reduced pressure. The residue was purified by column chromatography (SiO₂, Petroleum ether/Ethyl acetate=1/0) to give the title compound (27 g, 66.93 mmol) as colorless oil. ¹H NMR (400 MHz, CDCl₃) δ=7.71-7.63 (m, 4H), 7.48-7.34 (m, 6H), 5.22 (s, 1H), 5.00 (s, 1H), 3.98 (d, J=13.2 Hz, 2H), 3.86-3.77 (m, 2H), 2.47 (t, J=6.4 Hz, 2H), 1.06 (s, 9H).

Step E. Ethyl 6-((tert-butyldiphenylsilyl)oxy)-2,2-difluoro-4-methylenehexanoate. To a suspension of activated Zn (7.44 g, 113.78 mmol) in triglyme (200 mL) was added ethyl 2-bromo-2,2-difluoro-acetate (29.08 g, 143.27 mmol) dropwise under N₂. The mixture was sonicated at 35° C. until Zn was dissolved in triglyme completely. Then CuCN (10.57 g, 117.99 mmol) was added and the mixture was stirred at 15° C. for 0.5 h. To the suspension was added a solution of ((3-(bromomethyl)but-3-en-1-yl)oxy)(tert-butyl)diphenylsilane (17 g, 42.14 mmol) in THF (100 mL) and the mixture was stirred at 15° C. for 16 h. The solution was poured into sat. NH₄Cl (500 mL) and extracted with ethyl acetate (300 mL×2). The combined organic phase was concentrated. The residue was diluted with Petroleum ether (300 mL). The resulting solution was washed with brine (300 mL×3), dried over anhydrous Na₂SO₄, filtered and concentrated. The residue was purified by column chromatography (silica gel, Petroleum ether) to give the title compound (6 g, 13.43 mmol) as colorless oil. ¹H NMR (400 MHz, CDCl₃) δ=7.77-7.60 (m, 4H), 7.50-7.32 (m, 6H), 5.01 (d, J=14.3 Hz, 2H), 4.30 (q, J=7.2 Hz, 2H), 3.77 (t, J=6.5 Hz, 2H), 2.79 (t, J=16.3 Hz, 2H), 2.37 (t, J=6.5 Hz, 2H), 1.33 (t, J=7.2 Hz, 3H), 1.05 (s, 9H).

Step F. (2R)-tert-Butyl 5-(6-((tert-butyldiphenylsilyl)oxy)-2,2-difluoro-4-methylenehexanoyl)-2-methyl-4-oxopiperidine-1-carboxylate. To a solution of (R)-tert-butyl 2-methyl-4-oxopiperidine-1-carboxylate (8.5 g, 39.86 mmol) in THF (100 mL) was added LiHMDS (1 M, 51.81 mL) at −65° C. under N₂, followed by a solution of ethyl 6-((tert-butyldiphenylsilyl)oxy)-2,2-difluoro-4-methylenehexanoate (19.58 g, 43.84 mmol) in THF (20 mL) after 0.5 h. The mixture was stirred at 50° C. for 8 h. The mixture was quenched with saturated aq. NH₄Cl (500 mL) and extracted with EtOAc (300 mL). The organic phase was dried over Na₂SO₄, filtered and concentrated in vacuo to give the title compound (23.1 g, 37.63 mmol) as yellow oil,

Step G. (R)-tert-Butyl 3-(5-((tert-butyldiphenylsilyl)oxy)-1,1-difluoro-3-methylenepentyl)-6-methyl-6,7-dihydro-2H-pyrazolo[4,3-c]pyridine-5(4H)-carboxylate. To a solution of (2R)-tert-butyl 5-(6-((tert-butyldiphenylsilyl)oxy)-2,2-difluoro-4-methylenehexanoyl)-2-methyl-4-oxopiperidine-1-carboxylate (23.1 g, 37.63 mmol) in EtOH (200 mL) was added a solution of N₂H₄.H₂O (1.92 g, 37.63 mmol, 1.87 mL, 98% purity) in EtOH (20 mL) and the mixture was stirred at 15° C. for 2 h. The mixture was concentrated in vacuo. The residue was diluted with EtOAc (300 mL) and washed with HCl (1 M aq, 200 mL) and brine (200 mL). The organic phase was dried over Na₂SO₄, filtered and concentrated in vacuo. The residue was purified by silica gel column (Petroleum ether/Ethyl acetate=5/1) to give the title compound(13.2 g, 20.63 mmol, 54.81% yield, 95.3% purity) as yellow oil. MS (ESI): mass calcd. for C₃₄H₄₅F₂N₃O₃Si 609.3; m/z found, 610.4 [M+H]+.

Step H. (R)-tert-Butyl 3-(1,1-difluoro-5-hydroxy-3-methylenepentyl)-6-methyl-6,7-dihydro-2H-pyrazolo[4,3-c]pyridine-5(4H)-carboxylate. To a solution of (R)-tert-butyl 3-(5-((tert-butyldiphenylsilyl)oxy)-1,1-difluoro-3-methylenepentyl)-6-methyl-6,7-dihydro-2H-pyrazolo[4,3-c]pyridine-5(4H)-carboxylate (13.2 g, 21.65 mmol) in THF (130 mL) was added TBAF (1 M, 32.47 mL) and the mixture was stirred at 0° C. for 1 h The mixture was diluted with EtOAc (150 mL) and washed with HCl (1 M aq, 150 ml), brine(150 mL). The organic phase was dried over Na₂SO₄, filtered and concentrated in vacuo. The residue was purified by flash silica gel chromatography (Ethyl acetate/Petroleum ether=2/1) to give the title compound (6.4 g, 17.23 mmol, 79.61% yield) as yellow oil.

Step I. (R)-tert-Butyl3-(1,1-difluoro-3-methylene-5-((methylsulfonyl)oxy)pentyl)-6-methyl-6,7-dihydro-2H-pyrazolo[4,3-c]pyridine-5(4H)-carboxylate. To a mixture of (R)-tert-butyl3-(1,1-difluoro-5-hydroxy-3-methylenepentyl)-6-methyl-6,7-dihydro-2H-pyrazolo[4,3-c]pyridine-5(4H)-carboxylate (6.4 g, 17.23 mmol) and Et₃N (5.23 g, 51.69 mmol) in DCM (65 mL) was added MsCl (2.96 g, 25.85 mmol) at 0° C. under N₂, and the mixture was stirred at 20° C. for 1 h. The mixture was diluted with DCM (150 mL) and washed with HCl (1 M aq, 150 mL), NaHCO₃ (saturated aq, 150 mL) and brine (150 ml). The organic phase was dried over Na₂SO₄, filtered and concentrated in vacuo to give the title compound (7.7 g, 17.13 mmol, 99.41% yield) as yellow oil.

Step J. (R)-tert-Butyl 11,11-difluoro-3-methyl-9-methylene-3,4,8,9,10,11-hexahydro-1H-pyrido[4′,3′:3,4]pyrazolo[1,5-a]azepine-2(7H)-carboxylate. To a solution of (R)-tert-butyl 3-(1,1-difluoro-3-methylene-5-((methylsulfonyl)oxy)pentyl)-6-methyl-6,7-dihydro-2H-pyrazolo[4,3-c]pyridine-5(4H)-carboxylate (7.7 g, 17.13 mmol) in MeCN (300 mL) was added DBU (3.91 g, 25.69 mmol).The mixture was stirred at 20° C. for 16 h. The mixture was diluted with EtOAc (150 mL) and washed with brine (200 mL). The organic phase was dried over Na₂SO₄, filtered and concentrated in vacuo. The residue was purified by column chromatography(SiO₂, Petroleum ether/Ethyl acetate=10:1) to give the title compound (4.7 g, 13.30 mmol, 77.64% yield, 100% purity) as colorless solid. MS (ESI): mass calcd. for C₁₈H₂₅F₂N₃O₂ 353.1; m/z found, 354.3 [M+H]+. ¹H NMR (400 MHz, CDCl₃) δ=5.12 (s, 2H), 5.06-4.75 (m, 2H), 4.47-4.25 (m, 2H), 4.08 (br d, J=16.6 Hz, 1H), 3.17-2.81 (m, 3H), 2.70-2.45 (m, 3H), 1.49 (s, 9H), 1.14 (d, J=7.0 Hz, 3H).

### Intermediate 50: (3R,9R*)-tert-Butyl 11,11-difluoro-9-hydroxy-3-methyl-3,4,8,9,10,11-hexahydro-1H-pyrido[4′,3′:3,4]pyrazolo[1,5-a]azepine-2(7H)-carboxylate

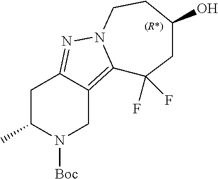 [SMILES: C[C@@H]1Cc2nn3c(c2CN1C(=O)OC(C)(C)C)C(F)(F)C[C@H](O)CC3]

### Step A. (3R)-tert-Butyl 11,11-difluoro-9-hydroxy-3-methyl-3,4,8,9,10,11-hexahydro-1H-pyrido[4′,3′:3,4]pyrazolo[1,5-a]azepine-2(7H)-carboxylate

To a solution of tert-butyl (3R)-11,11-difluoro-3-methyl-9-methylene-1,3,4,7,8,10-hexahydropyrido[2,3]pyrazolo[2,4-a]azepine-2-carboxylate (7 g, 19.81 mmol) in THF (260 mL)/H₂O (130 mL) was added NaIO₄ (16.95 g, 79.23 mmol) and OsO₄ (755.34 mg, 2.97 mmol) at 0° C. The mixture was stirred at 20° C. for 16 h. NaBH₄ (4.50 g, 118.84 mmol) was added at 0° C. and the mixture was stirred at 20° C. for 1 h. The mixture was quenched with Na₂SO₃ (saturated aq, 400 mL) and extracted with EtOAc (600 mL). The organic phase was dried over Na₂SO₄, filtered and concentrated in vacuo. The residue was purified column chromatography (SiO₂, Petroleum ether/Ethyl acetate=1/1 to 1/4) to give a mixture of the title compound and (3R)-tert-butyl 11-fluoro-9-hydroxy-3-methyl-3,4,8,9-tetrahydro-1H-pyrido[4′,3′:3,4]pyrazolo[1,5-a]azepine-2(7H)-carboxylate (5.7 g) as yellow solid. MS (ESI): mass calcd. for C₁₇H₂₅F₂N₃O₃ 357.1; m/z found, 358.1 [M+H]+.

### Step B. (3R,9R*)-tert-Butyl 11,11-difluoro-9-hydroxy-3-methyl-3,4,8,9,10,11-hexahydro-1H-pyrido[4′,3′:3,4]pyrazolo[1,5-a]azepine-2(7H)-carboxylate

A mixture of (3R)-tert-butyl 11,11-difluoro-9-hydroxy-3-methyl-3,4,8,9,10,11-hexahydro-1H-pyrido[4′,3′:3,4]pyrazolo[1,5-a]azepine-2(7H)-carboxylate and (3R)-tert-butyl 11-fluoro-9-hydroxyl-3-methyl-3,4,8,9-tetrahydro-1H-pyrido[4′,3′:3,4]pyrazolo[1,5-a]azepine-2(7H)-carboxylate (5.6 g) was resolved by SFC (condition: column: REGIS (s,s) WHELK-O1 (250 mm*50 mm,10 um); mobile phase: [0.1% NH₃H₂O IPA]; B %: 30%-30%, 2.65 min) to afford (3R,9R*)-tert-butyl 11,11-difluoro-9-hydroxy-3-methyl-3,4,8,9,10,11-hexahydro-1H-pyrido[4′,3′:3,4]pyrazolo[1,5-a]azepine-2(7H)-carboxylate (SFC condition A: retention time=2.171 min (Whelk-O1-3_10CM_IPA(DEA)_5_40_3ML_T35 Column: Chiralcel Whelk-o1-3 100×4.6 mm I.D., 3 um Mobile phase: IPA(0.05% DEA) in CO₂ from 5% to 40% Flow rate: 3 mL/min Wavelength: 220 nm); SFC condition B: retention time=1.145 min(Amycoat_IPA(DEA)_5_40_3mL-35T Column: Amycoat 50×4.6 mm I.D., 3 um Mobile phase: iso-propanol (0.05% DEA) in CO₂ from 5% to 40% Flow rate: 3 mL/min Wavelength: 220 nm); 1.65 g, 4.57 mmol, 29.17% yield, 98.9% purity) as yellow solid. [a]²⁵_{D}=+88.1 (c=1.0, MeOH); MS (ESI): mass calcd. for C₁₇H₂₅F₂N₃O₃ 357.1; m/z found, 358.1 [M+H]+. ¹H NMR (400 MHz, CDCl₃) δ=5.05-4.71 (m, 2H), 4.49 (br dd, J=6.4, 14.7 Hz, 1H), 4.34-3.98 (m, 3H), 2.90 (dd, J=5.9, 15.8 Hz, 1H), 2.77 (br dd, J=14.2, 18.2 Hz, 1H), 2.55 (d, J=15.7 Hz, 1H), 2.37-2.21 (m, 2H), 1.97-1.86 (m, 1H), 1.49 (s, 9H), 1.13 (d, J=7.0 Hz, 3H).

### Intermediate 51: (3R,9S*)-tert-Butyl 11,11-difluoro-9-hydroxy-3-methyl-3,4,8,9,10,11-hexahydro-1H-pyrido[4′,3′:3,4]pyrazolo[1,5-a]azepine-2(7H)-carboxylate

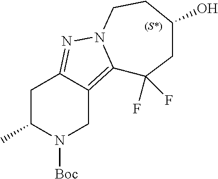 [SMILES: C[C@@H]1Cc2nn3c(c2CN1C(=O)OC(C)(C)C)C(F)(F)C[C@@H](O)CC3]

(3R,9S*)-tert-butyl 11,11-difluoro-9-hydroxy-3-methyl-3,4,8,9,10,11-hexahydro-1H-pyrido[4′,3′:3,4]pyrazolo[1,5-a]azepine-2(7H)-carboxylate was resolved from intermediate 50 as yellow solid (SFC condition A: retention time: 2.354 min; SFC condition B: retention time: 1.081 min); [a]²⁵_{D}=+48.98(c=0.85, MeOH)). MS (ESI): mass calcd. for C₁₇H₂₅F₂N₃O₃ 357.1; m/z found, 358.1 [M+H]+.

### Intermediate 52: (3R,9R*)-tert-Butyl 11-fluoro-9-hydroxy-3-methyl-3,4,8,9tetrahydro-1H-pyrido[4′,3′:3,4]pyrazolo[1,5-a]azepine-2(7H)-carboxylate

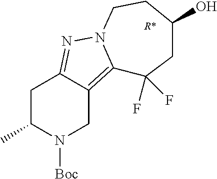 [SMILES: C[C@@H]1Cc2nn3c(c2CN1C(=O)OC(C)(C)C)C(F)(F)C[C@H](O)CC3]

Step A. (3R)-tert-Butyl11-fluoro-9-hydroxy-3-methyl-3,4,8,9-tetrahydro-1H-pyrido[4′,3′:3,4]pyrazolo[1,5-a]azepine-2(7H)-carboxylate. 1.63 g of (3R)-tert-butyl 11-fluoro-9-hydroxy-3-methyl-3,4,8,9-tetrahydro-1H-pyrido[4′,3′:3,4]pyrazolo[1,5-a]azepine-2(7H)-carboxylate was resolved from intermediate 50 via SFC.

Step B. (3R,9R*)-tert-Butyl 11-fluoro-9-hydroxy-3-methyl-3,4,8,9-tetrahydro-1H-pyrido[4′,3′:3,4]pyrazolo[1,5-a]azepine-2(7H)-carboxylate. (3R)-tert-Butyl 11-fluoro-9-hydroxy-3-methyl-3,4,8,9-tetrahydro-1H-pyrido[4′,3′:3,4]pyrazolo[1,5-a]azepine-2(7H)-carboxylate (1.6 g, 4.74 mmol) was resolved by SFC (column: DAICEL CHIRALPAK AD-H(250 mm*30 mm,5 um); mobile phase: [0.1% NH3H2O IPA];B %: 15%-15%,2 min;140 minmin) to afford intermediate 52 tert-butyl (3R,9R*)-11-fluoro-9-hydroxyl-3-methyl-1,3,4,7,8,9-hexahydropyrido[2,3]pyrazolo[2,4-a]azepine-2-carboxylate (0.499 g, peak 1 on SFC. retention time: 1.424 min(IC-3_IPA(DEA)_5_40_3mL-35T Column: Chiralpak IC-3 50×4.6 mm I.D., 3 um Mobile phase: iso-propanol (0.05% DEA) in CO₂ from 5% to 40% Flow rate: 3 mL/min Wavelength: 220 nm) as yellow oil.

### Intermediate 53: (3R,9S*)-tert-Butyl 11-fluoro-9-hydroxy-3-methyl-3,4,8,9tetrahydro-1H-pyrido[4′,3′:3,4]pyrazolo[1,5-a]azepine-2(7H)-carboxylate

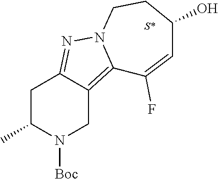 [SMILES: C[C@@H]1Cc2nn3c(c2CN1C(=O)OC(C)(C)C)C(F)=C[C@@H](O)CC3]

(3R,9 S*)-tert-butyl 11,11-difluoro-9 hydroxy-3-methyl-3,4,8,9,10,11-hexahydro-1H-pyrido[4′,3′:3,4]pyrazolo[1,5-a]azepine-2(7H)-carboxylate was resolved from intermediate 52 (0.8 g, peak 2 on SFC. retention time=1.505 min) as yellow oil.

### Intermediate 54: (3R,9R*)-tert-Butyl 11,11-difluoro-9-hydroxy-9-(hydroxymethyl)-3-methyl-3,4,8,9,10,11-hexahydro-1H-pyrido[4′,3′:3,4]pyrazolo[1,5-a]azepine-2(7H)-carboxylate

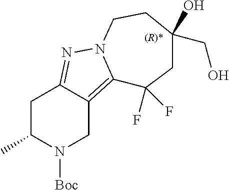 [SMILES: C[C@@H]1Cc2nn3c(c2CN1C(=O)OC(C)(C)C)C(F)(F)C[C@@](O)(CO)CC3]

### Step A. (3R)-tert-Butyl 11,11-difluoro-9-hydroxy-9-(hydroxymethyl)-3-methyl-3,4,8,9,10,11-hexahydro-1H-pyrido[4′,3′:3,4]pyrazolo[1,5-a]azepine-2(7H)-carboxylate

To a solution of (R)-tert-butyl 11,11-difluoro-3-methyl-9-methylene-3,4,8,9,10,11-hexahydro-1H-pyrido[4′,3′:3,4]pyrazolo[1,5-a]azepine-2(7H)-carboxylate (891 mg, 2.52 mmol) in THF (100 mL) and H₂O (50 mL) was added OsO₄ (64.10 mg, 252.12 μmol, 13.08 μL), NMO (413.48 mg, 3.53 mmol, 372.51 μL). The mixture was stirred at 15° C. for 16 hr. The mixture was diluted with H₂O (10 mL) and extracted with EtOAc (20 mL×3). The combined organic layer was washed by sat. aq. Na₂SO₃(10 mL×2), dried over Na₂SO₄, filtered and concentrated in vacuo. The residue was purified by column chromatography (SiO₂, Petroleum ether/Ethyl acetate=10/1 to 1/1) to give the title compound (871 mg, 2.25 mmol, 89.17% yield) was obtained as a white solid. MS (ESI): mass calcd. for C₁₈H₂₇F₂N₃O₄, 387.2; m/z found, 388.1 [M+H]⁺. ¹H NMR (400 MHz, CDCl₃-d) δ=5.10-4.74 (m, 2H), 4.63-4.49 (m, 1H), 4.44-4.31 (m, 1H), 4.13-4.00 (m, 1H), 3.57 (d, J=16.0 Hz, 2H), 2.99-2.87 (m, 1H), 2.76-2.36 (m, 3H), 2.17-1.95 (m, 3H), 1.50 (d, J=1.5 Hz, 9H), 1.15 (t, J=7.3 Hz, 3H).

### Step B. (3R,9R*)-tert-Butyl 11,11-difluoro-9-hydroxy-9-(hydroxymethyl)-3-methyl-3,4,8,9,10,11-hexahydro-1H-pyrido[4′,3′:3,4]pyrazolo[1,5-a]azepine-2(7H)-carboxylate

(3R)-tert-Butyl 11,11-difluoro-9-hydroxy-9-(hydroxymethyl)-3-methyl-3,4,8,9,10,11-hexahydro-1H-pyrido[4′,3′:3,4]pyrazolo[1,5-a]azepine-2(7H)-carboxylate was isolated by SFC (column: DAICEL CHIRALPAK IC (250 mm*30 mm, 10 um); mobile phase: [0.1% NH₃H₂₀ MEOH]; B %: 20%-20%, 2.75 min; 70 min) to give (3R,9R*)-tert-butyl 11,11-difluoro-9-hydroxy-9-(hydroxymethyl)-3-methyl-3,4,8,9,10,11-hexahydro-1H-pyrido[4′,3′:3,4]pyrazolo[1,5-a]azepine-2(7H)-carboxylate (57 mg, 99% purity, Peak 1 on SFC, retention time=1.084 min(IC-3_MeOH(DEA)_5_40_3mL-35T Column: Chiralpak IC-3 50×4.6 mm I.D., 3 um Mobile phase: methanol (0.05% DEA) in CO₂ from 5% to 40% Flow rate: 3 mL/min Wavelength: 220 nm)) as a white solid.

### Intermediate 55: (3R,9S*)-tert-Butyl11,11-difluoro-9-hydroxy-9-(hydroxymethyl)-3-methyl-3,4,8,9,10,11-hexahydro-1H-pyrido[4′,3′:3,4]pyrazolo[1,5-a]azepine-2(7H)-carboxylate

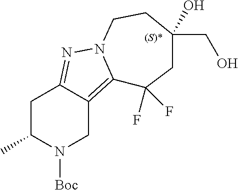 [SMILES: C[C@@H]1Cc2nn3c(c2CN1C(=O)OC(C)(C)C)C(F)(F)C[C@](O)(CO)CC3]

(3R,9S*)-tert-Butyl 11,11-difluoro-9-hydroxy-9-(hydroxymethyl)-3-methyl-3,4,8,9,10,11-hexahydro-1H-pyrido[4′,3′:3,4]pyrazolo[1,5-a]azepine-2(7H)-carboxylate was separated from intermediate 54 via SFC (Peak 2 on SFC, retention time=1.180 min, 63 mg, 97% purity) as a white solid.

### Intermediate 56: (3R,9R*)-tert-Butyl 11,11-difluoro-9-(fluoromethyl)-9-hydroxy-3-methyl-3,4,8,9,10,11-hexahydro-1H-pyrido[4′,3′:3,4]pyrazolo[1,5-a]azepine-2(7H)-carboxylate

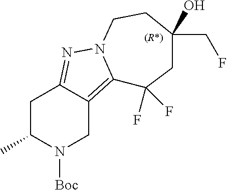 [SMILES: C[C@@H]1Cc2nn3c(c2CN1C(=O)OC(C)(C)C)C(F)(F)C[C@@](O)(CF)CC3]

### Step A. (3′R)-tert-Butyl 11′,11′-difluoro-3′-methyl-3′,4′,7′,8′,10′,11′-hexahydrospiro[oxirane-2,9′-pyrido[4′,3′:3,4]pyrazolo[1,5-a]azepine]-2′(1′H)-carboxylate

To a solution of tert-butyl (3R)-11,11-difluoro-9-hydroxy-9-(hydroxymethyl)-3-methyl-1,3,4,7,8,10-hexahydropyrido[2,3]pyrazolo[2,4-a]azepine-2-carboxylate (820 mg, 2.12 mmol) in THF (25 mL) was added DBU (644.44 mg, 4.23 mmol). The solution was cooled to 0° C., then perfluorobutane-1-sulphonyl fluoride (1.15 g, 3.81 mmol) was added dropwise. The reaction was stirred for 3 h at 20° C. Perfluorobutane-1-sulphonyl fluoride (383.64 mg, 1.27 mmol) and DBU (193.33 mg, 1.27 mmol,) was added to the mixture. The mixture was stirred at 20° C. for 1 hr. The residue was poured into water (150 mL). The aqueous phase was extracted with ethyl acetate (150 mL×3). The combined organic phase was washed with brine (100 mL), dried with anhydrous Na₂SO₄, filtered and concentrated in vacuum. The residue was purified by column chromatography (SiO₂, Petroleum ether/Ethyl acetate=20/1 to 1:1) to give the title compound (851 mg, 90% purity) as colourless oil. MS (ESI): mass calcd. for C₁₈H₂₅F₂N₃O₃ 369.2; m/z found, 370.2 [M+H]+.

### Step B. (3R)-tert-Butyl 11,11-difluoro-9-(fluoromethyl)-9-hydroxy-3-methyl-3,4,8,9,10,11-hexahydro-1H-pyrido[4′,3′:3,4]pyrazolo[1,5-a]azepine-2(7H)-carboxylate

A solution of tert-butyl (3R)-11,11-difluoro-3-methyl-spiro[1,3,4,7,8,10-hexahydropyrido[2,3]pyrazolo[2,4-a]azepine-9,2′-oxirane]-2-carboxylate (556 mg, 1.35 mmol) in Et₃N₃HF(2.97 g, 18.40 mmol, 3 mL,) was stirred at 100° C. for 5 hr. The mixture was continued stirring at 100° C. for another 14 hr. The mixture was combined with another batch(100 mg scale), and diluted in H₂O (50 mL) and extracted with EtOAc (30 mL×3). The combined organic layer was washed with brine (50 mL), dried over Na₂SO₄, filtered and concentrated. The residue was purified by column chromatography (SiO₂, Petroleum ether/Ethyl acetate=20/1 to 2:1) to give the title compound (306 mg, 52% yield, 90% purity) as a white solid. MS (ESI): mass calcd. for C₁₈H₂₆F₃N₃O₃ 389.2; m/z found, 390.2 [M+H]+.

### Step C. (3R,9R*)-tert-Butyl 11,11-difluoro-9-(fluoromethyl)-9-hydroxy-3-methyl-3,4,8,9,10,11-hexahydro-1H-pyrido[4′,3′:3,4]pyrazolo[1,5-a]azepine-2(7H)-carboxylate

(3R)-tert-butyl 11,11-difluoro-9-(fluoromethyl)-9-hydroxy-3-methyl-3,4,8,9,10,11-hexahydro-1H-pyrido[4′,3′:3,4]pyrazolo[1,5-a]azepine-2(7H)-carboxylate(305 mg) was separated by SFC (column: DAICEL CHIRALPAK IC(250 mm*30 mm,10 um); mobile phase: [0.1% NH₃H₂O MeOH]; B %: 15%-15%, 2.9 min; 200 min) to give title compound (91 mg, peak 1, retention time=0.878 min(IC-3_5CM_MeOH(DEA)_5_40_3ML_T35 Column: Chiralpak IC-3 50×4.6 mm I.D., 3 um; Mobile phase: methanol (0.05% DEA) in CO₂ from 5% to 40%; Flow rate: 3 mL/min; Wavelength: 220 nm)) as a white solid. [a]²⁵_{D}=+64.5 (c=0.38, MeOH).

### Intermediate 57: (3R,9S*)-tert-Butyl 11,11-difluoro-9-(fluoromethyl)-9-hydroxy-3-methyl-3,4,8,9,10,11-hexahydro-1H-pyrido[4′,3′:3,4]pyrazolo[1,5-a]azepine-2(7H)-carboxylate

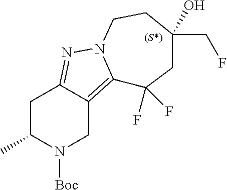 [SMILES: C[C@@H]1Cc2nn3c(c2CN1C(=O)OC(C)(C)C)C(F)(F)C[C@](O)(CF)CC3]

The title compound (140 mg, peak 2, retention time=0.969 min) as a white solid was resolved from intermediate 56 via SFC. [a]²⁵_{D}=+61.1 (c=0.46, MeOH)).

### Intermediate 58: (3R,9R*)-tert-Butyl 11,11-difluoro-9-(hydroxymethyl)-3-methyl-3,4,8,9,10,11-hexahydro-1H-pyrido[4′,3′:3,4]pyrazolo[1,5-a]azepine-2(7H)-carboxylate

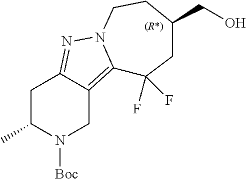 [SMILES: C[C@@H]1Cc2nn3c(c2CN1C(=O)OC(C)(C)C)C(F)(F)C[C@H](CO)CC3]

### Step A. (3R)-tert-Butyl 11,11-difluoro-9-(hydroxymethyl)-3-methyl-3,4,8,9,10,11-hexahydro-1H-pyrido[4′,3′:3,4]pyrazolo[1,5-a]azepine-2(7H)-carboxylate

To a solution of (R)-tert-butyl 11,11-difluoro-3-methyl-9-methylene-3,4,8,9,10,11-hexahydro-1H-pyrido[4′,3′:3,4]pyrazolo[1,5-a]azepine-2(7H)-carboxylate (0.5 g, 1.41 mmol) in THF (5 mL) was added 9-BBN (0.5 M, 56.40 mL) at 0° C. under N₂, and the mixture was stirred at 25° C. for 2 h. NaOH (563.96 mg, 14.10 mmol) in H₂O (4 mL) and H₂O₂ (1.92 g, 16.92 mmol, 1.63 mL, 30% purity) was added at −30° C. The mixture was stirred at 25° C. for 2 h. The mixture was diluted with Ethyl acetate(50 mL) and washed with saturated aq. Na₂SO₃(30 mL). The organic phase was dried over Na₂SO₄, filtered and concentrated in vacuo. The residue was purified by column chromatography (SiO₂, Petroleum ether/Ethyl acetate=1/1) to give title compound (0.4 g, 1.03 mmol, 73.25% yield, 95.9% purity) as yellow oil. MS (ESI): mass calcd. for C₁₈H₂₇F₂N₃O₃ 371.2; m/z found, 372.2 [M+H]+.

### Step B. (3R,9R*)-tert-Butyl 11,11-difluoro-9-(hydroxymethyl)-3-methyl-3,4,8,9,10,11-hexahydro-1H-pyrido[4′,3′:3,4]pyrazolo[1,5-a]azepine-2(7H)-carboxylate

(3R)-tert-Butyl 11,11-difluoro-9-(hydroxymethyl)-3-methyl-3,4,8,9,10,11-hexahydro-1H-pyrido[4′,3′:3,4]pyrazolo[1,5-a]azepine-2(7H)-carboxylate(400 mg) was resolved by SFC(column: DAICEL CHIRALPAK IC(250 mm*30 mm,10 um); mobile phase: [0.1% NH₃H₂O IPA]; B %: 25%-25%, 2.3 min; 80 min) to give title compound intermediate 58(170 mg, peak1, retention time=1.222 min(Column: Chiralpak IC-3 50 Á4.6 mm I.D., 3 um Mobile phase: iso-propanol (0.05% DEA) in CO₂ from 5% to 40%; Flow rate: 3 mL/min Wavelength: 220 nm)) as white solid. [a]25D =+52.0 (c=0.5,MeOH). MS (ESI): mass calcd. for C₁₈H₂₇F₂N₃O₃ 371.2; m/z found, 372.2 [M+H]+.

### Intermediate 59: (3R,9S*)-tert-Butyl 11,11-difluoro-9-(hydroxymethyl)-3-methyl-3,4,8,9,10,11-hexahydro-1H-pyrido[4′,3′:3,4]pyrazolo[1,5-a]azepine-2(7H)-carboxylate

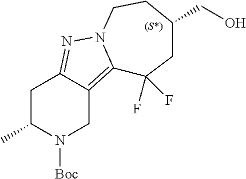 [SMILES: C[C@@H]1Cc2nn3c(c2CN1C(=O)OC(C)(C)C)C(F)(F)C[C@@H](CO)CC3]

The title compound intermediate 59 was resolved from intermediate 58 by SFC (190 mg, peak 2, retention time=1.395 min) as colorless oil. [a]²⁵_{D}=+73.368 (c=0.5, MeOH). MS (ESI): mass calcd. for C₁₈H₂₇F₂N₃O₃ 371.2; m/z found, 372.2 [M+H]+.

### Intermediate 60: tert-Butyl11,11-difluoro-9-methylene-3,4,8,9,10,11-hexahydro-1H-pyrido[4′,3′:3,4]pyrazolo[1,5-a]azepine-2(7H)-carboxylate

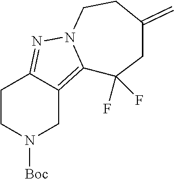 [SMILES: C=C1CCn2nc3c(c2C(F)(F)C1)CN(C(=O)OC(C)(C)C)CC3]

Intermediate 60 was prepared in a manner analogous to intermediate 49, however using tert-butyl 4-oxopiperidine-1-carboxylate for (R)-tert-butyl 2-methyl-4-oxopiperidine-1-carboxylate in Step F.

### Intermediate 61: (R*)-tert-Butyl 11,11-difluoro-9-(hydroxymethyl)-3,4,8,9,10,11-hexahydro-1H-pyrido[4′,3′:3,4]pyrazolo[1,5-a]azepine-2(7H)-carboxylate

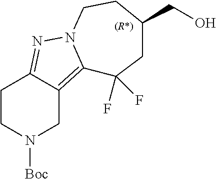 [SMILES: CC(C)(C)OC(=O)N1CCc2nn3c(c2C1)C(F)(F)C[C@H](CO)CC3]

To a solution of tert-butyl 11,11-difluoro-9-methylene-3,4,8,9,10,11-hexahydro-1H-pyrido[4′,3′:3,4]pyrazolo[1,5-a]azepine-2(7H)-carboxylate(900 mg, 2.57 mmol) in THF (10 mL) was added 9-BBN (0.5 M, 102.79 mL) at 0° C. The solution was stirred at 15° C. for 1 hr. NaOH (1.03 g, 25.70 mmol) and H₂O₂ (2.83 g, 30.84 mmol, 2.40 mL, 37% purity) was added at −30° C. The solution was stirred at 15° C. for 16 hr. The solution was poured into sat. Na₂S₂O₃(300 mL). The mixture was extracted with ethyl acetate (200 mL). The organic phase was washed with aq. 1 N HCl (200 mL), sat. NaHCO₃ (200 mL), and brine (200 mL), dried with anhydrous Na₂SO₄, filtered and concentrated. The residue was purified by column chromatography (silica gel, Petroleum ether/Ethyl acetate=5/1 to 1/2) to give racemic tert-butyl 11,11-difluoro-9-(hydroxymethyl)-3,4,7,8,9,10-hexahydro-1H-pyrido[2,3]pyrazolo[2,4-a]azepine-2-carboxylate (750 mg, 2.08 mmol, 80.85% yield) as white solid. MS (ESI): mass calcd. for C₁₇H₂₅F₂N₃O₃ 357.2; m/z found, 358.2 [M+H]⁺. ¹H NMR (400 MHz, CDCl₃) δ=4.57-4.46 (m, 2H), 4.24-4.12 (m, 1H), 3.81 (br t, J=9.0 Hz, 1H), 3.71 (br s, 1H), 3.59 (br d, J=6.0 Hz, 2H), 2.71 (br t, J=5.3 Hz, 2H), 2.56 (br t, J=15.2 Hz, 1H), 2.32-2.10 (m, 2H), 1.91-1.86 (m, 2H), 1.68-1.57 (m, 2H), 1.48 (s, 9H).

The resulting racemate (750 mg) was resolved by SFC (column: DAICEL CHIRALPAK AD(250 mm*30 mm,10 um); mobile phase: [0.1% NH₃H₂₀ EtOH]; B %: 40%-40%, 4 min; 50 min) to give (R*)-tert-butyl 11,11-difluoro-9-(hydroxymethyl)-3,4,8,9,10,11-hexahydro-1H-pyrido[4′,3′:3,4]pyrazolo[1,5-a]azepine-2(7H)-carboxylate (350 mg, 956.79 μmol, 100% ee, retention time=0.991 min, AD-3_5CM_ETOH(DEA)_5_40_3ML_T35; Column: Chiralpak AD-3 50×4.6 mm I.D., 3 um; Mobile phase: ethanol (0.05% DEA) in CO₂ from 5% to 40%; Flow rate: 3 mL/min; Wavelength: 220 nm) as a white solid. [a]²⁵_{D}=−2.575 (c=0.55,CHCl₃).

### Intermediate 62: (S*)-tert-Butyl 11,11-difluoro-9-(hydroxymethyl)-3,4,8,9,10,11-hexahydro-1H-pyrido[4′,3′:3,4]pyrazolo[1,5-a]azepine-2(7H)-carboxylate

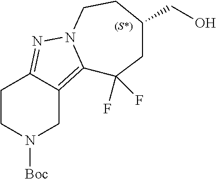 [SMILES: CC(C)(C)OC(=O)N1CCc2nn3c(c2C1)C(F)(F)C[C@@H](CO)CC3]

The title compound was resolved from Intermediate 61 via SFC (360 mg, retention time=1.443 min, 98.5% ee) as a white solid. [a]²⁵_{D}=+2.670 (c=0.6, CHCl₃).

### Intermediate 63: (R*)-Benzyl 11,11-difluoro-9-hydroxy-3,4,8,9,10,11-hexahydro-1H-pyrido[4′,3′:3,4]pyrazolo[1,5-a]azepine-2(7H)-carboxylate

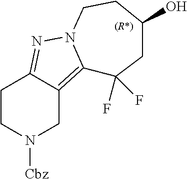 [SMILES: O=C(OCc1ccccc1)N1CCc2nn3c(c2C1)C(F)(F)C[C@H](O)CC3]

Step A. (But-3-en-1-yloxy)(tert-butyl)diphenylsilane. To a solution of but-3-en-1-ol (40 g, 554.74 mmol) in DCM (1000 mL) was added DMAP (6.78 g, 55.47 mmol), imidazole (56.65 g, 832.12 mmol) and TBDPSCl (160.10 g, 582.48 mmol) at 0° C. under N₂. The mixture was stirred at 15° C. for 16 h. The reaction mixture was quenched with 1 N HCl (800 mL) at 0° C., and then extracted with DCM (1000 mL). The organic layer was washed with brine (1500 mL), dried over Na₂SO₄, filtered and concentrated under reduced pressure. The residue was purified by column chromatography (SiO₂, Petroleum ether/Ethyl acetate=1/020/1) to give the title compound (169 g, 544.28 mmol, 98.11% yield) as colorless oil. ¹H NMR (400 MHz, CDCl₃) δ=7.73-7.65 (m, 4H), 7.47-7.33 (m, 6H), 5.92-5.79 (m, 1H), 5.13-4.99 (m, 2H), 3.73 (t, J=6.4 Hz, 2H), 2.39-2.28 (m, 2H), 1.07 (s, 9H).

Step B. Ethyl 6-((tert-butyldiphenylsilyl)oxy)-2,2-difluoro-4-iodohexanoate. To a solution of but-3-enoxy-tert-butyl-diphenyl-silane (47 g, 151.37 mmol) and ethyl 2,2-difluoro-2-iodo-acetate (45.41 g, 181.64 mmol) in MeCN (155 mL) and H₂O (155 mL) was added NaHCO₃ (27.97 g, 333.01 mmol, 12.95 mL) under N₂, then N₂ was bubbled into the reaction mixture for 10 min. Na₂S₂O₄ (55.34 g, 317.87 mmol, 69.18 mL) was added. The mixture was stirred at 25° C. for 16 h. The mixture was diluted with H₂₀(300 mL), and extracted with Ethyl acetate (400 mL×2). The combined organic layer was washed with brine (600 mL), dried over Na₂SO₄, filtered and concentrated in vacuo. The residue was purified by column chromatography (SiO₂, Petroleum ether/Ethyl acetate=1/0 to 40/1) to give the title compound (63 g, 84.84 mmol, 74.26% yield) as a colorless oil as a colorless oil. ¹H NMR (400 MHz, CDCl₃) δ=7.74-7.61 (m, 4H), 7.50-7.33 (m, 6H), 4.63-4.51 (m, 1H), 4.36 (q, J=7.1 Hz, 2H), 3.87-3.69 (m, 2H), 3.10-2.74 (m, 2H), 2.09-1.88 (m, 2H), 1.42-1.31 (m, 3H), 1.07 (s, 9H).

Step C. 5-(2-((tert-butyldiphenylsilyl)oxy)ethyl)-3,3-Difluorodihydrofuran-2(3H)-one. Ethyl 6-((tert-butyldiphenylsilyl)oxy)-2,2-difluoro-4-iodohexanoate (66 g, 117.75 mmol) in a 10% aqueous solution of Na₂CO₃ (660 mL) was refluxed for 7 h. The reaction was acidified with HCl (1N) to pH=˜3, and then extracted with EtOAc (400 mL×2). The combined organic layer was washed brine (500 mL×2), dried over Na₂SO₄, filtered and concentrated in vacuo. The residue was stood for 16 h, then the desired product was formed. The residue was purified by column chromatography (SiO₂, Petroleum ether/Ethyl acetate=1/0 to 10/1) to give the title compound (35.5 g, 87.76 mmol, 74.53% yield) as a yellow oil. ¹H NMR (400 MHz, CDCl₃) δ=7.64 (td, J=1.6, 7.9 Hz, 4H), 7.49-7.37 (m, 6H), 4.90-4.83 (m, 1H), 3.91-3.71 (m, 2H), 2.80 (ddt, J=6.5, 8.3, 14.8 Hz, 1H), 2.49-2.29 (m, 1H), 2.10-1.86 (m, 2H), 1.06 (s, 9H).

Step D. Benzyl 3-(6-((tert-butyldiphenylsilyl)oxy)-2,2-difluoro-4-hydroxyhexanoyl)-4-oxopiperidine-1-carboxylate. To a solution of benzyl 4-oxopiperidine-1-carboxylate (9 g, 38.58 mmol, 7.69 mL) in THF (90 mL) was added LiHMDS (1 M, 46.30 mL) at −70° C. under N₂. The mixture was stirred at −70° C. for 0.5 hr, then a solution of 5-(2-((tert-butyldiphenylsilyl)oxy)ethyl)-3,3-difluorodihydrofuran-2(3H)-one (18.73 g, 46.30 mmol) in THF (60 mL) was added. The mixture was stirred at 20° C. for 2 hr. The mixture was poured into HCl solution (0.5 N, 80 mL) and stirred for 1 min. The aqueous phase was extracted with ethyl acetate (100 mL×3). The combined organic phase was washed with brine (100 mL), dried with anhydrous Na₂SO₄, filtered and concentrated in vacuo to give the title compound (25 g, crude) as a yellow oil. MS (ESI): mass calcd. for C₃₅H₄₁F₂NO₆Si, 637.3; m/z found, 620.1 [M−18]+.

Step E. Benzyl 3-(5-((tert-butyldiphenylsilyl)oxy)-1,1-difluoro-3-hydroxypentyl)-6,7-dihydro-2H-pyrazolo[4,3-c]pyridine-5(4H)-carboxylate. To a solution of benzyl 3-(6-((tert-butyldiphenylsilyl)oxy)-2,2-difluoro-4-hydroxyhexanoyl)-4-oxopiperidine-1-carboxylate (25 g, crude) in EtOH (180 mL) was added N₂H₄H₂O (2 g, 39.15 mmol, 1.94 mL, 98% purity) at −40° C. The mixture was stirred at 25° C. for 12 hr. The mixture was concentrated in vacuo. The mixture was dissolved in HCl (80 mL, 1 N). The aqueous phase was extracted with ethyl acetate (90 mL×2). The combined organic phase was washed with brine (60 mL×2), dried with anhydrous Na₂SO₄, filtered and concentrated in vacuo. The residue was purified by column chromatography (SiO₂, Petroleum ether/Ethyl acetate=5/1 to 1:1) to give the title compound (17 g, 26.82 mmol) as a yellow oil. MS (ESI): mass calcd. for C₃₅H₄₁F₂N₃O₄Si, 633.3; m/z found, 634.3 [M+H]⁺. ¹H NMR (400 MHz, CDCl₃) δ=7.59 (td, J=1.8, 7.9 Hz, 4H), 7.32-7.29 (m, 6H), 5.11 (s, 2H), 4.57 (s, 2H), 4.31-4.19 (m, 1H), 3.82-3.66 (m, 4H), 2.71-2.61 (m, 3H), 2.50-2.37 (m, 2H), 1.85-1.64 (m, 2H), 0.97 (s, 9H)

Step F. Benzyl 3-(1,1-difluoro-3,5-dihydroxypentyl)-6,7-dihydro-2H-pyrazolo[4,3-c]pyridine-5(4H)-carboxylate. To a solution of benzyl 3-(5-((tert-butyldiphenylsilyl)oxy)-1,1-difluoro-3-hydroxypentyl)-6,7-dihydro-2H-pyrazolo[4,3-c]pyridine-5(4H)-carboxyl ate (17 g, 26.82 mmol) in THF (50 mL) was added TBAF (1 M, 32.97 mL). The mixture was stirred at 30° C. for 1 hr. The mixture was diluted with H₂O (100 mL), extracted with ethyl acetate (150 mL×2). The combined organic layer was washed brine (100 mL×2), dried over Na₂SO₄, filtered and concentrated in vacuo. The residue was purified by column chromatography (SiO₂, Petroleum ether/Ethyl acetate=1/1 to 0/1) to give the title compound (10 g, 25.29 mmol, 94.29% yield) as a yellow oil. MS (ESI): mass calcd. for C₁₉H₂₃F₂N₃O₄, 395.2; m/z found, 396.2 [M+H]+. ¹H NMR (400 MHz, CDCl₃) δ=7.45-7.29 (m, 5H), 5.19 (s, 2H), 4.71-4.60 (m, 2H), 4.28 (br s, 1H), 3.92-3.74 (m, 4H), 2.77 (br s, 2H), 2.62-2.37 (m, 2H), 1.91-1.75 (m, 2H).

Step G. Benzyl 3-(1,1-difluoro-3-hydroxy-5-((methylsulfonyl)oxy)pentyl)-6,7-dihydro-2H-pyrazolo[4,3-c]pyridine-5(4H)-carboxylate. To a solution of benzyl 3-(1,1-difluoro-3,5-dihydroxypentyl)-6,7-dihydro-2H-pyrazolo[4,3-c]pyridine-5(4H)-carboxylate (10 g, 25.29 mmol) and 2,4,6-trimethylpyridine (30.65 g, 252.91 mmol, 33.42 mL) in DCM (460 mL) was added MsCl (3.48 g, 30.35 mmol) at 0° C. under N₂. The mixture was stirred at 0° C. for 4 hr. Then the reaction mixture was moved to fridge (5° C.) and stood for 16 h. The reaction mixture was diluted with H₂O (100 mL) and extracted with DCM (100 mL×3). The combined organic layer was washed with brine (100 mL×2), dried over Na₂SO₄, filtered and concentrated in vacuo. The residue was purified by column chromatography (SiO₂, Petroleum ether/Ethyl acetate=2/1 to 1/1) to give the title compound (10 g, 17.95 mmol, 70.98% yield) as a yellow oil. MS (ESI): mass calcd. for C₂₀H₂₅F₂N₃O₆S, 473.1; m/z found, 474.2 [M+H]+.

Step H. Benzyl 11,11-difluoro-9-hydroxy-3,4,8,9,10,11-hexahydro-1H-pyrido[4′,3′:3,4]pyrazolo[1,5-a]azepine-2(7H)-carboxylate. To a solution of benzyl difluoro-3-hydroxy-5-((methylsulfonyl)oxy)pentyl)-6,7-dihydro-2H-pyrazolo[4,3-c]pyridine-5(4H)-carboxylate (10 g, 21.12 mmol) in MeCN (150 mL) was added DBU (10.00 g, 65.69 mmol, 9.90 mL). The mixture was stirred at 30° C. for 12 h. The reaction mixture was diluted with aq HCl (1 N, 30 mL) and extracted with EtOAc (60 mL×3). The combined organic layer was washed with brine(80 mL), dried over Na₂SO₄, filtered and concentrated in vacuo. The residue was purified by column chromatography (SiO₂, Petroleum ether/Ethyl acetate=5/1 to 1:1) to give the title compound (3.7 g, 9.80 mmol, 46.42% yield) as a colorless oil. MS (ESI): mass calcd. for C₁₉H₂₁F₂N₃O₃, 377.2; m/z found, 378.1 [M+H]+. ¹H NMR (400 MHz, CDCl₃) δ=7.41-7.30 (m, 5H), 5.17 (s, 2H), 4.62 (br s, 2H), 4.49 (br dd, J=6.7, 14.3 Hz, 1H), 4.31-4.21 (m, 1H), 4.14 (dd, J=10.6, 14.2 Hz, 1H), 3.83-3.64 (m, 2H), 2.79-2.65 (m, 3H), 2.38-2.19 (m, 2H), 2.00-1.88 (m, 2H).

Step I. (R*)-Benzyl 11,11-difluoro-9-hydroxy-3,4,8,9,10,11-hexahydro-1H-pyrido[4′,3′:3,4]pyrazolo[1,5-a]azepine-2(7H)-carboxylate. Benzyl 11,11-difluoro-9-hydroxy-3,4,8,9,10,11-hexahydro-1H-pyrido[4′,3′:3,4]pyrazolo[1,5-a]azepine-2(7H)-carboxylate (780 mg, 2 mmol) was resolved via SFC to give (R*)-benzyl 11,11-difluoro-9-hydroxy-3,4,8,9,10,11-hexahydro-1H-pyrido[4′,3′:3,4]pyrazolo[1,5-a]azepine-2(7H)-carboxylate (350 mg, 915.38 mol. retention time=1.450 min(AD-3_5CM_EtOH(DEA)_5_40_3mL-35T Column: Chiralpak AD-3 50×4.6 mm I.D., 3 um Mobile phase: ethanol (0.05% DEA) in CO₂ from 5% to 40%; Flow rate: 3 mL/min; Wavelength: 220 nm) as a white solid. MS (ESI): mass calcd. for C₁₉H₂₁F₂N₃O₃ 377.2; m/z found, 378.1 [M+H]+. [a]25_{D}=−9.911 (c=0.6,CHCl₃).

### Intermediate 64: (S*)-Benzyl 11,11-difluoro-9-hydroxy-3,4,8,9,10,11-hexahydro-1H-pyrido[4′,3′:3,4]pyrazolo[1,5-a]azepine-2(7H)-carboxylate

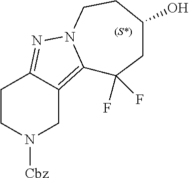 [SMILES: O=C(OCc1ccccc1)N1CCc2nn3c(c2C1)C(F)(F)C[C@@H](O)CC3]

The title compound (360 mg, retention time=2.063 min, 100% ee) was resolved by SFC from Intermediate 63. MS (ESI): mass calcd. for C₁₉H₂₁F₂N₃O₃ 377.2; m/z found, 378.1 [M+H]⁺. [a]²⁵_{D}=+8.964 (c=0.6, CHCl₃).

### Intermediate 65: tert-Butyl (3R)-11,11-difluoro-10-hydroxy-3-methyl-1,3,4,7,8,9,10,11-octahydro-2H-pyrido[4′,3′:3,4]pyrazolo[1,5-a]azepine-2-carboxylate

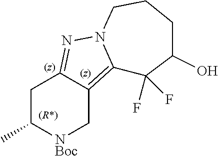 [SMILES: C[C@@H]1Cc2nn3c(c2CN1C(=O)OC(C)(C)C)C(F)(F)C(O)CCC3]

Step A. Ethyl 6-(benzyloxy)-2,2-difluoro-3-hydroxyhexanoate. To a refluxing suspension of activated zinc dust (2.20 g, 33.66 mmol) in dry THF (30 mL) was added ethyl bromodifluoroacetate (3.24 mL, 25.25 mmol). After 1 min, a solution of 4-(benzyloxy)butanal (3 g, 16.83 mmol) dissolved in 3 mL of THF was added dropwise, over 15 min. After complete addition the reaction was refluxed for a further 4 h. The mixture was cooled to rt and carefully poured into 100 ml 1 M HCl in ice bath. Stirring of the resultant mixture was continued for other 0.5 h. The organic layer was washed with brine (500 mL), dried over Na₂SO₄, filtered and concentrated in vacuo to give the title compound (5 g, crude) as an orange oil. MS (ESI): mass calcd. for C₁₅H₂₀F₂O₄, 302.3; m/z found, 303.1 [M+H]⁺.

Step B. Ethyl 3,6-bis(benzyloxy)-2,2-difluorohexanoate. To a solution of ethyl 6-(benzyloxy)-2,2-difluoro-3-hydroxyhexanoate (5 g, 16.87 mmol) in dry THF (40 mL) was treated with sodium hydride (60% dispersion in mineral oil, 0.88 g, 22 mmol) in ice bath under N₂. The mixture was stirred for 10 min. The benzyl bromide (2.21 mL, 18.60 mmol) was added dropwise to the mixture. The resultant mixture was stirred in ice bath for 30 min and then warmed to 25° C. and was stirred for 16 h. The mixture was diluted with H₂O (300 mL), and extracted with Ethyl acetate (200 mL×2). The combined organic layer was washed with brine (300 mL), dried over Na₂SO₄, filtered and concentrated in vacuo. The residue was purified by column chromatography (SiO₂, Heptane/Ethyl acetate=1/0 to 50/1) to give the title compound 1.65 g, 4.21 mmol, 25% yield) as a yellow oil. MS (ESI): mass calcd. for C₂₂H₂₆F₂O₄, 392.44; m/z found, 393.30 [M+H]+. ¹H NMR (400 MHz, CDCl₃) δ=7.35-7.24 (m, 10H), 4.71-4.54 (m, 2H), 4.45 (s, 2H), 4.30-4.22 (q, 2H), 3.96-3.87 (m, 1H), 3.46-3.38 (m, 2H), 1.85-1.61 (m, 4H), 1.26 (t, J=8 Hz, 3H).

Step C. tert-Butyl (2R)-5-(3,6-bis(benzyloxy)-2,2-difluorohexanoyl)-2-methyl-4-oxopiperidine-1-carboxylate. To a solution of tert-butyl (R)-2-methyl-4-oxopiperidine-1-carboxylate (0.7 g, 3.25 mmol) in THF (10 mL) was added LiHMDS (1 M, 4.22 mL) at −78° C. under N₂. The mixture was stirred at −78° C. for 0.5 h, then a solution of ethyl 3,6-bis(benzyloxy)-2,2-difluorohexanoate (1.4 g, 3.57 mmol) in THF (2 mL) was added into the mixture slowly. The mixture was warmed up to room temperature over 0.5 h and stirred at 60° C. for 12 h. The mixture was poured into HCl solution (0.5 N, 8 mL) and stirred for 1 min. The aqueous phase was extracted with ethyl acetate (100 mL). The combined organic phase was washed with brine (100 mL), dried with anhydrous Na₂SO₄, filtered and concentrated in vacuo to give the title compound (620 mg, crude) as a yellow oil. MS (ESI): mass calcd. for C₃₁H₃₉F₂NO₆, 559.651; m/z found, 577.3 [M+NH4]⁺.

Step D. tert-Butyl (6R)-3-(2,5-bis(benzyloxy)-1,1-difluoropentyl)-6-methyl-2,4,6,7-tetrahydro-5H-pyrazolo[4,3-c]pyridine-5-carboxylate. To a solution of tert-butyl (2R)-5-(3,6-bis(benzyloxy)-2,2-difluorohexanoyl)-2-methyl-4-oxopiperidine-1-carboxylate (1.3 g, crude, 2.3 mmol) in EtOH (20 mL) was added N₂H₄ (0.08 mL, 2.5 mmol) in ice bath. The mixture was warmed up and stirred at 25° C. for 12 hr. The mixture was concentrated in vacuo. The mixture was dissolved in HCl (80 mL, 1 N). The aqueous phase was extracted with ethyl acetate (50 mL×2). The combined organic phase was washed with brine (100 mL×2), dried with anhydrous Na₂SO₄, filtered and concentrated in vacuo to give the title compound (1290 mg, crude) as a yellow oil. MS (ESI): mass calcd. for C₃₁H₃₉F₂N₃O₄, 555.67; m/z found, 556.30 [M+H]⁺.

Step E. tert-Butyl (6R)-3-(1,1-difluoro-2,5-dihydroxypentyl)-6-methyl-2,4,6,7-tetrahydro-5H-pyrazolo[4,3-c]pyridine-5-carboxylate. To a solution of tert-butyl (6R)-3-(2,5-bis(benzyloxy)-1,1-difluoropentyl)-6-methyl-2,4,6,7-tetrahydro-5H-pyrazolo[4,3-c]pyridine-5-carboxylate (1290 mg, crude, 2.32 mmol) in EtOH (20 mL) was added 30% Pd/C (0.25 g, 0.23 mmol) at 25° C. The mixture was stirred under atmosphere of H₂ (50 psi) at 25° C. for 12 hr. The mixture was filtered through a micro pore filter with celite and then silica gel, and the filtrate was concentrated in vacuo to give the title compound as a yellow oil (270 mg, crude). MS (ESI): mass calcd. for C₁₇H₂₇F₂N₃O₄, 375.42; m/z found, 376.2 [M+H]⁺.

Step F. tert-Butyl (6R)-3-(1,1-difluoro-2-hydroxy-5-((methylsulfonyl)oxy)pentyl)-6-methyl-2,4,6,7-tetrahydro-5H-pyrazolo[4,3-c]pyridine-5-carboxylate. To a solution of tert-butyl (6R)-3-(1,1-difluoro-2,5-dihydroxypentyl)-6-methyl-2,4,6,7-tetrahydro-5H-pyrazolo[4,3-c]pyridine-5-carboxylate (250 mg, 0.67 mmol) and 2,4,6-trimethylpyridine (30.88 mL, 6.66 mmol) in DCM (7 mL) was added MsCl (0.06 mL, 0.73 mmol) at 0° C. under N₂. The mixture was stirred at 0° C. for 4 hr. Then the reaction mixture was moved to fridge (5° C.) and stood for 16 hr. The reaction mixture was diluted with H₂O (10 mL) and extracted with DCM (10 mL×3). The combined organic layer was washed with brine (20 mL), dried over Na₂SO₄, filtered and concentrated in vacuo to provide the title compound as a yellow oil (303 mg, crude). MS (ESI): mass calcd. for C₁₈H₂₉F₂N₃O₆S, 453.51; m/z found, 454.2 [M+H]+.

Step G. tert-Butyl (3R)-11,11-difluoro-10-hydroxy-3-methyl-1,3,4,7,8,9,10,11-octahydro-2H-pyrido[4′,3′:3,4]pyrazolo[1,5-a]azepine-2-carboxylate. To a solution of tert-butyl (6R)-3-(1,1-difluoro-2-hydroxy-5-((methylsulfonyl)oxy)pentyl)-6-methyl-2,4,6,7-tetrahydro-5H-pyrazolo[4,3-c]pyridine-5-carboxylate (303 mg, 0.67 mmol) in MeCN (5 mL) was added DBU (0.3 mL, 2.0 mmol). The mixture was stirred at 25° C. for 12 hr. The reaction mixture was diluted with aq HCl (1 N, 10 mL) and extracted with EtOAc (10 mL×3). The combined organic layer was washed with brine (20 mL), dried over Na₂SO₄, filtered and concentrated in vacuo to provide the title compound as a yellow oil (230 mg, crude) which was used without further purification. MS (ESI): mass calcd. for C₁₇H₂₅F₂N₃O₃, 357.40; m/z found, 358.2 [M+H]+.

### Example 1

### N-(3-Cyano-4-fluorophenyl)-11,11-difluoro-8-methylene-3,4,8,9,10,11-hexahydro-1H-pyrido[4′,3′:3,4]pyrazolo[1,5-a]azepine-2(7H)-carboxamide

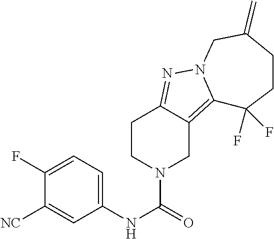 [SMILES: C=C1CCC(F)(F)c2c3c(nn2C1)CCN(C(=O)Nc1ccc(F)c(C#N)c1)C3]

Step A. 11,11-Difluoro-8-methylene-2,3,4,7,8,9,10,11-octahydro-1H-pyrido[4′,3′:3,4] pyrazolo[1,5-a]azepine. To a solution of tert-butyl 11,11-difluoro-8-methylene-3,4,8,9,10,11-hexahydro-1H-pyrido[4′,3′:3,4]pyrazolo[1,5-a]azepine-2(7H)-carboxylate (70 mg, 198.01 μmol) in DCM (3 mL) was added TFA (1.48 g, 12.97 mmol, 960.00 μL). The mixture was stirred at 20° C. for 1 h. The mixture was concentrated in vacuo to give the title compound (75 mg, crude, TFA salt) as yellow oil, which was used crude in the next step without further purification.

Step B. N-(3-Cyano-4-fluorophenyl)-11,11-difluoro-8-methylene-3,4,8,9,10,11-hexahydro-1H-pyrido[4′,3′:3,4]pyrazolo[1,5-a]azepine-2(7H)-carboxamide. To a solution of 11,11-difluoro-8-methylene-2,3,4,7,8,9,10,11-octahydro-1H-pyrido[4′,3′:3,4]pyrazolo[1,5-a]azepine (75 mg, crude, TFA salt) in DCM (5 mL) was added TEA (107.41 mg, 1.06 mmol, 147.74 μL) and phenyl (3-cyano-4-fluorophenyl) carbamate (51.68 mg, 201.68 μmol). The mixture was stirred at 20° C. for 14 h. The mixture was concentrated in vacuo. The residue was purified by RP HPLC (condition A) to give the title compound (36.82 mg, 90.82 μmol, 42.78% yield, 99% purity) as white solid. MS (ESI): mass calcd. for C₂₀H₁₈F₃N₅O, 401.2; m/z found, 402.1 [M+H]⁺. ¹H NMR (400 MHz, CDCl₃) δ=7.72 (dd, J=2.8, 5.4 Hz, 1H), 7.62-7.59 (m, 1H), 7.15 (t, J=8.7 Hz, 1H), 6.51 (s, 1H), 5.21 (s, 1H), 5.13 (s, 1H), 4.79 (s, 2H), 4.62 (s, 2H), 3.80 (t, J=5.8 Hz, 2H), 2.84 (t, J=5.7 Hz, 2H), 2.67-2.64 (m, 2H), 2.42-2.37 (m, 2H).

### Example 2

### 11,11-Difluoro-N-(4-fluoro-3-(trifluoromethyl)phenyl)-8-methylene-3,4,8,9,10,11-hexahydro-1H-pyrido[4′,3′:3,4]pyrazolo[1,5-a]azepine-2(7H)-carboxamide

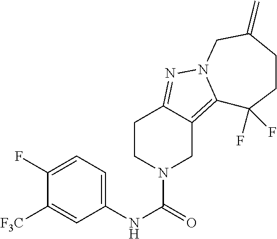 [SMILES: C=C1CCC(F)(F)c2c3c(nn2C1)CCN(C(=O)Nc1ccc(F)c(C(F)(F)F)c1)C3]

The title compound was prepared in a manner analogous to Example 1, however using phenyl (4-fluoro-3-(trifluoromethyl)phenyl)carbamate instead of phenyl (3-cyano-4-fluorophenyl)carbamate in Step B. MS (ESI): mass calcd. for C₂₀H₁₈F₆N₄O 444.1; m/z found, 445.1 [M+H]⁺. ¹H NMR (400 MHz, CDCl₃) δ=7.67-7.55 (m, 2H), 7.14 (t, J=9.3 Hz, 1H), 6.47 (s, 1H), 5.21 (s, 1H), 5.13 (s, 1H), 4.79 (s, 2H), 4.63 (s, 2H), 3.81 (t, J=5.8 Hz, 2H), 2.85 (t, J=5.7 Hz, 2H), 2.69-2.64 (m, 2H), 2.48-2.35 (m, 2H).

### Example 3

### (S)-N-(3-Cyano-4-fluorophenyl)-11,11-difluoro-8-hydroxy-3,4,8,9,10,11-hexahydro-1H-pyrido[4′,3′:3,4]pyrazolo[1,5-a]azepine-2(7H)-carboxamide

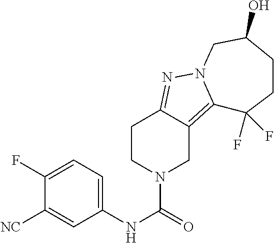 [SMILES: N#Cc1cc(NC(=O)N2CCc3nn4c(c3C2)C(F)(F)CC[C@H](O)C4)ccc1F]

The title compound was prepared in a manner analogous to Example 1, however using (5)-tert-butyl 11,11-difluoro-8-hydroxy-3,4,8,9,10,11-hexahydro-1H-pyrido[4′,3′:3,4]pyrazolo[1,5-a]azepine-2(7H)-carboxylate (Intermediate 2) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A. MS (ESI): mass calcd. for C₁₉H₁₈F₃N₅O₂ 405.1; m/z found, 406.1 [M+H]+. ¹H NMR (400 MHz, CD₃OD) δ=7.79 (dd, J=2.8, 5.6 Hz, 1H), 7.69-7.65 (m, 1H), 7.26 (t, J=9.0 Hz, 1H), 4.67 (d, J=2.2 Hz, 2H), 4.38-4.25 (m, 2H), 4.00 (t, J=7.0 Hz, 1H), 3.80 (br t, J=5.2 Hz, 2H), 2.79 (t, J=5.8 Hz, 2H), 2.61-2.44 (m, 1H), 2.36-2.24 (m, 1H), 2.22-2.14 (m, 1H), 2.09-1.98 (m, 1H).

### Example 4

### (S)-11,11-Difluoro-N-(4-fluoro-3-(trifluoromethyl)phenyl)-8-hydroxy-3,4,8,9,10,11-hexahydro-1H-pyrido[4′,3′:3,4]pyrazolo[1,5-a]azepine-2(7H)-carboxamide

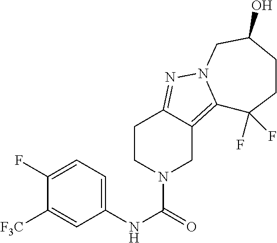 [SMILES: O=C(Nc1ccc(F)c(C(F)(F)F)c1)N1CCc2nn3c(c2C1)C(F)(F)CC[C@H](O)C3]

The title compound was prepared in a manner analogous to Example 1, however using (S)-tert-butyl 11,11-difluoro-8-hydroxy-3,4,8,9,10,11-hexahydro-1H-pyrido[4′,3′:3,4]pyrazolo[1,5-a]azepine-2(7H)-carboxylate (Intermediate 2) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (4-fluoro-3-(trifluoromethyl)phenyl)carbamate instead of phenyl (3-cyano-4-fluorophenyl) carbamate in Step B. MS (ESI): mass calcd. for C₁₉H₁₈F₆N₄O₂ 448.1; m/z found, 449.1 [M+H]+. ¹H NMR (400 MHz, CD₃OD) δ=7.77 (dd, J=2.6, 6.2 Hz, 1H), 7.69-7.65 (m, 1H), 7.22 (t, J=9.6 Hz, 1H), 4.67 (br d, J=2.0 Hz, 2H), 4.38-4.25 (m, 2H), 3.99 (br t, J=7.0 Hz, 1H), 3.80 (br t, J=5.3 Hz, 2H), 2.79 (t, J=5.7 Hz, 2H), 2.61-1.95 (m, 4H).

### Example 5

### (S)-N-(2-Bromo-3-fluoropyridin-4-yl)-11,11-difluoro-8-hydroxy-3,4,8,9,10,11-hexahydro-1H-pyrido[4′,3′:3,4]pyrazolo[1,5-a]azepine-2(7H)-carboxamide

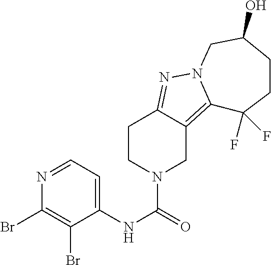 [SMILES: O=C(Nc1ccnc(Br)c1Br)N1CCc2nn3c(c2C1)C(F)(F)CC[C@H](O)C3]

The title compound was prepared in a manner analogous to Example 1, however using (5)-tert-butyl 11,11-difluoro-8-hydroxy-3,4,8,9,10,11-hexahydro-1H-pyrido[4′,3′:3,4]pyrazolo[1,5-a]azepine-2(7H)-carboxylate (Intermediate 2) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (2-bromo-3-fluoropyridin-4-yl)carbamate instead of phenyl (3-cyano-4-fluorophenyl) carbamate in Step B. MS (ESI): mass calcd. for C₁₇H₁₇BrF₃N₅O₂ 459.1/461.1; m/z found, 460.0/462.0 [M+H]+. ¹H NMR(400 MHz, CDCl₃) δ=8.21-8.17 (m, 1H), 8.09 (d, J=5.6 Hz, 1H), 6.95 (s, 1H), 4.76-4.63 (m, 2H), 4.50-4.49 (m, 2H), 4.19 (br s, 1H), 3.89-3.74 (m, 2H), 2.88 (t, J=5.6 Hz, 2H), 2.62-2.49 (m, 1H), 2.37-2.09 (m, 3H).

### Example 6

### (S)-N-(3-Cyano-2,4-difluorophenyl)-11,11-difluoro-8-hydroxy-3,4,8,9,10,11-hexahydro-1H-pyrido[4′,3′:3,4]pyrazolo[1,5-a]azepine-2(7H)-carboxamide

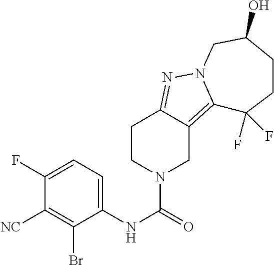 [SMILES: N#Cc1c(F)ccc(NC(=O)N2CCc3nn4c(c3C2)C(F)(F)CC[C@H](O)C4)c1Br]

The title compound was prepared in a manner analogous to Example 1, however using (5)-tert-butyl 11,11-difluoro-8-hydroxy-3,4,8,9,10,11-hexahydro-1H-pyrido[4′,3′:3,4]pyrazolo[1,5-a]azepine-2(7H)-carboxylate (Intermediate 2) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (3-cyano-2,4-difluorophenyl)carbamate instead of phenyl (3-cyano-4-fluorophenyl) carbamate in Step B. MS (ESI): mass calcd. for C₁₉H₁₇F₄N₅O₂ 423.1; m/z found, 424.1 [M+H]+. ¹H NMR (400 MHz, CDCl₃) δ=8.37-8.31 (m, 1H), 7.10-7.00 (m, 1H), 6.57 (br d, J=2.6 Hz, 1H), 4.69 (s, 2H), 4.48-4.38 (m, 2H), 4.19 (br s, 1H), 3.87-3.80 (m, 2H), 2.90-2.81 (m, 2H), 2.65-2.48 (m, 1H), 2.39-2.13 (m, 3H), 1.85 (br s, 1H).

### Example 7

### (S)-N-(3-Bromo-2,4-difluorophenyl)-11,11-difluoro-8-hydroxy-3,4,8,9,10,11-hexahydro-1H-pyrido[4′,3′:3,4]pyrazolo[1,5-a]azepine-2(7H)-carboxamide

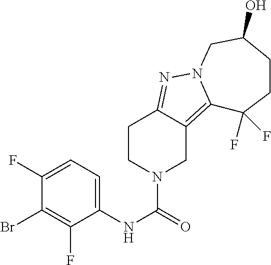 [SMILES: O=C(Nc1ccc(F)c(Br)c1F)N1CCc2nn3c(c2C1)C(F)(F)CC[C@H](O)C3]

The title compound was prepared in a manner analogous to Example 1, however using (5)-tert-butyl 11,11-difluoro-8-hydroxy-3,4,8,9,10,11-hexahydro-1H-pyrido[4′,3′:3,4]pyrazolo[1,5-a]azepine-2(7H)-carboxylate (Intermediate 2) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (3-bromo-2,4-difluorophenyl)carbamate instead of phenyl (3-cyano-4-fluorophenyl) carbamate in Step B. MS (ESI): mass calcd. for C₁₈H₁₇BrF₄N₄O₂ 476.1/478.1; m/z found, 477.0/479.0 [M+H]+. ¹H NMR (400 MHz, CDCl₃) δ=7.94-7.88 (m, 1H), 6.94-6.79 (m, 1H), 6.44 (br s, 1H), 4.67 (s, 2H), 4.35 (d, J=3.5 Hz, 2H), 4.09 (br s, 1H), 3.73 (t, J=5.9 Hz, 2H), 2.77 (t, J=5.7 Hz, 2H), 2.57-2.39 (m, 1H), 2.30-2.05 (m, 3H), 1.78 (br s,1H).

### Example 8

### (R)-N-(3-Cyano-4-fluorophenyl)-11,11-difluoro-8-hydroxy-3,4,8,9,10,11-hexahydro-1H-pyrido[4′,3′:3,4]pyrazolo[1,5-a]azepine-2(7H)-carboxamide

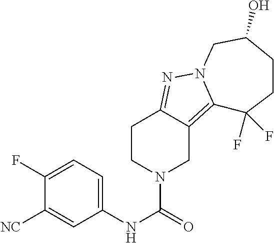 [SMILES: N#Cc1cc(NC(=O)N2CCc3nn4c(c3C2)C(F)(F)CC[C@@H](O)C4)ccc1F]

The title compound was prepared in a manner analogous to Example 1, however using (R)-tert-butyl 11,11-difluoro-8-hydroxy-3,4,8,9,10,11-hexahydro-1H-pyrido[4′,3′:3,4]pyrazolo[1,5-a]azepine-2(7H)-carboxylate (Intermediate 3) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A. MS (ESI): mass calcd. for C₁₉H₁₈F₃N₅O₂ 405.1; m/z found, 406.1 [M+H]⁺. ¹H NMR (400 MHz, CD₃OD) δ=7.81 (dd, J=2.8, 5.6 Hz, 1H), 7.74-7.68 (m, 1H), 7.28 (t, J=9.0 Hz, 1H), 4.62 (s, 2H), 4.40-4.28 (m, 2H), 4.02 (br t, J=7.1 Hz, 1H), 3.86-3.79 (m, 2H), 2.81 (t, J=5.7 Hz, 2H), 2.62-2.47 (m, 1H), 2.37-2.15 (m, 2H), 2.11-2.01 (m, 1H).

### Example 9

### (R)-11,11-Difluoro-N-(4-fluoro-3-(trifluoromethyl)phenyl)-8-hydroxy-3,4,8,9,10,11-hexahydro-1H-pyrido[4′,3′:3,4]pyrazolo[1,5-a]azepine-2(7H)-carboxamide

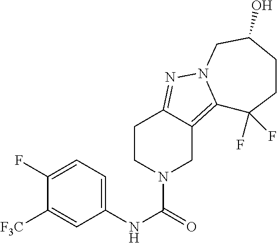 [SMILES: O=C(Nc1ccc(F)c(C(F)(F)F)c1)N1CCc2nn3c(c2C1)C(F)(F)CC[C@@H](O)C3]

The title compound was prepared in a manner analogous to Example 1, however using (R)-tert-butyl 11,11-difluoro-8-hydroxy-3,4,8,9,10,11-hexahydro-1H-pyrido[4′,3′:3,4]pyrazolo[1,5-a]azepine-2(7H)-carboxylate (Intermediate 3) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (4-fluoro-3-(trifluoromethyl)phenyl)carbamate instead of phenyl (3-cyano-4-fluorophenyl) carbamate in Step B. MS (ESI): mass calcd. for C₁₉H₁₈F₆N₄O₂ 448.1; m/z found, 449.1 [M+H]+. ¹H NMR (400 MHz, CD₃OD) δ=7.76 (dd, J=2.7, 6.2 Hz, 1H), 7.67-7.61 (m, 1H), 7.22 (t, J=9.6 Hz, 1H), 4.69 (s, 2H), 4.38-4.25 (m, 2H), 3.99 (br t, J=6.9 Hz, 1H), 3.86-3.75 (m, 2H), 2.78 (t, J=5.7 Hz, 2H), 2.59-2.41 (m, 1H), 2.36-2.12 (m, 2H), 2.08-1.99 (m, 1H).

### Example 10

### (R)-N-(2-Bromo-3-fluoropyridin-4-yl)-11,11-difluoro-8-hydroxy-3,4,8,9,10,11-hexahydro-1H-pyrido[4′,3′:3,4]pyrazolo[1,5-a]azepine-2(7H)-carboxamide

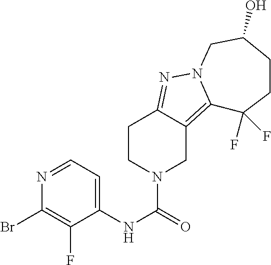 [SMILES: O=C(Nc1ccnc(Br)c1F)N1CCc2nn3c(c2C1)C(F)(F)CC[C@@H](O)C3]

The title compound was prepared in a manner analogous to Example 1, however using (R)-tert-butyl 11,11-difluoro-8-hydroxy-3,4,8,9,10,11-hexahydro-1H-pyrido[4′,3′:3,4]pyrazolo[1,5-a]azepine-2(7H)-carboxylate (Intermediate 3) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (2-bromo-3-fluoropyridin-4-yl)carbamate instead of phenyl (3-cyano-4-fluorophenyl) carbamate in Step B. MS (ESI): mass calcd. for C₁₇H₁₇BrF₃N₅O₂ 459.1/461.1; m/z found, 460.0/462.0 [M+H]+. ¹H NMR (400 MHz, CDCl₃) δ=8.22-8.14 (m, 1H), 8.09 (d, J=5.5 Hz, 1H), 6.96 (br d, J=3.8 Hz, 1H), 4.76-4.61 (m, 2H), 4.49-4.39 (m, 2H), 4.25-4.14 (m, 1H), 3.91-3.78 (m, 2H), 2.88 (t, J=5.8 Hz, 2H), 2.66-2.47 (m, 1H), 2.38-2.10 (m, 3H).

### Example 11

### (R)-N-(3-Cyano-2,4-difluorophenyl)-11,11-difluoro-8-hydroxy-3,4,8,9,10,11-hexahydro-1H-pyrido[4′,3′:3,4]pyrazolo[1,5-a]azepine-2(7H)-carboxamide

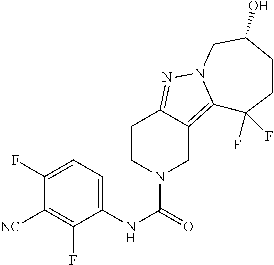 [SMILES: N#Cc1c(F)ccc(NC(=O)N2CCc3nn4c(c3C2)C(F)(F)CC[C@@H](O)C4)c1F]

The title compound was prepared in a manner analogous to Example 1, however using (R)-tert-butyl 11,11-difluoro-8-hydroxy-3,4,8,9,10,11-hexahydro-1H-pyrido[4′,3′:3,4]pyrazolo[1,5-a]azepine-2(7H)-carboxylate (Intermediate 3) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (3-cyano-2,4-difluorophenyl)carbamate instead of phenyl (3-cyano-4-fluorophenyl) carbamate in Step B. MS (ESI): mass calcd. for C₁₉H₁₇F₄N₅O₂ 423.1; m/z found, 424.1 [M+H]+. ¹H NMR (400 MHz, CDCl₃) δ=8.33 (dt, J=5.8, 9.1 Hz, 1H), 7.14-6.93 (m, 1H), 6.59 (br d, J=2.6 Hz, 1H), 4.77-4.56 (m, 2H), 4.49-4.32 (m, 2H), 4.18 (br s, 1H), 3.93-3.68 (m, 2H), 2.99-2.75 (m, 2H), 2.66-2.45 (m, 1H), 2.36-2.00 (m, 4H).

### Example 12

### (R)-N-(3-Bromo-2,4-difluorophenyl)-11,11-difluoro-8-hydroxy-3,4,8,9,10,11-hexahydro-1H-pyrido[4′,3′:3,4]pyrazolo[1,5-a]azepine-2(7H)-carboxamide

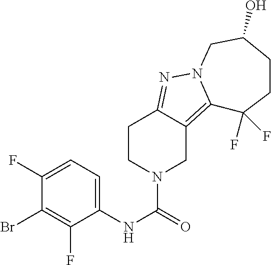 [SMILES: O=C(Nc1ccc(F)c(Br)c1F)N1CCc2nn3c(c2C1)C(F)(F)CC[C@@H](O)C3]

The title compound was prepared in a manner analogous to Example 1, however using (R)-tert-butyl 11,11-difluoro-8-hydroxy-3,4,8,9,10,11-hexahydro-1H-pyrido[4′,3′:3,4]pyrazolo[1,5-a]azepine-2(7H)-carboxylate (Intermediate 3) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (3-bromo-2,4-difluorophenyl)carbamate instead of phenyl (3-cyano-4-fluorophenyl) carbamate in Step B. MS (ESI): mass calcd. for C₁₈H₁₇BrF₄N₄O₂ 476.1/478.1; m/z found, 477.0/479.0 [M+H]+. ¹H NMR (400 MHz, CDCl₃) δ=8.01-7.93 (m, 1H), 6.96-6.92 (m, 1H), 6.50 (br d, J=2.9 Hz, 1H), 4.69-4.59 (m, 2H), 4.35 (d, J=4.0 Hz, 2H), 4.15 (br s, 1H), 3.79 (t, J=5.8 Hz, 2H), 2.83 (t, J=5.7 Hz, 2H), 2.65-2.45 (m, 1H), 2.28-2.01 (m, 3H), 1.86 (br s, 1H).

### Example 13

### (S)-N-(3-Cyano-4-fluorophenyl)-8-(2,2-difluoroethoxy)-11,11-difluoro-3,4,8,9,10,11-hexahydro-1H-pyrido[4′,3′:3,4]pyrazolo[1,5-a]azepine-2(7H)-carboxamide

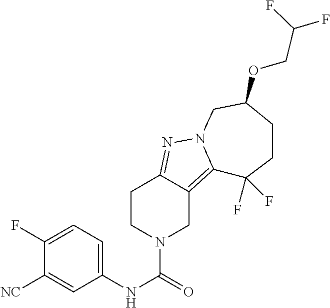 [SMILES: N#Cc1cc(NC(=O)N2CCc3nn4c(c3C2)C(F)(F)CC[C@H](OCC(F)F)C4)ccc1F]

The title compound was prepared in a manner analogous to Example 1, however using (S)-tert-butyl 8-(2,2-difluoroethoxy)-11,11-difluoro-3,4,8,9,10,11-hexahydro-1H-pyrido[4′,3′:3,4]pyrazolo[1,5-a]azepine-2(7H)-carboxylate (Intermediate 4) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A. MS (ESI): mass calcd. instead of C₂₁H₂₀N₅O₂F₅, 469.2; m/z found, 470.1 [M+H]⁺.

¹H NMR (400 MHz, CDCl₃) δ=7.78-7.71 (m, 1H), 7.69-7.59 (m, 1H), 7.16 (t, J=8.7 Hz, 1H), 6.58 (s, 1H), 5.98-5.62 (m, 1H), 4.65-4.51 (m, 3H), 4.36 (br d, J=14.6 Hz, 1H), 3.84-3.70 (m, 5H), 2.85 (br t, J=5.5 Hz, 2H), 2.64-2.41 (m, 1H), 2.34-2.14 (m, 3H).

### Example 14

### (R)-N-(3-Cyano-4-fluorophenyl)-8-(2,2-difluoroethoxy)-11,11-difluoro-3,4,8,9,10,11-hexahydro-1H-pyrido[4′,3′:3,4]pyrazolo[1,5-a]azepine-2(7H)-carboxamide

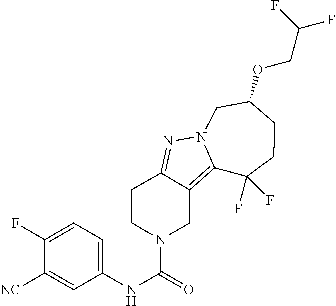 [SMILES: N#Cc1cc(NC(=O)N2CCc3nn4c(c3C2)C(F)(F)CC[C@@H](OCC(F)F)C4)ccc1F]

The title compound was prepared in a manner analogous to Example 1, however using (R)-tert-butyl 8-(2,2-difluoroethoxy)-11,11-difluoro-3,4,8,9,10,11-hexahydro-1H-pyrido[4′,3′:3,4]pyrazolo[1,5-a]azepine-2(7H)-carboxylate (Intermediate 5) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A. MS (ESI): mass calcd. for C₂₁H₂₀N₅O₂F₅, 469.2; m/z found, 470.1 [M+H]⁺. ¹H NMR (400 MHz, CDCl₃) δ=7.72 (dd, J=2.8, 5.4 Hz, 1H), 7.63-7.60 (m, 1H), 7.15 (t, J=8.7 Hz, 1H), 6.54 (s, 1H), 5.96-5.62 (m, 1H), 4.67-4.53 (m, 3H), 4.35 (d, J=14.5 Hz, 1H), 3.87-3.63 (m, 5H), 2.91-2.79 (m, 2H), 2.62-2.41 (m, 1H), 2.34-2.10 (m, 3H).

### Example 15

### (R)-N-(3-Cyano-4-fluorophenyl)-8,11,11-trifluoro-3,4,8,9,10,11-hexahydro-1H-pyrido[4′,3′:3,4]pyrazolo[1,5-a]azepine-2(7H)-carboxamide

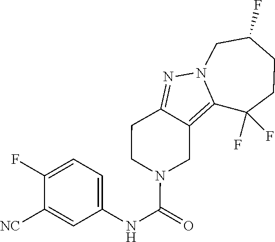 [SMILES: N#Cc1cc(NC(=O)N2CCc3nn4c(c3C2)C(F)(F)CC[C@@H](F)C4)ccc1F]

The title compound was prepared in a manner analogous to Example 1, however using (R)-tert-butyl 8,11,11-trifluoro-3,4,8,9,10 ,11-hexahydro-1H-pyrido[4′,3′:3,4]pyrazolo[1,5-a]azepine-2(7H)-carboxylate (Intermediate 6) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A. MS (ESI): mass calcd. for C₁₉H₁₇F₄N₅O, 407.1; m/z found, 408.1 [M+H]+. ¹H NMR (400 MHz, CDCl₃) δ=7.74-7.72 (m, 1H), 7.63-7.65 (m, 1H), 7.17 (t, J=8.7 Hz, 1H), 6.55 (s, 1H), 5.05-4.84 (m, 1H), 4.73-4.58 (m, 3H), 4.53-4.40 (m, 1H), 3.88-3.78 (m, 2H), 2.93-2.83 (m, 2H), 2.65-2.49 (m, 1H), 2.42-2.21 (m, 3H).

### Example 16

### (R)-8,11,11-Trifluoro-N-(4-fluoro-3-(trifluoromethyl)phenyl)-3,4,8,9,10,11-hexahydro-1H-pyrido[4′,3′:3,4]pyrazolo[1,5-a]azepine-2(7H)-carboxamide

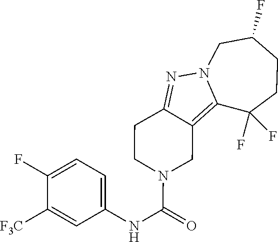 [SMILES: O=C(Nc1ccc(F)c(C(F)(F)F)c1)N1CCc2nn3c(c2C1)C(F)(F)CC[C@@H](F)C3]

The title compound was prepared in a manner analogous to Example 1, however using (R)-tert-butyl 8,11,11-trifluoro-3,4,8,9,10 ,11-hexahydro-1H-pyrido[4′,3′:3,4]pyrazolo[1,5-a]azepine-2(7H)-carboxylate (Intermediate 6) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (4-fluoro-3-(trifluoromethyl)phenyl)carbamate instead of phenyl (3-cyano-4-fluorophenyl) carbamate in Step B. MS (ESI): mass calcd. for C₁₉H₁₇F₇N₄O, 450.1; m/z found, 451.1 [M+H]+. ¹H NMR (400 MHz, CDCl₃) δ=7.66-7.56 (m, 2H), 7.14 (t, J=9.3 Hz, 1H), 6.51 (s, 1H), 5.02-4.83 (m, 1H), 4.74-4.57 (m, 3H), 4.52-4.40 (m, 1H), 3.87-3.73 (m, 2H), 2.91-2.80 (m, 2H), 2.62-2.45 (m, 1H), 2.42-2.20 (m, 3H).

### Example 17

### (S)-N-(3-Cyano-4-fluorophenyl)-8,11,11-trifluoro-3,4,8,9,10,11-hexahydro-1H-pyrido[4′,3′:3,4]pyrazolo[1,5-a]azepine-2(7H)-carboxamide

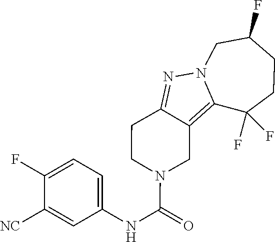 [SMILES: N#Cc1cc(NC(=O)N2CCc3nn4c(c3C2)C(F)(F)CC[C@H](F)C4)ccc1F]

The title compound was prepared in a manner analogous to Example 1, however using (5)-tert-butyl 8,11,11-trifluoro-3,4,8,9,10 ,11-hexahydro-1H-pyrido[4′,3′:3,4]pyrazolo[1,5-a]azepine-2(7H)-carboxylate (Intermediate 7) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A. MS (ESI): mass calcd. for C₁₉H₁₇F₄N₅O, 407.1; m/z found, 408.1 [M+H]+. ¹H NMR (400 MHz, CDCl₃) δ=7.72 (dd, J=2.8, 5.4 Hz, 1H), 7.62-7.59 (m, 1H), 7.15 (t, J=8.7 Hz, 1H), 6.51 (s, 1H), 5.05-4.82 (m, 1H), 4.72-4.56 (m, 3H), 4.53-4.38 (m, 1H), 3.87-3.75 (m, 2H), 2.85 (t, J=5.5 Hz, 2H), 2.63-2.16 (m, 4H).

### Example 18

### (S)-8,11,11-Trifluoro-N-(4-fluoro-3-(trifluoromethyl)phenyl)-3,4,8,9,10,11-hexahydro-1H-pyrido[4′,3′:3,4]pyrazolo[1,5-a]azepine-2(7H)-carboxamide

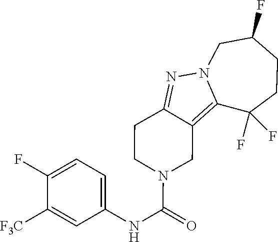 [SMILES: O=C(Nc1ccc(F)c(C(F)(F)F)c1)N1CCc2nn3c(c2C1)C(F)(F)CC[C@H](F)C3]

The title compound was prepared in a manner analogous to Example 1, however using (5)-tert-butyl 8,11,11-trifluoro-3,4,8,9,10 ,11-hexahydro-1H-pyrido[4′,3′:3,4]pyrazolo[1,5-a]azepine-2(7H)-carboxylate (Intermediate 7) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (4-fluoro-3-(trifluoromethyl)phenyl)carbamate instead of phenyl (3-cyano-4-fluorophenyl) carbamate in Step B. MS (ESI): mass calcd. for C₁₉H₁₇F₇N₄O, 450.1; m/z found, 451.0 [M+H]+. ¹H NMR (400 MHz, CDCl₃) δ=7.67-7.54 (m, 2H), 7.14 (t, J=9.3 Hz, 1H), 6.47 (s, 1H), 5.04-4.81 (m, 1H), 4.73-4.56 (m, 3H), 4.53-4.38 (m, 1H), 3.88-3.74 (m, 2H), 2.86 (t, J=5.8 Hz, 2H), 2.63-2.16 (m, 4H).

### Example 19

### (S*)-N-(3-Cyano-4-fluorophenyl)-11,11-difluoro-8-(hydroxymethyl)-3,4,8,9,10,11-hexahydro-1H-pyrido[4′,3′:3,4]pyrazolo[1,5-a]azepine-2(7H)-carboxamide

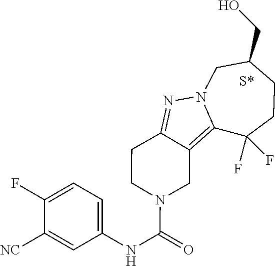 [SMILES: N#Cc1cc(NC(=O)N2CCc3nn4c(c3C2)C(F)(F)CC[C@H](CO)C4)ccc1F]

The title compound was prepared in a manner analogous to Example 1, however using (S*)-tert-butyl 11,11-difluoro-8-(hydroxymethyl)-3,4,8,9,10,11-hexahydro-1H-pyrido[4′,3′:3,4]pyrazolo[1,5-a]azepine-2(7H)-carboxylate (Intermediate 8) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A. MS (ESI): mass calcd. for C₂₀H₂₀F₃N₅O₂, 419.2; m/z found, 420.1 [M+H]+. ¹H NMR (400 MHz, CDCl₃) δ=7.72 (dd, J=2.8, 5.4 Hz, 1H), 7.62-7.59 (m, 1H), 7.15 (t, J=8.7 Hz, 1H), 6.56 (s, 1H), 4.63 (s, 2H), 4.53 (br d, J=14.3 Hz, 1H), 4.20 (dd, J=8.6, 14.2 Hz, 1H), 3.86-3.77 (m, 2H), 3.63-3.56 (m, 1H), 3.54-3.44 (m, 1H), 2.83 (t, J=5.8 Hz, 2H), 2.51-2.17 (m, 2H), 2.12-1.99 (m, 2H), 1.94-1.80 (m, 1H).

### Example 20

### (S*)-11,11-Difluoro-N-(4-fluoro-3-(trifluoromethyl)phenyl)-8-(hydroxymethyl)-3,4,8,9,10,11-hexahydro-1H-pyrido[4′,3′:3,4]pyrazolo[1,5-a]azepine-2(7H)-carboxamide

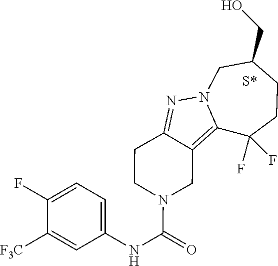 [SMILES: O=C(Nc1ccc(F)c(C(F)(F)F)c1)N1CCc2nn3c(c2C1)C(F)(F)CC[C@H](CO)C3]

The title compound was prepared in a manner analogous to Example 1, however using (S*)-tert-butyl 11,11-difluoro-8-(hydroxymethyl)-3,4,8,9,10,11-hexahydro-1H-pyrido[4′,3′:3,4]pyrazolo[1,5-a]azepine-2(7H)-carboxylate (Intermediate 8) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (4-fluoro-3-(trifluoromethyl)phenyl)carbamate instead of phenyl (3-cyano-4-fluorophenyl) carbamate in Step B. MS (ESI): mass calcd. for C₂₀H₂₀F₆N₄O₂, 462.2; m/z found, 463.1 [M+H]+. ¹H NMR (400 MHz, CDCl₃) δ=7.68-7.55 (m, 2H), 7.14 (t, J=9.3 Hz, 1H), 6.50 (s, 1H), 4.63 (s, 2H), 4.53 (br d, J=14.3 Hz, 1H), 4.21 (dd, J=8.6, 14.1 Hz, 1H), 3.81 (t, J=5.6 Hz, 2H), 3.65-3.44 (m, 2H), 2.84 (t, J=5.9 Hz, 2H), 2.50-2.20 (m, 2H), 2.13-1.99 (m, 2H), 1.94-1.80 (m, 1H).

### Example 21

### (S*)-N-(3-Cyano-2,4-difluorophenyl)-11,11-difluoro-8-(hydroxymethyl)-3,4,8,9,10,11-hexahydro-1H-pyrido[4′,3′:3,4]pyrazolo[1,5-a]azepine-2(7H)-carboxamide

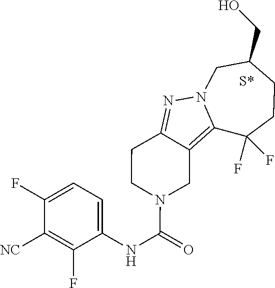 [SMILES: N#Cc1c(F)ccc(NC(=O)N2CCc3nn4c(c3C2)C(F)(F)CC[C@H](CO)C4)c1F]

The title compound was prepared in a manner analogous to Example 1, however using (S*)-tert-butyl 11,11-difluoro-8-(hydroxymethyl)-3,4,8,9,10,11-hexahydro-1H-pyrido[4′,3′:3,4]pyrazolo[1,5-a]azepine-2(7H)-carboxylate (Intermediate 8) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (3-cyano-2,4-difluorophenyl)carbamate instead of phenyl (3-cyano-4-fluorophenyl) carbamate in Step B. MS (ESI): mass calcd. for C₂₀H₁₉F₄N₅O₂, 437.2; m/z found, 438.1 [M+H]+. ¹H NMR (400 MHz, CDCl₃) δ=8.37-8.30 (m, 1H), 7.09-6.96 (m, 1H), 6.55 (d, J=2.8 Hz, 1H), 4.66 (s, 2H), 4.53 (d, J=14.2 Hz, 1H), 4.21 (dd, J=8.4, 14.3 Hz, 1H), 3.81 (t, J=5.8 Hz, 2H), 3.66-3.44 (m, 2H), 2.85 (t, J=5.8 Hz, 2H), 2.55-2.15 (m, 2H), 2.13-1.98 (m, 2H), 1.96-1.79 (m, 1H).

### Example 22

### (S*)-N-(2-Bromo-3-fluoropyridin-4-yl)-11,11-difluoro-8-(hydroxymethyl)-3,4,8,9,10,11-hexahydro-1H-pyrido[4′,3′:3,4]pyrazolo[1,5-a]azepine-2(7H)-carboxamide

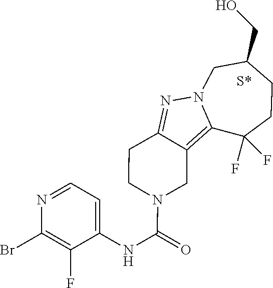 [SMILES: O=C(Nc1ccnc(Br)c1F)N1CCc2nn3c(c2C1)C(F)(F)CC[C@H](CO)C3]

The title compound was prepared in a manner analogous to Example 1, however using (S*)-tert-butyl 11,11-difluoro-8-(hydroxymethyl)-3,4,8,9,10,11-hexahydro-1H-pyrido[4′,3′:3,4]pyrazolo[1,5-a]azepine-2(7H)-carboxylate (Intermediate 8) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (2-bromo-3-fluoropyridin-4-yl)carbamate instead of phenyl (3-cyano-4-fluorophenyl) carbamate in Step B. MS (ESI): mass calcd. for C₁₈H₁₉BrF₃N₅O₂, 473.1/475.1; m/z found, 474.0/476.0 [M+H]+. ¹H NMR (400 MHz, CDCl₃) δ=8.18-8.06 (m, 1H), 8.00 (d, J=5.5 Hz, 1H), 7.02-6.81 (m, 1H), 4.60 (s, 2H), 4.46 (d, J=14.2 Hz, 1H), 4.13 (dd, J=8.5, 14.0 Hz, 1H), 3.74 (t, J=5.8 Hz, 2H), 3.53-3.42 (m, 2H), 2.78 (t, J=5.6 Hz, 2H), 2.45-2.05 (m, 2H), 2.05-1.90 (m, 2H), 1.85-1.73 (m, 1H).

### Example 23

### (R*)-N-(3-Cyano-4-fluorophenyl)-11,11-difluoro-8-(hydroxymethyl)-3,4,8,9,10,11-hexahydro-1H-pyrido[4′,3′:3,4]pyrazolo[1,5-a]azepine-2(7H)-carboxamide

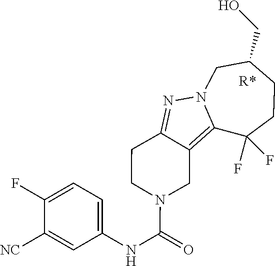 [SMILES: N#Cc1cc(NC(=O)N2CCc3nn4c(c3C2)C(F)(F)CC[C@@H](CO)C4)ccc1F]

The title compound was prepared in a manner analogous to Example 1, however using (R*)-tert-butyl 11,11-difluoro-8-(hydroxymethyl)-3,4,8,9,10,11-hexahydro-1H-pyrido[4′,3′:3,4]pyrazolo[1,5-a]azepine-2(7H)-carboxylate (Intermediate 9) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A. MS (ESI): mass calcd. for C₂₀H₂₀F₃N₅O₂, 419.2; m/z found, 420.1 [M+H]+. ¹H NMR (400 MHz, CD₃OD) δ=7.82 (dd, J=2.7, 5.6 Hz, 1H), 7.76-7.69 (m, 1H), 7.29 (t, J=8.8 Hz, 1H), 4.69 (s, 2H), 4.53 (d, J=14.2 Hz, 1H), 4.10 (dd, J=9.2, 14.4 Hz, 1H), 3.82 (t, J=5.9 Hz, 2H), 3.56-3.49 (m, 1H), 3.44-3.37 (m, 1H), 2.80 (t, J=5.6 Hz, 2H), 2.55-2.40 (m, 1H), 2.37-2.17 (m, 1H), 2.03-1.80 (m, 3H).

### Example 24

### (R*)-11,11-Difluoro-N-(4-fluoro-3-(trifluoromethyl)phenyl)-8-(hydroxymethyl)-3,4,8,9,10,11-hexahydro-1H-pyrido[4′,3′:3,4]pyrazolo[1,5-a]azepine-2(7H)-carboxamide

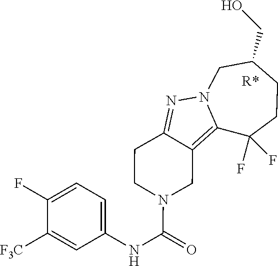 [SMILES: O=C(Nc1ccc(F)c(C(F)(F)F)c1)N1CCc2nn3c(c2C1)C(F)(F)CC[C@@H](CO)C3]

The title compound was prepared in a manner analogous to Example 1, however using (R*)-tert-butyl 11,11-difluoro-8-(hydroxymethyl)-3,4,8,9,10,11-hexahydro-1H-pyrido[4′,3′:3,4]pyrazolo[1,5-a]azepine-2(7H)-carboxylate (Intermediate 9) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (4-fluoro-3-(trifluoromethyl)phenyl)carbamate instead of phenyl (3-cyano-4-fluorophenyl) carbamate in Step B. MS (ESI): mass calcd. for C₂₀H₂₀F₆N₄O₂, 462.2; m/z found, 463.1 [M+H]+. ¹H NMR (400 MHz, CD₃OD) δ=7.79 (dd, J=2.8, 6.4 Hz, 1H), 7.71-7.63 (m,1H), 7.25 (t, J=9.5 Hz, 1H), 4.69(s, 2H), 4.53(d, J=14.3 Hz, 1H), 4.11(dd, J=9.2, 14.2 Hz, 1H), 3.86-3.79 (m, 2H), 3.55-3.48 (m, 1H), 3.45-3.38 (m, 1H), 2.80 (t, J=5.8 Hz, 2H), 2.58-2.41 (m, 1H), 2.39-2.21(m, 1H), 2.05-1.91 (m, 2H), 1.89-1.79(m, 1H).

### Example 25

### (R*)-N-(3-Cyano-2,4-difluorophenyl)-11,11-difluoro-8-(hydroxymethyl)-3,4,8,9,10,11-hexahydro-1H-pyrido[4′,3′:3,4]pyrazolo[1,5-a]azepine-2(7H)-carboxamide

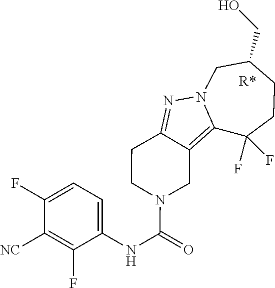 [SMILES: N#Cc1c(F)ccc(NC(=O)N2CCc3nn4c(c3C2)C(F)(F)CC[C@@H](CO)C4)c1F]

The title compound was prepared in a manner analogous to Example 1, however using (R*)-tert-butyl 11,11-difluoro-8-(hydroxymethyl)-3,4,8,9,10,11-hexahydro-1H-pyrido[4′,3′:3,4]pyrazolo[1,5-a]azepine-2(7H)-carboxylate (Intermediate 9) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (3-cyano-2,4-difluorophenyl)carbamate instead of phenyl (3-cyano-4-fluorophenyl) carbamate in Step B. MS (ESI): mass calcd. for C₂₀H₁₉F₄N₅O₂, 437.2; m/z found, 438.1 [M+H]+. ¹H NMR (400 MHz, CDCl₃) δ=8.35 (dt, J=5.8, 9.1 Hz, 1H), 7.05-7.00 (m, 1H), 6.57 (br d, J=2.9 Hz, 1H), 4.67 (s, 2H), 4.55 (d, J=14.3 Hz, 1H), 4.22 (dd, J=8.6, 14.3 Hz, 1H), 3.83 (t, J=5.8 Hz, 2H), 3.68-3.57 (m, 1H), 3.55-3.46 (m, 1H), 2.87 (t, J=5.9 Hz, 2H), 2.60-2.41 (m, 1H), 2.32-2.18 (m, 1H), 2.13-1.99 (m, 2H), 1.96-1.81 (m, 1H).

### Example 26

### (R*)-N-(2-Bromo-3-fluoropyridin-4-yl)-11,11-difluoro-8-(hydroxymethyl)-3,4,8,9,10,11-hexahydro-1H-pyrido[4′,3′:3,4]pyrazolo[1,5-a]azepine-2(7H)-carboxamide

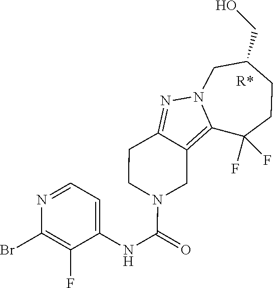 [SMILES: O=C(Nc1ccnc(Br)c1F)N1CCc2nn3c(c2C1)C(F)(F)CC[C@@H](CO)C3]

The title compound was prepared in a manner analogous to Example 1, however using (R*)-tert-butyl 11,11-difluoro-8-(hydroxymethyl)-3,4,8,9,10,11-hexahydro-1H-pyrido[4′,3′:3,4]pyrazolo[1,5-a]azepine-2(7H)-carboxylate (Intermediate 9) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (2-bromo-3-fluoropyridin-4-yl)carbamate instead of phenyl (3-cyano-4-fluorophenyl) carbamate in Step B. MS (ESI): mass calcd. for C₁₈H₁₉BrF₃N₅O₂, 473.1/475.1; m/z found, 474.0/476.0 [M+H]+. ¹H NMR (400 MHz, CDCl₃) δ=8.18 (t, J=5.5 Hz, 1H), 8.09 (d, J=5.5 Hz, 1H), 6.97 (d, J=3.6 Hz, 1H), 4.69 (s, 2H), 4.55 (d, J=14.4 Hz, 1H), 4.22 (dd, J=8.5, 14.2 Hz, 1H), 3.83 (t, J=5.8 Hz, 2H), 3.66-3.57 (m, 1H), 3.55-3.47 (m, 1H), 2.87 (t, J=5.8 Hz, 2H), 2.50-2.43 (m, 1H), 2.34-2.18 (m, 1H), 2.13-2.01 (m, 2H), 1.95-1.81 (m, 1H).

### Example 27

### (S*)-N-(3-Cyano-4-fluorophenyl)-84(2,2-difluoroethoxy)methyl)-11,11-difluoro-3,4,8,9,10,11-hexahydro-1H-pyrido[4′,3′:3,4]pyrazolo[1,5-a]azepine-2(7H)-carboxamide

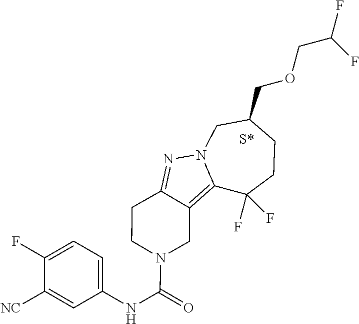 [SMILES: N#Cc1cc(NC(=O)N2CCc3nn4c(c3C2)C(F)(F)CC[C@H](COCC(F)F)C4)ccc1F]

The title compound was prepared in a manner analogous to Example 1, however using (S*)-tert-butyl 8-((2,2-difluoroethoxy)methyl)-11,11-difluoro-3,4,8,9,10,11-hexahydro-1H-pyrido[4′,3′:3,4]pyrazolo[1,5-a]azepine-2(7H)-carboxylate (Intermediate 10) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A. MS (ESI): mass calcd. for C₂₂H₂₂F₅N₅O₂, 483.2; m/z found, 484.1 [M+H]+. ¹H NMR (400 MHz, CDCl₃) δ=7.73 (dd, J=2.8, 5.4 Hz, 1H), 7.62-7.59 (m, 1H), 7.15 (t, J=8.7 Hz, 1H), 6.51 (s, 1H), 6.12-5.61 (m, 1H), 4.63 (s, 2H), 4.50 (br d, J=14.4 Hz, 1H), 4.14 (dd, J=9.1, 14.2 Hz, 1H), 3.80 (br t, J=5.7 Hz, 2H), 3.66 (dt, J=4.1, 13.9 Hz, 2H), 3.53-3.38 (m, 2H), 2.83 (t, J=5.7 Hz, 2H), 2.48-1.88 (m, 5H).

### Example 28

### (S*)-8-((2,2-Difluoroethoxy)methyl)-11,11-difluoro-N-(4-fluoro-3-(trifluoromethyl)phenyl)-3,4,8,9,10,11-hexahydro-1H-pyrido[4′,3′:3,4]pyrazolo[1,5-a]azepine-2(7H)-carboxamide

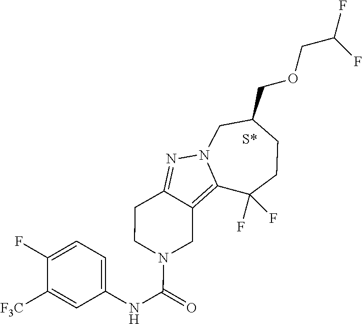 [SMILES: O=C(Nc1ccc(F)c(C(F)(F)F)c1)N1CCc2nn3c(c2C1)C(F)(F)CC[C@H](COCC(F)F)C3]

The title compound was prepared in a manner analogous to Example 1, however using (S*)-tert-butyl 8-((2,2-difluoroethoxy)methyl)-11,11-difluoro-3,4,8,9,10,11-hexahydro-1H-pyrido[4′,3′:3,4]pyrazolo[1,5-a]azepine-2(7H)-carboxylate (Intermediate 10) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (4-fluoro-3-(trifluoromethyl)phenyl)carbamate instead of phenyl (3-cyano-4-fluorophenyl) carbamate in Step B. MS (ESI): mass calcd. for C₂₂H₂₂F₈N₄O₂, 526.2; m/z found, 527.1 [M+H]+. ¹H NMR (400 MHz, CDCl₃) δ=7.67-7.55 (m, 2H), 7.14 (t, J=9.3 Hz, 1H), 6.49 (s, 1H), 6.08-5.64 (m, 1H), 4.63 (s, 2H), 4.50 (d, J=1 4.3 Hz, 1H), 4.14 (dd, J=9.0, 14.3 Hz, 1H), 3.81 (t, J=5.9 Hz, 2H), 3.66 (td, J=4.0, 13.9 Hz, 2H), 3.52-3.38 (m, 2H), 2.83 (t, J=5.8 Hz, 2H), 2.59-1.80 (m, 5H).

### Example 29

### (R*)-N-(3-Cyano-4-fluorophenyl)-8-((2,2-difluoroethoxy)methyl)-11,11-difluoro-3,4,8,9,10,11-hexahydro-1H-pyrido[4′,3′:3,4]pyrazolo[1,5-a]azepine-2(7H)-carboxamide

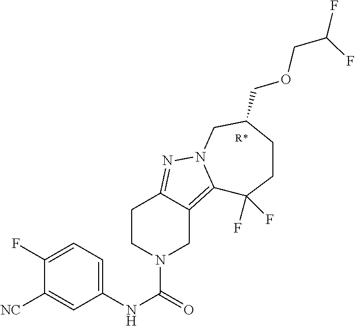 [SMILES: N#Cc1cc(NC(=O)N2CCc3nn4c(c3C2)C(F)(F)CC[C@@H](COCC(F)F)C4)ccc1F]

The title compound was prepared in a manner analogous to Example 1, however using (R*)-tert-butyl 8-((2,2-difluoroethoxy)methyl)-11,11-difluoro-3,4,8,9,10,11-hexahydro-1H-pyrido[4′,3′:3,4]pyrazolo[1,5-a]azepine-2(7H)-carboxylate (Intermediate 11) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A. MS (ESI): mass calcd. for C₂₂H₂₂F₅N₅O₂, 483.2; m/z found, 484.1 [M+H]+. ¹H NMR (400 MHz, CD₃OD) δ=7.82 (dd, J=2.7, 5.6 Hz, 1H), 7.75-7.70 (m, 1H), 7.29 (t, J=9.0 Hz, 1H), 6.13-5.80 (m, 1H), 4.69 (s, 2H), 4.50 (br d, J=13.7 Hz, 1H), 4.16 (dd, J=9.0, 14.2 Hz, 1H), 3.82 (t, J=5.9 Hz, 2H), 3.68 (dt, J=4.0, 14.4 Hz, 2H), 3.56-3.49 (m, 1H), 3.45-3.3 (m, 1H), 2.80 (t, J=5.9 Hz, 2H), 2.6-1.8 (m, 5H).

### Example 30

### (R*)-8-((2,2-Difluoroethoxy)methyl)-11,11-difluoro-N-(4-fluoro-3-(trifluoromethyl)phenyl)-3,4,8,9,10,11-hexahydro-1H-pyrido[4′,3′:3,4]pyrazolo[1,5-a]azepine-2(7H)-carboxamide

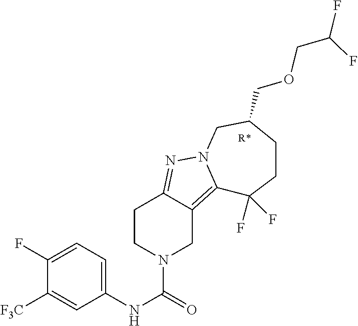 [SMILES: O=C(Nc1ccc(F)c(C(F)(F)F)c1)N1CCc2nn3c(c2C1)C(F)(F)CC[C@@H](COCC(F)F)C3]

The title compound was prepared in a manner analogous to Example 1, however using (R*)-tert-butyl 8-((2,2-difluoroethoxy)methyl)-11,11-difluoro-3,4,8,9,10,11-hexahydro-1H-pyrido[4′,3′:3,4]pyrazolo[1,5-a]azepine-2(7H)-carboxylate (Intermediate 11) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (4-fluoro-3-(trifluoromethyl)phenyl)carbamate instead of phenyl (3-cyano-4-fluorophenyl) carbamate in Step B. MS (ESI): mass calcd. for C₂₂H₂₂F₈N₄O₂, 526.2; m/z found, 527.1 [M+H]+. ¹H NMR (400 MHz, CD₃OD) δ=7.79 (dd, J=2.7, 6.4 Hz, 1H), 7.70-7.63 (m, 1H), 7.25 (t, J=9.7Hz, 1H), 6.13-5.81 (m, 1H), 4.69 (s, 2H), 4.50 (d, J=14.2 Hz, 1H), 4.16 (dd, J=9.0, 14.2 Hz, 1H), 3.82 (t, J=5.9 Hz, 2H), 3.68 (dt, J=3.9, 14.4 Hz, 2H), 3.56-3.49 (m, 1H), 3.46-3.40 (m, 1H), 2.80 (t, J=5.8 Hz, 2H), 2.6-1.75 (m, 5H).

### Example 31

### (R*)-N-(3-Cyano-4-fluorophenyl)-8-((2,2-difluoroethoxy)methyl)-11,11-difluoro-8-hydroxy-3,4,8,9,10,11-hexahydro-1H-pyrido[4′,3′:3,4]pyrazolo[1,5-a]azepine-2(7H)-carboxamide

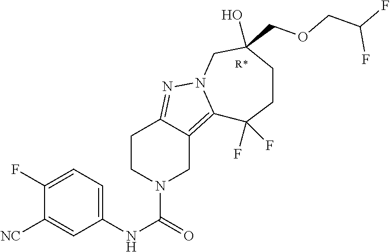 [SMILES: N#Cc1cc(NC(=O)N2CCc3nn4c(c3C2)C(F)(F)CC[C@](O)(COCC(F)F)C4)ccc1F]

The title compound was prepared in a manner analogous to Example 1, however using (R*)-tert-butyl 8-((2,2-difluoroethoxy)methyl)-11,11-difluoro-8-hydroxy-3,4,8,9,10,11-hexahydro-1H-pyrido[4′,3′:3,4]pyrazolo[1,5-a]azepine-2(7H)-carboxylate (Intermediate 12) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A. MS (ESI): mass calcd. for C₂₂H₂₂N₅F₅O₃, 499.2; m/z found, 500.1 [M+H]+. ¹H NMR (400 MHz, DMSO-d₆) δ=9.04 (s, 1H), 7.94 (dd, J=2.8, 5.8 Hz, 1H), 7.80-7.78 (m, 1H), 7.44 (t, J=9.2 Hz, 1H), 6.32-6.00 (m, 1H), 5.10 (s, 1H), 4.59 (br s, 2H), 4.29-4.15 (m, 2H), 3.82-3.70 (m, 4H), 3.50-3.42 (m, 2H), 2.70-2.67 (m, 2H), 2.44-2.23 (m, 2H), 2.05-1.98 (m, 1H), 1.80-1.70 (m, 1H).

### Example 32

### (R*)-N-(2-Bromo-3-fluoropyridin-4-yl)-8-((2,2-difluoroethoxy)methyl)-11,11-difluoro-8-hydroxy-3,4,8,9,10,11-hexahydro-1H-pyrido[4′,3′:3,4]pyrazolo[1,5-a]azepine-2(7H)-carboxamide

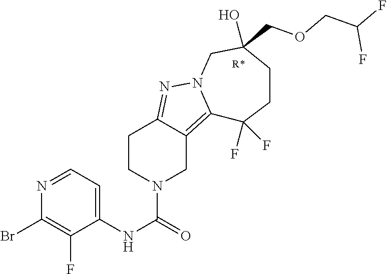 [SMILES: O=C(Nc1ccnc(Br)c1F)N1CCc2nn3c(c2C1)C(F)(F)CC[C@](O)(COCC(F)F)C3]

The title compound was prepared in a manner analogous to Example 1, however using (R*)-tert-butyl 8-((2,2-difluoroethoxy)methyl)-11,11-difluoro-8-hydroxy-3,4,8,9,10,11-hexahydro-1H-pyrido[4′,3′:3,4]pyrazolo[1,5-a]azepine-2(7H)-carboxylate (Intermediate 12) instead of tert-butyl 11,11-difluoro-8-methylene-3 ,4,8,9,10,11-hexahydro-1H-pyrido[4′,3′:3,4]pyrazolo[1,5-a]azepine-2(7H)-carboxylate (Intermediate 1) in Step A and phenyl (2-bromo-3-fluoropyridin-4-yl)carbamate instead of phenyl (3-cyano-4-fluorophenyl)carbamate in Step B. MS (ESI): mass calcd. for C₂₀H₂₁N₅F₅O₃Br, 553.1/555.1; m/z found, 554.0/556.0 [M+H]+. ¹H NMR (400 MHz, DMSO-d₆) δ=9.23 (br s, 1H), 8.05 (d, J=5.4 Hz, 1H), 7.69 (t, J=5.6 Hz, 1H), 6.34-5.98 (m, 1H), 5.11 (br s, 1H), 4.60 (br s, 2H), 4.30-4.13 (m, 2H), 3.84-3.68 (m, 4H), 3.49-3.43 (m, 2H), 2.74-2.66 (m, 2H), 2.41-2.24 (m, 2H), 2.07-1.97 (m, 1H), 1.85-1.65 (m, 1H).

### Example 33

### (R*)-8-((2,2-Difluoroethoxy)methyl)-N-(2-(difluoromethyl)-3-fluoropyridin-4-yl)-11,11-difluoro-8-hydroxy-3,4,8,9,10,11-hexahydro-1H-pyrido[4′,3′:3,4]pyrazolo[1,5-a]azepine-2 (7H)-carboxamide

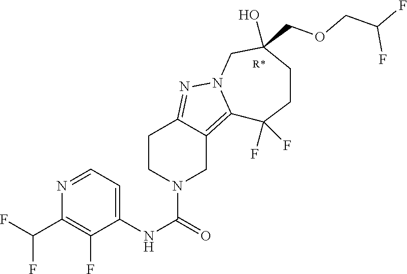 [SMILES: O=C(Nc1ccnc(C(F)F)c1F)N1CCc2nn3c(c2C1)C(F)(F)CC[C@](O)(COCC(F)F)C3]

The title compound was prepared in a manner analogous to Example 1, however using (R*)-tert-butyl 8-((2,2-difluoroethoxy)methyl)-11,11-difluoro-8-hydroxy-3,4,8,9,10,11-hexahydro-1H-pyrido[4′,3′:3,4]pyrazolo[1,5-a]azepine-2(7H)-carboxylate (Intermediate 12) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (2-(difluoromethyl)-3-fluoropyridin-4-yl) carbamate instead of phenyl (3-cyano-4-fluorophenyl) carbamate in Step B. MS (ESI): mass calcd. for C₂₁H₂₂F₇N₅O₃, 525.2; m/z found, 526.1 [M+H]+. ¹H NMR (400 MHz, DMSO-d₆) δ=9.23 (s, 1H), 8.31 (d, J=5.4 Hz, 1H), 7.87 (t, J=5.7 Hz, 1H), 7.10 (t, J=53.6 Hz, 1H), 6.34-5.99 (m, 1H), 5.10 (s, 1H), 4.62 (s, 2H), 4.30-4.13 (m, 2H), 3.83-3.70 (m, 4H), 3.51-3.43 (m, 2H), 2.75-2.69 (m, 2H), 2.37-2.26 (m, 2H), 2.05-1.98 (m, 1H), 1.83-1.78 (m, 1H).

### Example 34

### (S*)-N-(3-Cyano-4-fluorophenyl)-8-((2,2-difluoroethoxy)methyl)-11,11-difluoro-8-hydroxy-3,4,8,9,10,11-hexahydro-1H-pyrido[4′,3′:3,4]pyrazolo[1,5-a]azepine-2(7H)-carboxamide

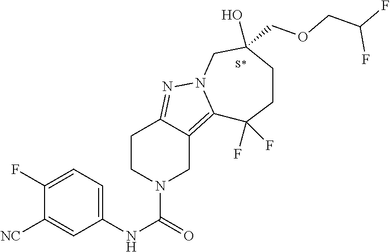 [SMILES: N#Cc1cc(NC(=O)N2CCc3nn4c(c3C2)C(F)(F)CC[C@@](O)(COCC(F)F)C4)ccc1F]

The title compound was prepared in a manner analogous to Example 1, however using (S*)-tert-butyl 8-((2,2-difluoroethoxy)methyl)-11,11-difluoro-8-hydroxy-3,4,8,9,10,11-hexahydro-1H-pyrido[4′,3′:3,4]pyrazolo[1,5-a]azepine-2(7H)-carboxylate (Intermediate 13) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A. MS (ESI): mass calcd. for C₂₂H₂₂N₅F₅O₃, 499.2; m/z found, 500.1 [M+H]+. ¹H NMR (400 MHz, CDCl₃) δ=7.64 (dd, J=2.7, 5.4 Hz, 1H), 7.55-7.52 (m, 1H), 7.07 (t, J=8.7 Hz, 1H), 6.45 (s, 1H), 6.03-5.55 (m, 1H), 4.68-4.49 (m, 2H), 4.43 (d, J=14.2 Hz, 1H), 4.17 (d, J=14.5 Hz, 1H), 3.75-3.62 (m, 4H), 3.51-3.36 (m, 2H), 2.76 (t, J=5.8 Hz, 2H), 2.60-2.38 (m, 2H), 2.30-2.17 (m, 1H), 2.02-1.83 (m, 2H).

### Example 35

### (S*)-N-(2-Bromo-3-fluoropyridin-4-yl)-8-((2,2-difluoroethoxy)methyl)-11,11-difluoro-8-hydroxy-3,4,8,9,10,11-hexahydro-1H-pyrido[4′,3′:3,4]pyrazolo[1,5-a]azepine-2(7H)-carboxamide

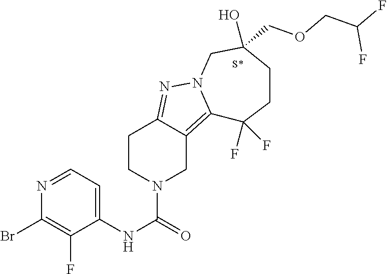 [SMILES: O=C(Nc1ccnc(Br)c1F)N1CCc2nn3c(c2C1)C(F)(F)CC[C@@](O)(COCC(F)F)C3]

The title compound was prepared in a manner analogous to Example 1, however using (S*)-tert-butyl 8-((2,2-difluoroethoxy)methyl)-11,11-difluoro-8-hydroxy-3,4,8,9,10,11-hexahydro-1H-pyrido[4′,3′:3,4]pyrazolo[1,5-a]azepine-2(7H)-carboxylate (Intermediate 13) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (2-bromo-3-fluoropyridin-4-yl)carbamate instead of phenyl (3-cyano-4-fluorophenyl) carbamate in Step B. MS (ESI): mass calcd. for C₂₀H₂₁N₅F₅O₃Br, 553.1/555.1; m/z found, 554.0/556.0 [M+H]+. ¹H NMR (400 MHz, CDCl₃) δ=8.09 (t, J=5.5 Hz, 1H), 8.00 (d, J=5.5 Hz, 1H), 6.86 (br d, J=3.8 Hz, 1H), 6.12-5.61 (m, 1H), 4.67-4.53 (m, 2H), 4.43 (d, J=14.3 Hz, 1H), 4.18 (d, J=14.4 Hz, 1H), 3.83-3.60 (m, 4H), 3.49-3.33 (m, 2H), 2.79 (t, J=5.8 Hz, 2H), 2.58-2.18 (m, 3H), 2.05-1.83 (m, 2H).

### Example 36

### (S*)-8-((2,2-Difluoroethoxy)methyl)-N-(2-(difluoromethyl)-3-fluoropyridin-4-yl)-11,11-difluoro-8-hydroxy-3,4,8,9,10,11-hexahydro-1H-pyrido[4′,3′:3,4]pyrazolo[1,5-a]azepine-2 (7H)-carboxamide

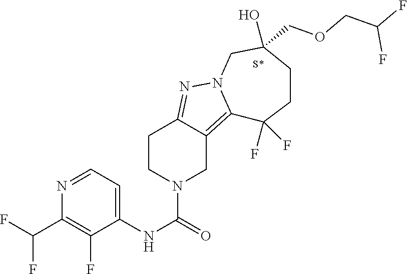 [SMILES: O=C(Nc1ccnc(C(F)F)c1F)N1CCc2nn3c(c2C1)C(F)(F)CC[C@@](O)(COCC(F)F)C3]

The title compound was prepared in a manner analogous to Example 1, however using (S*)-tert-butyl 8-((2,2-difluoroethoxy)methyl)-11,11-difluoro-8-hydroxy-3,4,8,9,10,11-hexahydro-1H-pyrido[4′,3′:3,4]pyrazolo[1,5-a]azepine-2(7H)-carboxylate (Intermediate 13) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (2-(difluoromethyl)-3-fluoropyridin-4-yl) carbamate instead of phenyl (3-cyano-4-fluorophenyl) carbamate in Step B. MS (ESI): mass calcd. for C₂₁H₂₂F₇N₅O₃, 525.2; m/z found, 526.1 [M+H]+. ¹H NMR (400 MHz, CDCl₃) δ=8.45-8.25 (m, 2H), 7.09-7.00 (m, 1H), 6.75 (t, J=53.6 Hz, 1H), 6.18-5.70 (m, 1H), 4.84-4.61 (m, 2H), 4.53 (d, J=14.8 Hz, 1H), 4.27 (d, J=14.4 Hz, 1H), 3.94-3.69 (m, 4H), 3.56-3.40 (m, 2H), 2.89 (t, J=5.7 Hz, 2H), 2.74-2.43 (m, 2H), 2.40-2.24 (m, 1H), 2.05-1.92 (m, 2H).

### Example 37

### N-(3-Cyano-4-fluorophenyl)-11,11-difluoro-8-(fluoromethyl)-8-hydroxy-3,4,8,9,10,11-hexahydro-1H-pyrido[4′,3′:3,4]pyrazolo[1,5-a]azepine-2(7H)-carboxamide

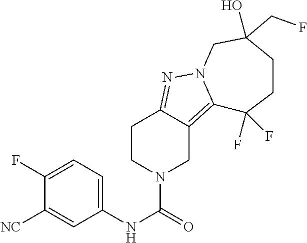 [SMILES: N#Cc1cc(NC(=O)N2CCc3nn4c(c3C2)C(F)(F)CCC(O)(CF)C4)ccc1F]

The title compound was prepared in a manner analogous to Example 1, however using tert-butyl 11,11-difluoro-8-(fluoromethyl)-8-hydroxy-3,4,8,9,10,11-hexahydro-1H-pyrido[4′,3′:3,4]pyrazolo[1,5-a]azepine-2(7H)-carboxylate (Intermediate 14) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A. MS (ESI): mass calcd. for C₂₀H₁₉F₄N₅O₂, 437.2; m/z found, 438.1 [M+H]+. ¹H NMR (400 MHz, CDCl₃) δ=7.72 (dd, J=2.8, 5.4 Hz, 1H), 7.65-7.58 (m, 1H), 7.15 (t, J=8.7 Hz, 1H), 6.49 (s, 1H), 4.72-4.18 (m, 6H), 3.86-3.75 (m, 2H), 2.85 (t, J=5.8 Hz, 2H), 2.68-2.15 (m, 3H), 2.12-1.86 (m, 2H).

### Example 38

### (S*)-N-(2-Bromo-3-fluoropyridin-4-yl)-11,11-di fluoro-8-(fluoromethyl) -8-hydroxy-3,4,8,9,10,11-hexahydro-1H-pyrido[4′,3′:3,4]pyrazolo[1,5-a]azepine-2(7H)-carboxamide

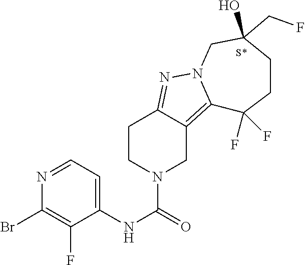 [SMILES: O=C(Nc1ccnc(Br)c1F)N1CCc2nn3c(c2C1)C(F)(F)CC[C@@](O)(CF)C3]

The title compound was prepared in a manner analogous to Example 1, however using (S*)-tert-butyl 11,11-difluoro-8-(fluoromethyl)-8-hydroxy-3,4,8,9,10,11-hexahydro-1H-pyrido[4′,3′:3,4]pyrazolo[1,5-a]azepine-2(7H)-carboxylate (Intermediate 15) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (2-bromo-3-fluoropyridin-4-yl)carbamate instead of phenyl (3-cyano-4-fluorophenyl) carbamate in Step B. MS (ESI): mass calcd. for C₁₈H₁₈BrF₄N₅O₂, Exact Mass: 491.1/493.1; m/z found, 492.1/494.1 [M+H]+. ¹H NMR (400 MHz, CDCl₃) δ=8.20-8.15 (m, 1H), 8.08 (d, J=5.5 Hz, 1H), 6.94 (br d, J=3.4 Hz, 1H), 4.75-4.62 (m, 2H), 4.51 (d, J=14.5 Hz, 1H), 4.42-4.21 (m, 3H), 3.91-3.75 (m, 2H), 2.87 (t, J=5.7 Hz, 2H), 2.67-2.47 (m, 1H), 2.44-2.27 (m, 1H), 2.10-1.88 (m, 2H).

### Example 39

### (R*)-N-(2-Bromo-3-fluoropyridin-4-yl)-11,11-difluoro-8-(fluoromethyl) -8-hydroxy-3,4,8,9,10,11-hexahydro-1H-pyrido[4′,3′:3,4]pyrazolo[1,5-a]azepine-2(7H)-carboxamide

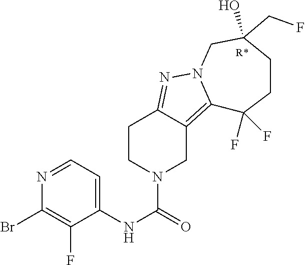 [SMILES: O=C(Nc1ccnc(Br)c1F)N1CCc2nn3c(c2C1)C(F)(F)CC[C@](O)(CF)C3]

The title compound was prepared in a manner analogous to Example 1, however using (R*)-tert-butyl 11,11-difluoro-8-(fluoromethyl)-8-hydroxy-3,4,8,9,10,11-hexahydro-1H-pyrido[4′,3′:3,4]pyrazolo[1,5-a]azepine-2(7H)-carboxylate (Intermediate 16) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (2-bromo-3-fluoropyridin-4-yl)carbamate instead of phenyl (3-cyano-4-fluorophenyl) carbamate in Step B. MS (ESI): mass calcd. for C₁₈H₁₈BrF₄N₅O₂, Exact Mass: 491.2/493.2; m/z found, 492.0/494.1 [M+H]+. ¹H NMR (400 MHz, CDCl₃) δ=8.18 (t, J=5.5 Hz, 1H), 8.09 (d, J=5.5 Hz, 1H), 6.95 (br d, J=3.7 Hz, 1H), 4.70 (s, 2H), 4.52 (d, J=14.7 Hz, 1H), 4.43-4.22 (m, 3H), 3.91-3.77 (m, 2H), 2.88 (t, J=5.8 Hz, 2H), 2.69-2.48 (m, 1H), 2.45-2.19 (m, 1H), 2.12-1.90 (m, 2H).

### Example 40

### N-(3-Cyano-4-fluorophenyl)-8-(cyanomethyl)-11,11-difluoro-8-hydroxy-3,4,8,9,10,11-hexahydro-1H-pyrido[4′,3′:3,4]pyrazolo[1,5-a]azepine-2(7H)-carboxamide

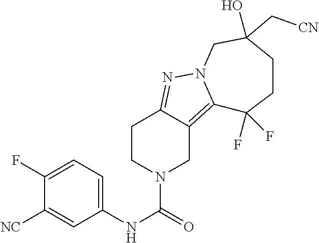 [SMILES: N#CCC1(O)CCC(F)(F)c2c3c(nn2C1)CCN(C(=O)Nc1ccc(F)c(C#N)c1)C3]

The title compound was prepared in a manner analogous to Example 1, however using tert-butyl 8-(cyanomethyl)-11,11-difluoro-8-hydroxy-3,4,8,9,10,11-hexahydro-1H-pyrido[4′,3′:3,4]pyrazolo[1,5-a]azepine-2(7H)-carboxylate (Intermediate 17) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A. MS (ESI): mass calcd. for C₂₁H₁₉F₃N₆O₂, 444.2; m/z found, 445.2 [M+H]+. ¹H NMR (400 MHz, CDCl₃) δ=7.71 (dd, J=2.8, 5.4 Hz, 1H), 7.60-7.58 (m, 1H), 7.15 (t, J=8.7 Hz, 1H), 6.50 (s, 1H), 4.64 (s, 2H), 4.51-4.35 (m, 2H), 3.86-3.73 (m, 2H), 2.83 (t, J=5.7 Hz, 2H), 2.72-2.46 (m, 3H), 2.43-2.11 (m, 4H).

### Example 41

### (R*)-N-(2-Bromo-3-fluoropyridin-4-yl)-8-(cyanomethyl)-11,11-difluoro-8-hydroxy-3,4,8,9,10,11-hexahydro-1H-pyrido[4′,3′:3,4]pyrazolo[1,5-a]azepine-2(7H)-carboxamide

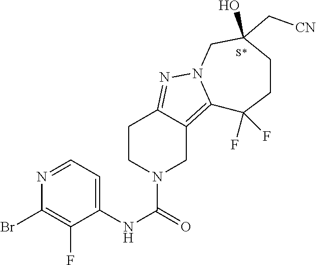 [SMILES: N#CC[C@]1(O)CCC(F)(F)c2c3c(nn2C1)CCN(C(=O)Nc1ccnc(Br)c1F)C3]

The title compound was prepared in a manner analogous to Example 1, however using (R*)-tert-butyl 8-(cyanomethyl)-11,11-difluoro-8-hydroxy-3,4,8,9,10,11-hexahydro-1H-pyrido[4′,3′:3,4]pyrazolo[1,5-a]azepine-2(7H)-carboxylate (Intermediate 18) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (2-bromo-3-fluoropyridin-4-yl)carbamate instead of phenyl (3-cyano-4-fluorophenyl) carbamate in Step B. MS(ESI): mass calcd. for C₁₉H₁₈BrF₃N₆O₂, 498.1/500.1 ; m/z found, 499.0/501.1 [M+H]+. ¹H NMR (400 MHz, CDCl₃) δ=8.23-8.13 (m, 1H), 8.08 (d, J=5.5 Hz, 1H), 6.93 (br s, 1H), 4.68 (s, 2H), 4.51-4.36 (m, 2H), 3.88-3.76 (m, 2H), 2.86 (br t, J=5.9 Hz, 2H), 2.71-2.51 (m, 3H), 2.40-2.26 (m, 2H), 2.24-2.15 (m, 2H).

### Example 42

### (R*)-8-(Cyanomethyl)-N-(2-(difluoromethyl)-3-fluoropyridin-4-yl)-11,11-difluoro-8-hydroxy-3,4,8,9,10,11-hexahydro-1H-pyrido[4′,3′:3,4]pyrazolo[1,5-a]azepine-2(7H)-carboxamide

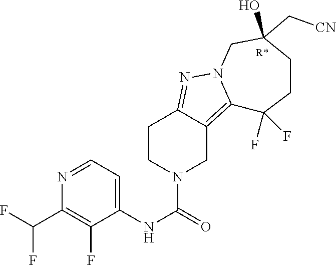 [SMILES: N#CC[C@]1(O)CCC(F)(F)c2c3c(nn2C1)CCN(C(=O)Nc1ccnc(C(F)F)c1F)C3]

The title compound was prepared in a manner analogous to Example 1, however using (R*)-tert-butyl 8-(cyanomethyl)-11,11-difluoro-8-hydroxy-3,4,8,9,10,11-hexahydro-1H-pyrido[4′,3′:3,4]pyrazolo[1,5-a]azepine-2(7H)-carboxylate(Intermediate 18) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate(Intermediate 1) in Step A and phenyl (2-(difluoromethyl)-3-fluoropyridin-4-yl) carbamate instead of phenyl (3-cyano-4-fluorophenyl) carbamate in Step B. MS (ESI): mass calcd. for C₂₀H₁₉F₅N₆O₂, 470.2; m/z found, 471.2 [M+H]+. ¹H NMR (400 MHz, CDCl₃) δ=8.33-8.30 (m, 2H), 7.01 (br d, J=4.0 Hz, 1H), 6.75 (t, J=53.6 Hz, 1H), 4.69 (s, 2H), 4.54-4.36 (m, 2H), 3.94-3.68 (m, 2H), 2.86 (br t, J=5.7 Hz, 2H), 2.72-2.48 (m, 3H), 2.40-2.15 (m, 4H).

### Example 43

### (S*)-N-(2-Bromo-3-fluoropyridin-4-yl)-8-(cyanomethyl)-11,11-difluoro-8-hydroxy-3,4,8,9,10,11-hexahydro-1H-pyrido[4′,3′:3,4]pyrazolo[1,5-a]azepine-2(7H)-carboxamide

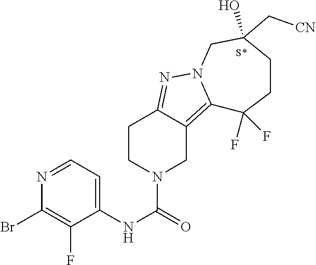 [SMILES: N#CC[C@@]1(O)CCC(F)(F)c2c3c(nn2C1)CCN(C(=O)Nc1ccnc(Br)c1F)C3]

The title compound was prepared in a manner analogous to Example 1, however using (S*)-tert-butyl 8-(cyanomethyl)-11,11-difluoro-8-hydroxy-3,4,8,9,10,11-hexahydro-1H-pyrido[4′,3′:3,4]pyrazolo[1,5-a]azepine-2(7H)-carboxylate (Intermediate 19) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (2-bromo-3-fluoropyridin-4-yl)carbamate instead of phenyl (3-cyano-4-fluorophenyl) carbamate in Step B. MS (ESI): mass calcd. for C₁₉H₁₈BrF₃N₆O₂, 498.1/500.1; m/z found, 499.0/501.1 [M+H]+. ¹H NMR (400 MHz, CDCl₃) δ=8.21-8.14 (m, 1H), 8.08 (d, J=5.6 Hz, 1H), 6.93 (br d, J=3.9 Hz, 1H), 4.68 (s, 2H), 4.51-4.37 (m, 2H), 3.82-3.75 (m, 2H), 2.86 (t, J=5.7 Hz, 2H), 2.69-2.50 (m, 3H), 2.35-2.27 (m, 2H), 2.26-2.15 (m, 2H).

### Example 44

### (S*)-8-(Cyanomethyl)-N-(2-(difluoromethyl)-3-fluoropyridin-4-yl)-11,11-difluoro-8-hydroxy-3,4,8,9,10,11-hexahydro-1H-pyrido[4′,3′:3,4]pyrazolo[1,5-a]azepine-2(7H)-carboxamide

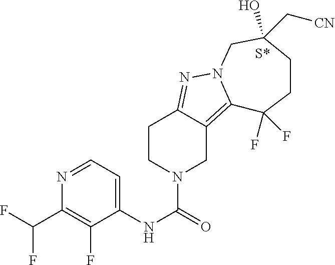 [SMILES: N#CC[C@@]1(O)CCC(F)(F)c2c3c(nn2C1)CCN(C(=O)Nc1ccnc(C(F)F)c1F)C3]

The title compound was prepared in a manner analogous to Example 1, however using (S*)-tert-butyl 8-(cyanomethyl)-11,11-difluoro-8-hydroxy-3,4,8,9,10,11-hexahydro-1H-pyrido[4′,3′:3,4]pyrazolo[1,5-a]azepine-2(7H)-carboxylate (Intermediate 19) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (2-(difluoromethyl)-3-fluoropyridin-4-yl) carbamate instead of phenyl (3-cyano-4-fluorophenyl) carbamate in Step B. MS (ESI): mass calcd. for C₂₀H₁₉F₅N₆O₂, 470.2; m/z found, 471.2 [M+H]+. ¹H NMR (400 MHz, CDCl₃) δ=8.32-8.30 (m, 2H), 7.03 (d, J=4.0 Hz, 1H), 6.75 (t, J=53.6 Hz, 1H), 4.70 (s, 2H), 4.56-4.36 (m, 2H), 3.92-3.75 (m, 2H), 2.88 (br t, J=5.3 Hz, 2H), 2.71-2.50 (m, 3H), 2.49-2.29 (m, 2H), 2.28-2.17 (m, 2H).

### Example 45

### N-(3-Cyano-4-fluorophenyl)-8,11,11-trifluoro-8-(hydroxymethyl)-3,4,8,9,10,11-hexahydro-1H-pyrido[4′,3′:3,4]pyrazolo[1,5-a]azepine-2(7H)-carboxamide

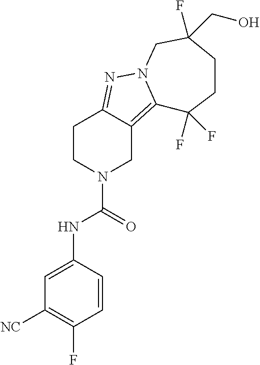 [SMILES: N#Cc1cc(NC(=O)N2CCc3nn4c(c3C2)C(F)(F)CCC(F)(CO)C4)ccc1F]

The title compound was prepared in a manner analogous to Example 1, however using tert-butyl 8,11,11-trifluoro-8-(hydroxymethyl)-3,4,8,9,10,11-hexahydro-1H-pyrido[4′,3′:3,4]pyrazolo[1,5-a]azepine-2(7H)-carboxylate (Intermediate 20) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A. MS (ESI): mass calcd. for C₂₀H₁₉F₄N₅O₂, 437.1; m/z found, 438.1 [M+H]+. ¹H NMR (400 MHz, CD₃OD) δ=7.80 (dd, J=2.8, 5.6 Hz, 1H), 7.7-7.68 (m, 1H), 7.26 (t, J=9.0 Hz, 1H), 4.76-4.60 (m, 3H), 4.45-4.30 (m, 1H), 3.88-3.75 (m, 2H), 3.69-3.53 (m, 2H), 2.80 (t, J=5.7 Hz, 2H), 2.57-2.33 (m, 2H), 2.26-2.08 (m, 2H).

### Example 46

### N-(2-Bromo-3-fluoropyridin-4-yl)-8,11,11-trifluoro-8-(hydroxymethyl)-3,4,8,9,10,11-hexahydro-1H-pyrido[4′,3′:3,4]pyrazolo[1,5-a]azepine-2(7H)-carboxamide

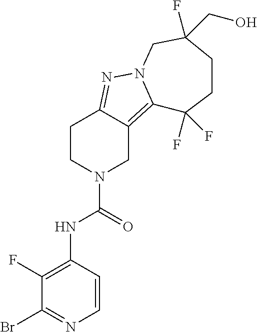 [SMILES: O=C(Nc1ccnc(Br)c1F)N1CCc2nn3c(c2C1)C(F)(F)CCC(F)(CO)C3]

The title compound was prepared in a manner analogous to Example 1, however using tert-butyl 8,11,11-trifluoro-8-(hydroxymethyl)-3,4,8,9,10,11-hexahydro-1H-pyrido[4′,3′:3,4]pyrazolo[1,5-a]azepine-2(7H)-carboxylate (Intermediate 20) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (2-bromo-3-fluoropyridin-4-yl)carbamate instead of phenyl (3-cyano-4-fluorophenyl) carbamate in Step B. MS (ESI): mass calcd. for C₁₈H₁₈BrF₄N₅O₂, 491.1/493.1; m/z found, 492.0/494.0 [M+H]+. ¹H NMR (400 MHz, CDCl₃) δ=8.17 (t, J=5.6 Hz, 1H), 8.08 (d, J=5.4 Hz, 1H), 6.94 (br d, J=4.0 Hz, 1H), 4.86-4.61 (m, 3H), 4.44-4.27 (m, 1H), 3.84-3.68 (m, 4H), 2.88 (t, J=5.9 Hz, 2H), 2.39-2.01 (m, 5H).

### Example 47

### (S*)-N-(2-Bromo-3-fluoropyridin-4-yl)-8,11,11-trifluoro-8-(hydroxymethyl)-3,4,8,9,10,11-hexahydro-1H-pyrido[4′,3′:3,4]pyrazolo[1,5-a]azepine-2(7H)-carboxamide

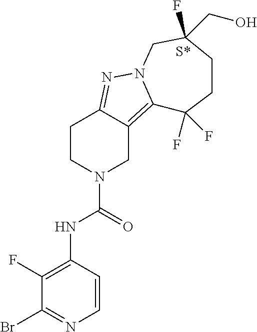 [SMILES: O=C(Nc1ccnc(Br)c1F)N1CCc2nn3c(c2C1)C(F)(F)CC[C@@](F)(CO)C3]

The title compound was prepared in a manner analogous to Example 1, however using (S*)-tert-butyl 8,11,11-trifluoro-8-(hydroxymethyl)-3,4,8,9,10,11-hexahydro-1H-pyrido[4′,3′:3,4]pyrazolo[1,5-a]azepine-2(7H)-carboxylate (Intermediate 21) instead of tert-butyl 11,11-difluoro-8-methylene-3,4,8,9,10,11-hexahydro-1H-pyrido[4′,3′:3,4] pyrazolo[1,5-a]azepine-2(7H)-carboxylate (Intermediate 1) in Step A and phenyl (2-bromo-3-fluoropyridin-4-yl)carbamate instead of phenyl(3-cyano-4-fluorophenyl)carbamate in Step B. MS (ESI): mass calcd. for C₁₈H₁₈BrF₄N₅O₂, 491.1/493.1; m/z found, 492.1/494.0 [M+H]+. ¹H NMR (400 MHz, CDCl₃) δ=8.17 (t, J=5.6 Hz, 1H), 8.08 (d, J=5.5 Hz, 1H), 6.93 (d, J=3.7 Hz, 1H), 4.79 (d, J=14.7 Hz, 1H), 4.72-4.66 (m, 2H), 4.41-4.27 (m, 1H), 3.85-3.80 (m, 2H), 3.78-3.59 (m, 2H), 2.88 (t, J=5.9 Hz, 2H), 2.60-2.35 (m, 2H), 2.27-2.06 (m, 2H), 2.02-1.96 (m, 1H).

### Example 48

### (R*)-N-(2-Bromo-3-fluoropyridin-4-yl)-8,11,11-trifluoro-8-(hydroxymethyl)-3,4,8,9,10,11-hexahydro-1H-pyrido[4′,3′:3,4]pyrazolo[1,5-a]azepine-2(7H)-carboxamide

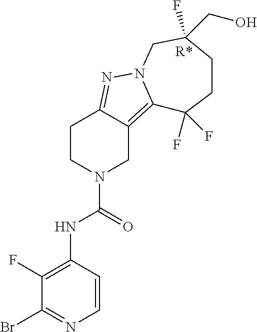 [SMILES: O=C(Nc1ccnc(Br)c1F)N1CCc2nn3c(c2C1)C(F)(F)CC[C@](F)(CO)C3]

The title compound was prepared in a manner analogous to Example 1, however using (R*)-tert-butyl 8,11,11-trifluoro-8-(hydroxymethyl)-3,4,8,9,10,11-hexahydro-1H-pyrido[4′,3′:3,4]pyrazolo[1,5-a]azepine-2(7H)-carboxylate (Intermediate 22) instead of tert-butyl 11,11-difluoro-8-methylene-3,4,8,9,10,11-hexahydro-1H-pyrido[4′,3′:3,4] pyrazolo[1,5-a]azepine-2(7H)-carboxylate (Intermediate 1) in Step A and phenyl (2-bromo-3-fluoropyridin-4-yl)carbamate instead of phenyl(3-cyano-4-fluorophenyl)carbamate in Step B. MS (ESI): mass calcd. for C₁₈H₁₈BrF₄N₅O₂, 491.1/493.1; m/z found, 492.1/494.0 [M+H]+. ¹H NMR (400 MHz, CDCl₃) δ=8.17 (t, J=5.6 Hz 1H), 8.08 (d, J=5.5 Hz, 1H), 6.93 (d, J=3.5 Hz, 1H), 4.79 (d, J=13.0 Hz, 1H), 4.71-4.67 (m, 2H), 4.41-4.28 (m, 1H), 3.85-3.80 (m, 2H), 3.74-3.58 (m, 2H), 2.88 (t, J=5.8 Hz, 2H), 2.60-2.34 (m, 2H), 2.26-1.96 (m, 3H).

### Example 49

### 8-Acetamidomethyl)-N-(3-cyano-4-fluorophenyl)-11,11-difluoro-3,4,8,9,10,11-hexahydro-1H-pyrido[4′,3′:3,4]pyrazolo[1,5-a]azepine-2(7H)-carboxamide

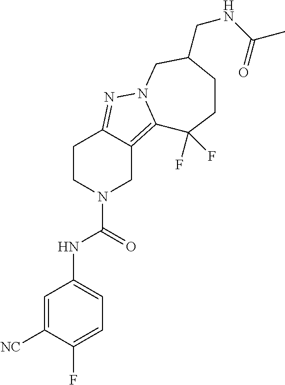 [SMILES: CC(=O)NCC1CCC(F)(F)c2c3c(nn2C1)CCN(C(=O)Nc1ccc(F)c(C#N)c1)C3]

The title compound was prepared in a manner analogous to Example 1, however using tert-butyl 8-(acetamidomethyl)-11,11-difluoro-3,4,8,9,10,11-hexahydro-1H-pyrido[4′,3′:3,4]pyrazolo[1,5-a]azepine-2(7H)-carboxylate (Intermediate 23) instead of tert-butyl 11,11-difluoro-8-methylene-3,4,8,9,10,11-hexahydro-1H-pyrido[4′,3′:3,4] pyrazolo[1,5-a]azepine-2(7H)-carboxylate (Intermediate 1) in Step A. MS (ESI): mass calcd. for C₂₂H₂₃F₃N₆O₂, 460.2; m/z found, 461.2 [M+H]+. ¹H NMR (400 MHz, CDCl₃) δ=7.73 (dd, J=2.8, 5.4 Hz, 1H), 7.62-7.59 (m, 1H), 7.15 (t, J=8.7 Hz, 1H), 6.50 (s, 1H), 5.70-5.60 (m, 1H), 4.62 (s, 2H), 4.39 (d, J=13.9 Hz, 1H), 4.19 (dd, J=7.9, 14.5 Hz, 1H), 3.88-3.76 (m, 2H), 3.41-3.32 (m, 1H), 2.94-2.87 (m, 1H), 2.83 (t, J=5.9 Hz, 2H), 2.51-2.09 (m, 4H), 2.03 (s, 3H), 1.91-1.81 (m, 1H).

### Example 50

### 8-(Acetamidomethyl)-N-(2-bromo-3-fluoropyridin-4-yl)-11,11-difluoro-3,4,8,9,10,11-hexahydro-1H-pyrido[4′,3′:3,4]pyrazolo[1,5-a]azepine-2(7H)-carboxamide

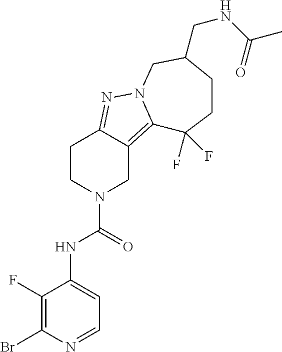 [SMILES: CC(=O)NCC1CCC(F)(F)c2c3c(nn2C1)CCN(C(=O)Nc1ccnc(Br)c1F)C3]

The title compound was prepared in a manner analogous to Example 1, however using tert-butyl 8-(acetamidomethyl)-11,11-difluoro-3,4,8,9,10,11-hexahydro-1H-pyrido[4′,3′:3,4]pyrazolo[1,5-a]azepine-2(7H)-carboxylate (Intermediate 23) instead of tert-butyl 11,11-difluoro-8-methylene-3,4,8,9,10,11-hexahydro-1H-pyrido[4′,3′:3,4] pyrazolo[1,5-a]azepine-2(7H)-carboxylate (Intermediate 1) in Step A and phenyl (2-bromo-3-fluoropyridin-4-yl)carbamate instead of phenyl(3-cyano-4-fluorophenyl) carbamate in Step B. MS (ESI): mass calcd. for C₂₀H₂₂BrF₃N₆O₂, 514.1/516.1; m/z found, 515.1/517.1[M+H]+. ¹H NMR (400 MHz, CDCl₃) δ=8.18 (t, J=5.5 Hz, 1H), 8.08 (d, J=5.5 Hz, 1H), 6.94 (d, J=4.2 Hz, 1H), 5.70-5.60 (m, 1H), 4.67 (s, 2H), 4.39 (d, J=14.8 Hz, 1H), 4.19 (dd, J=8.1, 14.2 Hz, 1H), 3.86-3.79 (m, 2H), 3.41-3.31 (m, 1H), 2.95-2.83 (m, 3H), 2.51-2.35 (m, 1H), 2.35-2.22 (m, 1H), 2.21-2.05 (m, 2H), 2.05(s, 3H), 1.91-1.80 (m, 1H).

### Example 51

### N-(3-Cyano-4-fluorophenyl)-11,11-difluoro-8-((2,2,2-trifluoroacetamido) methyl)-3,4,8,9,10,11-hexahydro-1H-pyrido[4′,3′:3,4]pyrazolo[1,5-a]azepine-2(7H)-carboxamide

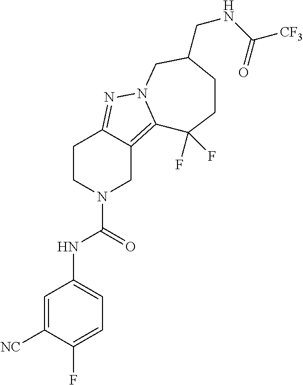 [SMILES: N#Cc1cc(NC(=O)N2CCc3nn4c(c3C2)C(F)(F)CCC(CNC(=O)C(F)(F)F)C4)ccc1F]

The title compound was prepared in a manner analogous to Example 1, however using tert-butyl 11,11-difluoro-8-((2,2,2-trifluoroacetamido)methyl)-3,4,8, 9,10,11-hexahydro-1H-pyrido[4′,3′:3,4]pyrazolo[1,5-a]azepine-2(7H)-carboxylate (Intermediate 24) instead of tert-butyl 11,11-difluoro-8-methylene-3,4,8,9,10,11-hexahydro-1H-pyrido[4′,3′:3,4] pyrazolo[1,5-a]azepine-2(7H)-carboxylate (Intermediate 1) in Step A. MS (ESI): mass calcd. for C₂₂H₂ON₆F₆O₂, 514.2; m/z found, 515.2 [M+H]+. ¹H NMR (400 MHz, CD30D) δ=7.81 (dd, J=2.7, 5.6 Hz, 1H), 7.71-7.68 (m, 1H), 7.28 (t, J=9.0 Hz, 1H), 4.69 (s, 2H), 4.39 (d, J=15.0 Hz, 1H), 4.15 (dd, J=8.7, 14.4 Hz, 1H), 3.82 (t, J=5.9 Hz, 2H), 3.23 (d, J=7.1 Hz, 2H), 2.80 (t, J=5.6 Hz, 2H), 2.60-2.00 (m, 4H), 1.93-1.77 (m, 1H).

### Example 52

### N-(2-Bromo-3-fluoropyridin-4-yl)-11,11-difluoro-8-((2,2,2-trifluoroacetamido)methyl)-3,4,8,9,10,11-hexahydro-1H-pyrido[4′,3′:3,4]pyrazolo[1,5-a]azepine-2(7H)-carboxamide

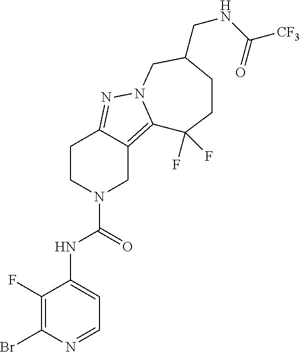 [SMILES: O=C(Nc1ccnc(Br)c1F)N1CCc2nn3c(c2C1)C(F)(F)CCC(CNC(=O)C(F)(F)F)C3]

The title compound was prepared in a manner analogous to Example 1, however using tert-butyl 11,11-difluoro-8-((2,2,2-trifluoroacetamido)methyl)-3,4,8, 9,10,11-hexahydro-1H-pyrido[4′,3′:3,4]pyrazolo[1,5-a]azepine-2(7H)-carboxylate (Intermediate 24) instead of tert-butyl 11,11-difluoro-8-methylene-3,4,8,9,10,11-hexahydro-1H-pyrido[4′,3′:3,4] pyrazolo[1,5-a]azepine-2(7H)-carboxylate (Intermediate 1) in Step A and phenyl (2-bromo-3-fluoropyridin-4-yl)carbamate instead of phenyl(3-cyano-4-fluorophenyl) carbamate in Step B. MS (ESI): mass calcd. for C₂₀H₁₉N₆F₆O₂Br, 568.1/570.1; m/z found, 569.0/571.1 [M+H]+. ¹H NMR (400 MHz, CDCl₃) δ=8.23-8.15 (m, 1H), 8.09 (d, J=5.5 Hz, 1H), 6.94 (br d, J=4.3 Hz, 1H), 6.53 (br s, 1H), 4.69 (s, 2H), 4.48-4.37 (m, 1H), 4.27 (dd, J=7.2, 14.7 Hz, 1H), 3.89-3.78 (m, 2H), 3.48-3.45(m, 1H), 3.12-2.98 (m, 1H), 2.88 (t, J=5.9 Hz, 2H), 2.52-2.23 (m, 3H), 2.21-2.08 (m, 1H), 1.96-1.82 (m, 1H).

### Example 53

### Methyl ((2-((3-cyano-4-fluorophenyl)carbamoyl)-11,11-difluoro-2,3,4,7,8,9,10,11-octahydro-1H-pyrido[4′,3′:3,4]pyrazolo[1,5-a]azepin-8-yl)methyl)-carbamate

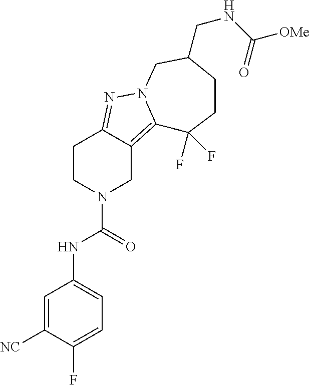 [SMILES: COC(=O)NCC1CCC(F)(F)c2c3c(nn2C1)CCN(C(=O)Nc1ccc(F)c(C#N)c1)C3]

The title compound was prepared in a manner analogous to Example 1, however using tert-butyl 11,11-difluoro-8-(((methoxycarbonyl)amino)methyl)-3,4,8,9,10,11-hexahydro-1H-pyrido[4′,3′:3,4]pyrazolo[1,5-a]azepine-2(7H)-carboxylate (Intermediate 25) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A. MS (ESI): mass calcd. for C₂₂H₂₃F₃N₆O₃, 476.2; m/z found, 477.2 [M+H]+. ¹H NMR (400 MHz, DMSO-d₆) δ=9.04 (s, 1H), 7.93 (dd, J=2.8, 5.8 Hz, 1H), 7.78-7.76 (m, 1H), 7.48-7.32 (m, 2H), 4.57 (s, 2H), 4.31 (d, J=14.1 Hz, 1H), 3.96 (dd, J=9.5, 14.1 Hz, 1H), 3.75-3.65 (m, 2H), 3.53 (s, 3H), 2.92 (t, J=6.4 Hz, 2H), 2.71-2.65 (m, 2H), 2.44-2.38 (m, 1H), 2.31-2.16 (m, 1H), 1.95-1.83 (m, 2H), 1.73-1.60 (m, 1H).

### Example 54

### Methyl ((2-((2-bromo-3-fluoropyridin-4-yl)carbamoyl)-11,11-difluoro-2,3,4,7,8,9,10,11-octahydro-1H-pyrido[4′,3′:3,4]pyrazolo[1,5-a]azepin-8-yl)methyl)-carbamate

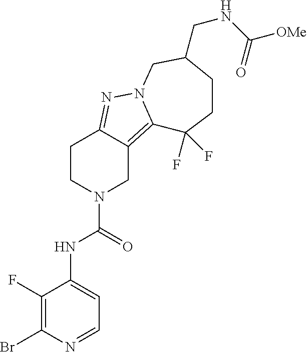 [SMILES: COC(=O)NCC1CCC(F)(F)c2c3c(nn2C1)CCN(C(=O)Nc1ccnc(Br)c1F)C3]

The title compound was prepared in a manner analogous to Example 1, however using tert-butyl 11,11-difluoro-8-(((methoxycarbonyl)amino)methyl)-3,4,8,9,10,11-hexahydro-1H-pyrido[4′,3′:3,4]pyrazolo[1,5-a]azepine-2(7H)-carboxylate (Intermediate 25) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A phenyl (2-bromo-3-fluoropyridin-4-yl)carbamate instead of phenyl(3-cyano-4-fluorophenyl) carbamate in Step B. MS (ESI): mass calcd. for C₂₀H₂₂BrF₃N₆O₃, 530.1/532.1; m/z found, 531.1/533.1[M+H]+. ¹H NMR (400 MHz, CDCl₃) δ=8.18 (t, J=5.6 Hz, 1H), 8.08 (d, J=5.5 Hz, 1H), 6.93 (d, J=3.9 Hz, 1H), 4.86-480 (m, 1H), 4.67 (s, 2H), 4.42 (d, J=14.5 Hz, 1H), 4.16 (dd, J=8.1, 14.3 Hz, 1H), 3.82 (t, J=5.9 Hz, 2H), 3.69 (s, 3H), 3.27-3.15 (m, 1H), 3.01-2.91 (m, 1H), 2.86 (t, J=5.8 Hz, 2H), 2.53-2.35 (m, 1H), 2.34-2.17 (m, 1H), 2.14-2.02 (m, 2H), 1.91-1.78 (m, 1H).

### Example 55

### N-(3-Cyano-4-fluorophenyl)-11,11-difluoro-8-hydroxy-8-methyl-3,4,8,9,10,11-hexahydro-1H-pyrido[4′,3′:3,4]pyrazolo[1,5-a]azepine-2(7H)-carboxamide

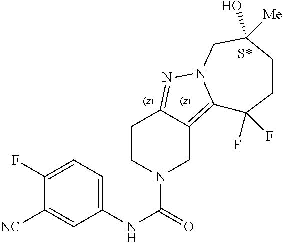 [SMILES: C[C@@]1(O)CCC(F)(F)c2c3c(nn2C1)CCN(C(=O)Nc1ccc(F)c(C#N)c1)C3]

The title compound was prepared from Intermediate 26 in a manner analogous to Example 1. However, the title compound was purified by RP HPLC (condition E).-MS (ESI): mass calcd. for C₂₀H₂₀F₃N₅O₂, 419.2; m/z found, 420.1 [M+H]+. ¹H NMR (400 MHz, CDCl₃) δ=7.74-7.72(m, 1H), 7.63-7.61 (m, 1H), 7.15 (t, J=8.7 Hz, 1H), 6.50 (s, 1H), 4.71-4.55 (m, 2H), 4.28 (s, 2H), 3.81 (t, J=5.8 Hz, 2H), 2.87-2.84 (m, 2H), 2.57-2.20 (m, 2H), 2.13-1.97 (m, 2H), 1.35 (s, 3H).

### Example 56

### (R*)-N-(3-Cyano-4-fluorophenyl)-11,11-difluoro-8-hydroxy-8-vinyl-3,4,8,9,10,11-hexahydro-1H-pyrido[4′,3′:3,4]pyrazolo[1,5-a]azepine-2(7H)-carboxamide

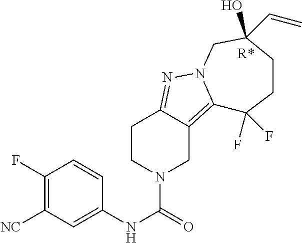 [SMILES: C=C[C@]1(O)CCC(F)(F)c2c3c(nn2C1)CCN(C(=O)Nc1ccc(F)c(C#N)c1)C3]

Step A. 11,11-Difluoro-8-vinyl-2,3,4,7,8,9,10,11-octahydro-1H-pyrido[4′,3′:3,4] pyrazolo[1,5-a]azepin-8-ol. To a solution of tert-butyl 11,11-difluoro-8-hydroxy-8-vinyl-3,4,8,9,10,11-hexahydro-1H-pyrido[4′,3′:3,4]pyrazolo[1,5-a]azepine-2(7H)-carboxylate (0.112 g, 303.19 μmol) in DCM (8 mL) was added TFA (1.73 g, 15.16 mmol, 1.12 mL) in DCM (2 mL) at 0° C. under N₂, and the mixture was stirred at 15° C. for 2 h. The solution was used directly for the next step.

Step B. N-(3-Cyano-4-fluorophenyl)-11,11-difluoro-8-hydroxy-8-vinyl-3,4,8,9,10,11-hexahydro-1H-pyrido[4′,3′:3,4]pyrazolo[1,5-a]azepine-2(7H)-carboxamide. To the above reaction solution (from Step A, Example 56) were added a solution of Et₃N (1.59 g, 15.74 mmol, 2.19 mL) in DCM (15 mL) and phenyl (3-cyano-4-fluorophenyl)carbamate (83.38 mg, 302.62 μmol). The mixture was stirred at 15° C. for 2 h. The mixture was diluted with DCM (20 mL) and washed with brine (20 mL). The organic phase was dried over Na₂SO₄, filtered and concentrated in vacuo. The residue was purified by silica gel column (petroleum ether/ethyl acetate=1/0 to 1/2) to give the title compound (0.084 g, 188.67 μmol, 62.35% yield, 96.9% purity) as white solid. MS (ESI): mass calcd. for C₂₁H₂₀F₃N₅O₂, 431.2; m/z found, 432.4 [M+H]+.

Step C. (R*)-N-(3-Cyano-4-fluorophenyl)-11,11-difluoro-8-hydroxy-8-vinyl-3,4,8,9,10,11-hexahydro-1H-pyrido[4′,3′:3,4]pyrazolo[1,5-a]azepine-2(7H)-carboxamide. N-(3-cyano-4-fluorophenyl)-11,11-difluoro-8-hydroxy-8-vinyl-3,4,8,9,10,11-hexahydro-1H-pyrido[4′,3′:3,4]pyrazolo[1,5-a]azepine-2(7H)-carboxamide (84 mg) was resolved by SFC (condition: column: DAICEL CHIRALPAK AD(250 mm*30 mm,10 um); mobile phase: [0.1% NH₃.H₂O EtOH]; B %: 35%-35%,4.0 min; 50 min) to give (R*)-N-(3-cyano-4-fluorophenyl)-11,11-difluoro-8-hydroxy-8-vinyl-3,4,8,9,10,11-hexahydro-1H-pyrido[4′,3′:3,4]pyrazolo[1,5-a]azepine-2(7H)-carboxamide (Peak 1 on SFC (AD-3_S_5_30_3.0ML Column: Chiralpak AD-3 50×4.6 mm I.D., 3 um Mobile phase: 30% ethanol (0.05% DEA) in CO₂ Flow rate: 3.0 mL/min Wavelength: 220 nm″), retention time=0.640 min, 0.034 g) which was combined with another batch of (R*)-N-(3-cyano-4-fluorophenyl)-11,11-difluoro-8-hydroxy-8-vinyl-3,4,8,9,10,11-hexahydro-1H-pyrido[4′,3′:3,4]pyrazolo[1,5-a]azepine-2(7H)-carboxamide (18 mg) to repurify by RP HPLC(condition E) to give the title compound (0.033 g, 99.2% purity) as white solid. MS (ESI): mass calcd. for C₂₁H₂₀F₃N₅O₂, 431.2; m/z found, 432.1 [M+H]+. ¹H NMR (400 MHz, CD₃OD) δ=7.79 (dd, J=2.7, 5.6 Hz, 1H), 7.70-7.67 (m, 1H), 7.26 (t, J=9.0 Hz, 1H), 5.94 (dd, J=10.9, 17.4 Hz, 1H), 5.41 (dd, J=0.9, 17.4 Hz, 1H), 5.19 (dd, J=0.9, 10.9 Hz, 1H), 4.67 (s, 2H), 4.36-4.15 (m, 2H), 3.86-3.73 (m, 2H), 2.78 (t, J=5.8 Hz, 2H), 2.61-2.41 (m, 1H), 2.30 (br s, 1H), 2.22-2.09 (m, 1H), 2.02-1.91 (m, 1H).

### Example 57

### (S*)-N-(3-Cyano-4-fluorophenyl)-11,11-difluoro-8-hydroxy-8-vinyl-3,4,8,9,10,11-hexahydro-1H-pyrido[4′,3′:3,4]pyrazolo[1,5-a]azepine-2(7H)-carboxamide

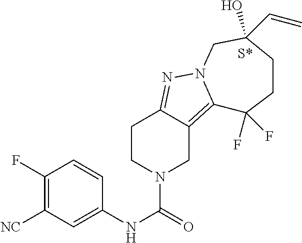 [SMILES: C=C[C@@]1(O)CCC(F)(F)c2c3c(nn2C1)CCN(C(=O)Nc1ccc(F)c(C#N)c1)C3]

The title compound was separated from title compound Example 56 via SFC in a manner analogous to Example 56 (Peak 2 on SFC (AD-3_S_5_30_3.0ML Column: Chiralpak AD-3 50×4.6 mm I.D., 3 um Mobile phase: 30% ethanol (0.05% DEA) in CO₂ Flow rate: 3.0 mL/min Wavelength: 220 nm), retention time=0.917 min). MS (ESI): mass calcd. for C₂₁H₂₀F₃N₅O₂, 431.2; m/z found, 432.1 [M+H]+. ¹H NMR (400 MHz, CD₃OD) δ=7.79 (dd, J=2.7, 5.6 Hz, 1H), 7.70-7.67 (m, 1H), 7.26 (t, J=9.0 Hz, 1H), 5.94 (dd, J=10.9, 17.4 Hz, 1H), 5.41 (dd, J=0.9, 17.4 Hz, 1H), 5.19 (dd, J=0.9, 10.9 Hz, 1H), 4.67 (s, 2H), 4.37-4.15 (m, 2H), 3.86-3.73 (m, 2H), 2.78 (t, J=5.7 Hz, 2H), 2.60-2.40 (m, 1H), 2.38-2.23 (m, 1H), 2.25-2.10 (m, 1H), 2.03-1.88 (m, 1H).

### Example 58

### N-(3-Cyano-4-fluorophenyl)-8-ethynyl-11,11-di fluoro-8-hydroxy-3,4,8,9,10,11-hexahydro-1H-pyrido[4′,3′:3,4]pyrazolo[1,5-a]azepine-2(7H)-carboxamide

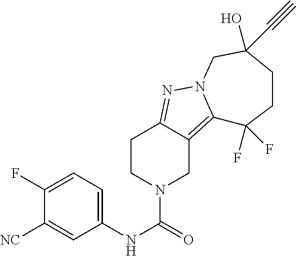 [SMILES: C#CC1(O)CCC(F)(F)c2c3c(nn2C1)CCN(C(=O)Nc1ccc(F)c(C#N)c1)C3]

The title compound was prepared in a manner analogous to Example 1, however using tert-butyl 8-ethynyl-11,11-difluoro-8-hydroxy-3,4,8,9,10,11-hexahydro-1H-pyrido[4′,3′:3,4]pyrazolo[1,5-a]azepine-2(7H)-carboxylate (Intermediate 28) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A, and purified via RP HPLC(condition E). MS (ESI): mass calcd. for C₂₁H₁₈F₃N₅O₂ 429.1; m/z found, 430.1 [M+H]+. ¹H NMR (400 MHz, CD₃OD) δ=7.81-7.79 (m, 1H), 7.71-7.68 (m, 1H), 7.30-7.25 (t, J=9.2 Hz, 1H), 4.67 (s, 2H), 4.44-4.32 (m, 2H), 3.83-3.79 (m, 2H), 2.93 (s, 1H), 2.81-2.78 (m, 2H), 2.60-2.35 (m, 2H). 2.26-2.23 (m, 2H).

### Example 59

### (R*)-N-(3-Cyano-4-fluorophenyl)-8-ethynyl-11,11-difluoro-8-hydroxy-3,4,8,9,10,11-hexahydro-1H-pyrido[4′,3′:3,4]pyrazolo[1,5-a]azepine-2(7H)-carboxamide

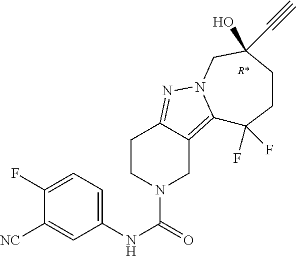 [SMILES: C#C[C@]1(O)CCC(F)(F)c2c3c(nn2C1)CCN(C(=O)Nc1ccc(F)c(C#N)c1)C3]

N-(3-cyano-4-fluorophenyl)-8-ethynyl-11,11-difluoro-8-hydroxy-3,4,8,9,10,11-hexahydro-1H-pyrido[4′,3′:3,4]pyrazolo[1,5-a]azepine-2(7H)-carboxamide (300 mg) was resolved by SFC (condition: column: DAICEL CHIRALCEL OD (250 mm*30 mm,10 um); mobile phase: [Neu-MeOH]; B %: 40%-40%,2.25 min; 60 min) to give (R*)-N-(3-cyano-4-fluorophenyl)-8-ethynyl-11,11-difluoro-8-hydroxy-3,4,8,9,10,11-hexahydro-1H-pyrido[4′,3′:3,4]pyrazolo[1,5-a]azepine-2(7H)-carboxamide (Peak 1 on SFC (Cellucoat_MeOH(DEA)_5_40_3mL-35T Column: Cellucoat 50×4.6 mm I.D., 3 um Mobile phase: methanol (0.05% DEA) in CO₂ from 5% to 40%, Flow rate: 3 mL/min Wavelength: 220 nm), retention time=1.644 min, 0.14 g, 318.54 μmol, 47.05% yield, 97.7% purity) as white solid, which was repurified by RP HPLC(condition E) to give the title compound (0.037 g, 86.17 μmol, 61.67% yield) as white solid. MS (ESI): mass calcd. for C₂₁H₁₈F₃N₅O₂ 429.1; m/z found, 430.1 [M+H]+. ¹H NMR (400 MHz, CD₃OD) δ=7.80-7.77 (m, 1H), 7.69-7.67 (m, 1H), 7.28 (t, J=9.2 Hz, 1H), 4.66 (s, 2H), 4.32-4.30 (m, 2H), 3.81-3.77 (m, 2H), 2.90 (s, 1H), 2.79-2.76 (m, 2H), 2.55-2.35 (m, 2H), 2.25-2.22 (m, 2H).

### Example 60

### (S*)-N-(3-Cyano-4-fluorophenyl)-8-ethynyl-11,11-difluoro-8-hydroxy-3,4,8,9,10,11-hexahydro-1H-pyrido[4′,3′:3,4]pyrazolo[1,5-a]azepine-2(7H)-carboxamide

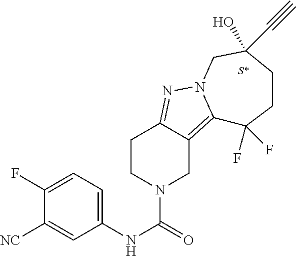 [SMILES: C#C[C@@]1(O)CCC(F)(F)c2c3c(nn2C1)CCN(C(=O)Nc1ccc(F)c(C#N)c1)C3]

The title compound was separated from Example 59 in a manner analogous to Example 59 (Peak 2 on SFC (Cellucoat_MeOH(DEA)_5_40_3mL-35T Column: Cellucoat 50×4.6 mm I.D., 3 um Mobile phase: methanol (0.05% DEA) in CO₂ from 5% to 40% Flow rate: 3 mL/min Wavelength: 220 nm), retention time=1.835 min, 0.14 g, 320.50 μmol, 47.34% yield, 98.3% purity, optical rotation [a]²⁰_{D}=+3.259(c=0.494, MeOH)), which was repurified by RP HPLC (condition E) to give 0.03 g, 69.45 μmol, 49.70% yield, 99.4% purity) as white solid and (0.01 g, 21.54 μmol, 15.42% yield, 92.5% purity) as white solid. MS (ESI): mass calcd. for C₂₁H₁₈F₃N₅O₂ 429.1; m/z found, 430.1 [M+H]+. ¹H NMR (400 MHz, CD₃OD) δ=7.82-7.80 (m, 1H), 7.72-7.69 (m, 1 \H), 7.28 (t, J=9.2 Hz, 1H), 4.68 (s, 2H), 4.59-4.33 (m, 2H), 3.83-3.80 (m, 2H), 2.93 (s, 1H), 2.82-2.79 (m, 2H), 2.55-2.35 (m, 2H). 2.27-2.24 (m, 2H).

### Example 61

### (S*)-N-(3-Cyano-4-fluorophenyl)-8-ethyl-11,11-difluoro-8-hydroxy-3,4,8,9,10,11-hexahydro-1H-pyrido[4′,3′:3,4]pyrazolo[1,5-a]azepine-2(7H)-carboxamide

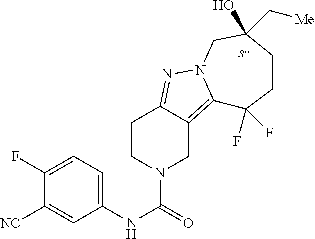 [SMILES: CC[C@]1(O)CCC(F)(F)c2c3c(nn2C1)CCN(C(=O)Nc1ccc(F)c(C#N)c1)C3]

To a solution of (S*)-N-(3-cyano-4-fluorophenyl)-8-ethynyl-11,11-difluoro-8-hydroxy-3,4,8,9,10,11-hexahydro-1H-pyrido[4′,3′:3,4]pyrazolo[1,5-a]azepine-2(7H)-carboxamide (0.08 g, 186.31 μmol) in THF (8 mL) was added Pd/C (0.015 g, 186.31 μmol, 10% purity) under N₂. The mixture was stirred at 15° C. under H₂ (15 psi) for 0.5 h. The mixture was filtered and concentrated in vacuo. The residue was purified by RP HPLC (condition E) to give the title compound. MS (ESI): mass calcd. for C₂₁H₂₂F₃N₅O₂, 433.2; m/z found, 434.1 [M+H]+. ¹H NMR (400 MHz, CD₃OD) δ=7.80 (dd, J=2.8, 5.6 Hz, 1H), 7.70-7.67 (m, 1H), 7.26 (t, J=9.0 Hz, 1H), 4.67 (s, 2H), 4.21 (s, 2H), 3.90-3.68 (m, 2H), 2.79 (t, J=5.7 Hz, 2H), 2.55-2.35 (m, 1H), 2.35-2.25 (m, 1H), 2.06-1.90 (m, 2H), 1.53-1.32 (m, 2H), 0.97 (t, J=7.5 Hz, 3H).

### Example 62

### (R*)-N-(3-Cyano-4-fluorophenyl)-8-ethyl-11,11-difluoro-8-hydroxy-3,4,8,9,10,11-hexahydro-1H-pyrido[4′,3′:3,4]pyrazolo[1,5-a]azepine-2(7H)-carboxamide

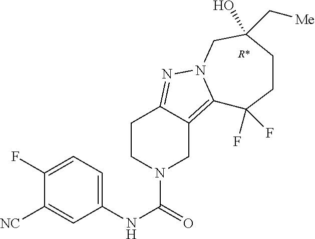 [SMILES: CC[C@@]1(O)CCC(F)(F)c2c3c(nn2C1)CCN(C(=O)Nc1ccc(F)c(C#N)c1)C3]

The title compound was prepared in a manner analogous to Example 61, except substituting (R*)-N-(3-cyano-4-fluorophenyl)-8-ethynyl-11,11-difluoro-8-hydroxy-3,4,8,9,10,11-hexahydro-1H-pyrido[4′,3′:3,4]pyrazolo[1,5-a]azepine-2(7H)-carboxamide instead of (S*)-N-(3-cyano-4-fluorophenyl)-8-ethynyl-11,11-difluoro-8-hydroxy-3,4,8,9,10,11-hexahydro-1H-pyrido[4′,3′:3,4]pyrazolo[1,5-a]azepine-2(7H)-carboxamide. MS (ESI): mass calcd. for C₂₁H₂₂F₃N₅O₂, 433.2; m/z found, 434.1 [M+H]+. ¹H NMR (400 MHz, CD₃OD) δ=7.79 (dd, J=2.7, 5.6 Hz, 1H), 7.73-7.66 (m, 1H), 7.26 (t, J=9.2 Hz, 1H), 4.67 (s, 2H), 4.20 (s, 2H), 3.84-3.73 (m, 2H), 2.85-2.74 (m, 2H), 2.54-2.37 (m, 1H), 2.33-2.17 (m, 1H), 2.06-1.89 (m, 2H), 1.53-1.33 (m, 2H), 0.96 (t, J=7.5 Hz, 3H).

### Example 63

### (R*)-N-(2-Bromo-3-fluoropyridin-4-yl)-8-ethynyl-11,11-difluoro-8-hydroxy-3,4,8,9,10,11-hexahydro-1H-pyrido[4′,3′:3,4]pyrazolo[1,5-a]azepine-2(7H)-carboxamide

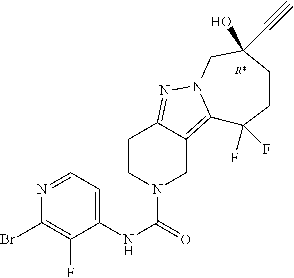 [SMILES: C#C[C@]1(O)CCC(F)(F)c2c3c(nn2C1)CCN(C(=O)Nc1ccnc(Br)c1F)C3]

The title compound was prepared in a manner analogous to Example 1, however using (R*)-tert-butyl 8-ethynyl-11,11-difluoro-8-hydroxy-3,4,8,9,10,11-hexahydro-1H-pyrido[4′,3′:3,4]pyrazolo[1,5-a]azepine-2(7H)-carboxylate(Intermediate 29) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate(Intermediate 1) in Step A and phenyl (2-bromo-3-fluoropyridin-4-yl)carbamate instead of phenyl (3-cyano-4-fluorophenyl) carbamate in Step B, and purified by RP HPLC (condition D, and then condition E). MS (ESI): mass calcd. for C₁₉H₁₇BrF₃N₅O₂ 483.0/485.0; m/z found, 484.1/486.1 [M+H]+. ¹H NMR (400 MHz, CDCl₃) δ=8.22-8.14 (m, 1H), 8.08 (d, J=5.5 Hz, 1H), 6.93 (d, J=3.7 Hz, 1H), 4.78-4.60 (m, 2H), 4.50 (d, J=2.3 Hz, 2H), 3.95-3.72 (m, 2H), 2.88 (t, J=5.8 Hz, 2H), 2.61-2.26 (m, 6H).

### Example 64

### (R*)-N-(2-(Difluoromethyl)-3-fluoropyridin-4-yl)-8-ethynyl-11,11-difluoro-8-hydroxy-3,4,8,9,10,11-hexahydro-1H-pyrido[4′,3′:3,4]pyrazolo[1,5-a]azepine-2(7H)-carboxamide

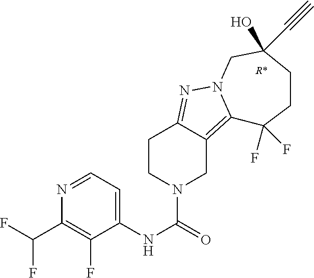 [SMILES: C#C[C@]1(O)CCC(F)(F)c2c3c(nn2C1)CCN(C(=O)Nc1ccnc(C(F)F)c1F)C3]

The title compound was prepared in a manner analogous to Example 1, however using (R*)-tert-butyl 8-ethynyl-11,11-difluoro-8-hydroxy-3,4,8,9,10,11-hexahydro-1H-pyrido[4′,3′:3,4]pyrazolo[1,5-a]azepine-2(7H)-carboxylate (Intermediate 29) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate(Intermediate 1) in Step A and phenyl (2-(difluoromethyl)-3-fluoropyridin-4-yl) carbamate instead of phenyl (3-cyano-4-fluorophenyl)carbamate in Step B, and purification via RP HPLC (condition E). MS (ESI): mass calcd. for C₂₀H₁₈F₅N₅O₂ 455.1; m/z found, 456.1 [M+H]+. ¹H NMR (400 MHz, CDCl₃) δ=8.37-8.29 (m, 2H), 7.01 (d, J=3.5 Hz, 1H), 6.75 (t, J=53.6 Hz, 1H), 4.80-4.62 (m, 2H), 4.50 (s, 2H), 3.95-3.74 (m, 2H), 2.88 (t, J=5.9 Hz, 2H), 2.63-2.21 (m, 6H).

### Example 65

### (S*)-N-(2-Bromo-3-fluoropyridin-4-yl)-8-ethynyl-11,11-difluoro-8-hydroxy-3,4,8,9,10,11-hexahydro-1H-pyrido[4′,3′:3,4]pyrazolo[1,5-a]azepine-2(7H)-carboxamide

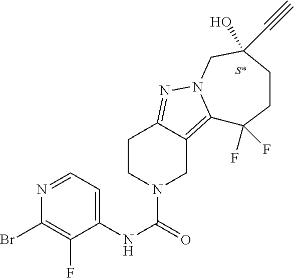 [SMILES: C#C[C@@]1(O)CCC(F)(F)c2c3c(nn2C1)CCN(C(=O)Nc1ccnc(Br)c1F)C3]

The title compound was prepared in a manner analogous to Example 1, however using (S*)-tert-butyl 8-ethynyl-11,11-difluoro-8-hydroxy-3,4,8,9,10,11-hexahydro-1H-pyrido[4′,3′:3,4]pyrazolo[1,5-a]azepine-2(7H)-carboxylate (Intermediate 30) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate(Intermediate 1) in Step A and phenyl (2-bromo-3-fluoropyridin-4-yl)carbamate instead of phenyl (3-cyano-4-fluorophenyl) carbamate in Step B, and purification via RP HPLC (condition E). MS (ESI): mass calcd. for C₁₉H₁₇BrF₃N₅O₂ 483.1/485.1; m/z found, 484.1/486.1 [M+H]+. ¹H NMR (400 MHz, CDCl₃) δ=8.17 (t, J=5.6 Hz, 1H), 8.08 (d, J=5.5 Hz, 1H), 6.93 (br d, J=4.0 Hz, 1H), 4.77-4.60 (m, 2H), 4.51 (s, 2H), 3.91-3.73 (m, 2H), 2.88 (t, J=5.8 Hz, 2H), 2.73 (s, 1H), 2.60-2.46 (m, 2H), 2.45-2.26 (m, 3H).

### Example 66

### (S*)-N-(2-(Difluoromethyl)-3-fluoropyridin-4-yl)-8-ethynyl-11,11-difluoro-8-hydroxy-3,4,8,9,10,11-hexahydro-1H-pyrido[4′,3′:3,4]pyrazolo[1,5-a]azepine-2(7H)-carboxamide

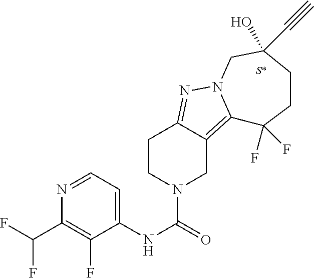 [SMILES: C#C[C@@]1(O)CCC(F)(F)c2c3c(nn2C1)CCN(C(=O)Nc1ccnc(C(F)F)c1F)C3]

The title compound was prepared in a manner analogous to Example 1, however using (S*)-tert-butyl 8-ethynyl-11,11-difluoro-8-hydroxy-3,4,8,9,10,11-hexahydro-1H-pyrido[4′,3′:3,4]pyrazolo[1,5-a]azepine-2(7H)-carboxylate(Intermediate 30) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate(Intermediate 1) in Step A and phenyl (2-(difluoromethyl)-3-fluoropyridin-4-yl) carbamate instead of phenyl (3-cyano-4-fluorophenyl)carbamate in Step B, and purification via RP HPLC (condition E). MS (ESI): mass calcd. for C₂₀H₁₈F₅N₅O₂ 455.1; m/z found, 456.2 [M+H]+. ¹H NMR (400 MHz, CDCl₃) δ=8.34-8.28 (m, 2H), 6.98 (br d, J=4.4 Hz, 1H), 6.76 (J=53.6 Hz, 1H), 4.73-4.59 (m, 2H), 4.56 (s, 2H), 3.89-3.72 (m, 2H), 2.85 (t, J=5.8 Hz, 2H), 2.64 (s, 1H), 2.57-2.46 (m, 2H), 2.41-2.23 (m, 3H).

### Example 67

### (3R,8R)-N-(3-cyano-4-fluorophenyl)-11,11-difluoro-8-hydroxy-3-methyl-3,4,8,9,10,11-hexahydro-1H-pyrido[4′,3′:3,4]pyrazolo[1,5-a]azepine-2(7H)-carboxamide

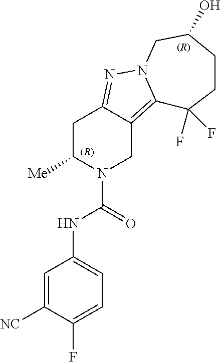

The title compound was prepared in a manner analogous to Example 1, however using ((3R,8R)-tert-butyl 11,11-difluoro-8-hydroxy-3-methyl-3,4,8,9,10,11-hexahydro-1H-pyrido[4′,3′:3,4]pyrazolo[1,5-a]azepine-2(7H)-carboxylate (Intermediate 31) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A. MS (ESI): mass calcd. for C₂₀H₂₀F₃N₅O₂ 419.2; m/z found, 420.1 [M+H]+. ¹HNMR (400 MHz, CDCl₃) δ=7.73 (dd, J=2.8, 5.4 Hz, 1H), 7.63-7.61 (m, 1H), 7.16 (t, J=8.8 Hz, 1H), 6.51 (s, 1H), 5.0-4.97 (m, 1H), 4.83-4.79 (m, 1H), 4.47-4.34 (m, 3H), 4.23 (br m, 1H), 3.03 (dd, J=5.8, 15.9 Hz, 1H), 2.70-2.50 (m, 2H), 2.31-2.17 (m, 3H), 1.23 (d, J=6.85 Hz, 3H).

### Example 68

### (3R,8R)-N-(2-Bromo-3-fluoropyridin-4-yl)-11,11-di fluoro-8-hydroxy-3-methyl-3,4,8,9,10,11-hexahydro-1H-pyrido[4′,3′:3,4]pyrazolo[1,5-a]azepine-2(7H)-carboxamide

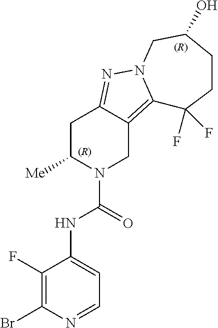

The title compound was prepared in a manner analogous to Example 1, however using ((3R,8R)-tert-butyl 11,11-difluoro-8-hydroxy-3-methyl-3,4,8,9,10,11-hexahydro-1H-pyrido[4′,3′:3,4]pyrazolo[1,5-a]azepine-2(7H)-carboxylate (Intermediate 31) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (2-bromo-3-fluoropyridin-4-yl)carbamate instead of phenyl (3-cyano-4-fluorophenyl)carbamate in Step B. MS (ESI): mass calcd. for C₁₈H₁₉BrF₃N₅O₂ 473.1/475.1; m/z found, 474.0/476.0 [M+H]+. ¹HNMR (400 MHz, CDCl₃) δ=8.17 (t, J=5.5 Hz, 1H), 8.10-8.06 (m, 1H), 6.93 (d, J=4.0 Hz, 1H), 5.04-4.76 (m, 2H), 4.52-4.42 (m, 3H), 4.22 (br m, 1H), 3.08-3.03 (m, 1H), 2.75-2.45 (m, 2H), 2.37-2.07 (m, 3H), 1.24 (d, J=7.0 Hz, 3H).

### Example 69

### (3R,8R)-11,11-Difluoro-N-(4-fluoro-3-(trifluoromethyl)phenyl)-8-hydroxy-3-methyl-3,4,8,9,10,11-hexahydro-1H-pyrido[4′,3′:3,4]pyrazolo[1,5-a]azepine-2(7H)-carboxamide

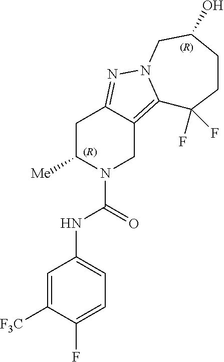

The title compound was prepared in a manner analogous to Example 1, however using ((3R,8R)-tert-butyl 11,11-difluoro-8-hydroxy-3-methyl-3,4,8,9,10,11-hexahydro-1H-pyrido[4′,3′:3,4]pyrazolo[1,5-a]azepine-2(7H)-carboxylate (Intermediate 31) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (4-fluoro-3-(trifluoromethyl)phenyl)carbamate instead of phenyl (3-cyano-4-fluorophenyl)carbamate in Step B. MS (ESI): mass calcd. for C₂₀H₂₀F₆N₄O₂ 462.2; m/z found, 463.1 [M+H]+. ¹H NMR (400 MHz, CDCl₃) δ=7.62-7.46 (m, 2H), 7.06 (t, J=9.4 Hz, 1H), 6.38 (s, 1H), 4.98-4.85 (m, 1H), 4.73 (br d, J=15.3 Hz, 1H), 4.43-4.24 (m, 3H), 4.13 (br m, 1H), 2.94 (dd, J=5.7, 15.9 Hz, 1H), 2.64-2.39 (m, 2H), 2.30-2.01 (m, 3H), 1.13 (d, J=6.8 Hz, 3H).

### Example 70

### (3R,8R)-N-(2-(Difluoromethyl)-3-fluoropyridin-4-yl)-11,11-difluoro-8-hydroxy-3-methyl-3,4,8,9,10,11-hexahydro-1H-pyrido[4′,3′:3,4]pyrazolo[1,5-a]azepine-2(7H)-carboxamide

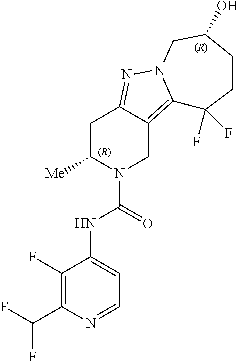

The title compound was prepared in a manner analogous to Example 1, however using ((3R,8R)-tert-butyl 11,11-difluoro-8-hydroxy-3-methyl-3,4,8,9,10,11-hexahydro-1H-pyrido[4′,3′:3,4]pyrazolo[1,5-a]azepine-2(7H)-carboxylate (Intermediate 31) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (2-(difluoromethyl)-3-fluoropyridin-4-yl)carbamate instead of phenyl (3-cyano-4-fluorophenyl)carbamate in Step B. MS (ESI): mass calcd. for C₁₉H₂₀F₅N₅O₂ 445.2; m/z found, 446.1 [M+H]+. ¹H NMR (400 MHz, CDCl₃) δ=8.35-8.32 (m, 2H), 7.00 (br d, J=4.40 Hz, 1H), 6.75 (t, J=53.6 Hz, 1H), 4.97-4.84 (m, 2H), 4.50-4.41 (m, 3H), 4.22 (br m, 1H), 3.05 (dd, J=5.81 Hz, 15.71 Hz, 1H), 2.75-2.47 (m, 2H), 2.39-2.10 (m, 3H), 1.84 (br s, 1H), 1.25 (d, J=6.85 Hz, 3H).

### Example 71

### (3R,8 S)-N-(3-Cyano-4-fluorophenyl)-11,11-difluoro-8-hydroxy-3-methyl-3,4,8,9,10,11-hexahydro-1H-pyrido[4′,3′:3,4]pyrazolo[1,5-a]azepine-2(7H)-carboxamide

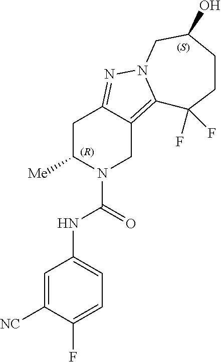

The title compound was prepared in a manner analogous to Example 1, however using (3R,85)-tert-butyl 11,11-difluoro-8-hydroxy-3-methyl-3,4,8,9,10,11-hexahydro-1H-pyrido[4′,3′:3,4]pyrazolo[1,5-a]azepine-2(7H)-carboxylate (Intermediate 32) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A. MS (ESI): mass calcd. for C₂₀H₂₀F₃N₅O₂ 419.2; m/z found, 420.1 [M+H]+. ¹H NMR (400 MHz, CDCl₃) δ=7.74 (dd, J=2.7, 5.4 Hz, 1H), 7.61-7.59 (m, 1H), 7.16 (t, J=8.7 Hz, 1H), 6.55 (s, 1H), 5.02-4.93 (m, 1H), 4.83 (br d, J=15.3 Hz, 1H), 4.49-4.33 (m, 3H), 4.20-4.08 (m, 1H), 3.01-2.96 (m, 1H), 2.74-2.46 (m, 2H), 2.37-2.12 (m, 3H), 1.23 (d, J=6.8 Hz, 3H).

### Example 72

### (3R,8S)-N-(2-Bromo-3-fluoropyridin-4-yl)-11,11-difluoro-8-hydroxy-3-methyl-3,4,8,9,10,11-hexahydro-1H-pyrido[4′,3′:3,4]pyrazolo[1,5-a]azepine-2(7H)-carboxamide

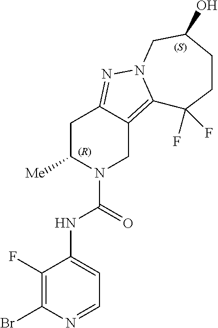

The title compound was prepared in a manner analogous to Example 1, however using (3R,8S)-tert-butyl 11,11-difluoro-8-hydroxy-3-methyl-3,4,8,9,10,11-hexahydro-1H-pyrido[4′,3′:3,4]pyrazolo[1,5-a]azepine-2(7H)-carboxylate (Intermediate 32) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (2-bromo-3-fluoropyridin-4-yl)carbamate instead of phenyl (3-cyano-4-fluorophenyl)carbamate in Step B. MS (ESI): mass calcd. for C₁₈H₁₉BrF₃N₅O₂ 473.1/475.1; m/z found, 474.0/476.0 [M+H]+. ¹H NMR (400 MHz, CDCl₃) δ=8.19 (t, J=5.5 Hz, 1H), 8.09 (d, J=5.5 Hz, 1H), 6.94 (s, 1H), 5.03-4.81 (m, 2H), 4.51-4.35 (m, 3H), 4.18-4.11 (m, 1H), 3.03 (dd, J=5.7, 15.8 Hz, 1H), 2.76-2.50 (m, 2H), 2.38-2.10 (m, 3H), 1.26 (d, J=6.8 Hz, 3H).

### Example 73

### (3R,8S)-11,11-Difluoro-N-(4-fluoro-3-(trifluoromethyl)phenyl)-8-hydroxy-3-methyl-3,4,8,9,10,11-hexahydro-1H-pyrido[4′,3′:3,4]pyrazolo[1,5-a]azepine-2(7H)-carboxamide

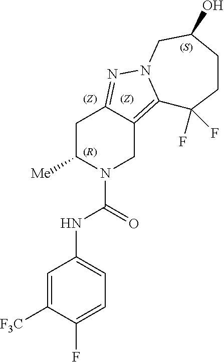

The title compound was prepared in a manner analogous to Example 1, however using (3R,85)-tert-butyl 11,11-difluoro-8-hydroxy-3-methyl-3,4,8,9,10,11-hexahydro-1H-pyrido[4′,3′:3,4]pyrazolo[1,5-a]azepine-2(7H)-carboxylate (Intermediate 32) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (4-fluoro-3-(trifluoromethyl)phenyl)carbamate instead of phenyl (3-cyano-4-fluorophenyl)carbamate in Step B. MS (ESI): mass calcd. for C₂₀H₂₀F₆N₄O₂ 462.2; m/z found, 463.2 [M+H]+. ¹H NMR (400 MHz, CDCl₃) δ=7.64-7.62 (m, 1H), 7.59-7.56 (m, 1H), 7.15-7.12 (m, 1H), 6.48 (s, 1H), 5.05-4.94 (m, 1H), 4.84 (br d, J=15.5 Hz, 1H), 4.47-4.31 (m, 3H), 4.14 (br m, 1H), 3.02 (dd, J=5.7, 15.9 Hz, 1H), 2.75-2.49 (m, 2H), 2.38-2.11 (m, 3H), 1.23 (d, J=6.8 Hz, 3H).

### Example 74

### (3R,8S)-N-(2-(Difluoromethyl)-3-fluoropyridin-4-yl)-11,11-difluoro-8-hydroxy-3-methyl-3,4,8,9,10,11-hexahydro-1H-pyrido[4′,3′:3,4]pyrazolo[1,5-a]azepine-2(7H)-carboxamide

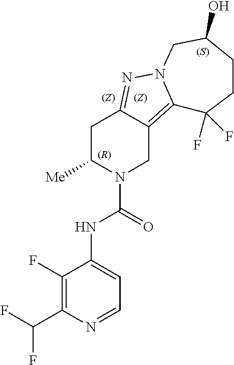

The title compound was prepared in a manner analogous to Example 1, however using (3R,85)-tert-butyl 11,11-difluoro-8-hydroxy-3-methyl-3,4,8,9,10,11-hexahydro-1H-pyrido[4′,3′:3,4]pyrazolo[1,5-a]azepine-2(7H)-carboxylate (Intermediate 32) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (2-(difluoromethyl)-3-fluoropyridin-4-yl) carbamate instead of phenyl (3-cyano-4-fluorophenyl)carbamate in Step B. MS (ESI): mass calcd. for C₁₉H₂₀F₅N₅O₂ 445.2; m/z found, 446.1 [M+H]+. ¹H NMR (400 MHz, CDCl₃) δ=8.39-8.30 (m, 2H), 7.02 (br d, J=4.3 Hz, 1H), 6.77 (t, J=54.0 Hz, 1H), 5.02-4.83 (m, 2H), 4.51-4.36 (m, 3H), 4.14 (br m, 1H), 3.04 (dd, J=5.7 Hz, 15.8 Hz, 1H), 2.77-2.49 (m, 2H), 2.36-2.10 (m, 3H), 1.27 (d, J=6.8 Hz, 3H).

### Example 75

### (3R,8S*)-N-(3-Cyano-4-fluorophenyl)-11,11-difluoro-8-(hydroxymethyl) -3-methyl-3,4,8,9,10,11-hexahydro-1H-pyrido[4′,3′:3,4]pyrazolo[1,5-a]azepine-2(7H)-carboxamide

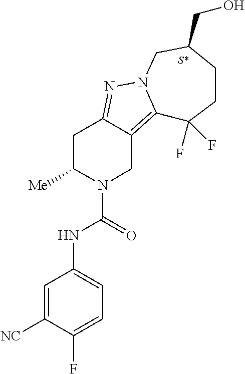

The title compound was prepared in a manner analogous to Example 1, however using (3R,8S*)-tert-butyl 11,11-difluoro-8-(hydroxymethyl)-3-methyl-3,4,8,9,10,11-hexahydro-1H-pyrido[4′,3′:3,4]pyrazolo[1,5-a]azepine-2(7H)-carboxylate (Intermediate 33) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A. MS (ESI): mass calcd. for C₂₁H₂₂F₃N₅O₂ 433.2; m/z found, 434.1 [M+H]+. ¹H NMR (400 MHz, CDCl₃) δ=7.77-7.72 (m, 1H), 7.64-7.58 (m, 1H), 7.15 (t, J=8.7 Hz, 1H), 6.46 (s, 1H), 5.05-4.92 (m, 1H), 4.85-4.76 (m, 1H), 4.55-4.52 (m, 1H), 4.39-4.19 (m, 2H), 3.64-3.44 (m, 2H), 3.03-2.99 (m, 1H), 2.66 (d, J=15.8 Hz, 1H), 2.47-2.22 (m, 2H), 2.17-2.03 (m, 2H), 1.95-1.82 (m, 1H), 1.56-1.50 (m, 1H), 1.21 (d, J=6.8 Hz, 3H).

### Example 76

### (3R,8S*)-11,11-Difluoro-N-(4-fluoro-3-(trifluoromethyl)phenyl)-8-(hydroxymethyl)-3-methyl-3,4,8,9,10,11-hexahydro-1H-pyrido[4′,3′:3,4]pyrazolo[1,5-a]azepine-2(7H)-carboxamide

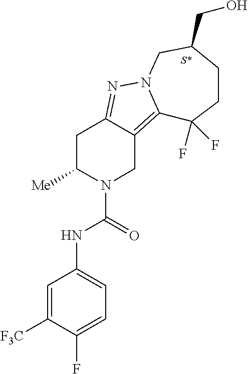

The title compound was prepared in a manner analogous to Example 1, however using (3R,8S*)-tert-butyl 11,11-difluoro-8-(hydroxymethyl)-3-methyl-3,4,8,9,10,11-hexahydro-1H-pyrido[4′,3′:3,4]pyrazolo[1,5-a]azepine-2(7H)-carboxylate (Intermediate 33) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (4-fluoro-3-(trifluoromethyl)phenyl) carbamate instead of phenyl (3-cyano-4-fluorophenyl)carbamate in Step B. MS (ESI): mass calcd. for C₂₁H₂₂F₆N₄O₂ 476.2; m/z found, 477.1 [M+H]+. ¹H NMR (400 MHz, CDCl₃) δ=7.67-7.62 (m, 1H), 7.62-7.56 (m, 1H), 7.18-7.10 (m, 1H), 6.45(s, 1H), 5.05-4.90 (m, 1H), 4.89-4.76 (m, 1H), 4.60-4.47 (m, 1H), 4.41-4.21 (m, 2H), 3.63-3.43 (m, 2H), 3.08-2.95 (m, 1H), 2.66 (d, J=15.9 Hz, 1H), 2.48-2.21 (m, 2H), 2.17-2.05 (m, 2H), 1.93-1.85 (m, 1H), 1.56-1.42 (m, 1H), 1.21 (d, J=6.8 Hz, 3H).

### Example 77

### (3R,8S*)-N-(2-Bromo-3-fluoropyridin-4-yl)-11,11-difluoro-8-(hydroxymethyl)-3-methyl-3,4,8,9,10,11-hexahydro-1H-pyrido[4′,3′:3,4]pyrazolo[1,5-a]azepine-2(7H)-carboxamide

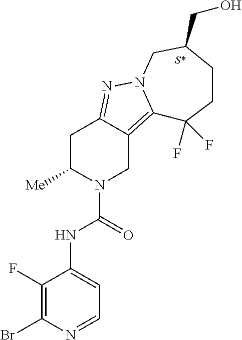

The title compound was prepared in a manner analogous to Example 1, however using (3R,8S*)-tert-butyl 11,11-difluoro-8-(hydroxymethyl)-3-methyl-3,4,8,9,10,11-hexahydro-1H-pyrido[4′,3′:3,4]pyrazolo[1,5-a]azepine-2(7H)-carboxylate (Intermediate 33) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (2-bromo-3-fluoropyridin-4-yl)carbamate instead of phenyl (3-cyano-4-fluorophenyl)carbamate in Step B. MS (ESI): mass calcd. for C₁₉H₂₁BrF₃N₅O₂ 487.1/489.1; m/z found, 488.0/490.0 [M+H]+. ¹H NMR (400 MHz, CDCl₃) δ=8.22-8.19 (m, 1H), 8.11-8.10 (m, 1H), 6.94(d, J=4.0 Hz, 1H), 4.96-4.86 (m, 2H), 4.55 (d, J=10.4 Hz, 1H), 4.39 (d, J=15.6 Hz, 1H), 4.41-4.27 (m, 1H), 3.67-3.42 (m, 2H), 3.09-2.98 (m, 1H), 2.71 (d, J=15.9 Hz, 1H), 2.52-2.28 (m, 1H), 2.07-2.01 (m, 1H), 1.97-1.77 (m, 2H), 1.70-1.41 (m, 2H), 1.24 (d, J=6.8 Hz, 3H).

### Example 78

### (3R,8S*)-N-(2-(Difluoromethyl)-3-fluoropyridin-4-yl)-11,11-difluoro-8-(hydroxymethyl)-3-methyl-3,4,8,9,10,11-hexahydro-1H-pyrido[4′,3′:3,4]pyrazolo[1,5-a]azepine-2 (7H)-carboxamide

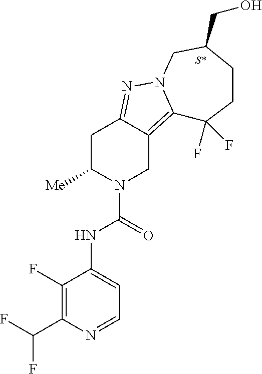

The title compound was prepared in a manner analogous to Example 1, however using (3R,8S*)-tert-butyl 11,11-difluoro-8-(hydroxymethyl)-3-methyl-3,4,8,9,10,11-hexahydro-1H-pyrido[4′,3′:3,4]pyrazolo[1,5-a]azepine-2(7H)-carboxylate (Intermediate 33) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (2-(difluoromethyl)-3-fluoropyridin-4-yl) carbamate instead of phenyl (3-cyano-4-fluorophenyl)carbamate in Step B. MS (ESI): mass calcd. for C₂₀H₂₂F₅N₅O₂ 459.2; m/z found, 460.2 [M+H]+. ¹H NMR (400 MHz, CDCl₃) δ=8.43-8.31 (m, 2H), 7.03-7.00 (m, 1H), 6.78 (t, J=53.6 Hz, 1H), 5.05-4.82 (m, 2H), 4.57-4.54 (m, 1H), 4.46-4.36 (m, 1H), 4.34-4.20 (m, 1H), 3.65-3.44 (m, 2H), 3.14-2.97 (m, 1H), 2.71 (d, J=15.9 Hz, 1H), 2.44-2.12 (m, 5H), 1.96-1.77 (m, 1H), 1.25 (d, J=7.0 Hz, 3H).

### Example 79

### (3R,8R*)-N-(3-Cyano-4-fluorophenyl)-11,11-difluoro-8-(hydroxymethyl) -3-methyl-3,4,8,9,10,11-hexahydro-1H-pyrido[4′,3′:3,4]pyrazolo[1,5-a]azepine-2(7H)-carboxamide

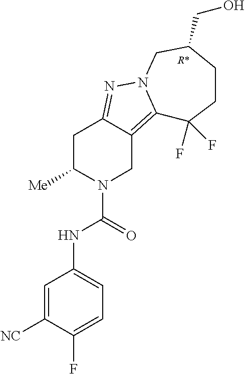

The title compound was prepared in a manner analogous to Example 1, however using (3R,8R*)-tert-butyl 11,11-difluoro-8-(hydroxymethyl)-3-methyl-3,4,8,9,10,11-hexahydro-1H-pyrido[4′,3′:3,4]pyrazolo[1,5-a]azepine-2(7H)-carboxylate (Intermediate 34) instead of (tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A. MS (ESI): mass calcd. for C₂₁H₂₂F₃N₅O₂ 433.2; m/z found, 434.1 [M+H]+. ¹H NMR (400 MHz, CDCl₃) δ=7.74-7.72 (m, 1H), 7.65-7.58 (m, 1H), 7.15 (t, J=8.7 Hz, 1H), 6.45 (s, 1H), 4.98-4.81 (m, 1H), 4.79 (br d, J=15.2 Hz, 1H), 4.56 (br d, J=14.8 Hz, 1H), 4.38 (br d, J=15.5 Hz, 1H), 4.21-4.15 (m, 1H), 3.66-3.57 (m, 1H), 3.56-3.47 (m, 1H), 3.00 (dd, J=5.4, 15.8 Hz, 1H), 2.67 (d, J=16.0 Hz, 1H), 2.48 (br m, 1H), 2.34-2.14 (m, 1H), 2.02 (br m, 2H), 1.89-1.86 (m, 1H), 1.49-1.46(m, 1H), 1.22 (d, J=6.8 Hz, 3H).

### Example 80

### (3R,8R*)-11,11-Difluoro-N-(4-fluoro-3-(trifluoromethyl)phenyl)-8-(hydroxymethyl)-3-methyl-3,4,8,9,10,11-hexahydro-1H-pyrido[4′,3′:3,4]pyrazolo[1,5-a]azepine-2(7H)-carboxamide

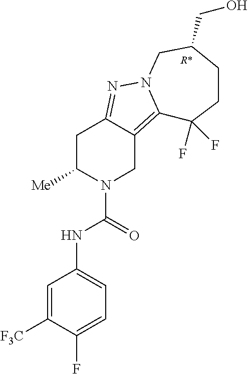

The title compound was prepared in a manner analogous to Example 1, however using (3R,8R*)-tert-butyl 11,11-difluoro-8-(hydroxymethyl)-3-methyl-3,4,8,9,10,11-hexahydro-1H-pyrido[4′,3′:3,4]pyrazolo[1,5-a]azepine-2(7H)-carboxylate (Intermediate 34) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (4-fluoro-3-(trifluoromethyl)phenyl) carbamate instead of phenyl (3-cyano-4-fluorophenyl)carbamate in Step B. MS (ESI): mass calcd. for C₂₁H₂₂F₆N₄O₂ 476.2; m/z found, 477.1 [M+H]+. ¹H NMR (400 MHz, CDCl₃) δ=7.65-7.64 (m, 1H), 7.61-7.58 (m, 1H), 7.14 (t, J=9.4 Hz, 1H), 6.44 (s, 1H), 5.05-4.91 (m, 1H), 4.81 (br d, J=15.0 Hz, 1H), 4.56 (br d, J=14.3 Hz, 1H), 4.37 (br d, J=15.3 Hz, 1H), 4.20-4.20 (m, 1H), 3.67-3.58 (m, 1H), 3.53-3.52 (m, 1H), 3.01 (dd, J=5.9, 15.7 Hz, 1H), 2.67 (d, J=15.7 Hz, 1H), 2.46-2.15 (m, 1H), 2.34-2.14 (m, 1H), 2.02 (br m, 2H), 1.95-1.81 (m, 1H), 1.49-1.46(m, 1H), 1.22 (d, J=6.8 Hz, 3H).

### Example 81

### (3R,8R*)-N-(2-Bromo-3-fluoropyridin-4-yl)-11,11-difluoro-8-(hydroxymethyl)-3-methyl-3,4,8,9,10,11-hexahydro-1H-pyrido[4′,3′:3,4]pyrazolo[1,5-a]azepine-2(7H)-carboxamide

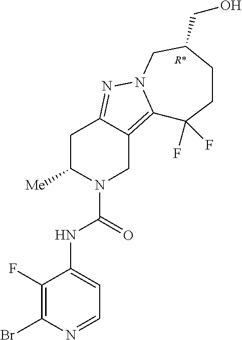

The title compound was prepared in a manner analogous to Example 1, however using (3R,8R*)-tert-butyl 11,11-difluoro-8-(hydroxymethyl)-3-methyl-3,4,8,9,10,11-hexahydro-1H-pyrido[4′,3′:3,4]pyrazolo[1,5-a]azepine-2(7H)-carboxylate (Intermediate 34) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (2-bromo-3-fluoropyridin-4-yl)carbamate instead of phenyl (3-cyano-4-fluorophenyl)carbamate in Step B. MS (ESI): mass calcd. for C₁₉H₂₁BrF₃N₅O₂ 487.1/489.1; m/z found, 488.0/490.0 [M+H]+. ¹H NMR (400 MHz, CDCl₃) δ=8.11 (t, J=5.5 Hz, 1H), 8.00 (d, J=5.5 Hz, 1H), 6.85 (d, J=4.3 Hz, 1H), 4.95-4.70 (m, 2H), 4.49 (br d, J=14.2 Hz, 1H), 4.34 (br d, J=15.2 Hz, 1H), 4.12-4.08 (m, 1H), 3.56-3.53 (m, 1H), 3.47-3.44 (m, 1H), 2.94 (dd, J=5.9, 15.9 Hz, 1H), 2.62 (d, J=15.7 Hz, 1H), 2.50-2.33 (m, 1H), 2.25-2.07 (m, 1H), 2.01-1.90 (m, 2H), 1.87-1.74 (m, 1H), 1.49-1.44 (m, 1H), 1.17 (d, J=6.8 Hz, 3H).

### Example 82

### (3R,8R*)-N-(2-(Difluoromethyl)-3-fluoropyridin-4-yl)-11,11-difluoro-8-(hydroxymethyl)-3-methyl-3,4,8,9,10,11-hexahydro-1H-pyrido[4′,3′:3,4]pyrazolo[1,5-a]azepine-2 (7H)-carboxamide

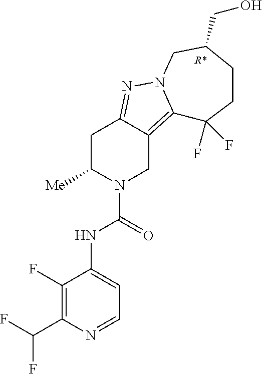

The title compound was prepared in a manner analogous to Example 1, however using (3R,8R*)-tert-butyl 11,11-difluoro-8-(hydroxymethyl)-3-methyl-3,4,8,9,10,11-hexahydro-1H-pyrido[4′,3′:3,4]pyrazolo[1,5-a]azepine-2(7H)-carboxylate (Intermediate 34) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (2-(difluoromethyl)-3-fluoropyridin-4-yl) carbamate instead of phenyl (3-cyano-4-fluorophenyl)carbamate in Step B. MS (ESI): mass calcd. for C₂₀H₂₂F₅N₅O₂ 459.2; m/z found, 460.1 [M+H]+. ¹H NMR (400 MHz, CDCl₃) δ=8.40-8.24 (m, 2H), 6.98 (d, J=3.8 Hz, 1H), 6.75 (t, J=53.6Hz, 1H), 5.01-4.75 (m, 2H), 4.54 (br d, J=14.2 Hz, 1H), 4.40 (br d, J=15.4 Hz, 1H), 4.21-4.08 (m, 1H), 3.63-3.50 (m, 2H), 2.99 (dd, J=5.7, 15.8 Hz, 1H), 2.68 (d, J=15.7 Hz, 1H), 2.49-2.44 (m, 1H), 2.33-2.12 (m, 1H), 2.09-1.95 (m, 2H), 1.91-1.81 (m, 1H), 1.49-1.44 (m, 1H), 1.23 (d, J=6.8 Hz, 3H).

### Example 83

### (3R,8R*)-N-(3-Cyano-4-fluorophenyl)-11,11-difluoro-8-(fluoromethyl)-8-hydroxy-3-methyl-3,4, 8,9,10,11-hexahydro-1H-pyrido[4′,3′:3,4]pyrazolo[1,5-a]azepine-2(7H)-carboxamide

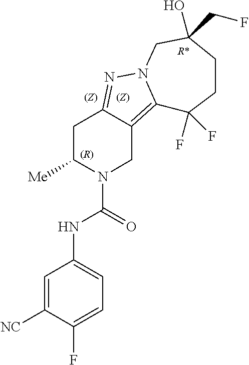

The title compound was prepared in a manner analogous to Example 1, however using (3R,8R*)-tert-butyl 11,11-difluoro-8-(fluoromethyl)-8-hydroxy-3-methyl-3,4,8,9,10,11-hexahydro-1H-pyrido[4′,3′:3,4]pyrazolo[1,5-a]azepine-2(7H)-carboxylate (Intermediate 35) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A. MS (ESI): mass calcd. for C₂₁H₂₁F₄N₅O₂ 451.2; m/z found, 452.2 [M+H]+. ¹H NMR (400 MHz, CDCl₃) δ=7.74 (dd, J=2.8, 5.4 Hz, 1H), 7.62-7.60 (m, 1H), 7.15 (t, J=8.7 Hz, 1H), 6.48 (s, 1H), 5.05-4.92 (m, 1H), 4.84 (br d, J=15.3 Hz, 1H), 4.51 (d, J=14.7 Hz, 1H), 4.41-4.30 (m, 3H), 4.24 (q, J=9.7 Hz, 1H), 3.01 (dd, J=5.6, 15.8 Hz, 1H), 2.74-2.50 (m, 2H), 2.45-2.18 (m, 2H), 2.12-1.87 (m, 2H), 1.21 (d, J=7.0 Hz, 3H).

### Example 84

### (3R,8R*)-N-(2-Bromo-3-fluoropyridin-4-yl)-11,11-difluoro-8-(fluoromethyl)-8-hydroxy-3-methyl-3,4,8,9,10,11-hexahydro-1H-pyrido[4′,3′:3,4]pyrazolo[1,5-a]azepine-2(7H)-carboxamide

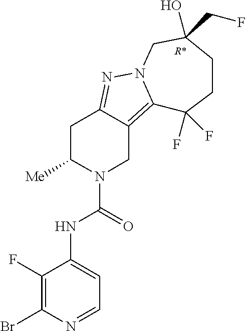

The title compound was prepared in a manner analogous to Example 1, however using (3R,8R*)-tert-butyl 11,11-difluoro-8-(fluoromethyl)-8-hydroxy-3-methyl-3,4,8,9,10,11-hexahydro-1H-pyrido[4′,3′:3,4]pyrazolo[1,5-a]azepine-2(7H)-carboxylate (Intermediate 35) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (2-bromo-3-fluoropyridin-4-yl)carbamate instead of phenyl (3-cyano-4-fluorophenyl)carbamate in Step B. MS (ESI): mass calcd. for C₁₉H₂₀BrF₄N₅O₂ 505.1/507.1; m/z found, 506.1/508.1 [M+H]+. ¹H NMR (400 MHz, CDCl₃) δ=8.24-8.14 (m, 1H), 8.08 (d, J=5.5 Hz, 1H), 6.92 (d, J=3.8 Hz, 1H), 5.04-4.81 (m, 2H), 4.51 (d, J=14.5 Hz, 1H), 4.44-4.30 (m, 3H), 4.24 (q, J=9.5 Hz, 1H), 3.03 (dd, J=5.6, 15.7 Hz, 1H), 2.79-2.48 (m, 2H), 2.43-2.20 (m, 2H), 2.12-1.86 (m, 2H), 1.25 (d, J=6.8 Hz, 3H).

### Example 85

### (3R,8R*)-N-(2-(Difluoromethyl)-3-fluoropyridin-4-yl)-11,11-difluoro-8-(fluoromethyl)-8-hydroxy-3-methyl-3,4,8,9,10,11-hexahydro-1H-pyrido[4′,3′:3,4]-pyrazolo[1,5-a]azepine-2(7H)-carboxamide

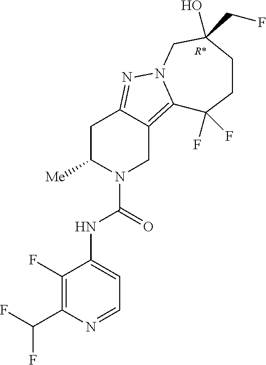

The title compound was prepared in a manner analogous to Example 1, however using (3R,8R*)-tert-butyl 11,11-difluoro-8-(fluoromethyl)-8-hydroxy-3-methyl-3,4,8,9,10,11-hexahydro-1H-pyrido[4′,3′:3,4]pyrazolo[1,5-a]azepine-2(7H)-carboxylate (Intermediate 35) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (2-(difluoromethyl)-3-fluoropyridin-4-yl) carbamate instead of phenyl (3-cyano-4-fluorophenyl)carbamate in Step B. MS (ESI): mass calcd. for C₂₀H₂₁F₆N₅O₂ 477.2; m/z found, 478.2 [M+H]+. ¹H NMR (400 MHz, CDCl₃) δ=8.40-8.29 (m, 2H), 7.01 (d, J=4.2 Hz, 1H), 6.65 (t, J=53.6 Hz, 1H), 5.05-4.84 (m, 2H), 4.51 (d, J=14.7 Hz, 1H), 4.45-4.31 (m, 3H), 4.24 (q, J=9.6 Hz, 1H), 3.04 (dd, J=5.9, 15.8 Hz, 1H), 2.75-2.50 (m, 2H), 2.46-2.18 (m, 2H), 2.10-1.89 (m, 2H), 1.25 (d, J=6.8 Hz, 3H).

### Example 86

### (3R,8S*)-N-(3-Cyano-4-fluorophenyl)-11,11-difluoro-8-(fluoromethyl)-8-hydroxy-3-methyl-3,4,8,9,10,11-hexahydro-1H-pyrido[4′,3′:3,4]pyrazolo[1,5-a]azepine-2(7H)-carboxamide

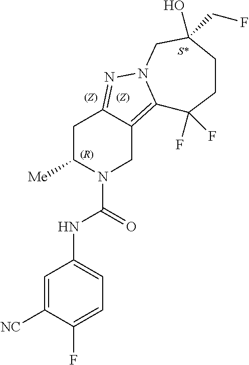

The title compound was prepared in a manner analogous to Example 1, however using (3R,8S*)-tert-butyl 11,11-difluoro-8-(fluoromethyl)-8-hydroxy-3-methyl-3,4,8,9,10,11-hexahydro-1H-pyrido[4′,3′:3,4]pyrazolo[1,5-a]azepine-2(7H)-carboxylate (Intermediate 36) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A. MS (ESI): mass calcd. for C₂₁H₂₁F₄N₅O₂ 451.2; m/z found, 452.1 [M+H]+. ¹H NMR (400 MHz, CDCl₃) δ=7.76-7.68 (m, 1H), 7.62-7.59 (m, 1H), 7.15 (t, J=8.7 Hz, 1H), 6.47 (s, 1H), 4.96 (t, J=6.1 Hz, 1H), 4.80 (d, J=15.8 Hz, 1H), 4.54 (d, J=14.8 Hz, 1H), 4.45-4.31 (m, 3H), 4.30-4.19 (m, 1H), 3.00 (dd, J=5.9, 15.8 Hz, 1H), 2.72-2.49 (m, 2H), 2.46-2.27 (m, 2H), 2.12-1.84 (m, 2H), 1.22 (d, J=7.0 Hz, 3H).

### Example 87

### (3R,8S*)-N-(2-Bromo-3-fluoropyridin-4-yl)-11,11-difluoro-8-(fluoromethyl)-8-hydroxy-3-methyl-3,4,8,9,10,11-hexahydro-1H-pyrido[4′,3′:3,4]pyrazolo[1,5-a]azepine-2(7H)-carboxamide

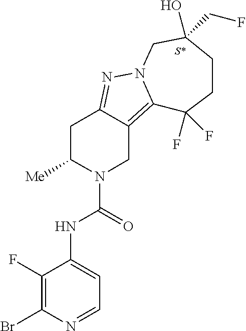

The title compound was prepared in a manner analogous to Example 1, however using (3R,8S*)-tert-butyl 11,11-difluoro-8-(fluoromethyl)-8-hydroxy-3-methyl-3,4,8,9,10,11-hexahydro-1H-pyrido[4′,3′:3,4]pyrazolo[1,5-a]azepine-2(7H)-carboxylate (Intermediate 36) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (2-bromo-3-fluoropyridin-4-yl)carbamate instead of phenyl (3-cyano-4-fluorophenyl)carbamate in Step B. MS (ESI): mass calcd. for C₁₉H₂₀BrF₄N₅O₂ 505.1/507.1; m/z found, 506.1/508.1 [M+H]+. ¹H NMR (400 MHz, CDCl₃) δ=8.22-8.14 (m, 1H), 8.08 (d, J=5.5 Hz, 1H), 6.91 (d, J=3.7 Hz, 1H), 5.07-4.77 (m, 2H), 4.54 (d, J=14.4 Hz, 1H), 4.48-4.31 (m, 3H), 4.30-4.20 (m, 1H), 3.12-2.94 (m, 1H), 2.76-2.48 (m, 2H), 2.40-2.25 (m, 1H), 2.26 (br s, 1H), 2.12-1.85 (m, 2H), 1.25 (d, J=6.8 Hz, 3H).

### Example 88

### (3R,8S*)-N-(2-(Difluoromethyl)-3-fluoropyridin-4-yl)-11,11-difluoro-8-(fluoromethyl)-8-hydroxy-3-methyl-3,4,8,9,10,11-hexahydro-1H-pyrido[4′,3′:3,4]pyrazolo[1,5-a]azepine-2(7H)-carboxamide

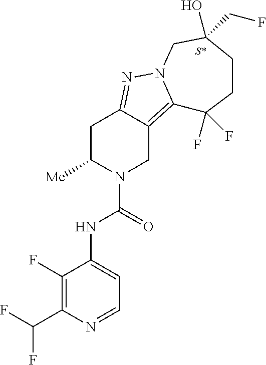

The title compound was prepared in a manner analogous to Example 1, however using (3R,8S*)-tert-butyl 11,11-difluoro-8-(fluoromethyl)-8-hydroxy-3-methyl-3,4,8,9,10,11-hexahydro-1H-pyrido[4′,3′:3,4]pyrazolo[1,5-a]azepine-2(7H)-carboxylate (Intermediate 36) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (2-(difluoromethyl)-3-fluoropyridin-4-yl) carbamate instead of phenyl (3-cyano-4-fluorophenyl)carbamate in Step B. MS (ESI): mass calcd. for C₂₀H₂₁F₆N₅O₂ 477.2; m/z found, 478.2 [M+H]+. ¹H NMR (400 MHz, CDCl₃) δ=8.44-8.16 (m, 2H), 7.00 (d, J=4.8 Hz, 1H), 6.74 (t, J=53.6 H, 1H), 5.10-4.77 (m, 2H), 4.54 (d, J=14.8 Hz, 1H), 4.48-4.31 (m, 3H), 4.30-4.21 (m, 1H), 3.11-2.94 (m, 1H), 2.76-2.47 (m, 2H), 2.46-2.31 (m, 1H), 2.28 (br s, 1H), 2.11-1.80 (m, 2H), 1.26 (d, J=6.8 Hz, 3H).

### Example 89

### (3R,8S*)-N-(2-Bromo-3-fluoropyridin-4-yl)-8-(cyanomethyl)-11,11-difluoro-8-hydroxy-3-methyl-3,4,8,9,10,11-hexahydro-1H-pyrido[4′,3′:3,4]pyrazolo[1,5-a]azepine-2(7H)-carboxamide

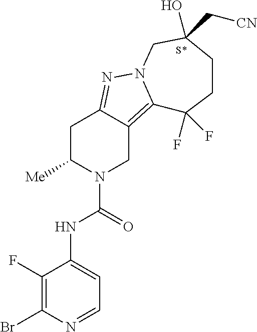 [SMILES: C[C@@H]1Cc2nn3c(c2CN1C(=O)Nc1ccnc(Br)c1F)C(F)(F)CC[C@](O)(CC#N)C3]

The title compound was prepared in a manner analogous to Example 1, however using (3R,8S*)-tert-butyl 8-(cyanomethyl)-11,11-difluoro-8-hydroxy-3-methyl-3,4,8,9,10,11-hexahydro-1H-pyrido[4′,3′:3,4]pyrazolo[1,5-a]azepine-2(7H)-carboxylate (Intermediate 37) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (2-bromo-3-fluoropyridin-4-yl)carbamate instead of phenyl (3-cyano-4-fluorophenyl)carbamate in Step B. MS (ESI): mass calcd. for C₂₀H₂₀BrF₃N₆O₂ 512.1/514.1; m/z found, 513.1/515.1 [M+H]+. ¹H NMR (400 MHz, CDCl₃) δ=8.14 (t, J=5.4 Hz, 1H), 8.11-8.02 (m, 1H), 6.94 (d, J=3.7 Hz, 1H), 5.03-4.77 (m, 2H), 4.60-4.33 (m, 3H), 2.99-2.93 (m, 1H), 2.82 (br s, 1H), 2.75-2.33 (m, 5H), 2.29-2.13 (m, 2H), 1.26 (d, J=6.8 Hz, 3H).

### Example 90

### (3R,8S*)-8-(Cyanomethyl)-N-(2-(difluoromethyl)-3-fluoropyridin-4-yl)-11,11-difluoro-8-hydroxy-3-methyl-3,4,8,9,10,11-hexahydro-1H-pyrido[4′,3′:3,4]pyrazolo[1,5-a]azepine-2 (7H)-carboxamide

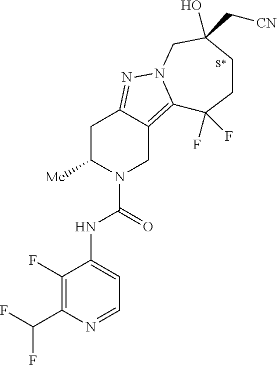 [SMILES: C[C@@H]1Cc2nn3c(c2CN1C(=O)Nc1ccnc(C(F)F)c1F)C(F)(F)CC[C@](O)(CC#N)C3]

The title compound was prepared in a manner analogous to Example 1, however using (3R,8S*)-tert-butyl 8-(cyanomethyl)-11,11-difluoro-8-hydroxy-3-methyl-3,4,8,9,10,11-hexahydro-1H-pyrido[4′,3′:3,4]pyrazolo[1,5-a]azepine-2(7H)-carboxylate (Intermediate 37) instead of tert-butyl 11,11-difluoro-8-methylene-3 ,4,8,9,10,11-hexahydro-1H-pyrido[4′,3′:3,4]pyrazolo[1,5-a]azepine-2(7H)-carboxylate (Intermediate 1) in Step A and phenyl (2-(difluoromethyl)-3-fluoropyridin-4-yl) carbamate instead of phenyl (3-cyano-4-fluorophenyl)carbamate in Step B. MS (ESI): mass calcd. for C₂₁H₂₁F₆N₆O₂ 484.2; m/z found, 485.2 [M+H]+. ¹H NMR (400 MHz, CDCl₃) δ=8.30-8.26 (m, 2H), 7.01-7.00 (m, 1H), 6.75 (t, J=53.6 Hz, 1H), 4.92-4.84 (m, 2H), 4.54-4.50 (m, 1H), 4.45-4.38 (m, 2H), 2.92-2.91 (m, 1H), 2.71-2.59 (m, 5H), 2.24-2.18 (m, 2H), 1.24 (d, J=6.8 Hz, 3H).

### Example 91

### (3R,8R*)-N-(2-Bromo-3-fluoropyridin-4-yl)-8-(cyanomethyl)-11,11-difluoro-8-hydroxy-3-methyl-3,4,8,9,10,11-hexahydro-1H-pyrido[4′,3′:3,4]pyrazolo[1,5-a]azepine-2(7H)-carboxamide

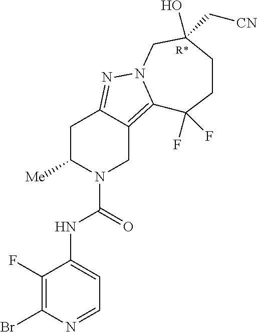 [SMILES: C[C@@H]1Cc2nn3c(c2CN1C(=O)Nc1ccnc(Br)c1F)C(F)(F)CC[C@@](O)(CC#N)C3]

The title compound was prepared in a manner analogous to Example 1, however using (3R,8R*)-tert-butyl 8-(cyanomethyl)-11,11-difluoro-8-hydroxy-3-methyl-3,4,8,9,10,11-hexahydro-1H-pyrido[4′,3′:3,4]pyrazolo[1,5-a]azepine-2(7H)-carboxylate (Intermediate 38) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (2-bromo-3-fluoropyridin-4-yl)carbamate instead of phenyl (3-cyano-4-fluorophenyl)carbamate in Step B. MS (ESI): mass calcd. for C₂₀H₂₀BrF₃N₆O₂ 512.1/514.1; m/z found, 513.1/515.1 [M+H]+. ¹H NMR (400 MHz, CDCl₃) δ=8.21-8.14 (m, 1H), 8.08 (d, J=5.5 Hz, 1H), 6.92 (d, J=3.6 Hz, 1H), 4.99-4.82 (m, 2H), 4.52-4.32 (m, 3H), 3.05-2.99 (m, 1H), 2.74-2.43 (m, 4H), 2.39-2.19 (m, 3H), 1.24 (d, J=6.8 Hz, 3H).

### Example 92

### (3R,8R*)-8-(Cyanomethyl)-N-(2-(difluoromethyl)-3-fluoropyridin-4-yl) -11,11-difluoro-8-hydroxy-3-methyl-3,4,8,9,10,11-hexahydro-1H-pyrido[4′,3′:3,4]pyrazolo[1,5-a]azepine-2 (7H)-carboxamide

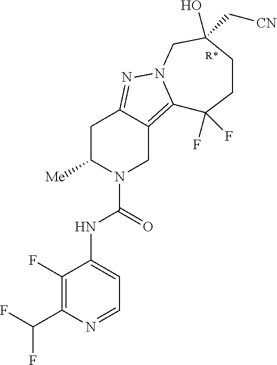 [SMILES: C[C@@H]1Cc2nn3c(c2CN1C(=O)Nc1ccnc(C(F)F)c1F)C(F)(F)CC[C@@](O)(CC#N)C3]

The title compound was prepared in a manner analogous to Example 1, however using (3R,8R*)-tert-butyl 8-(cyanomethyl)-11,11-difluoro-8-hydroxy-3-methyl-3,4,8,9,10,11-hexahydro-1H-pyrido[4′,3′:3,4]pyrazolo[1,5-a]azepine-2(7H)-carboxylate (Intermediate 38) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (2-(difluoromethyl)-3-fluoropyridin-4-yl) carbamate instead of phenyl (3-cyano-4-fluorophenyl)carbamate in Step B. MS (ESI): mass calcd. for C₂₁H₂₁F₆N₆O₂ 484.2; m/z found, 485.2 [M+H]+. ¹H NMR (400 MHz, CDCl₃) δ=8.35-8.31 (m, 2H), 7.00 (d, J=4.4 Hz, 1H), 6.75 (t, J=53.6 Hz, 1H), 5.04-4.81 (m, 2H), 4.54-4.25 (m, 3H), 3.03 (dd, J=5.9, 15.9 Hz, 1H), 2.78-2.42 (m, 5H), 2.30-2.18 (m, 3H), 1.25 (d, J=6.8 Hz, 3H).

### Example 93

### (3R,8R*)-N-(3-Cyano-4-fluorophenyl)-8-((2,2-difluoroethoxy)methyl)-11,11-difluoro-8-hydroxy-3-methyl-3,4,8,9,10,11-hexahydro-1H-pyrido[4′,3′:3,4]pyrazolo[1,5-a]azepine-2 (7H)-carboxamide

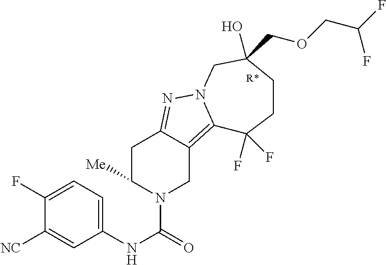 [SMILES: C[C@@H]1Cc2nn3c(c2CN1C(=O)Nc1ccc(F)c(C#N)c1)C(F)(F)CC[C@](O)(COCC(F)F)C3]

The title compound was prepared in a manner analogous to Example 1, however using (3R,8R*)-tert-butyl 8-((2,2-difluoroethoxy)methyl)-11,11-difluoro-8-hydroxy-3-methyl-3,4,8,9,10,11-hexahydro-1H-pyrido[4′,3′:3,4]pyrazolo[1,5-a]azepine-2(7H)-carboxylate (Intermediate 39) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A. MS (ESI): mass calcd. for C₂₃H₂₄F₅N₅O₃ 513.2; m/z found, 514.2 [M+H]+. ¹H NMR (400 MHz, CDCl₃) δ=7.75-7.73 (m, 1H), 7.62-7.60 (m, 1H), 7.15 (t, J=8.7 Hz, 1H), 6.47 (s, 1H), 6.06-5.74 (m, 1H), 5.01-4.80 (m, 1H), 4.83 (d, J=15.4 Hz, 1H), 4.51 (d, J=14.4 Hz, 1H), 4.39-4.24 (m, 2H), 3.76 (dt, J=3.9, 13.8 Hz, 2H), 3.50-3.40 (m, 2H), 3.01 (dd, J=5.9, 15.8 Hz, 1H), 2.67 (d, J=15.9 Hz, 1H), 2.64-2.44 (m, 2H), 2.38-2.21 (m, 1H), 2.06-1.94 (m, 2H), 1.21 (d, J=6.8 Hz, 3H).

### Example 94

### (3R,8S*)-N-(3-Cyano-4-fluorophenyl)-8-((2,2-difluoroethoxy)methyl)-11,11-difluoro-8-hydroxy-3-methyl-3,4,8,9,10,11-hexahydro-1H-pyrido[4′,3′:3,4]pyrazolo[1,5-a]azepine-2(7H)-carboxamide

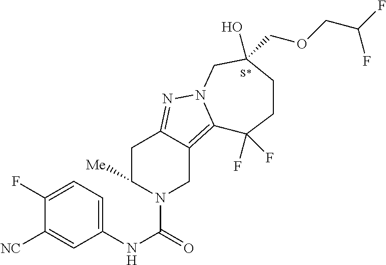 [SMILES: C[C@@H]1Cc2nn3c(c2CN1C(=O)Nc1ccc(F)c(C#N)c1)C(F)(F)CC[C@@](O)(COCC(F)F)C3]

The title compound was prepared in a manner analogous to Example 1, however using (3R,8S*)-tert-butyl 8-((2,2-difluoroethoxy)methyl)-11,11-difluoro-8-hydroxy-3-methyl-3,4,8,9,10,11-hexahydro-1H-pyrido[4′,3′:3,4]pyrazolo[1,5-a]azepine-2(7H)-carboxylate (Intermediate 40) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A. MS (ESI): mass calcd. for C₂₃H₂₄F₅N₅O₃ 513.2; m/z found, 514.2 [M+H]+. ¹H NMR (400 MHz, CDCl₃) δ=7.74-7.69 (m, 1H), 7.64-7.58 (m, 1H), 7.15 (t, J=8.7 Hz, 1H), 6.43 (s, 1H), 6.17-5.67 (m, 1H), 5.01-4.98 (m, 1H), 4.79-4.77 (m, 1H), 4.54 (d, J=14.7 Hz, 1H), 4.45-4.41 (m, 1H), 4.26 (d, J=14.4 Hz, 1H), 3.84-3.72 (m, 2H), 3.58-3.43 (m, 2H), 3.07-2.98 (m, 1H), 2.71-2.25 (m, 4H), 2.08-1.87 (m, 2H), 1.22 (d, J=7.0 Hz, 3H).

### Example 95

### (3R,8R*)-N-(3-Cyano-4-fluorophenyl)-8-ethynyl-11,11-difluoro-8-hydroxy-3-methyl-3,4,8,9,10,11-hexahydro-1H-pyrido[4′,3′:3,4]pyrazolo[1,5-a]azepine-2(7H)-carboxamide

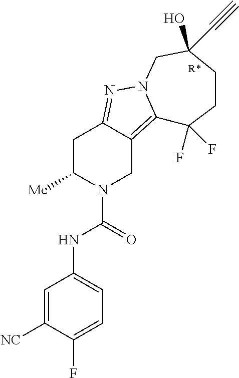 [SMILES: C#C[C@]1(O)CCC(F)(F)c2c3c(nn2C1)C[C@@H](C)N(C(=O)Nc1ccc(F)c(C#N)c1)C3]

The title compound was prepared in a manner analogous to Example 1, however using (3R,8R*)-tert-butyl 8-ethynyl-11,11-di fluoro-8-hydroxy-3-methyl-3,4,8,9,10,11-hexahydro-1H-pyrido[4′,3′:3,4]pyrazolo[1,5-a]azepine-2(7H)-carboxylate (Intermediate 41) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A, and purification via RP HPLC(condition D). MS (ESI): mass calcd. for C₂₂H₂₀F₃N₅O₂ 443.2; m/z found, 444.2 [M+H]+. ¹H NMR (400 MHz, CDCl₃) δ=7.74 (dd, J=2.8, 5.4 Hz, 1H), 7.64-7.61 (m, 1H), 7.16 (t, J=8.7 Hz, 1H), 6.49 (s, 1H), 5.0-4.95 (m, 1H), 4.82 (d, J=15.3 Hz, 1H), 4.59-4.46 (m, 2H), 4.36 (d, J=15.0 Hz, 1H), 3.02 (dd, J=5.8, 15.8 Hz, 1H), 2.90 (s, 1H), 2.69 (d, J=15.7 Hz, 1H), 2.61-2.49 (m, 2H), 2.46-2.27 (m, 3H), 1.21 (d, J=6.8 Hz, 3H).

### Example 96

### (3R,8R*)-N-(2-Bromo-3-fluoropyridin-4-yl)-8-ethynyl-11,11-difluoro-8-hydroxy-3-methyl-3,4, 8,9,10,11-hexahydro-1H-pyrido[4′,3′:3,4]pyrazolo[1,5-a]azepine-2(7H)-carboxamide

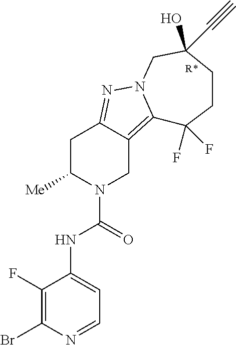 [SMILES: C#C[C@]1(O)CCC(F)(F)c2c3c(nn2C1)C[C@@H](C)N(C(=O)Nc1ccnc(Br)c1F)C3]

The title compound was prepared in a manner analogous to Example 1, however using (3R,8R*)-tert-butyl 8-ethynyl-11,11-difluoro-8-hydroxy-3-methyl-3,4,8,9,10,11-hexahydro-1H-pyrido[4′,3′:3,4]pyrazolo[1,5-a]azepine-2(7H)-carboxylate (Intermediate 41) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (2-bromo-3-fluoropyridin-4-yl)carbamate instead of phenyl (3-cyano-4-fluorophenyl)carbamate in Step B. the title compound was purified via RP HPLC(condition D). MS (ESI): mass calcd. for C₂₀H₁₉BrF₃N₅O₂ 497.1/499.1; m/z found, 498.1/500.0 [M+H]+. ¹H NMR (400 MHz, CDCl₃) δ=8.21-8.16 (m, 1H), 8.08 (d, J=5.5 Hz, 1H), 6.92 (d, J=3.8 Hz, 1H), 5.01-4.81 (m, 2H), 4.57-4.36 (m, 3H), 3.06-3.01 (m, 1H), 2.77-2.61 (m, 2H), 2.59-2.46 (m, 2H), 2.43-2.26 (m, 3H), 1.22 (d, J=7.2 Hz, 3H).

### Example 97

### (3R,8R*)-N-(2-(Difluoromethyl)-3-fluoropyridin-4-yl)-8-ethynyl-11,11-difluoro-8-hydroxy-3-methyl-3,4,8,9,10,11-hexahydro-1H-pyrido[4′,3′:3,4]pyrazolo[1,5-a]azepine-2(7H)-carboxamide

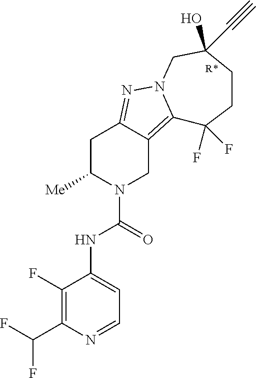 [SMILES: C#C[C@]1(O)CCC(F)(F)c2c3c(nn2C1)C[C@@H](C)N(C(=O)Nc1ccnc(C(F)F)c1F)C3]

The title compound was prepared in a manner analogous to Example 1, however using (3R,8R*)-tert-butyl 8-ethynyl-11,11-difluoro-8-hydroxy-3-methyl-3,4,8,9,10,11-hexahydro-1H-pyrido[4′,3′:3,4]pyrazolo[1,5-a]azepine-2(7H)-carboxylate (Intermediate 41) instead of tert-butyl 11,11-difluoro-8-methylene-3,4,8,9,10,11-hexahydro-1H-pyrido[4′,3′:3,4]pyrazolo-[1,5-a]azepine-2(7H)-carboxylate (Intermediate 1) in Step A and phenyl (2-(difluoromethyl)-3-fluoropyridin-4-yl) carbamate instead of phenyl (3-cyano-4-fluorophenyl)carbamate in Step B, purification via RP HPLC(condition D). MS (ESI): mass calcd. for C₂₁H₂₀F₅N₅O₂ 469.2; m/z found, 470.1 [M+H]+. ¹H NMR (400 MHz, CDCl₃) δ=8.38-8.30 (m, 2H), 7.04-6.98 (m, 1H), 6.75 (t, J=53.6 Hz, 1H), 5.03-4.83 (m, 2H), 4.57-4.40 (m, 3H), 3.05-3.01 (m, 1H), 2.76-2.67 (m, 2H), 2.52-2.24 (m, 5H), 1.25 (d, J=6.8 Hz, 3H).

### Example 98

### (3R,8S*)-N-(3-Cyano-4-fluorophenyl)-8-ethynyl-11,11-difluoro-8-hydroxy-3-methyl-3,4,8,9,10,11-hexahydro-1H-pyrido[4′,3′:3,4]pyrazolo[1,5-a]azepine-2(7H)-carboxamide

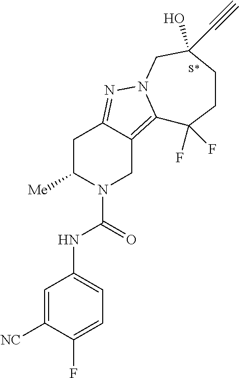 [SMILES: C#C[C@@]1(O)CCC(F)(F)c2c3c(nn2C1)C[C@@H](C)N(C(=O)Nc1ccc(F)c(C#N)c1)C3]

The title compound was prepared in a manner analogous to Example 1, however using (3R,8S*)-tert-butyl 8-ethynyl-11,11-difluoro-8-hydroxy-3-methyl-3,4,8,9,10,11-hexahydro-1H-pyrido[4′,3′:3,4]pyrazolo[1,5-a]azepine-2(7H)-carboxylate (Intermediate 42) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A, and purification via RP HPLC(condition E). MS (ESI): mass calcd. for C₂₂H₂₀F₃N₅O₂ 443.2; m/z found, 444.2 [M+H]+. ¹H NMR (400 MHz, DMSO-d₆) δ=9.01 (s, 1H), 7.96-7.92 (m, 1H), 7.78-7.76 (m, 1H), 7.43 (t, J=9.2 Hz, 1H), 6.25 (s, 1H), 4.97 (d, J=16.4 Hz, 1H), 4.89-4.76 (m, 1H), 4.43-4.19 (m, 2H), 4.10 (d, J=16.0 Hz, 1H), 3.48 (s, 1H), 2.87-2.82 (m, 1H), 2.57 (d, J=15.6 Hz, 1H), 2.41-2.25 (m, 2H), 2.20-2.09 (m, 2H), 1.09 (d, J=6.8 Hz, 3H).

### Example 99

### (3R,8S*)-N-(2-Bromo-3-fluoropyridin-4-yl)-8-ethynyl-11,11-difluoro-8-hydroxy-3-methyl-3,4,8,9,10,11-hexahydro-1H-pyrido[4′,3′:3,4]pyrazolo[1,5-a]azepine-2(7H)-carboxamide

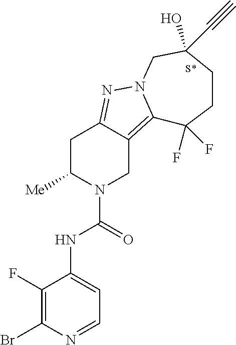 [SMILES: C#C[C@@]1(O)CCC(F)(F)c2c3c(nn2C1)C[C@@H](C)N(C(=O)Nc1ccnc(Br)c1F)C3]

The title compound was prepared in a manner analogous to Example 1, however using (3R,8S*)-tert-butyl 8-ethynyl-11,11-difluoro-8-hydroxy-3-methyl-3,4,8,9,10,11-hexahydro-1H-pyrido[4′,3′:3,4]pyrazolo[1,5-a]azepine-2(7H)-carboxylate (Intermediate 42) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (2-bromo-3-fluoropyridin-4-yl)carbamate instead of phenyl (3-cyano-4-fluorophenyl)carbamate in Step B and purification via RP HPLC(condition E). MS (ESI): mass calcd. for C₂₀H₁₉BrF₃N₅O₂ 497.1/499.1; m/z found, 498.1/500.0 [M+H]+. ¹H NMR (400 MHz, CDCl₃) δ=8.21-8.02 (m, 2H), 6.91 (s, 1H), 5.01-4.75 (m, 2H), 4.54(s, 2H), 4.41(d, J=14.8 Hz, 1H), 3.63-3.32 (m, 1H), 3.04-2.98 (m, 1H), 2.72 (d, J=16.0 Hz, 1H), 2.51-2.27 (m, 5H), 1.21 (d, J=6.8 Hz, 3H).

### Example 100

### (3R,8S*)-N-(2-Difluoromethyl)-3-fluoropyridin-4-yl)-8-ethynyl-11,11-difluoro-8-hydroxy-3-methyl-3,4,8,9,10,11-hexahydro-1H-pyrido[4′,3′:3,4]pyrazolo[1,5-a]azepine-2(7H)-carboxamide

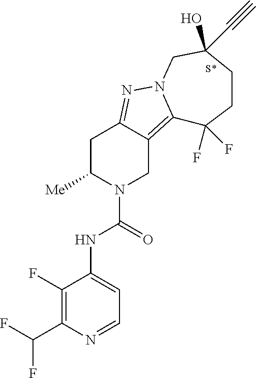 [SMILES: C#C[C@]1(O)CCC(F)(F)c2c3c(nn2C1)C[C@@H](C)N(C(=O)Nc1ccnc(C(F)F)c1F)C3]

The title compound was prepared in a manner analogous to Example 1, however using (3R,8S*)-tert-butyl 8-ethynyl-11,11-difluoro-8-hydroxy-3-methyl-3,4,8,9,10,11-hexahydro-1H-pyrido[4′,3′:3,4]pyrazolo[1,5-a]azepine-2(7H)-carboxylate (Intermediate 42) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (2-(difluoromethyl)-3-fluoropyridin-4-yl) carbamate instead of phenyl (3-cyano-4-fluorophenyl)carbamate in Step B, and purification via RP HPLC(condition E). MS (ESI): mass calcd. for C₂₁H₂₀F₅N₅O₂ 469.2; m/z found, 470.2 [M+H]+. ¹H NMR (400 MHz, CDCl₃) δ=8.40-8.28 (m, 2H), 7.09 (d, J=4.4 Hz, 1H), 6.76 (t, J=53.6 Hz, 1H), 5.02-4.82 (m, 2H), 4.57 (s, 2H), 4.43 (d, J=15.6 Hz, 1H), 3.79 (s, 1H), 3.06-3.01 (m, 1H), 2.75 (d, J=15.6 Hz, 1H), 2.64-2.28 (m, 5H), 1.24 (d, J=6.8 Hz, 3H).

### Example 101

### (3R,8S*)-N-(3-Cyano-4-fluorophenyl)-11,11-difluoro-8-(2-hydroxypropan-2-yl)-3-methyl-3,4,8,9,10,11-hexahydro-1H-pyrido[4′,3′:3,4]pyrazolo[1,5-a]azepine-2(7H)-carboxamide

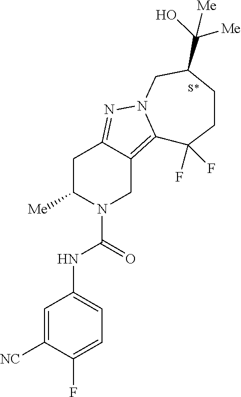 [SMILES: C[C@@H]1Cc2nn3c(c2CN1C(=O)Nc1ccc(F)c(C#N)c1)C(F)(F)CC[C@H](C(C)(C)O)C3]

The title compound was prepared in a manner analogous to Example 1, however using (3R,8S*)-tert-butyl 11,11-difluoro-8-(2-hydroxypropan-2-yl)-3-methyl-3,4,8,9,10,11-hexahydro-1H-pyrido[4′,3′:3,4]pyrazolo[1,5-a]azepine-2(7H)-carboxylate (Intermediate 43) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A, and purification via RP HPLC(condition E) MS (ESI): mass calcd. for C₂₃H₂₆F₃N₅O₂ 461.20; m/z found, 462.2 [M+H]+. ¹H NMR (400 MHz, CDCl₃) δ=7.75-7.73 (m, 1H), 7.65-7.57 (m, 1H), 7.16 (t, J=8.6 Hz, 1H), 6.43 (s, 1 H), 5.06-4.95 (m, 1H), 4.77 (m, 2H), 4.40-4.30 (m, 1H), 4.08 (dd, J=9.4, 14.2 Hz, 1H), 3.08-2.96 (m, 1H), 2.73-2.50 (m, 2H), 2.25-2.10 (m, 2H), 1.96-1.70 (m, 2H), 1.32-1.28 (m, 6H), 1.20 (d, J=6.8 Hz, 3H).

### Example 102

### (3R,8S*)-N-(2-Bromo-3-fluoropyridin-4-yl)-11,11-difluoro-8-(2-hydroxypropan-2-yl)-3-methyl-3,4,8,9,10,11-hexahydro-1H-pyrido[4′,3′:3,4]pyrazolo[1,5-a]azepine-2(7H)-carboxamide

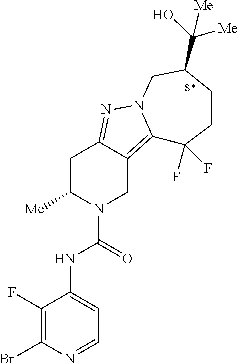 [SMILES: C[C@@H]1Cc2nn3c(c2CN1C(=O)Nc1ccnc(Br)c1F)C(F)(F)CC[C@H](C(C)(C)O)C3]

The title compound was prepared in a manner analogous to Example 1, however using (3R,8S*)-tert-butyl 11,11-difluoro-8-(2-hydroxypropan-2-yl)-3-methyl-3,4,8,9,10,11-hexahydro-1H-pyrido[4′,3′:3,4]pyrazolo[1,5-a]azepine-2(7H)-carboxylate (Intermediate 43) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (2-bromo-3-fluoropyridin-4-yl)carbamate instead of phenyl (3-cyano-4-fluorophenyl)carbamate in Step B, and purification via RP HPLC(condition E) MS (ESI): mass calcd. for C₂₁H₂₅BrF₃N₅O₂ 515.1/517.1; m/z found, 516.1/518.1 [M+H]+. ¹H NMR (400 MHz, CDCl₃) δ=8.22-8.15 (m, 1H), 8.07 (d, J=5.6 Hz, 1H), 6.91 (d, J=3.6 Hz, 1H), 5.02-4.68 (m, 3H), 4.38 (br d, J=14.7 Hz, 1H), 4.16-4.03 (m, 1H), 3.08-2.97 (m, 1H), 2.70-2.50 (m, 2H), 2.30-2.5 (m, 2H), 1.94-1.71 (m, 2H), 1.32-1.28 (m, 6H), 1.25-1.21 (m, 3H).

### Example 103

### (3R,8R*)-N-(3-Cyano-4-fluorophenyl)-11,11-difluoro-8-(2-hydroxypropan-2-yl)-3-methyl-3,4,8,9,10,11-hexahydro-1H-pyrido[4′,3′:3,4]pyrazolo[1,5-a]azepine-2(7H)-carboxamide

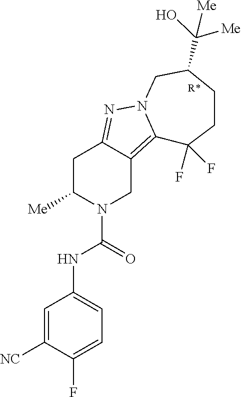 [SMILES: C[C@@H]1Cc2nn3c(c2CN1C(=O)Nc1ccc(F)c(C#N)c1)C(F)(F)CC[C@@H](C(C)(C)O)C3]

The title compound was prepared in a manner analogous to Example 1, however using (3R,8R*)-tert-butyl 11,11-difluoro-8-(2-hydroxypropan-2-yl)-3-methyl-3,4,8,9,10,11-hexahydro-1H-pyrido[4′,3′:3,4]pyrazolo[1,5-a]azepine-2(7H)-carboxylate (Intermediate 44) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A, and purification via RP HPLC(condition E). MS (ESI): mass calcd. for C₂₃H₂₆F₃N₅O₂ 461.20; m/z found, 462.2 [M+H]+. ¹H NMR (400 MHz, CDCl₃) δ=7.75 (dd, J=2.9, 5.3 Hz, 1H), 7.67-7.59 (m, 1H), 7.17 (t, J=8.7 Hz, 1H), 6.43 (s, 1H), 4.97-4.90 (m, 1H), 4.78 (br d, J=14.4 Hz, 2H), 4.40 (br d, J=15.0 Hz, 1H), 4.09 (dd, J=9.7, 13.8 Hz, 1H), 3.02 (dd, J=6.0, 16.0 Hz, 1H), 2.73-2.53 (m, 2H), 2.32-2.12 (m, 2H), 1.96-1.82 (m, 1H), 1.78-1.68 (m, 1H), 1.34-1.25 (m, 9H).

### Example 104

### (3R,8R*)-N-(2-Bromo-3-fluoropyridin-4-yl)-11,11-difluoro-8-(2-hydroxypropan-2-yl)-3-methyl-3,4,8,9,10,11-hexahydro-1H-pyrido[4′,3′:3,4]pyrazolo[1,5-a]azepine-2(7H)-carboxamide

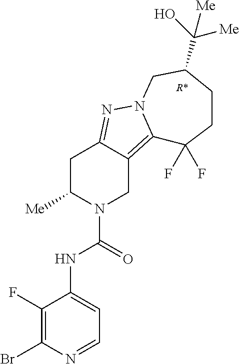 [SMILES: C[C@@H]1Cc2nn3c(c2CN1C(=O)Nc1ccnc(Br)c1F)C(F)(F)CC[C@@H](C(C)(C)O)C3]

The title compound was prepared in a manner analogous to Example 1, however using (3R,8R*)-tert-butyl 11,11-difluoro-8-(2-hydroxypropan-2-yl)-3-methyl-3,4,8,9,10,11-hexahydro-1H-pyrido[4′,3′:3,4]pyrazolo[1,5-a]azepine-2(7H)-carboxylate (Intermediate 44) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (2-bromo-3-fluoropyridin-4-yl)carbamate instead of phenyl (3-cyano-4-fluorophenyl)carbamate in Step B, and purification via RP HPLC(condition E). MS (ESI): mass calcd. for C₂₁H₂₅BrF₃N₅O₂ 515.1/517.1; m/z found, 516.1/518.1 [M+H]+. ¹H NMR (400 MHz, CDCl₃) δ=8.20 (t, J=5.5 Hz, 1H), 8.09 (d, J=5.5 Hz, 1H),6.92-6.91 (d, J=4.4 Hz, 1H), 5.08-4.70 (m, 3H), 4.44 (br d, J=16.0 Hz, 1H), 4.09 (dd, J=9.8, 14.1 Hz, 1H), 3.03 (dd, J=5.8, 15.7 Hz, 1H), 2.80-2.49 (m, 2H), 2.37-2.09 (m, 2H), 2.01-1.79 (m, 1H), 1.79-1.66 (m, 1H), 1.37-1.26 (m, 9H).

### Example 105

### methyl (((3R,8R*)-2-((3-cyano-4-fluorophenyl)carbamoyl)-11,11-difluoro-3-methyl-2,3,4,7,8,9,10,11-octahydro-1H-pyrido[4′,3′:3,4]pyrazolo[1,5-a]azepin-8-yl)-methyl)carbamate

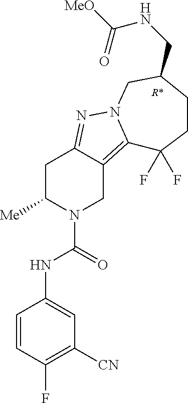 [SMILES: COC(=O)NC[C@H]1CCC(F)(F)c2c3c(nn2C1)C[C@@H](C)N(C(=O)Nc1ccc(F)c(C#N)c1)C3]

The title compound was prepared in a manner analogous to Example 1, however using (3R,8R*)-tert-butyl 11,11-difluoro-8-(((methoxycarbonyl)amino)methyl) -3-methyl-3,4,8,9,10,11-hexahydro-1H-pyrido[4′,3′:3,4]pyrazolo[1,5-a]azepine-2(7H)-carboxylate (Intermediate 45) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A. MS (ESI): mass calcd. for C₂₃H₂₅F₃N₆O₃ 490.2; m/z found, 491.2 [M+H]+. ¹H NMR (400 MHz, CDCl₃) δ=7.74-7.72 (m, 1H), 7.62-7.59 (m, 1H), 7.15 (t, J=8.7 Hz, 1H), 6.44 (s, 1H), 5.02-4.92 (m, 1H), 4.90-4.83 (m, 1H), 4.78 (br d, J=15.0 Hz, 1H), 4.48-4.31 (m, 2H), 4.21-4.08 (m, 1H), 3.69 (s, 3H), 3.30-3.15 (m, 1H), 3.00 (br dd, J=5.8, 15.8 Hz, 2H), 2.66 (d, J=16.1 Hz, 1H), 2.56-2.38 (m, 1H), 2.36-2.15 (m, 1H), 2.08-2.01 (m, 2H), 1.87-1.85 (m, 1H), 1.22 (d, J=6.8 Hz, 3H).

### Example 106

### Methyl (((3R,8R*)-2-((2-bromo-3-fluoropyridin-4-yl)carbamoyl)-11,11-difluoro-3-methyl-2,3,4,7,8,9,10,11-octahydro-1H-pyrido[4′,3′:3,4]pyrazolo[1,5-a]azepin-8-yl)methyl)carbamate

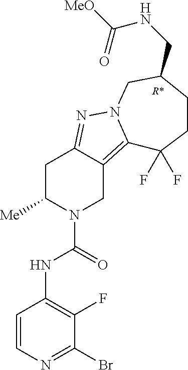 [SMILES: COC(=O)NC[C@H]1CCC(F)(F)c2c3c(nn2C1)C[C@@H](C)N(C(=O)Nc1ccnc(Br)c1F)C3]

The title compound was prepared in a manner analogous to Example 1, however using (3R,8R*)-tert-butyl 11,11-difluoro-8-(((methoxycarbonyl)amino)methyl) -3-methyl-3,4,8,9,10,11-hexahydro-1H-pyrido[4′,3′:3,4]pyrazolo[1,5-a]azepine-2(7H)-carboxylate (Intermediate 45) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (2-bromo-3-fluoropyridin-4-yl)carbamate instead of phenyl (3-cyano-4-fluorophenyl)carbamate in step B. MS (ESI): mass calcd. for C₂₁H₂₄BrF₃N₆O₃ 544.1/546.1; m/z found, 545.1/547.1 [M+H]+. ¹H NMR (400 MHz, CDCl₃) δ=8.18 (t, J=5.4 Hz, 1H), 8.08 (d, J=5.6 Hz, 1H), 6.91 (d, J=4.0 Hz, 1H), 5.02-4.78 (m, 3H), 4.50-4.35 (m, 2H), 4.16-4.10 (m, 1H), 3.69 (s, 3H), 3.22-3.14 (m, 1H), 3.04-2.98 (m, 2H), 2.69 (d, J=15.5 Hz, 1H), 2.56-2.39 (m, 1H), 2.34-2.13 (m, 1H), 2.09-2.00 (m, 2H), 1.90-1.75 (m, 1H), 1.27-1.25 (d, J=6.8 Hz, 3H).

### Example 107

### Methyl (((3R,8R*)-2-((2-(difluoromethyl)-3-fluoropyridin-4-yl) carbamoyl)-11,11-difluoro-3-methyl-2,3,4,7,8,9,10,11-octahydro-1H-pyrido[4′,3′:3,4]pyrazolo[1,5-a]azepin-8-yl)methyl)carbamate

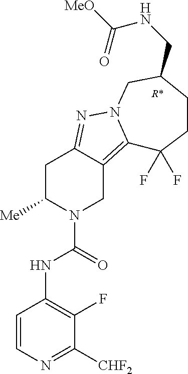 [SMILES: COC(=O)NC[C@H]1CCC(F)(F)c2c3c(nn2C1)C[C@@H](C)N(C(=O)Nc1ccnc(C(F)F)c1F)C3]

The title compound was prepared in a manner analogous to Example 1, however using (3R,8R*)-tert-butyl 11,11-difluoro-8-(((methoxycarbonyl)amino)methyl)-3-methyl-3,4,8,9,10,11-hexahydro-1H-pyrido[4′,3′:3,4]pyrazolo[1,5-a]azepine-2(7H)-carboxylate (Intermediate 45) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (2-(difluoromethyl)-3-fluoropyridin-4-yl)carbamate instead of phenyl (3-cyano-4-fluorophenyl)carbamate in step B. MS (ESI): mass calcd. for C₂₂H₂₅F₅N₆O₃ 516.2; m/z found, 517.2 [M+H]+. ¹H NMR (400 MHz, CDCl₃) δ=8.37-8.31 (m, 2H), 6.99 (br d, J=4.0 Hz, 1H), 6.74 (t, J=53.6 Hz, 1H), 4.96-4.83 (m, 3H), 4.49-4.37 (m, 2H), 4.18-4.08 (m, 1H), 3.69 (s, 3H), 3.27-3.15 (m, 1H), 3.09-2.96 (m, 2H), 2.69 (d, J=15.7 Hz, 1H), 2.58-2.40 (m, 1H), 2.33-2.16 (m, 1H), 2.10-2.04 (m, 2H), 1.85-1.80 (m, 1H), 1.26 (d, J=6.8 Hz, 3H).

### Example 108

### Methyl (((3R,8S*)-2-((3-cyano-4-fluorophenyl)carbamoyl)-11,11-difluoro-3-methyl-2,3,4,7,8,9,10,11-octahydro-1H-pyrido[4′,3′:3,4]pyrazolo[1,5-a]azepin-8-yl)methyl)carbamate

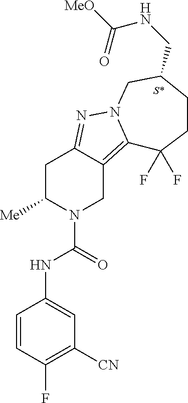 [SMILES: COC(=O)NC[C@@H]1CCC(F)(F)c2c3c(nn2C1)C[C@@H](C)N(C(=O)Nc1ccc(F)c(C#N)c1)C3]

The title compound was prepared in a manner analogous to Example 1, however using (3R,8S*)-tert-butyl 11,11-difluoro-8-(((methoxycarbonyl)amino)methyl) -3-methyl-3,4,8,9,10,11-hexahydro-1H-pyrido[4′,3′:3,4]pyrazolo[1,5-a]azepine-2(7H)-carboxylate (Intermediate 46) instead of (tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A. MS (ESI): mass calcd. for C₂₃H₂₅F₃N₆O₃ 490.2; m/z found, 491.2 [M+H]+. ¹H NMR (400 MHz, CDCl₃) δ=7.66 (dd, J=2.8, 5.4 Hz, 1H), 7.55-7.52 (m, 1H), 7.07 (t, J=8.7 Hz, 1H), 6.45 (s, 1H), 5.03-4.63 (m, 3H), 4.46-4.21 (m, 2H), 4.12 (dd, J=7.8, 14.4 Hz, 1H), 3.61 (s, 3H), 3.25-3.05 (m, 1H), 2.99-2.74 (m, 2H), 2.57 (d, J=15.9 Hz, 1H), 2.43-1.92 (m, 4H), 1.89-1.69 (m, 1H), 1.12 (d, J=7.0 Hz, 3H).

### Example 109

### Methyl (((3R,8S*)-2-((2-bromo-3-fluoropyridin-4-yl)carbamoyl)-11,11-difluoro-3-methyl-2,3,4,7,8,9,10,11-octahydro-1H-pyrido[4′,3′:3,4]pyrazolo[1,5-a]azepin-8-yl)methyl)carbamate

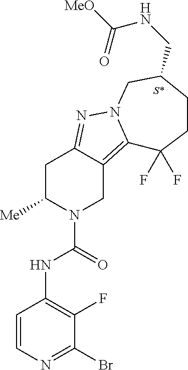 [SMILES: COC(=O)NC[C@@H]1CCC(F)(F)c2c3c(nn2C1)C[C@@H](C)N(C(=O)Nc1ccnc(Br)c1F)C3]

The title compound was prepared in a manner analogous to Example 1, however using (3R,8S*)-tert-butyl 11,11-difluoro-8-(((methoxycarbonyl)amino)methyl) -3-methyl-3,4,8,9,10,11-hexahydro-1H-pyrido[4′,3′:3,4]pyrazolo[1,5-a]azepine-2(7H)-carboxylate (Intermediate 46) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (2-bromo-3-fluoropyridin-4-yl)carbamate instead of phenyl (3-cyano-4-fluorophenyl)carbamate in step B. MS (ESI): mass calcd. for C₂₁H₂₄BrF₃N₆O₃ 544.1/546.1; m/z found, 545.1/547.1 [M+H]+. ¹H NMR (400 MHz, CDCl₃) δ=8.26-8.14 (m, 1H), 8.08 (d, J=5.5 Hz, 1H), 6.92 (d, J=3.8 Hz, 1H), 5.02-4.76 (m, 3H), 4.53-4.32 (m, 2H), 4.20 (dd, J=8.0, 14.4 Hz, 1H), 3.68 (s, 3H), 3.33-3.12 (m, 1H), 3.02 (dd, J=5.9, 15.9 Hz, 1H), 2.96-2.81 (m, 1H), 2.68 (d, J=15.8 Hz, 1H), 2.49-2.02 (m, 4H), 1.97-1.76 (m, 1H), 1.23 (d, J=7.0 Hz, 3H).

### Example 110

### Methyl (((3R,8S*)-2-42-(difluoromethyl)-3-fluoropyridin-4-yl) carbamoyl)-11,11-difluoro-3-methyl-2,3,4,7,8,9,10,11-octahydro-1H-pyrido[4′,3′:3,4]pyrazolo[1,5-a]azepin-8-yl)methyl)carbamate

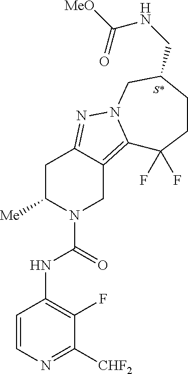 [SMILES: COC(=O)NC[C@@H]1CCC(F)(F)c2c3c(nn2C1)C[C@@H](C)N(C(=O)Nc1ccnc(C(F)F)c1F)C3]

The title compound was prepared in a manner analogous to Example 1, however using (3R,8S*)-tert-butyl 11,11-difluoro-8-(((methoxy carbonyl)amino)methyl)-3-methyl-3,4,8,9,10,11-hexahydro-1H-pyrido[4′,3′:3,4]pyrazolo[1,5-a]azepine-2(7H)-carboxylate (Intermediate 46) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (2-(difluoromethyl)-3-fluoropyridin-4-yl)carbamate instead of phenyl (3-cyano-4-fluorophenyl)carbamate in step B. MS (ESI): mass calcd. for C₂₂H₂₅F₅N₆O₃ 516.2; m/z found, 517.2 [M+H]+. ¹H NMR (400 MHz, CDCl₃) δ=8.50-8.23 (m, 2H), 7.00 (d, J=4.0 Hz, 1H), 6.75 (t, J=53.6 Hz, 1H), 5.07-4.76 (m, 3H), 4.52-4.31 (m, 2H), 4.20 (dd, J=7.9, 14.4 Hz, 1H), 3.68 (s, 3H), 3.30-3.14 (m, 1H), 3.02 (dd, J=5.6, 15.9 Hz, 1H), 2.96-2.83 (m, 1H), 2.68 (d, J=15.9 Hz, 1H), 2.53-2.02 (m, 4H), 1.94-1.77 (m, 1H), 1.24 (d, J=6.8 Hz, 3H).

### Example 111

### (3R,8S*)-N-(3-Cyano-4-fluorophenyl)-8,11,11-trifluoro-8-(hydroxymethyl)-3-methyl-3,4,8,9,10,11-hexahydro-1H-pyrido[4′,3′:3,4]pyrazolo[1,5-a]azepine-2(7H)-carboxamide

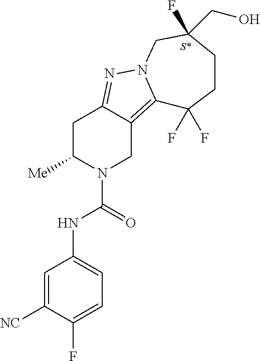 [SMILES: C[C@@H]1Cc2nn3c(c2CN1C(=O)Nc1ccc(F)c(C#N)c1)C(F)(F)CC[C@@](F)(CO)C3]

The title compound was prepared in a manner analogous to Example 1, however using (3R,8S*)-tert-butyl 8,11,11-trifluoro-8-(hydroxymethyl)-3-methyl-3,4,8,9,10,11-hexahydro-1H-pyrido[4′,3′:3,4]pyrazolo[1,5-a]azepine-2(7H)-carboxylate (Intermediate 47) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A. MS (ESI): mass calcd. for C₂₁H₂₁F₄N₅O₂ 451.2; m/z found, 452.2 [M+H]+. ¹H NMR (400 MHz, CDCl₃) δ=7.73 (dd, J=2.8, 5.4 Hz, 1H), 7.62-7.59 (m, 1H), 7.15 (t, J=8.7 Hz, 1H), 6.47 (s, 1H), 5.01-4.90 (m, 1H), 4.89-4.72 (m, 2H), 4.46-4.30 (m, 2H), 3.75-3.58 (m, 2H), 3.01 (dd, J=5.7, 15.7 Hz, 1H), 2.69 (d, J=15.9 Hz, 1H), 2.60-2.01 (m, 5H),1.22 (d, J=6.8 Hz, 3H).

### Example 112

### (3R,8S*)-N-(2-Bromo-3-fluoropyridin-4-yl)-8,11,11-trifluoro-8-(hydroxymethyl)-3-methyl-3,4,8,9,10,11-hexahydro-1H-pyrido[4′,3′:3,4]pyrazolo[1,5-a]azepine-2(7H)-carboxamide

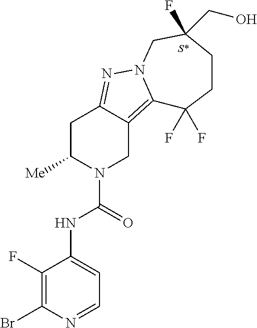 [SMILES: C[C@@H]1Cc2nn3c(c2CN1C(=O)Nc1ccnc(Br)c1F)C(F)(F)CC[C@@](F)(CO)C3]

The title compound was prepared in a manner analogous to Example 1, however using (3R,8S*)-tert-butyl 8,11,11-tri fluoro-8-(hydroxymethyl)-3-methyl-3 ,4,8, 9,10,11-hexahydro-1H-pyrido[4′,3′:3,4]pyrazolo[1,5-a]azepine-2(7H)-carboxylate (Intermediate 47) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (2-bromo-3-fluoropyridin-4-yl)carbamate instead of phenyl (3-cyano-4-fluorophenyl)carbamate in step B. MS (ESI): mass calcd. for C₁₉H₂₀BrF₄N₅O₂ 505.1/507.1; m/z found, 506.1/508.1 [M+H]+. ¹H NMR (400 MHz, CDCl₃) δ=8.18 (t, J=5.6 Hz, 1H), 8.08 (d, J=5.5 Hz, 1H),6.92 (d, J=4.2 Hz, 1H), 4.99-4.85 (m, 2H), 4.76 (dd, J=2.2, 14.8 Hz, 1H), 4.49-4.29 (m, 2H), 3.77-3.55 (m, 2H), 3.03 (dd, J=5.9, 15.8 Hz, 1H), 2.72 (d, J=16.3 Hz, 1H), 2.61-1.88 (m, 5H), 1.25 (d, J=6.8 Hz, 3H).

### Example 113

### (3R,8S*)-N-(2-(Difluoromethyl)-3-fluoropyridin-4-yl)-8,11,11-trifluoro-8-(hydroxymethyl)-3-methyl-3,4,8,9,10,11-hexahydro-1H-pyrido[4′,3′:3,4]pyrazolo[1,5-a]azepine-2(7H)-carboxamide

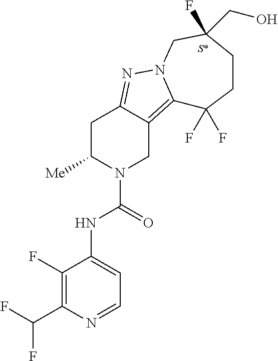 [SMILES: C[C@@H]1Cc2nn3c(c2CN1C(=O)Nc1ccnc(C(F)F)c1F)C(F)(F)CC[C@@](F)(CO)C3]

The title compound was prepared in a manner analogous to Example 1, however using (3R,8S*)-tert-butyl 8,11,11-trifluoro-8-(hydroxymethyl)-3-methyl-3,4,8, 9,10,11-hexahydro-1H-pyrido[4′,3′:3,4]pyrazolo[1,5-a]azepine-2(7H)-carboxylate (Intermediate 47) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (2-(difluoromethyl)-3-fluoropyridin-4-yl)carbamate instead of phenyl (3-cyano-4-fluorophenyl)carbamate in step B. MS (ESI): mass calcd. for C₂₀H₂₁F₆N₅O₂ 477.2; m/z found, 478.2 [M+H]+. ¹H NMR (400 MHz, CDCl₃) δ=8.39-8.25 (m, 2H), 7.01 (d, J=4.0 Hz, 1H), 6.72 (t, J=53.6 Hz, 1H), 5.11-4.85 (m, 2H), 4.76 (dd, J=2.2, 14.7 Hz, 1H), 4.54-4.26 (m, 2H), 3.77-3.54 (m, 2H), 3.04 (dd, J=5.7 , 16.0 Hz, 1H), 2.72 (d, J=16.4 Hz, 1H), 2.61-2.05 (m, 5H), 1.25 (d, J=7.0 Hz, 3H).

### Example 114

### (3R,8R*)-N-(3-Cyano-4-fluorophenyl)-8,11,11-trifluoro-8-(hydroxyl methyl)-3-methyl-3,4,8,9,10,11-hexahydro-1H-pyrido[4′,3′:3,4]pyrazolo[1,5-a]azepine-2(7H)-carboxamide

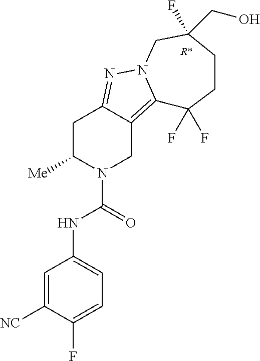 [SMILES: C[C@@H]1Cc2nn3c(c2CN1C(=O)Nc1ccc(F)c(C#N)c1)C(F)(F)CC[C@](F)(CO)C3]

The title compound was prepared in a manner analogous to Example 1, however using (3R,8R*)-tert-butyl 8,11,11-trifluoro-8-(hydroxymethyl)-3-methyl-3,4,8, 9,10,11-hexahydro-1H-pyrido[4′,3′:3,4]pyrazolo[1,5-a]azepine-2(7H)-carboxylate (Intermediate 48) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A. MS (ESI): mass calcd. for C₂₁H₂₁F₄N₅O₂ 451.2; m/z found, 452.2 [M+H]+. ¹H NMR (400 MHz, CDCl₃) δ=7.72 (dd, J=2.8, 5.3 Hz, 1H), 7.64-7.60 (m, 1H), 7.15 (t, J=8.7 Hz, 1H), 6.49 (s, 1H), 4.95 (br t, J=6.6 Hz, 1H), 4.82 (br d, J=15.2 Hz, 2H), 4.45-4.20 (m, 2H), 3.84-3.57 (m, 2H), 3.03 (dd, J=5.9, 15.9 Hz, 1H), 2.69 (d, J=15.8 Hz, 1H), 2.59-2.34 (m, 2H), 2.31-2.08 (m, 2H), 1.22 (d, J=6.8 Hz, 3H).

### Example 115

### (3R,8R*)-N-(2-Bromo-3-fluoropyridin-4-yl)-8,11,11-trifluoro-8-(hydroxylmethyl)-3-methyl-3,4,8,9,10,11-hexahydro-1H-pyrido[4′,3′:3,4]pyrazolo[1,5-a]azepine-2(7H)-carboxamide

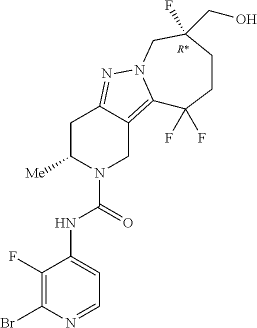 [SMILES: C[C@@H]1Cc2nn3c(c2CN1C(=O)Nc1ccnc(Br)c1F)C(F)(F)CC[C@](F)(CO)C3]

The title compound was prepared in a manner analogous to Example 1, however using (3R,8R*)-tert-butyl 8,11,11-trifluoro-8-(hydroxymethyl)-3-methyl-3,4,8, 9,10,11-hexahydro-1H-pyrido[4′,3′:3,4]pyrazolo[1,5-a]azepine-2(7H)-carboxylate (Intermediate 48) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (2-bromo-3-fluoropyridin-4-yl)carbamate instead of phenyl (3-cyano-4-fluorophenyl)carbamate. MS (ESI): mass calcd. for C₁₉H₂₀BrF₄N₅O₂ 505.1/507.1; m/z found, 506.1/508.1 [M+H]+. ¹H NMR (400 MHz, CDCl₃) δ=8.27-8.13 (m, 1H), 8.08 (d, J=5.5 Hz, 1H), 6.92 (d, J=4.0 Hz, 1H), 5.06-4.78 (m, 3H), 4.50-4.28 (m, 2H), 3.75-3.64 (m, 2H), 3.05 (dd, J=5.8, 16.0 Hz, 1H), 2.71 (d, J=15.7 Hz, 1H), 2.64-2.34 (m, 2H), 2.29-2.08 (m, 2H), 1.25 (d, J=7.0 Hz, 3H).

### Example 116

### (3R,8R*)-N-(2-(Difluoromethyl)-3-fluoropyridin-4-yl)-8,11,11-trifluoro-8-(hydroxymethyl)-3-methyl-3,4,8,9,10,11-hexahydro-1H-pyrido[4′,3′:3,4]pyrazolo[1,5-a]azepine-2 (7H)-carboxamide

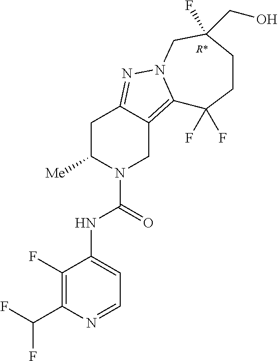 [SMILES: C[C@@H]1Cc2nn3c(c2CN1C(=O)Nc1ccnc(C(F)F)c1F)C(F)(F)CC[C@](F)(CO)C3]

The title compound was prepared in a manner analogous to Example 1, however using (3R,8R*)-tert-butyl 8,11,11-trifluoro-8-(hydroxymethyl)-3-methyl-3,4,8, 9,10,11-hexahydro-1H-pyrido[4′,3′:3,4]pyrazolo[1,5-a]azepine-2(7H)-carboxylate (Intermediate 48) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A and phenyl (2-(difluoromethyl)-3-fluoropyridin-4-yl)carbamate instead of phenyl (3-cyano-4-fluorophenyl)carbamate in step B. MS (ESI): mass calcd. for C₂₀H₂₁F₆N₅O₂ 477.2; m/z found, 478.2 [M+H]+. ¹H NMR (400 MHz, CDCl₃) δ=8.53-8.32 (m, 2H), 7.00 (br d, J=4.4 Hz, 1H), 6.75 (t, J=53.6 Hz, 1H), 5.94-4.82 (m, 3H), 4.57-4.28 (m, 2H), 3.74-3.64 (m, 2H), 3.05 (dd, J=5.9, 15.9 Hz, 1H), 2.72 (d, J=15.9 Hz, 1H), 2.61-2.35 (m, 2H), 2.30-2.09 (m, 2H), 1.26 (d, J=7.0 Hz, 3H).

### Example 117

### (3R,9S*)-N-(2-Bromo-3-fluoropyridin-4-yl)-11,11-difluoro-9-hydroxyl-3-methyl-3,4,8,9,10,11-hexahydro-1H-pyrido[4′,3′:3,4]pyrazolo[1,5-a]azepine-2(7H) carboxamide

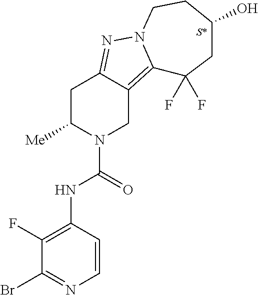 [SMILES: C[C@@H]1Cc2nn3c(c2CN1C(=O)Nc1ccnc(Br)c1F)C(F)(F)C[C@@H](O)CC3]

The title compound was prepared from intermediate 51 in a manner analogous to Example 1, however using phenyl (2-bromo-3-fluoropyridin-4-yl)carbamate instead of phenyl (3-cyano-4-fluorophenyl)carbamate in Step B. MS (ESI): mass calcd. for C₁₈H₁₉BrF₃N₅O₂ 473.1; m/z found, 474.0/476.1 [M+H]+. ¹H NMR (400 MHz, CDCl₃) δ=8.19-8.16 (m, 1H), 8.08 (d, J=5.6 Hz, 1H), 6.92 (s, 1H), 4.97-4.83 (m, 2H), 4.60-4.51 (m, 1H), 4.43-4.31 (m, 2H), 4.26-4.18 (m, 1H), 3.09-2.97 (m, 1H), 2.80-2.64 (m, 2H), 2.46-2.22 (m, 2H), 2.10-2.01 (m, 1H), 1.89 (s, 1H), 1.24 (d, J=6.8 Hz, 3H).

### Example 118

### (3R,9R*)-N-(2-Bromo-3-fluoropyridin-4-yl)-11,11-difluoro-9-hydroxyl-3-methyl-3,4,8,9,10,11-hexahydro-1H-pyrido[4′,3′:3,4]pyrazolo[1,5-a]azepine-2(7H)-carboxamide

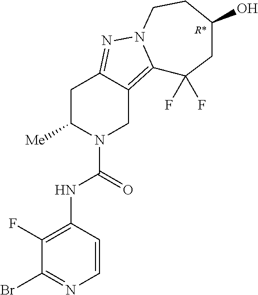 [SMILES: C[C@@H]1Cc2nn3c(c2CN1C(=O)Nc1ccnc(Br)c1F)C(F)(F)C[C@H](O)CC3]

The title compound was prepared from intermediate 50 in a manner analogous to Example 1, however using phenyl (2-bromo-3-fluoropyridin-4-yl)carbamate instead of phenyl (3-cyano-4-fluorophenyl)carbamate in Step B. MS (ESI): mass calcd. for C₁₈H₁₉BrF₃N₅O₂ 473.1; m/z found, 474.1/476.1 [M+H]+. ¹H NMR (400 MHz, CDCl₃) δ=8.19-8.16 (m, 1H), 8.08 (d, J=5.6 Hz, 1H), 6.92 (s, 1H), 4.96-4.80 (m, 2H), 4.58-4.13 (m, 4H), 3.09-2.97 (m, 1H), 2.84-2.64 (m, 2H), 2.34-2.26 (m, 2H), 2.04-1.86 (m, 2H), 1.24 (d, J=6.8 Hz, 3H).

### Example 119

### (3R,9S*)-N-(3-Cyano-4-fluorophenyl)-11,11-difluoro-9-hydroxyl-3-methyl-3,4,8,9,10,11-hexahydro-1H-pyrido[4′,3′:3,4]pyrazolo[1,5-a]azepine-2(7H)-carboxamide

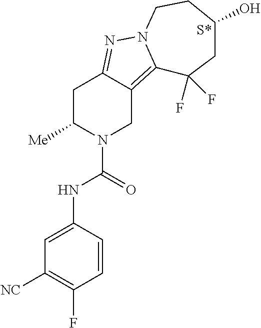 [SMILES: C[C@@H]1Cc2nn3c(c2CN1C(=O)Nc1ccc(F)c(C#N)c1)C(F)(F)C[C@@H](O)CC3]

The title compound was prepared from intermediate 51 in a manner analogous to Example 1. MS (ESI): mass calcd. for C₂₀H₂₀F₃N₅O₂ 419.1; m/z found, 420.2 [M+H]+. ¹H NMR (400 MHz, CDCl₃) δ=7.77-7.70 (m, 1H), 7.65-7.56 (m, 1H), 7.15 (t, J=8.8 Hz, 1H), 6.48 (s, 1H), 5.03-4.93 (m, 1H), 4.82 (d, J=15.6 Hz, 1H), 4.58-4.50 (m, 1H), 4.39-4.28 (m, 2H), 4.26-4.13 (m, 1H), 3.07-2.96 (m, 1H), 2.80-2.61 (m, 2H), 2.41-2.22 (m, 2H), 2.08-1.99 (m, 1H), 1.21 (d, J=6.8 Hz, 3H).

### Example 120

### (3R,9R*)-N-(3-Cyano-4-fluorophenyl)-11,11-difluoro-9-hydroxy-3-methyl-3,4,8,9,10,11-hexahydro-1H-pyrido[4′,3′:3,4]pyrazolo[1,5-a]azepine-2(7H)-carboxamide

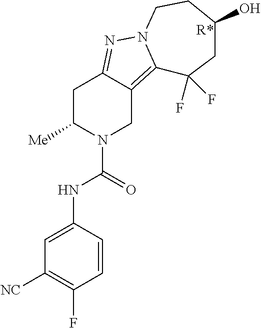 [SMILES: C[C@@H]1Cc2nn3c(c2CN1C(=O)Nc1ccc(F)c(C#N)c1)C(F)(F)C[C@H](O)CC3]

The title compound was prepared in a manner analogous to Example 1. MS (ESI): mass calcd. for C₂₀H₂₀F₃N₅O₂ 419.1; m/z found, 420.2 [M+H]+. ¹H NMR (400 MHz, CDCl₃) δ=7.79-7.70 (m, 1H), 7.66-7.58 (m, 1H), 7.16 (t, J=8.8 Hz, 1H), 6.49 (s, 1H), 5.03-4.94 (m, 1H), 4.86-4.76 (m, 1H), 4.57-4.49 (m, 1H), 4.44-4.14 (m, 3H), 3.08-2.97 (m, 1H), 2.85-2.63 (m, 2H), 2.37-2.26 (m, 2H), 2.01-1.89 (m, 2H), 1.23 (d, J=6.8 Hz, 3H).

### Example 121

### (3R,9S*)-N-(2-(Difluoromethyl)-3-fluoropyridin-4-yl)-11,11-difluoro-9-hydroxy-3-methyl-3,4,8,9,10,11-hexahydro-1H-pyrido[4′,3′:3,4]pyrazolo[1,5-a]azepine-2(7H)-carboxamide

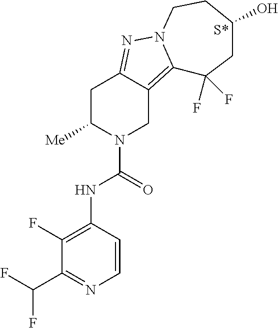 [SMILES: C[C@@H]1Cc2nn3c(c2CN1C(=O)Nc1ccnc(C(F)F)c1F)C(F)(F)C[C@@H](O)CC3]

The title compound was prepared from intermediate 51 in a manner analogous to Example 1, however using phenyl (2-(difluoromethyl)-3-fluoropyridin-4-yl)carbamate instead of phenyl (3-cyano-4-fluorophenyl)carbamate in Step B. MS (ESI): mass calcd. for C₁₉H₂₀F₅N₅O₂ 445.1; m/z found, 446.2 [M+H]+. ¹H NMR (400 MHz, CDCl₃) δ=8.40-8.25 (m, 2H), 7.00 (d, J=4.8 Hz, 1H), 6.75 (t, J=53.6 Hz, 1H), 5.00-4.84 (m, 2H), 4.58-4.51 (m, 1H), 4.44-4.29 (m, 2H), 4.25-4.14 (m, 1H), 3.10-2.98 (m, 1H), 2.80-2.63 (m, 2H), 2.46-2.22 (m, 2H), 2.09-1.97 (m, 1H), 1.25 (d, J=6.8 Hz, 3H).

### Example 122

### (3R,9R*)-N-(2-(Difluoromethyl)-3-fluoropyridin-4-yl)-11,11difluoro-9-hydroxy-3-methyl-3,4,8,9,10,11-hexahydro-1H-pyrido[4′,3′:3,4]pyrazolo[1,5-a]azepine-2(7H)-carboxamide

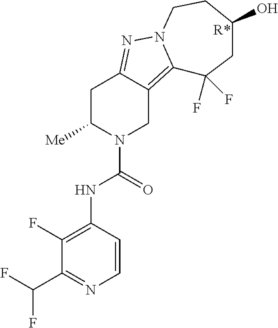 [SMILES: C[C@@H]1Cc2nn3c(c2CN1C(=O)Nc1ccnc(C(F)F)c1F)C(F)(F)C[C@H](O)CC3]

The title compound was prepared from 50 in a manner analogous to Example 1, however using phenyl (2-(difluoromethyl)-3-fluoropyridin-4-yl)carbamate instead of phenyl (3-cyano-4-fluorophenyl)carbamate in Step B. MS (ESI): mass calcd. for C₁₉H₂₀F₅N₅O₂ 445.1; m/z found, 446.2 [M+H]+. ¹H NMR (400 MHz, CDCl₃) δ=8.39-8.28 (m, 2H), 7.00 (d, J=3.6 Hz, 1H), 6.72 (t, J=53.6 Hz, 1H), 4.98-4.81 (m, 2H), 4.59-4.37 (m, 2H), 4.33-4.14 (m, 2H), 3.09-2.97 (m, 1H), 2.84-2.65 (m, 2H), 2.38-2.25 (m, 2H), 2.00-1.87 (m, 2H), 1.25 (d, J=6.8 Hz, 3H).

### Example 123

### (3R,9R*)-N-(2-Bromo-3-fluoropyridin-4-yl)-11-fluoro-9-hydroxy-3-methyl-3,4,8,9-tetrahydro-1H-pyrido[4′,3′:3,4]pyrazolo[1,5-a]azepine-2(7H)-carboxamide

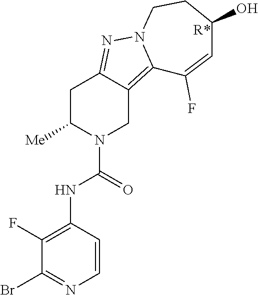 [SMILES: C[C@@H]1Cc2nn3c(c2CN1C(=O)Nc1ccnc(Br)c1F)C(F)=C[C@H](O)CC3]

The title compound was prepared from intermediate 52 in a manner analogous to Example 1. however using phenyl (2-bromo-3-fluoropyridin-4-yl)carbamate instead of phenyl (3-cyano-4-fluorophenyl)carbamate in Step B. MS (ESI): mass calcd. for C₁₈H₁₈BrF₂N₅O₂ 453.0; m/z found, 454.1/456.0 [M+H]+. ¹H NMR (400 MHz, CDCl₃) δ=8.18 (dt, J=2.7, 5.5 Hz, 1H), 8.07 (d, J=5.6 Hz, 1H), 6.94 (br s, 1H), 5.89-5.75 (m, 1H), 5.03-4.82 (m, 2H), 4.65 (dd, J=3.2, 5.1 Hz, 1H), 4.49-4.36 (m, 3H), 3.04 (br dd, J=5.6, 15.9 Hz, 1H), 2.76-2.64 (m, 1H), 2.33 (br dd, J=5.1, 9.3 Hz, 1H), 2.10 (br d, J=2.8 Hz, 1H), 1.24 (dd, J=6.8, 9.2 Hz, 3H).

### Example 124

### (3R,9S*)-N-(2-Bromo-3-fluoropyridin-4-yl)-11-fluoro-9-hydroxy-3-methyl-3,4,8,9-tetrahydro-1H-pyrido[4′,3′:3,4]pyrazolo[1,5-a]azepine-2(7H)-carboxamide

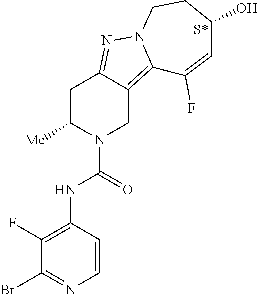 [SMILES: C[C@@H]1Cc2nn3c(c2CN1C(=O)Nc1ccnc(Br)c1F)C(F)=C[C@@H](O)CC3]

The title compound was prepared from intermediate 53 in a manner analogous to Example 1. however using phenyl (2-bromo-3-fluoropyridin-4-yl)carbamate instead of phenyl (3-cyano-4-fluorophenyl)carbamate in Step B. MS (ESI): mass calcd. for C₁₈H₁₈BrF₂N₅O₂ 453.0; m/z found, 454.0/456.0 [M+H]+. ¹H NMR (400 MHz, CDCl₃) δ=8.23-8.13 (m, 1H), 8.07 (d, J=5.6 Hz, 1H), 6.94 (br s, 1H), 5.89-5.75 (m, 1H), 5.03-4.82 (m, 2H), 4.65 (dd, J=3.2, 5.1 Hz, 1H), 4.49-4.36 (m, 3H), 3.04 (br dd, J=5.6, 15.9 Hz, 1H), 2.76-2.64 (m, 1H), 2.33 (m, 1H), 2.10 (m, 1H), 1.24 (dd, J=6.8, 9.2 Hz, 3H).

### Example 125

### (3R,9R*)-N-(3-Cyano-4-fluorophenyl)-11,11-difluoro-9-hydroxy-9-(hydroxymethyl)-3-methyl-3,4,8,9,10,11-hexahydro-1H-pyrido[4′,3′:3,4]pyrazolo[1,5-a]azepine-2(7H)-carboxamide

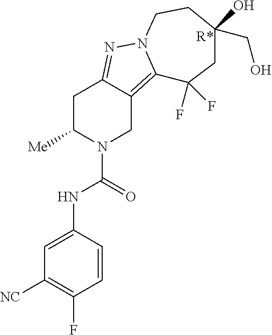 [SMILES: C[C@@H]1Cc2nn3c(c2CN1C(=O)Nc1ccc(F)c(C#N)c1)C(F)(F)C[C@@](O)(CO)CC3]

The title compound was prepared from intermediate 54 in a manner analogous to Example 1. MS (ESI): mass calcd. for C₂₁H₂₂N₅F₃O₃, 449.4; m/z found, 450.2 [M+H]⁺. ¹H NMR (400 MHz, MeOD-d₄) δ=7.80 (dd, J=2.8, 5.6 Hz, 1H), 7.70 (ddd, J=2.8, 4.7, 9.1 Hz, 1H), 7.28 (t, J=9.0 Hz, 1H), 5.00 (br d, J=16.5 Hz, 1H), 4.95-4.88 (m, 1H), 4.63 (ddd, J=3.3, 9.0, 14.6 Hz, 1H), 4.37-4.26 (m, 2H), 3.47-3.40 (m, 2H), 3.00 (dd, J=5.9, 15.8 Hz, 1H), 2.64 (d, J=15.9 Hz, 1H), 2.59-2.44 (m, 2H), 2.03-1.95 (m, 2H), 1.23 (d, J=6.8 Hz, 3H).

### Example 126

### (3R,9S*)-N-(3-Cyano-4-fluorophenyl)-11,11-difluoro-9-hydroxy-9-(hydroxymethyl)-3-methyl-3,4,8,9,10,11-hexahydro-1H-pyrido[4′,3′:3,4]pyrazolo[1,5-a]azepine-2(7H)-carboxamide

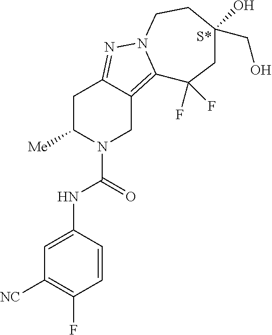 [SMILES: C[C@@H]1Cc2nn3c(c2CN1C(=O)Nc1ccc(F)c(C#N)c1)C(F)(F)C[C@](O)(CO)CC3]

The title compound was prepared from intermediate 55 in a manner analogous to Example 1. MS (ESI): mass calcd. for C₂₁H₂₂N₅F₃O₃, 449.4; m/z found, 450.2 [M+H]⁺. ¹H NMR (400 MHz, MeOD-d₄) δ=7.81 (dd, J=2.8, 5.6 Hz, 1H), 7.71 (ddd, J=2.8, 4.8, 9.2 Hz, 1H), 7.28 (t, J=9.0 Hz, 1H), 5.05 (br d, J=16.8 Hz, 1H), 4.96-4.90 (m, 1H), 4.60-4.54 (m, 1H), 4.39-4.23 (m, 2H), 3.45 (s, 2H), 3.01 (dd, J=5.9, 15.8 Hz, 1H), 2.64 (d, J=15.7 Hz, 1H), 2.58-2.46 (m, 2H), 2.08-1.94 (m, 2H), 1.21 (d, J=6.8 Hz, 3H).

### Example 127

### (3R,9R*)-N-(2-Bromo-3-fluoropyridin-4-yl)-11,11-difluoro-9-(fluoromethyl)-9-hydroxy-3-methyl-3,4,8,9,10,11-hexahydro-1H-pyrido[4′,3′:3,4]pyrazolo[1,5-a]azepine-2(7H)-carboxamide

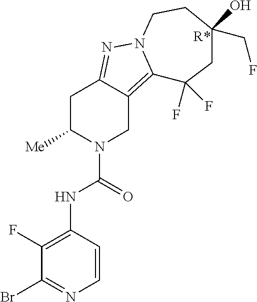 [SMILES: C[C@@H]1Cc2nn3c(c2CN1C(=O)Nc1ccnc(Br)c1F)C(F)(F)C[C@@](O)(CF)CC3]

The title compound was prepared from intermediate 54 in a manner analogous to Example 1. however using phenyl (2-bromo-3-fluoropyridin-4-yl)carbamate instead of phenyl (3-cyano-4-fluorophenyl)carbamate in Step B. MS (ESI): mass calcd. for C₁₉H₂₀BrF₄N₅O₂ 505.0/507.0; m/z found, 506.0/508.0 [M+H]+. ¹H NMR (400 MHz, CDCl₃) δ=8.17 (t, J=5.5 Hz, 1H), 8.07 (d, J=5.5 Hz, 1H), 6.92 (br d, J=3.5 Hz, 1H), 5.02-4.78 (m, 2H), 4.64 (dd, J=10.3, 14.2 Hz, 1H), 4.50-4.32 (m, 3H), 4.30-4.20 (m, 1H), 3.02 (dd, J=5.7, 15.9 Hz, 1H), 2.76-2.41 (m, 4H), 2.19-1.92 (m, 2H), 1.26 (d, J=7.0 Hz, 3H).

### Example 128

### (3R,9R*)-N-(3-Cyano-4-fluorophenyl)-11,11-difluoro-9-(fluoromethyl)-9-hydroxy-3-methyl-3,4,8,9,10,11-hexahydro-1H-pyrido[4′,3′:3,4]pyrazolo[1,5-a]azepine-2(7H)-carboxamide

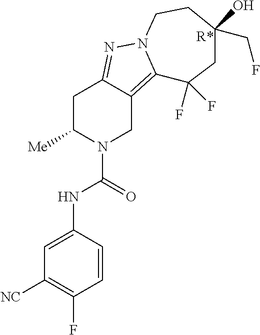 [SMILES: C[C@@H]1Cc2nn3c(c2CN1C(=O)Nc1ccc(F)c(C#N)c1)C(F)(F)C[C@@](O)(CF)CC3]

The title compound was prepared from intermediate 56 in a manner analogous to Example 1. MS (ESI): mass calcd. for C₂₁H₂₁F₄N₅O₂ 451.2; m/z found, 452.2 [M+H]⁺. ¹H NMR (400 MHz, CDCl₃) δ=7.72 (dd, J=2.7, 5.4 Hz, 1H), 7.61 (ddd, J=2.8, 4.6, 9.1 Hz, 1H), 7.15 (t, J=8.7 Hz, 1H), 6.50 (s, 1H), 4.94 (br t, J=6.4 Hz, 1H), 4.80 (br d, J=15.5 Hz, 1H), 4.69-4.58 (m, 1H), 4.48-4.33 (m, 3H), 4.25 (d, J=2.7 Hz, 1H), 3.01 (dd, J=6.0, 15.8 Hz, 1H), 2.75-2.42 (m, 4H), 2.17-1.97 (m, 2H), 1.23 (d, J=6.8 Hz, 3H).

### Example 129

### (3R,9 S*)-N-(2-Bromo-3-fluoropyridin-4-yl)-11,11-difluoro-9-(fluoromethyl)-9-hydroxy-3-methyl-3,4,8,9,10,11-hexahydro-1H-pyrido[4′,3′:3,4]pyrazolo[1,5-a]azepine-2(7H)-carboxamide

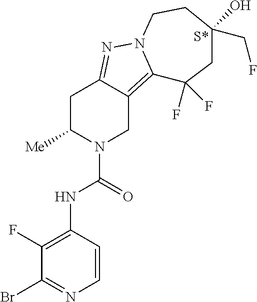 [SMILES: C[C@@H]1Cc2nn3c(c2CN1C(=O)Nc1ccnc(Br)c1F)C(F)(F)C[C@](O)(CF)CC3]

The title compound was prepared from intermediate 57 in a manner analogous to Example 1, however using phenyl (2-bromo-3-fluoropyridin-4-yl)carbamate instead of phenyl (3-cyano-4-fluorophenyl)carbamate in Step B. MS (ESI): mass calcd. for C₁₉H₂₀BrF₄N₅O₂ 505.0/507.0; m/z found, 506.0/508.0 [M+H]+. ¹H NMR (400 MHz, CDCl₃) δ=8.18 (t, J=5.5 Hz, 1H), 8.07 (d, J=5.5 Hz, 1H), 6.92 (br d, J=3.5 Hz, 1H), 4.95-4.87 (m, 2H), 4.44 (dd, J=10.3, 14.2 Hz, 1H), 4.42-4.39 (m, 3H), 4.36-4.29 (m, 1H), 3.02 (dd, J=5.7, 15.9 Hz, 1H), 2.76-2.54 (m, 4H), 2.12-2.09 (m, 2H), 1.25 (d, J=7.0 Hz, 3H).

### Example 130

### (3R,9S*)-N-(3-Cyano-4-fluorophenyl)-11,11-difluoro-9-(fluoromethyl) -9-hydroxy-3-methyl-3,4,8,9,10,11-hexahydro-1H-pyrido[4′,3′:3,4]pyrazolo[1,5-a]azepine-2(7H)-carboxamide

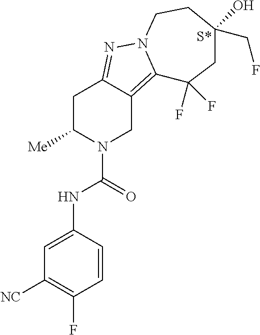 [SMILES: C[C@@H]1Cc2nn3c(c2CN1C(=O)Nc1ccc(F)c(C#N)c1)C(F)(F)C[C@](O)(CF)CC3]

The title compound was prepared from intermediate 57 in a manner analogous to Example 1. MS (ESI): mass calcd. for C₂₁H₂₁F₄N₅O₂ 451.2; m/z found, 452.2 [M+H]⁺. ¹H NMR (400 MHz, CDCl₃) δ=7.75 (dd, J=2.7, 5.4 Hz, 1H), 7.61 (ddd, J=2.8, 4.6, 9.1 Hz, 1H), 7.15 (t, J=8.7 Hz, 1H), 6.48 (s, 1H), 4.86 (br t, J=6.4 Hz, 1H), 4.51 (br d, J=15.5 Hz, 1H), 4.44-4.26 (m, 1H), 4.42-4.31 (m, 3H), 4.29 (d, J=2.7 Hz, 1H), 3.02 (dd, J=6.0, 15.8 Hz, 1H), 2.69-2.53 (m, 4H), 2.12-2.08 (m, 2H), 1.21 (d, J=6.8 Hz, 3H).

### Example 131

### (3R,9R)-N-(3-Cyano-4-fluorophenyl)-11,11-difluoro-9-(fluoromethyl) -3-methyl-3,4,8,9,10,11-hexahydro-1H-pyrido[4′,3′:3,4]pyrazolo[1,5-a]azepine-2(7H)-carboxamide

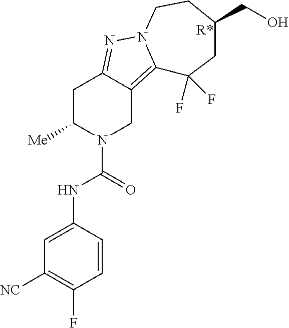 [SMILES: C[C@@H]1Cc2nn3c(c2CN1C(=O)Nc1ccc(F)c(C#N)c1)C(F)(F)C[C@H](CO)CC3]

The title compound was prepared from intermediate 58 in a manner analogous to Example 1. MS (ESI): mass calcd. for C₂₁H₂₂F₃N₅O₂ 433.2; m/z found, 434.2 [M+H]⁺. ¹H NMR (400 MHz, CDCl₃) δ=7.72 (dd, J=2.8, 5.4 Hz, 1H), 7.61 (ddd, J=2.9, 4.6, 9.1 Hz, 1H), 7.15 (t, J=8.7 Hz, 1H), 6.53 (s, 1H), 5.00-4.91 (m, 1H), 4.78 (br d, J=15.0 Hz, 1H), 4.54 (br dd, J=4.2, 14.5 Hz, 1H), 4.39 (br d, J=15.0 Hz, 1H), 4.27-4.18 (m, 1H), 3.62 (d, J=6.0 Hz, 2H), 3.00 (dd, J=5.7, 15.5 Hz, 1H), 2.71-2.55 (m, 2H), 2.34-1.92 (m, 3H), 1.66 (br s, 2H), 1.22 (d, J=6.8 Hz, 3H).

### Example 132

### (3R,9S*)-N-(3-Cyano-4-fluorophenyl)-11,11-difluoro-9-(fluoromethyl) -3-methyl-3,4,8,9,10,11-hexahydro-1H-pyrido[4′,3′:3,4]pyrazolo[1,5-a]azepine-2(7H)-carboxamide

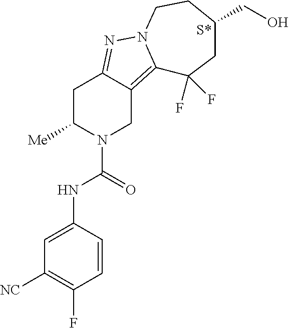 [SMILES: C[C@@H]1Cc2nn3c(c2CN1C(=O)Nc1ccc(F)c(C#N)c1)C(F)(F)C[C@@H](CO)CC3]

The title compound was prepared from intermediate 59 in a manner analogous to Example 1. MS (ESI): mass calcd. for C₂₁H₂₂F₃N₅O₂ 433.2; m/z found, 434.2 [M+H]⁺. ¹H NMR (400 MHz, CDCl₃) δ=7.74 (dd, J=2.8, 5.4 Hz, 1H), 7.61 (ddd, J=2.8, 4.5, 9.1 Hz, 1H), 7.15 (t, J=8.7 Hz, 1H), 6.52 (s, 1H), 5.01 (quin, J=6.4 Hz, 1H), 4.83 (br d, J=15.2 Hz, 1H), 4.60-4.51 (m, 1H), 4.33 (br d, J=15.0 Hz, 1H), 4.27-4.15 (m, 1H), 3.64 (d, J=6.0 Hz, 2H), 3.01 (dd, J=5.8, 15.6 Hz, 1H), 2.69-2.55 (m, 2H), 2.30-1.97 (m, 3H), 1.76-1.64 (m, 2H), 1.20 (d, J=6.8 Hz, 3H).

### Example 133

### (S*)-N-(2-Bromo-3-fluoropyridin-4-yl)-11,11-difluoro-9-(hydroxymethyl)-3,4,8,9,10,11-hexahydro-1H-pyrido[4′,3′:3,4]pyrazolo[1,5-a]azepine-2(7H)-carboxamide

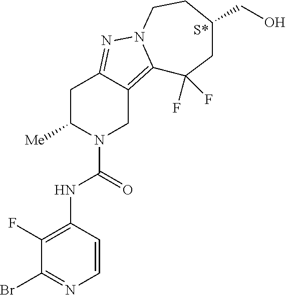 [SMILES: C[C@@H]1Cc2nn3c(c2CN1C(=O)Nc1ccnc(Br)c1F)C(F)(F)C[C@@H](CO)CC3]

The title compound was prepared from intermediate 62 in a manner analogous to Example 1, however using phenyl (2-bromo-3-fluoropyridin-4-yl)carbamate instead of phenyl (3-cyano-4-fluorophenyl)carbamate in Step B. MS (ESI): mass calcd. for C₁₈H₁₉BrF₃N₅O₂ 473.0/475.0; m/z found, 474.0/476.0 [M+H]+. ¹H NMR (400 MHz, CDCl₃) δ=8.17 (t, J=5.5 Hz, 1H), 8.07 (d, J=5.5 Hz, 1H), 6.94 (d, J=3.3 Hz, 1H), 4.68 (s, 2H), 4.53 (dd, J=4.0, 14.4 Hz, 1H), 4.26-4.14 (m, 1H), 3.91-3.74 (m, 2H), 3.62 (d, J=6.0 Hz, 2H), 2.86 (t, J=5.7 Hz, 2H), 2.67-2.52 (m, 1H), 2.35-1.92 (m, 3H), 1.69-1.63 (m, 1H).

### Example 134

### (S*)-N-(3-Cyano-4-fluorophenyl)-11,11-difluoro-9-(hydroxymethyl)-3,4,8,9,10,11-hexahydro-1H-pyrido[4′,3′:3,4]pyrazolo[1,5-a]azepine-2(7H)-carboxamide

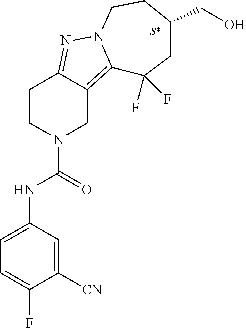 [SMILES: N#Cc1cc(NC(=O)N2CCc3nn4c(c3C2)C(F)(F)C[C@@H](CO)CC4)ccc1F]

The title compound was prepared from intermediate 62 in a manner analogous to Example 1. MS (ESI): mass calcd. for C₂₀H₂₀F₃N₅O₂ 419.2; m/z found, 420.2 [M+H]⁺. ¹H NMR (400 MHz, CDCl₃) δ=7.81-7.68 (m, 1H), 7.65-7.55 (m, 1H), 7.15 (t, J=8.7 Hz, 1H), 6.53 (s, 1H), 4.63 (s, 2H), 4.51 (d, J=4.4 Hz, 1H), 4.26-4.14 (m, 1H), 3.85-3.76 (m, 2H), 3.62 (d, J=5.7 Hz, 2H), 2.83 (t, J=5.7 Hz, 2H), 2.67-2.52 (m, 1H), 2.27 (s, 1H), 2.20-2.11 (m, 1H), 2.10-1.97 (m, 1H), 1.71-1.59 (m, 2H).

### Example 135

### (R*)-N-(2-Bromo-3-fluoropyridin-4-yl)-11,11-difluoro-9-(hydroxymethyl)-3,4,8,9,10,11-hexahydro-1H-pyrido[4′,3′:3,4]pyrazolo[1,5-a]azepine-2(7H)-carboxamide

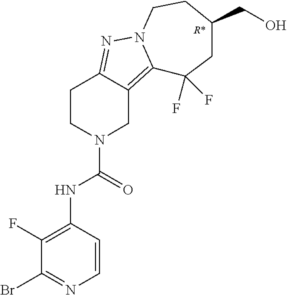 [SMILES: O=C(Nc1ccnc(Br)c1F)N1CCc2nn3c(c2C1)C(F)(F)C[C@H](CO)CC3]

The title compound was prepared from intermediate 61 in a manner analogous to Example 1, however using phenyl (2-bromo-3-fluoropyridin-4-yl)carbamate instead of phenyl (3-cyano-4-fluorophenyl)carbamate in Step B. MS (ESI): mass calcd. for C₁₈H₁₉BrF₃N₅O₂ 473.0/475.0; m/z found, 474.0/476.0 [M+H]+. ¹H NMR (400 MHz, CDCl₃) δ=8.19 (t, J=5.5 Hz, 1H), 8.09 (d, J=5.5 Hz, 1H), 6.96 (d, J=3.8 Hz, 1H), 4.69 (s, 2H), 4.61-4.49 (m, 1H), 4.29-4.16 (m, 1H), 3.92-3.75 (m, 2H), 3.64 (d, J=6.0 Hz, 2H), 2.87 (t, J=5.8 Hz, 2H), 2.69-2.54 (m, 1H), 2.38-2.14 (m, 2H), 2.13-1.94 (m, 1H), 1.72-1.65 (m, 1H).

### Example 136

### (R*)-N-(3-Cyano-4-fluorophenyl)-11,11-difluoro-9-(hydroxymethyl)-3,4,8,9,10,11-hexahydro-1H-pyrido[4′,3′:3,4]pyrazolo[1,5-a]azepine-2(7H)-carboxamide

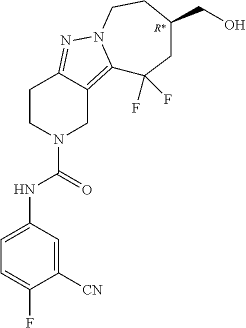 [SMILES: N#Cc1cc(NC(=O)N2CCc3nn4c(c3C2)C(F)(F)C[C@H](CO)CC4)ccc1F]

The title compound was prepared from intermediate 61 in a manner analogous to Example 1. MS (ESI): mass calcd. for C₂₀H₂₀F₃N₅O₂ 419.2; m/z found, 420.2 [M+H]⁺. ¹H NMR (400 MHz, CDCl₃) δ=7.73 (dd, J=2.8, 5.4 Hz, 1H), 7.61 (ddd, J=2.9, 4.5, 9.1 Hz, 1H), 7.15 (t, J=8.7 Hz, 1H), 6.48 (s, 1H), 4.63 (br s, 2H), 4.53 (dd, J=4.5, 14.7 Hz, 1H), 4.30-4.14 (m, 1H), 3.81 (t, J=6.0 Hz, 2H), 3.62 (d, J=4.6 Hz, 2H), 2.84 (t, J=5.7 Hz, 2H), 2.69-2.52 (m, 1H), 2.27 (s, 1H), 2.20-1.92 (m, 2H), 1.74-1.56 (m, 2H), 1.48 (s, 1H), 1.50-1.41 (m, 1H), 1.49-1.39 (m, 1H).

### Example 137

### (R*)-N-(2-Bromo-3-fluoropyridin-4-yl)-11,11-difluoro-9-hydroxy-3,4,8,9,10,11-hexahydro-1H-pyrido[4′,3′:3,4]pyrazolo[1,5-a]azepine-2(7H)-carboxamide

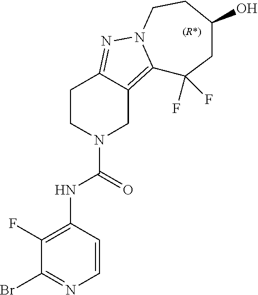 [SMILES: O=C(Nc1ccnc(Br)c1F)N1CCc2nn3c(c2C1)C(F)(F)C[C@H](O)CC3]

### Step A. (R*)-tert-Butyl 11,11-difluoro-9-hydroxy-3,4,8,9,10,11-hexahydro-1H-pyrido[4′,3′:3,4]pyrazolo[1,5-a]azepine-2(7H)-carboxylate

To a solution of (R*)-benzyl 11,11-difluoro-9-hydroxy-3,4,8,9,10,11-hexahydro-1H-pyrido[4′,3′:3,4]pyrazolo[1,5-a]azepine-2(7H)-carboxylate (intermediate 63, 300 mg, 794.95 μmol) in EtOH (10 mL) was added Pd/C (70 mg, 10% purity) and Boc₂O (346.99 mg, 1.59 mmol, 365.26 4). The mixture was stirred at 20° C. under H₂ (15 psi) for 16 h. The mixture was filtered and concentrated under reduced pressure to afford (R*)-tert-butyl 11,11-difluoro-9-hydroxy-3,4,8,9,10,11-hexahydro-1H-pyrido[4′,3′:3,4]pyrazolo[1,5-a]azepine-2(7H)-carboxylate (300 mg, crude) as yellow oil.

### Step B. (R*)-11,11-Difluoro-2,3,4,7,8,9,10,11-octahydro-1H-pyrido[4′,3′:3,4]pyrazolo[1,5-a]azepin-9-ol

To a solution of (R*)-tert-butyl11,11-difluoro-9-hydroxy-3,4,8,9,10,11-hexahydro-1H-pyrido[4′,3′:3,4]pyrazolo[1,5-a]azepine-2(7H)-carboxylate (300 mg, crude) in DCM (2 mL) was added TFA (1.54 g, 13.51 mmol, 1 mL) and stirred at 20° C. for 2 h. The mixture was filtered and concentrated under reduced pressure to afford (R*)-11,11-difluoro-2,3,4,7,8,9,10,11-octahydro-1H-pyrido[4′,3′:3,4]pyrazolo[1,5-a]azepin-9-ol (360 mg, crude, TFA) as yellow oil.

### Step C. (R*)-N-(2-Bromo-3-fluoropyridin-4-yl)-11,11-difluoro-9-hydroxy-3,4,8,9,10,11-hexahydro-1H-pyrido[4′,3′:3,4]pyrazolo[1,5-a]azepine-2(7H)-carboxamide

To a solution of (R*)-11,11-difluoro-2,3,4,7,8,9,10,11-octahydro-1H-pyrido[4′,3′:3,4] pyrazolo[1,5-a]azepin-9-ol (120 mg) in DCM (2 mL) was added phenyl N-(2-bromo-3-fluoro-4-pyridyl)carbamate (82.44 mg, 265 μmol) and TEA (134.08 mg, 1.33 mmol, 184.42 μL). The mixture was stirred at 25° C. for 16 h. The reaction mixture was adjusted to pH-6 by formic acid, filtered and concentrated under reduced pressure. The residue was purified by RP HPLC (condition A) to afford the title compound (73.3 mg, 157.67 μmol, 59.50% yield, 99% purity) as white solid. MS (ESI): mass calcd. for C₁₇H₁₇BrF₃N₅O₂, 459.0; m/z found, 460.0, 462.0 [M+H]⁺. ¹H NMR (400 MHz, CDCl₃) δ=8.21-8.13 (m, 1H), 8.08 (d, J=5.5 Hz, 1H), 6.94 (br d, J=3.5 Hz, 1H), 4.68 (s, 2H), 4.53 (dd, J=6.1, 14.7 Hz, 1H), 4.36-4.13 (m, 2H), 3.88-3.76 (m, 2H), 2.91-2.69 (m, 3H), 2.42-2.22 (m, 2H), 2.04-1.89 (m, 1H).

### Example 138

### (R*)-N-(3-Cyano-4-fluorophenyl)-11,11-difluoro-9-hydroxy-3,4,8,9,10,11-hexahydro-1H-pyrido[4′,3′:3,4]pyrazolo[1,5-a]azepine-2(7H)-carboxamide

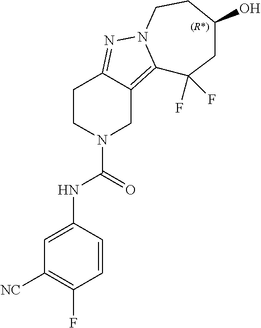 [SMILES: N#Cc1cc(NC(=O)N2CCc3nn4c(c3C2)C(F)(F)C[C@H](O)CC4)ccc1F]

The title compound was prepared from (R*)-11,11-difluoro-2,3,4,7,8,9,10,11-octahydro-1H-pyrido[4′,3′:3,4]pyrazolo[1,5-a]azepin-9-ol in a manner analogous to Example 137, however using phenyl (3-cyano-4-fluorophenyl)carbamate instead of phenyl (2-bromo-3-fluoropyridin-4-yl)carbamate . MS (ESI): mass calcd. for C₁₉H₁₈F₃N₅O₂ 405.1; m/z found, 406.1 [M+H]⁺. ¹H NMR (400 MHz, CDCl₃) δ=7.74 (m, 1H), 7.62 (m, 1H), 7.17 (t, J=8.7 Hz, 1H), 6.50 (s, 1H), 4.64 (s, 2H), 4.36-4.13 (m, 2H), 3.82 (t, J=5.9 Hz, 2H), 2.90-2.72 (m, 3H), 2.45-2.25 (m, 2H), 2.06-1.83 (m, 2H).

### Example 139

### (R*)-N-(2-(Difluoromethyl)-3-fluoropyridin-4-yl)-11,11-difluoro-9-hydroxy-3,4,8,9,10,11-hexahydro-1H-pyrido[4′,3′:3,4]pyrazolo[1,5-a]azepine-2(7H)-carboxamide

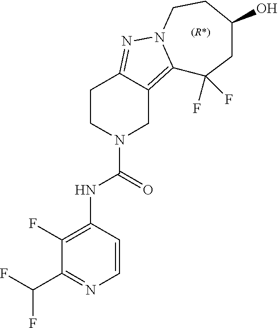 [SMILES: O=C(Nc1ccnc(C(F)F)c1F)N1CCc2nn3c(c2C1)C(F)(F)C[C@H](O)CC3]

The title compound was prepared from (R*)-11,11-difluoro-2,3,4,7,8,9,10,11-octahydro-1H-pyrido[4′,3′:3,4]pyrazolo[1,5-a]azepin-9-ol in a manner analogous to Example 137, however using phenyl (2-(difluoromethyl)-3-fluoropyridin-4-yl)carbamate instead of phenyl (2-bromo-3-fluoropyridin-4-yl)carbamate. MS (ESI): mass calcd. for C₁₈H₁₈F₅N₅O₂ 431.1; m/z found, 432.1 [M+H]⁺. ¹H NMR (400 MHz, CDCl₃) δ=8.40-8.24 (m, 2H), 7.02 (d, J=3.8 Hz, 1H), 6.91-6.58 (m, 1H), 4.69 (s, 2H), 4.54 (dd, J=6.8, 14.2 Hz, 1H), 4.39-4.11 (m, 2H), 3.91-3.75 (m, 2H), 2.91-2.69 (m, 3H), 2.44-2.19 (m, 2H), 2.05-1.82 (m, 2H).

### Example 140

### (S *)-N-(2-Bromo-3-fluoropyridin-4-yl)-11,11-difluoro-9-hydroxy-3,4,8,9,10,11-hexahydro-1H-pyrido[4′,3′:3,4]pyrazolo[1,5-a]azepine-2(7H)-carboxamide

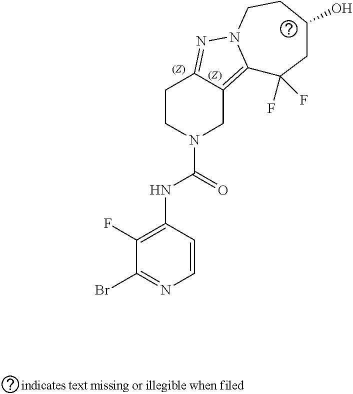

The title compound was prepared from intermediate 64 in a manner analogous to Example 137. MS (ESI): mass calcd. for C₁₇H₁₇BrF₃N₅O₂, 459.0; m/z found, 460.0, 462.0 [M+H]⁺. ¹H NMR (400 MHz, CDCl₃) δ=8.16 (t, J=5.6 Hz, 1H), 8.06 (d, J=5.6 Hz, 1H), 6.95-6.82 (m, 1H), 4.66 (s, 2H), 4.52 (dd, J=6.2, 14.7 Hz, 1H), 4.34-4.23 (m, 1H), 4.17 (dd, J=10.3, 14.3 Hz, 1H), 3.86-3.72 (m, 2H), 2.90-2.67 (m, 3H), 2.41-2.21 (m, 2H), 1.98-1.90 (m, 1H).

### Example 141

### (S*)-N-(3-Cyano-4-fluorophenyl)-11,11-difluoro-9-hydroxy-3,4,8,9,10,11-hexahydro-1H-pyrido[4′,3′:3,4]pyrazolo[1,5-a]azepine-2(7H)-carboxamide

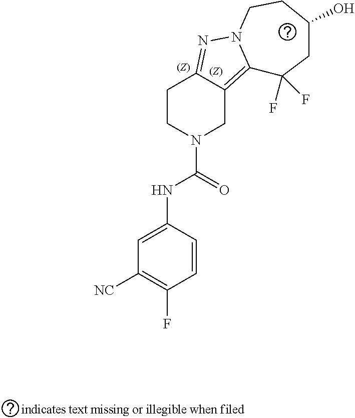

The title compound was prepared from intermediate 64 in a manner analogous to Example 137, substituting phenyl (3-cyano-4-fluorophenyl)carbamate instead of phenyl (2-bromo-3-fluoropyridin-4-yl)carbamate . MS (ESI): mass calcd. for C₁₉H₁₈F₃N₅O₂ 405.1; m/z found, 406.1 [M+H]⁺. ¹H NMR (400 MHz, CDCl₃) δ=7.72 (dd, J=2.8, 5.4 Hz, 1H), 7.61 (ddd, J=2.9, 4.5, 9.1 Hz, 1H), 7.15 (t, J=8.7 Hz, 1H), 6.51 (s, 1H), 4.63 (s, 2H), 4.53 (dd, J=6.1, 14.6 Hz, 1H), 4.30 (br d, J=4.3 Hz, 1H), 4.18 (dd, J=10.7, 13.9 Hz, 1H), 3.81 (t, J=5.9 Hz, 2H), 2.89-2.70 (m, 3H), 2.42-2.23 (m, 2H), 2.03-1.85 (m, 2H).

### Example 142

### (S*)-N-(2-(Difluoromethyl)-3-fluoropyridin-4-yl)-11,11-difluoro-9-hydroxy-3,4,8,9,10,11-hexahydro-1H-pyrido[4′,3′:3,4]pyrazolo[1,5-a]azepine-2(7H)-carboxamide

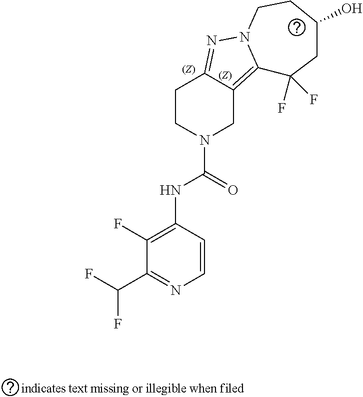

The title compound was prepared from intermediate 64 in a manner analogous to Example 137, however using phenyl (2-(difluoromethyl)-3-fluoropyridin-4-yl)carbamate instead of phenyl (2-bromo-3-fluoropyridin-4-yl)carbamate. MS (ESI): mass calcd. for C₁₈H₁₈F₅N₅O₂ 431.1; m/z found, 432.1 [M+H]⁺. ¹H NMR (400 MHz, CDCl₃) δ=8.40-8.28 (m, 2H), 7.10-6.99 (m, 1H), 6.91-6.59 (m, 1H), 4.69 (s, 2H), 4.53 (dd, J=6.2, 14.7 Hz, 1H), 4.37-4.25 (m, 1H), 4.19 (dd, J=10.5, 14.4 Hz, 1H), 3.91-3.77 (m, 2H), 2.87 (t, J=5.8 Hz, 2H), 2.83-2.69 (m, 1H), 2.45-2.23 (m, 2H), 2.04-1.91 (m, 1H).

### Example 143

### (3R)-N-(3-Cyano-4-fluorophenyl)-11,11-difluoro-10-hydroxy-3-methyl-1,3,4,7,8,9,10,11-octahydro-2H-pyrido[4′,3′:3,4]pyrazolo[1,5-a]azepine-2-carboxamide

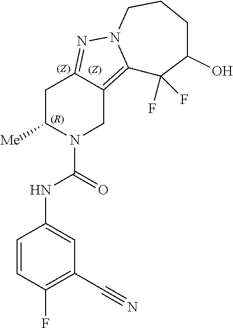

The title compound was prepared in a manner analogous to Example 1, however using tert-butyl (3R)-11,11-difluoro-10-hydroxy-3-methyl-1,3,4,7,8,9,10,11-octahydro-2H-pyrido[4′,3′:3,4]pyrazolo[1,5-a]azepine-2-carboxylate (Intermediate 65) instead of tert-butyl 11,11-difluoro-8-methylene-3,4,8,9,10,11-hexahydro-1H-pyrido[4′,3′:3,4]pyrazolo[1,5-a]azepine-2(7H)-carboxylate (Intermediate 1) in Step A. MS (ESI): mass calcd. for C₂₀H₂₀F₃N₅O₂ 419.4; m/z found, 420.3 [M+H]+. ¹HNMR (400 MHz, CDCl₃) δ=7.75-7.58 (m, 2H), 7.13 (t, J=8.8 Hz, 1H), 6.94 (br s, 1H), 5.04-4.99 (m, 1H), 4.81-4.73 (m, 1H), 4.59-4.48 (m, 1H), 4.36-4.32 (m, 1H), 3.94-3.75 (m, 2H), 3.09-2.98 (m, 1H), 2.64-2.58 (m, 1H), 2.23-2.09 (m, 2H), 1.97-1.81 (m, 2H), 1.22 (d, J=6.6 Hz, 3H).

### Biological Data

### HBV Replication Inhibition Assay

HBV replication inhibition by the disclosed compounds were determined in cells infected or transfected with HBV or cells with stably integrated HBV, such as HepG2.2.15 cells (Sells et al. 1987). In this example, HepG2.2.15 cells were maintained in cell culture medium containing 10% fetal bovine serum (FBS), Geneticin, L-glutamine, penicillin and streptomycin. HepG2.2.15 cells were seeded in 96-well plates at a density of 40,000 cells/well and were treated with serially diluted compounds at a final DMSO concentration of 0.5% either alone or in combination by adding drugs in a checker box format. Cells were incubated with compounds for three days, after which medium was removed and fresh medium containing compounds was added to cells and incubated for another three days. At day 6, supernatant was removed and treated with DNase at 37° C. for 60 minutes, followed by enzyme inactivation at 75° C. for 15 minutes. Encapsidated HBV DNA was released from the virions and covalently linked HBV polymerase by incubating in lysis buffer (Affymetrix QS0010) containing 2.5 μg proteinase K at 50° C. for 40 minutes. HBV DNA was denatured by addition of 0.2 M NaOH and detected using a branched DNA (BDNA) QuantiGene assay kit according to manufacturer recommendation (Affymetrix). HBV DNA levels were also quantified using qPCR, based on amplification of encapsidated HBV DNA extraction with QuickExtraction Solution (Epicentre Biotechnologies) and amplification of HBV DNA using HBV specific PCR probes that can hybridize to HBV DNA and a fluorescently labeled probe for quantitation. In addition, cell viability of HepG2.2.15 cells incubated with test compounds alone or in combination was determined by using CellTitre-Glo reagent according to the manufacturer protocol (Promega). The mean background signal from wells containing only culture medium was subtracted from all other samples, and percent inhibition at each compound concentration was calculated by normalizing to signals from HepG2.2.15 cells treated with 0.5% DMSO using equation E1.

% inhibition=(DMSOave−Xi)/DMSOave×100% E1

where DMSOave is the mean signal calculated from the wells that were treated with DMSO control (0% inhibition control) and Xi is the signal measured from the individual wells. EC₅₀ values, effective concentrations that achieved 50% inhibitory effect, were determined by non-linear fitting using Graphpad Prism software (San Diego, Calif.) and equation E2.

Y=Ymin+(Ymax−Ymin)/(1+10(Log EC50−X)×HillSlope) E2

where Y represents percent inhibition values and X represents the logarithm of compound concentrations.

Selected disclosed compounds were assayed in the HBV replication assay (BDNA assay), as described above, and a representative group of these active compounds is shown in Table 3. Table 3 shows EC₅₀ values obtained by the BDNA assay for a group of select compounds.

TABLE 3
Activity in BDNA-assay (EC₅₀)

| | | HepG2.2.15 |
|---|---|---|
| | | EC50 HBV |
| Ex # | Compound_Name | DNA (uM) |
| 1 | N-(3-Cyano-4-fluorophenyl)-11,11-difluoro-8-methylene- | 0.044 |
| | 3,4,8,9,10,11-hexahydro-1H-pyrido[4′,3′:3,4]pyrazolo[1,5- | |
| | a]azepine-2(7H)-carboxamide; | |
| 2 | 11,11-Difluoro-N-(4-fluoro-3-(trifluoromethyl)phenyl)-8- | 0.051 |
| | methylene-3,4,8,9,10,11-hexahydro-1H- | |
| | pyrido[4′,3′:3,4]pyrazolo[1,5-a]azepine-2(7H)-carboxamide; | |
| 3 | (S)-N-(3-Cyano-4-fluorophenyl)-11,11-difluoro-8-hydroxy- | 0.022 |
| | 3,4,8,9,10,11-hexahydro-1H-pyrido[4′,3′:3,4]pyrazolo[1,5- | |
| | a]azepine-2(7H)-carboxamide; | |
| 4 | (S)-11,11-Difluoro-N-(4-fluoro-3-(trifluoromethyl)phenyl)-8- | 0.037 |
| | hydroxy-3,4,8,9,10,11-hexahydro-1H- | |
| | pyrido[4′,3′:3,4]pyrazolo[1,5-a]azepine-2(7H)-carboxamide; | |
| 5 | (S)-N-(2-Bromo-3-fluoropyridin-4-yl)-11,11-difluoro-8- | 0.057 |
| | hydroxy-3,4,8,9,10,11-hexahydro-1H- | |
| | pyrido[4′,3′:3,4]pyrazolo[1,5-a]azepine-2(7H)-carboxamide; | |
| 6 | (S)-N-(3-Cyano-2,4-difluorophenyl)-11,11-difluoro-8-hydroxy- | 0.044 |
| | 3,4,8,9,10,11-hexahydro-1H-pyrido[4′,3′:3,4]pyrazolo[1,5- | |
| | a]azepine-2(7H)-carboxamide; | |
| 7 | (S)-N-(3-Bromo-2,4-difluorophenyl)-11,11-difluoro-8-hydroxy- | 0.031 |
| | 3,4,8,9,10,11-hexahydro-1H-pyrido[4′,3′:3,4]pyrazolo[1,5- | |
| | a]azepine-2(7H)-carboxamide; | |
| 8 | (R)-N-(3-Cyano-4-fluorophenyl)-11,11-difluoro-8-hydroxy- | 0.01 |
| | 3,4,8,9,10,11-hexahydro-1H-pyrido[4′,3′:3,4]pyrazolo[1,5- | |
| | a]azepine-2(7H)-carboxamide; | |
| 9 | (R)-11,11-Difluoro-N-(4-fluoro-3-(trifluoromethyl)phenyl)-8- | 0.028 |
| | hydroxy-3,4,8,9,10,11-hexahydro-1H- | |
| | pyrido[4′,3′:3,4]pyrazolo[1,5-a]azepine-2(7H)-carboxamide; | |
| 10 | (R)-N-(2-Bromo-3-fluoropyridin-4-yl)-11,11-difluoro-8- | 0.032 |
| | hydroxy-3,4,8,9,10,11-hexahydro-1H- | |
| | pyrido[4′,3′:3,4]pyrazolo[1,5-a]azepine-2(7H)-carboxamide; | |
| 11 | (R)-N-(3-Cyano-2,4-difluorophenyl)-11,11-difluoro-8-hydroxy- | 0.024 |
| | 3,4,8,9,10,11-hexahydro-1H-pyrido[4′,3′:3,4]pyrazolo[1,5- | |
| | a]azepine-2(7H)-carboxamide; | |
| 12 | (R)-N-(3-Bromo-2,4-difluorophenyl)-11,11-difluoro-8-hydroxy- | 0.019 |
| | 3,4,8,9,10,11-hexahydro-1H-pyrido[4′,3′:3,4]pyrazolo[1,5- | |
| | a]azepine-2(7H)-carboxamide; | |
| 13 | (S)-N-(3-Cyano-4-fluorophenyl)-8-(2,2-difluoroethoxy)-11,11- | 0.042 |
| | difluoro-3,4,8,9,10,11-hexahydro-1H- | |
| | pyrido[4′,3′:3,4]pyrazolo[1,5-a]azepine-2(7H)-carboxamide; | |
| 14 | (R)-N-(3-Cyano-4-fluorophenyl)-8-(2,2-difluoroethoxy)-11,11- | 0.051 |
| | difluoro-3,4,8,9,10,11-hexahydro-1H- | |
| | pyrido[4′,3′:3,4]pyrazolo[1,5-a]azepine-2(7H)-carboxamide; | |
| 15 | (R)-N-(3-Cyano-4-fluorophenyl)-8,11,11-trifluoro- | NT |
| | 3,4,8,9,10,11-hexahydro-1H-pyrido[4′,3′:3,4]pyrazolo[1,5- | |
| | a]azepine-2(7H)-carboxamide; | |
| 16 | (R)-8,11,11-Trifluoro-N-(4-fluoro-3-(trifluoromethyl)phenyl)- | NT |
| | 3,4,8,9,10,11-hexahydro-1H-pyrido[4′,3′:3,4]pyrazolo[1,5- | |
| | a]azepine-2(7H)-carboxamide; | |
| 17 | (S)-N-(3-Cyano-4-fluorophenyl)-8,11,11-trifluoro- | NT |
| | 3,4,8,9,10,11-hexahydro-1H-pyrido[4′,3′:3,4]pyrazolo[1,5- | |
| | a]azepine-2(7H)-carboxamide; | |
| 18 | (S)-8,11,11-Trifluoro-N-(4-fluoro-3-(trifluoromethyl)phenyl)- | NT |
| | 3,4,8,9,10,11-hexahydro-1H-pyrido[4′,3′:3,4]pyrazolo[1,5- | |
| | a]azepine-2(7H)-carboxamide; | |
| 19 | (S*)-N-(3-Cyano-4-fluorophenyl)-11,11-difluoro-8- | 0.004 |
| | (hydroxymethyl)-3,4,8,9,10,11-hexahydro-1H- | |
| | pyrido[4′,3′:3,4]pyrazolo[1,5-a]azepine-2(7H)-carboxamide; | |
| 20 | (S*)-11,11-Difluoro-N-(4-fluoro-3-(trifluoromethyl)phenyl)-8- | 0.004 |
| | (hydroxymethyl)-3,4,8,9,10,11-hexahydro-1H- | |
| | pyrido[4′,3′:3,4]pyrazolo[1,5-a]azepine-2(7H)-carboxamide; | |
| 21 | (S*)-N-(3-Cyano-2,4-difluorophenyl)-11,11-difluoro-8- | 0.001 |
| | (hydroxymethyl)-3,4,8,9,10,11-hexahydro-1H- | |
| | pyrido[4′,3′:3,4]pyrazolo[1,5-a]azepine-2(7H)-carboxamide; | |
| 22 | (S*)-N-(2-Bromo-3-fluoropyridin-4-yl)-11,11-difluoro-8- | 0.004 |
| | (hydroxymethyl)-3,4,8,9,10,11-hexahydro-1H- | |
| | pyrido[4′,3′:3,4]pyrazolo[1,5-a]azepine-2(7H)-carboxamide; | |
| 23 | (R*)-N-(3-Cyano-4-fluorophenyl)-11,11-difluoro-8- | 0.018 |
| | (hydroxymethyl)-3,4,8,9,10,11-hexahydro-1H- | |
| | pyrido[4′,3′:3,4]pyrazolo[1,5-a]azepine-2(7H)-carboxamide; | |
| 24 | (R*)-11,11-Difluoro-N-(4-fluoro-3-(trifluoromethyl)phenyl)-8- | 0.035 |
| | (hydroxymethyl)-3,4,8,9,10,11-hexahydro-1H- | |
| | pyrido[4′,3′:3,4]pyrazolo[1,5-a]azepine-2(7H)-carboxamide; | |
| 25 | (R*)-N-(3-Cyano-2,4-difluorophenyl)-11,11-difluoro-8- | 0.034 |
| | (hydroxymethyl)-3,4,8,9,10,11-hexahydro-1H- | |
| | pyrido[4′,3′:3,4]pyrazolo[1,5-a]azepine-2(7H)-carboxamide; | |
| 26 | (R*)-N-(2-Bromo-3-fluoropyridin-4-yl)-11,11-difluoro-8- | 0.025 |
| | (hydroxymethyl)-3,4,8,9,10,11-hexahydro-1H- | |
| | pyrido[4′,3′:3,4]pyrazolo[1,5-a]azepine-2(7H)-carboxamide; | |
| 27 | (S*)-N-(3-Cyano-4-fluorophenyl)-8-((2,2- | 0.006 |
| | difluoroethoxy)methyl)-11,11-difluoro-3,4,8,9,10,11-hexahydro- | |
| | 1H-pyrido[4′,3′:3,4]pyrazolo[1,5-a]azepine-2(7H)-carboxamide; | |
| 28 | (S*)-8-((2,2-Difluoroethoxy)methyl)-11,11-difluoro-N-(4- | 0.022 |
| | fluoro-3-(trifluoromethyl)phenyl)-3,4,8,9,10,11-hexahydro-1H- | |
| | pyrido[4′,3′:3,4]pyrazolo[1,5-a]azepine-2(7H)-carboxamide; | |
| 29 | (R*)-N-(3-Cyano-4-fluorophenyl)-8-((2,2- | 0.078 |
| | difluoroethoxy)methyl)-11,11-difluoro-3,4,8,9,10,11-hexahydro- | |
| | 1H-pyrido[4′,3′:3,4]pyrazolo[1,5-a]azepine-2(7H)-carboxamide; | |
| 30 | (R*)-8-((2,2-Difluoroethoxy)methyl)-11,11-difluoro-N-(4- | 0.267 |
| | fluoro-3-(trifluoromethyl)phenyl)-3,4,8,9,10,11-hexahydro-1H- | |
| | pyrido[4′,3′:3,4]pyrazolo[1,5-a]azepine-2(7H)-carboxamide; | |
| 31 | (R*)-N-(3-Cyano-4-fluorophenyl)-8-((2,2- | 0.042 |
| | difluoroethoxy)methyl)-11,11-difluoro-8-hydroxy-3,4,8,9,10,11- | |
| | hexahydro-1H-pyrido[4′,3′:3,4]pyrazolo[1,5-a]azepine-2(7H)- | |
| | carboxamide; | |
| 32 | (R*)-N-(2-Bromo-3-fluoropyridin-4-yl)-8-((2,2- | 0.138 |
| | difluoroethoxy)methyl)-11,11-difluoro-8-hydroxy-3,4,8,9,10,11- | |
| | hexahydro-1H-pyrido[4′,3′:3,4]pyrazolo[1,5-a]azepine-2(7H)- | |
| | carboxamide; | |
| 33 | (R*)-8-((2,2-Difluoroethoxy)methyl)-N-(2-(difluoromethyl)-3- | 0.356 |
| | fluoropyridin-4-yl)-11,11-difluoro-8-hydroxy-3,4,8,9,10,11- | |
| | hexahydro-1H-pyrido[4′,3′:3,4]pyrazolo[1,5-a]azepine-2(7H)- | |
| | carboxamide; | |
| 34 | (S*)-N-(3-Cyano-4-fluorophenyl)-8-((2,2- | 0.017 |
| | difluoroethoxy)methyl)-11,11-difluoro-8-hydroxy- | |
| | 3,4,8,9,10,11-hexahydro-1H-pyrido[4′,3′:3,4]pyrazolo[1,5- | |
| | a]azepine-2(7H)-carboxamide; | |
| 35 | (S*)-N-(2-Bromo-3-fluoropyridin-4-yl)-8-((2,2- | 0.098 |
| | difluoroethoxy)methyl)-11,11-difluoro-8-hydroxy- | |
| | 3,4,8,9,10,11-hexahydro-1H-pyrido[4′,3′:3,4]pyrazolo[1,5- | |
| | a]azepine-2(7H)-carboxamide; | |
| 36 | (S*)-8-((2,2-Difluoroethoxy)methyl)-N-(2-(difluoromethyl)-3- | 0.039 |
| | fluoropyridin-4-yl)-11,11-difluoro-8-hydroxy-3,4,8,9,10,11- | |
| | hexahydro-1H-pyrido[4′,3′:3,4]pyrazolo[1,5-a]azepine-2(7H)- | |
| | carboxamide; | |
| 37 | N-(3-Cyano-4-fluorophenyl)-11,11-difluoro-8-(fluoromethyl)- | 0.015 |
| | 8-hydroxy-3,4,8,9,10,11-hexahydro-1H- | |
| | pyrido[4′,3′:3,4]pyrazolo[1,5-a]azepine-2(7H)-carboxamide; | |
| 38 | (S*)-N-(2-Bromo-3-fluoropyridin-4-yl)-11,11-difluoro-8- | 0.084 |
| | (fluoromethyl)-8-hydroxy-3,4,8,9,10,11-hexahydro-1H- | |
| | pyrido[4′,3′:3,4]pyrazolo[1,5-a]azepine-2(7H)-carboxamide; | |
| 39 | (R*)-N-(2-Bromo-3-fluoropyridin-4-yl)-11,11-difluoro-8- | 0.144 |
| | (fluoromethyl)-8-hydroxy-3,4,8,9,10,11-hexahydro-1H- | |
| | pyrido[4′,3′:3,4]pyrazolo[1,5-a]azepine-2(7H)-carboxamide; | |
| 40 | N-(3-Cyano-4-fluorophenyl)-8-(cyanomethyl)-11,11-difluoro-8- | 0.013 |
| | hydroxy-3,4,8,9,10,11-hexahydro-1H- | |
| | pyrido[4′,3′:3,4]pyrazolo[1,5-a]azepine-2(7H)-carboxamide; | |
| 41 | (R*)-N-(2-Bromo-3-fluoropyridin-4-yl)-8-(cyanomethyl)- | 0.052 |
| | 11,11-difluoro-8-hydroxy-3,4,8,9,10,11-hexahydro-1H- | |
| | pyrido[4′,3′:3,4]pyrazolo[1,5-a]azepine-2(7H)-carboxamide; | |
| 42 | (R*)-8-(Cyanomethyl)-N-(2-(difluoromethyl)-3-fluoropyridin- | 0.163 |
| | 4-yl)-11,11-difluoro-8-hydroxy-3,4,8,9,10,11-hexahydro-1H- | |
| | pyrido[4′,3′:3,4]pyrazolo[1,5-a]azepine-2(7H)-carboxamide; | |
| 43 | (S*)-N-(2-Bromo-3-fluoropyridin-4-yl)-8-(cyanomethyl)-11,11- | 0.282 |
| | difluoro-8-hydroxy-3,4,8,9,10,11-hexahydro-1H- | |
| | pyrido[4′,3′:3,4]pyrazolo[1,5-a]azepine-2(7H)-carboxamide; | |
| 44 | (S*)-8-(Cyanomethyl)-N-(2-(difluoromethyl)-3-fluoropyridin-4- | 0.333 |
| | yl)-11,11-difluoro-8-hydroxy-3,4,8,9,10,11-hexahydro-1H- | |
| | pyrido[4′,3′:3,4]pyrazolo[1,5-a]azepine-2(7H)-carboxamide; | |
| 45 | N-(3-Cyano-4-fluorophenyl)-8,11,11-trifluoro-8- | 0.005 |
| | (hydroxymethyl)-3,4,8,9,10,11-hexahydro-1H- | |
| | pyrido[4′,3′:3,4]pyrazolo[1,5-a]azepine-2(7H)-carboxamide; | |
| 46 | N-(2-Bromo-3-fluoropyridin-4-yl)-8,11,11-trifluoro-8- | 0.012 |
| | (hydroxymethyl)-3,4,8,9,10,11-hexahydro-1H- | |
| | pyrido[4′,3′:3,4]pyrazolo[1,5-a]azepine-2(7H)-carboxamide; | |
| 47 | (S*)-N-(2-Bromo-3-fluoropyridin-4-yl)-8,11,11-trifluoro-8- | 0.156 |
| | (hydroxymethyl)-3,4,8,9,10,11-hexahydro-1H- | |
| | pyrido[4′,3′:3,4]pyrazolo[1,5-a]azepine-2(7H)-carboxamide; | |
| 48 | (R*)-N-(2-Bromo-3-fluoropyridin-4-yl)-8,11,11-trifluoro-8- | 0.011 |
| | (hydroxymethyl)-3,4,8,9,10,11-hexahydro-1H- | |
| | pyrido[4′,3′:3,4]pyrazolo[1,5-a]azepine-2(7H)-carboxamide; | |
| 49 | 8-Acetamidomethyl)-N-(3-cyano-4-fluorophenyl)-11,11- | 0.033 |
| | difluoro-3,4,8,9,10,11-hexahydro-1H- | |
| | pyrido[4′,3′:3,4]pyrazolo[1,5-a]azepine-2(7H)-carboxamide; | |
| 50 | 8-(Acetamidomethyl)-N-(2-bromo-3-fluoropyridin-4-yl)-11,11- | 0.11 |
| | difluoro-3,4,8,9,10,11-hexahydro-1H- | |
| | pyrido[4′,3′:3,4]pyrazolo[1,5-a]azepine-2(7H)-carboxamide; | |
| 51 | N-(3-Cyano-4-fluorophenyl)-11,11-difluoro-8-((2,2,2- | 0.032 |
| | trifluoroacetamido)methyl)-3,4,8,9,10,11-hexahydro-1H- | |
| | pyrido[4′,3′:3,4]pyrazolo[1,5-a]azepine-2(7H)-carboxamide; | |
| 52 | N-(2-Bromo-3-fluoropyridin-4-yl)-11,11-difluoro-8-((2,2,2- | 0.102 |
| | trifluoroacetamido)methyl)-3,4,8,9,10,11-hexahydro-1H- | |
| | pyrido[4′,3′:3,4]pyrazolo[1,5-a]azepine-2(7H)-carboxamide; | |
| 53 | Methyl ((2-((3-cyano-4-fluorophenyl)carbamoyl)-11,11- | 0.01 |
| | difluoro-2,3,4,7,8,9,10,11-octahydro-1H- | |
| | pyrido[4′,3′:3,4]pyrazolo[1,5-a]azepin-8-yl)methyl)carbamate; | |
| 54 | Methyl ((2-((2-bromo-3-fluoropyridin-4-yl)carbamoyl)-11,11- | 0.008 |
| | difluoro-2,3,4,7,8,9,10,11-octahydro-1H- | |
| | pyrido[4′,3′:3,4]pyrazolo[1,5-a]azepin-8-yl)methyl)carbamate; | |
| 55 | N-(3-Cyano-4-fluorophenyl)-11,11-difluoro-8-hydroxy-8- | 0.029 |
| | methyl-3,4,8,9,10,11-hexahydro-1H- | |
| | pyrido[4′,3′:3,4]pyrazolo[1,5-a]azepine-2(7H)-carboxamide; | |
| 56 | (R*)-N-(3-Cyano-4-fluorophenyl)-11,11-difluoro-8-hydroxy-8- | 0.016 |
| | vinyl-3,4,8,9,10,11-hexahydro-1H- | |
| | pyrido[4′,3′:3,4]pyrazolo[1,5-a]azepine-2(7H)-carboxamide; | |
| 57 | (S*)-N-(3-Cyano-4-fluorophenyl)-11,11-difluoro-8-hydroxy-8- | 0.009 |
| | vinyl-3,4,8,9,10,11-hexahydro-1H- | |
| | pyrido[4′,3′:3,4]pyrazolo[1,5-a]azepine-2(7H)-carboxamide; | |
| 58 | N-(3-Cyano-4-fluorophenyl)-8-ethynyl-11,11-difluoro-8- | 0.037 |
| | hydroxy-3,4,8,9,10,11-hexahydro-1H- | |
| | pyrido[4′,3′:3,4]pyrazolo[1,5-a]azepine-2(7H)-carboxamide; | |
| 59 | (R*)-N-(3-Cyano-4-fluorophenyl)-8-ethynyl-11,11-difluoro-8- | 0.005 |
| | hydroxy-3,4,8,9,10,11-hexahydro-1H- | |
| | pyrido[4′,3′:3,4]pyrazolo[1,5-a]azepine-2(7H)-carboxamide; | |
| 60 | (S*)-N-(3-Cyano-4-fluorophenyl)-8-ethynyl-11,11-difluoro-8- | 0.017 |
| | hydroxy-3,4,8,9,10,11-hexahydro-1H- | |
| | pyrido[4′,3′:3,4]pyrazolo[1,5-a]azepine-2(7H)-carboxamide; | |
| 61 | (S*)-N-(3-Cyano-4-fluorophenyl)-8-ethyl-11,11-difluoro-8- | 0.04 |
| | hydroxy-3,4,8,9,10,11-hexahydro-1H- | |
| | pyrido[4′,3′:3,4]pyrazolo[1,5-a]azepine-2(7H)-carboxamide; | |
| 62 | (R*)-N-(3-Cyano-4-fluorophenyl)-8-ethyl-11,11-difluoro-8- | 0.042 |
| | hydroxy-3,4,8,9,10,11-hexahydro-1H- | |
| | pyrido[4′,3′:3,4]pyrazolo[1,5-a]azepine-2(7H)-carboxamide; | |
| 63 | (R*)-N-(2-Bromo-3-fluoropyridin-4-yl)-8-ethynyl-11,11- | 0.119 |
| | difluoro-8-hydroxy-3,4,8,9,10,11-hexahydro-1H- | |
| | pyrido[4′,3′:3,4]pyrazolo[1,5-a]azepine-2(7H)-carboxamide; | |
| 64 | (R*)-N-(2-(Difluoromethyl)-3-fluoropyridin-4-yl)-8-ethynyl- | 0.143 |
| | 11,11-difluoro-8-hydroxy-3,4,8,9,10,11-hexahydro-1H- | |
| | pyrido[4′,3′:3,4]pyrazolo[1,5-a]azepine-2(7H)-carboxamide; | |
| 65 | (S*)-N-(2-Bromo-3-fluoropyridin-4-yl)-8-ethynyl-11,11- | 0.144 |
| | difluoro-8-hydroxy-3,4,8,9,10,11-hexahydro-1H- | |
| | pyrido[4′,3′:3,4]pyrazolo[1,5-a]azepine-2(7H)-carboxamide; | |
| 66 | (S*)-N-(2-(Difluoromethyl)-3-fluoropyridin-4-yl)-8-ethynyl- | 0.149 |
| | 11,11-difluoro-8-hydroxy-3,4,8,9,10,11-hexahydro-1H- | |
| | pyrido[4′,3′:3,4]pyrazolo[1,5-a]azepine-2(7H)-carboxamide; | |
| 67 | (3R,8R)-N-(3-cyano-4-fluorophenyl)-11,11-difluoro-8-hydroxy- | 0.004 |
| | 3-methyl-3,4,8,9,10,11-hexahydro-1H- | |
| | pyrido[4′,3′:3,4]pyrazolo[1,5-a]azepine-2(7H)-carboxamide; | |
| 68 | (3R,8R)-N-(2-Bromo-3-fluoropyridin-4-yl)-11,11-difluoro-8- | 0.021 |
| | hydroxy-3-methyl-3,4,8,9,10,11-hexahydro-1H- | |
| | pyrido[4′,3′:3,4]pyrazolo[1,5-a]azepine-2(7H)-carboxamide; | |
| 69 | (3R,8R)-11,11-Difluoro-N-(4-fluoro-3- | 0.007 |
| | (trifluoromethyl)phenyl)-8-hydroxy-3-methyl-3,4,8,9,10,11- | |
| | hexahydro-1H-pyrido[4′,3′:3,4]pyrazolo[1,5-a]azepine-2(7H)- | |
| | carboxamide; | |
| 70 | (3R,8R)-N-(2-(Difluoromethyl)-3-fluoropyridin-4-yl)-11,11- | 0.031 |
| | difluoro-8-hydroxy-3-methyl-3,4,8,9,10,11-hexahydro-1H- | |
| | pyrido[4′,3′:3,4]pyrazolo[1,5-a]azepine-2(7H)-carboxamide; | |
| 71 | (3R,8S)-N-(3-Cyano-4-fluorophenyl)-11,11-difluoro-8- | 0.002 |
| | hydroxy-3-methyl-3,4,8,9,10,11-hexahydro-1H- | |
| | pyrido[4′,3′:3,4]pyrazolo[1,5-a]azepine-2(7H)-carboxamide; | |
| 72 | (3R,8S)-N-(2-Bromo-3-fluoropyridin-4-yl)-11,11-difluoro-8- | 0.009 |
| | hydroxy-3-methyl-3,4,8,9,10,11-hexahydro-1H- | |
| | pyrido[4′,3′:3,4]pyrazolo[1,5-a]azepine-2(7H)-carboxamide; | |
| 73 | (3R,8S)-11,11-Difluoro-N-(4-fluoro-3-(trifluoromethyl)phenyl)- | 0.002 |
| | 8-hydroxy-3-methyl-3,4,8,9,10,11-hexahydro-1H- | |
| | pyrido[4′,3′:3,4]pyrazolo[1,5-a]azepine-2(7H)-carboxamide; | |
| 74 | (3R,8S)-N-(2-(Difluoromethyl)-3-fluoropyridin-4-yl)-11,11- | 0.011 |
| | difluoro-8-hydroxy-3-methyl-3,4,8,9,10,11-hexahydro-1H- | |
| | pyrido[4′,3′:3,4]pyrazolo[1,5-a]azepine-2(7H)-carboxamide; | |
| 75 | (3R,8S*)-N-(3-Cyano-4-fluorophenyl)-11,11-difluoro-8- | 0.005 |
| | (hydroxymethyl)-3-methyl-3,4,8,9,10,11-hexahydro-1H- | |
| | pyrido[4′,3′:3,4]pyrazolo[1,5-a]azepine-2(7H)-carboxamide; | |
| 76 | (3R,8S*)-11,11-Difluoro-N-(4-fluoro-3- | 0.005 |
| | (trifluoromethyl)phenyl)-8-(hydroxymethyl)-3-methyl- | |
| | 3,4,8,9,10,11-hexahydro-1H-pyrido[4′,3′:3,4]pyrazolo[1,5- | |
| | a]azepine-2(7H)-carboxamide; | |
| 77 | (3R,8S*)-N-(2-Bromo-3-fluoropyridin-4-yl)-11,11-difluoro-8- | 0.014 |
| | (hydroxymethyl)-3-methyl-3,4,8,9,10,11-hexahydro-1H- | |
| | pyrido[4′,3′:3,4]pyrazolo[1,5-a]azepine-2(7H)-carboxamide; | |
| 78 | (3R,8S*)-N-(2-(Difluoromethyl)-3-fluoropyridin-4-yl)-11,11- | 0.03 |
| | difluoro-8-(hydroxymethyl)-3-methyl-3,4,8,9,10,11-hexahydro- | |
| | 1H-pyrido[4′,3′:3,4]pyrazolo[1,5-a]azepine-2(7H)-carboxamide; | |
| 79 | (3R,8R*)-N-(3-Cyano-4-fluorophenyl)-11,11-difluoro-8- | 0.001 |
| | (hydroxymethyl)-3-methyl-3,4,8,9,10,11-hexahydro-1H- | |
| | pyrido[4′,3′:3,4]pyrazolo[1,5-a]azepine-2(7H)-carboxamide; | |
| 80 | (3R,8R*)-11,11-Difluoro-N-(4-fluoro-3- | 0.001 |
| | (trifluoromethyl)phenyl)-8-(hydroxymethyl)-3-methyl- | |
| | 3,4,8,9,10,11-hexahydro-1H-pyrido[4′,3′:3,4]pyrazolo[1,5- | |
| | a]azepine-2(7H)-carboxamide; | |
| 81 | (3R,8R*)-N-(2-Bromo-3-fluoropyridin-4-yl)-11,11-difluoro-8- | 0.002 |
| | (hydroxymethyl)-3-methyl-3,4,8,9,10,11-hexahydro-1H- | |
| | pyrido[4′,3′:3,4]pyrazolo[1,5-a]azepine-2(7H)-carboxamide; | |
| 82 | (3R,8R*)-N-(2-(Difluoromethyl)-3-fluoropyridin-4-yl)-11,11- | 0.001 |
| | difluoro-8-(hydroxymethyl)-3-methyl-3,4,8,9,10,11-hexahydro- | |
| | 1H-pyrido[4′,3′:3,4]pyrazolo[1,5-a]azepine-2(7H)-carboxamide; | |
| 83 | (3R,8R*)-N-(3-Cyano-4-fluorophenyl)-11,11-difluoro-8- | 0.01 |
| | (fluoromethyl)-8-hydroxy-3-methyl-3,4,8,9,10,11-hexahydro- | |
| | 1H-pyrido[4′,3′:3,4]pyrazolo[1,5-a]azepine-2(7H)-carboxamide; | |
| 84 | (3R,8R*)-N-(2-Bromo-3-fluoropyridin-4-yl)-11,11-difluoro-8- | 0.069 |
| | (fluoromethyl)-8-hydroxy-3-methyl-3,4,8,9,10,11-hexahydro- | |
| | 1H-pyrido[4′,3′:3,4]pyrazolo[1,5-a]azepine-2(7H)-carboxamide; | |
| 85 | (3R,8R*)-N-(2-(Difluoromethyl)-3-fluoropyridin-4-yl)-11,11- | 0.045 |
| | difluoro-8-(fluoromethyl)-8-hydroxy-3-methyl-3,4,8,9,10,11- | |
| | hexahydro-1H-pyrido[4′,3′:3,4]pyrazolo[1,5-a]azepine-2(7H)- | |
| | carboxamide; | |
| 86 | (3R,8S*)-N-(3-Cyano-4-fluorophenyl)-11,11-difluoro-8- | 0.02 |
| | (fluoromethyl)-8-hydroxy-3-methyl-3,4,8,9,10,11-hexahydro- | |
| | 1H-pyrido[4′,3′:3,4]pyrazolo[1,5-a]azepine-2(7H)-carboxamide; | |
| 87 | (3R,8S*)-N-(2-Bromo-3-fluoropyridin-4-yl)-11,11-difluoro-8- | 0.039 |
| | (fluoromethyl)-8-hydroxy-3-methyl-3,4,8,9,10,11-hexahydro- | |
| | 1H-pyrido[4′,3′:3,4]pyrazolo[1,5-a]azepine-2(7H)-carboxamide; | |
| 88 | (3R,8S*)-N-(2-(Difluoromethyl)-3-fluoropyridin-4-yl)-11,11- | 0.118 |
| | difluoro-8-(fluoromethyl)-8-hydroxy-3-methyl-3,4,8,9,10,11- | |
| | hexahydro-1H-pyrido[4′,3′:3,4]pyrazolo[1,5-a]azepine-2(7H)- | |
| | carboxamide; | |
| 89 | (3R,8S*)-N-(2-Bromo-3-fluoropyridin-4-yl)-8-(cyanomethyl)- | 0.028 |
| | 11,11-difluoro-8-hydroxy-3-methyl-3,4,8,9,10,11-hexahydro- | |
| | 1H-pyrido[4′,3′:3,4]pyrazolo[1,5-a]azepine-2(7H)-carboxamide; | |
| 90 | (3R,8S*)-8-(Cyanomethyl)-N-(2-(difluoromethyl)-3- | 0.054 |
| | fluoropyridin-4-yl)-11,11-difluoro-8-hydroxy-3-methyl- | |
| | 3,4,8,9,10,11-hexahydro-1H-pyrido[4′,3′:3,4]pyrazolo[1,5- | |
| | a]azepine-2(7H)-carboxamide; | |
| 91 | (3R,8R*)-N-(2-Bromo-3-fluoropyridin-4-yl)-8-(cyanomethyl)- | 0.03 |
| | 11,11-difluoro-8-hydroxy-3-methyl-3,4,8,9,10,11-hexahydro- | |
| | 1H-pyrido[4′,3′:3,4]pyrazolo[1,5-a]azepine-2(7H)-carboxamide; | |
| 92 | (3R,8R*)-8-(Cyanomethyl)-N-(2-(difluoromethyl)-3- | 0.191 |
| | fluoropyridin-4-yl)-11,11-difluoro-8-hydroxy-3-methyl- | |
| | 3,4,8,9,10,11-hexahydro-1H-pyrido[4′,3′:3,4]pyrazolo[1,5- | |
| | a]azepine-2(7H)-carboxamide; | |
| 93 | (3R,8R*)-N-(3-Cyano-4-fluorophenyl)-8-((2,2- | 0.045 |
| | difluoroethoxy)methyl)-11,11-difluoro-8-hydroxy-3-methyl- | |
| | 3,4,8,9,10,11-hexahydro-1H-pyrido[4′,3′:3,4]pyrazolo[1,5- | |
| | a]azepine-2(7H)-carboxamide; | |
| 94 | (3R,8S*)-N-(3-Cyano-4-fluorophenyl)-8-((2,2- | 0.015 |
| | difluoroethoxy)methyl)-11,11-difluoro-8-hydroxy-3-methyl- | |
| | 3,4,8,9,10,11-hexahydro-1H-pyrido[4′,3′:3,4]pyrazolo[1,5- | |
| | a]azepine-2(7H)-carboxamide; | |
| 95 | (3R,8R*)-N-(3-Cyano-4-fluorophenyl)-8-ethynyl-11,11- | 0.013 |
| | difluoro-8-hydroxy-3-methyl-3,4,8,9,10,11-hexahydro-1H- | |
| | pyrido[4′,3′:3,4]pyrazolo[1,5-a]azepine-2(7H)-carboxamide; | |
| 96 | (3R,8R*)-N-(2-Bromo-3-fluoropyridin-4-yl)-8-ethynyl-11,11- | 0.035 |
| | difluoro-8-hydroxy-3-methyl-3,4,8,9,10,11-hexahydro-1H- | |
| | pyrido[4′,3′:3,4]pyrazolo[1,5-a]azepine-2(7H)-carboxamide; | |
| 97 | (3R,8R*)-N-(2-(Difluoromethyl)-3-fluoropyridin-4-yl)-8- | 0.035 |
| | ethynyl-11,11-difluoro-8-hydroxy-3-methyl-3,4,8,9,10,11- | |
| | hexahydro-1H-pyrido[4′,3′:3,4]pyrazolo[1,5-a]azepine-2(7H)- | |
| | carboxamide; | |
| 98 | (3R,8S*)-N-(3-Cyano-4-fluorophenyl)-8-ethynyl-11,11- | 0.051 |
| | difluoro-8-hydroxy-3-methyl-3,4,8,9,10,11-hexahydro-1H- | |
| | pyrido[4′,3′:3,4]pyrazolo[1,5-a]azepine-2(7H)-carboxamide; | |
| 99 | (3R,8S*)-N-(2-Bromo-3-fluoropyridin-4-yl)-8-ethynyl-11,11- | 0.275 |
| | difluoro-8-hydroxy-3-methyl-3,4,8,9,10,11-hexahydro-1H- | |
| | pyrido[4′,3′:3,4]pyrazolo[1,5-a]azepine-2(7H)-carboxamide; | |
| 100 | (3R,8S*)-N-(2-Difluoromethyl)-3-fluoropyridin-4-yl)-8- | 0.168 |
| | ethynyl-11,11-difluoro-8-hydroxy-3-methyl-3,4,8,9,10,11- | |
| | hexahydro-1H-pyrido[4′,3′:3,4]pyrazolo[1,5-a]azepine-2(7H)- | |
| | carboxamide; | |
| 101 | (3R,8S*)-N-(3-Cyano-4-fluorophenyl)-11,11-difluoro-8-(2- | 0.044 |
| | hydroxypropan-2-yl)-3-methyl-3,4,8,9,10,11-hexahydro-1H- | |
| | pyrido[4′,3′:3,4]pyrazolo[1,5-a]azepine-2(7H)-carboxamide; | |
| 102 | (3R,8S*)-N-(2-Bromo-3-fluoropyridin-4-yl)-11,11-difluoro-8- | 0.088 |
| | (2-hydroxypropan-2-yl)-3-methyl-3,4,8,9,10,11-hexahydro-1H- | |
| | pyrido[4′,3′:3,4]pyrazolo[1,5-a]azepine-2(7H)-carboxamide; | |
| 103 | (3R,8R*)-N-(3-Cyano-4-fluorophenyl)-11,11-difluoro-8-(2- | 0.018 |
| | hydroxypropan-2-yl)-3-methyl-3,4,8,9,10,11-hexahydro-1H- | |
| | pyrido[4′,3′:3,4]pyrazolo[1,5-a]azepine-2(7H)-carboxamide; | |
| 104 | (3R,8R*)-N-(2-Bromo-3-fluoropyridin-4-yl)-11,11-difluoro-8- | 0.036 |
| | (2-hydroxypropan-2-yl)-3-methyl-3,4,8,9,10,11-hexahydro-1H- | |
| | pyrido[4′,3′:3,4]pyrazolo[1,5-a]azepine-2(7H)-carboxamide; | |
| 105 | methyl (((3R,8R*)-2-((3-cyano-4-fluorophenyl)carbamoyl)- | 0.003 |
| | 11,11-difluoro-3-methyl-2,3,4,7,8,9,10,11-octahydro-1H- | |
| | pyrido[4′,3′:3,4]pyrazolo[1,5-a]azepin-8-yl)methyl)carbamate; | |
| 106 | Methyl (((3R,8R*)-2-((2-bromo-3-fluoropyridin-4- | 0.01 |
| | yl)carbamoyl)-11,11-difluoro-3-methyl-2,3,4,7,8,9,10,11- | |
| | octahydro-1H-pyrido[4′,3′:3,4]pyrazolo[1,5-a]azepin-8- | |
| | yl)methyl)carbamate; | |
| 107 | Methyl (((3R,8R*)-2-((2-(difluoromethyl)-3-fluoropyridin-4-yl) | 0.008 |
| | carbamoyl)-11,11-difluoro-3-methyl-2,3,4,7,8,9,10,11- | |
| | octahydro-1H-pyrido[4′,3′:3,4]pyrazolo[1,5-a]azepin-8- | |
| | yl)methyl)carbamate; | |
| 108 | Methyl (((3R,8S*)-2-((3-cyano-4-fluorophenyl)carbamoyl)- | 0.012 |
| | 11,11-difluoro-3-methyl-2,3,4,7,8,9,10,11-octahydro-1H- | |
| | pyrido[4′,3′:3,4]pyrazolo[1,5-a]azepin-8-yl)methyl)carbamate; | |
| 109 | Methyl (((3R,8S*)-2-((2-bromo-3-fluoropyridin-4- | 0.032 |
| | yl)carbamoyl)-11,11-difluoro-3-methyl-2,3,4,7,8,9,10,11- | |
| | octahydro-1H-pyrido[4′,3′:3,4]pyrazolo[1,5-a]azepin-8- | |
| | yl)methyl)carbamate; | |
| 110 | Methyl (((3R,8S*)-2-((2-(difluoromethyl)-3-fluoropyridin-4-yl) | 0.018 |
| | carbamoyl)-11,11-difluoro-3-methyl-2,3,4,7,8,9,10,11- | |
| | octahydro-1H-pyrido[4′,3′:3,4]pyrazolo[1,5-a]azepin-8- | |
| | yl)methyl)carbamate; | |
| 111 | (3R,8S*)-N-(3-Cyano-4-fluorophenyl)-8,11,11-trifluoro-8- | 0.007 |
| | (hydroxymethyl)-3-methyl-3,4,8,9,10,11-hexahydro-1H- | |
| | pyrido[4′,3′:3,4]pyrazolo[1,5-a]azepine-2(7H)-carboxamide; | |
| 112 | (3R,8S*)-N-(2-Bromo-3-fluoropyridin-4-yl)-8,11,11-trifluoro-8- | 0.045 |
| | (hydroxymethyl)-3-methyl-3,4,8,9,10,11-hexahydro-1H- | |
| | pyrido[4′,3′:3,4]pyrazolo[1,5-a]azepine-2(7H)-carboxamide; | |
| 113 | (3R,8S*)-N-(2-(Difluoromethyl)-3-fluoropyridin-4-yl)-8,11,11- | 0.154 |
| | trifluoro-8-(hydroxymethyl)-3-methyl-3,4,8,9,10,11-hexahydro- | |
| | 1H-pyrido[4′,3′:3,4]pyrazolo[1,5-a]azepine-2(7H)-carboxamide; | |
| 114 | (3R,8R*)-N-(3-Cyano-4-fluorophenyl)-8,11,11-trifluoro-8- | 0.001 |
| | (hydroxyl methyl)-3-methyl-3,4,8,9,10,11-hexahydro-1H- | |
| | pyrido[4′,3′:3,4]pyrazolo[1,5-a]azepine-2(7H)-carboxamide; | |
| 115 | (3R,8R*)-N-(2-Bromo-3-fluoropyridin-4-yl)-8,11,11-trifluoro-8- | 0.005 |
| | (hydroxylmethyl)-3-methyl-3,4,8,9,10,11-hexahydro-1H- | |
| | pyrido[4′,3′:3,4]pyrazolo[1,5-a]azepine-2(7H)-carboxamide; | |
| 116 | (3R,8R*)-N-(2-(Difluoromethyl)-3-fluoropyridin-4-yl)-8,11,11- | 0.01 |
| | trifluoro-8-(hydroxymethyl)-3-methyl-3,4,8,9,10,11-hexahydro- | |
| | 1H-pyrido[4′,3′:3,4]pyrazolo[1,5-a]azepine-2(7H)-carboxamide; | |
| 117 | (3R,9S*)-N-(2-Bromo-3-fluoropyridin-4-yl)-11,11-difluoro-9- | 0.018 |
| | hydroxyl-3-methyl-3,4,8,9,10,11-hexahydro-1H- | |
| | pyrido[4′,3′:3,4]pyrazolo[1,5-a]azepine-2(7H)-carboxamide; | |
| 118 | (3R,9R*)-N-(2-Bromo-3-fluoropyridin-4-yl)-11,11-difluoro-9- | 0.003 |
| | hydroxyl-3-methyl-3,4,8,9,10,11-hexahydro-1H- | |
| | pyrido[4′,3′:3,4]pyrazolo[1,5-a]azepine-2(7H)-carboxamide; | |
| 119 | (3R,9S*)-N-(3-Cyano-4-fluorophenyl)-11,11-difluoro-9- | 0.001 |
| | hydroxyl-3-methyl-3,4,8,9,10,11-hexahydro-1H- | |
| | pyrido[4′,3′:3,4]pyrazolo[1,5-a]azepine-2(7H)-carboxamide; | |
| 120 | (3R,9R*)-N-(3-Cyano-4-fluorophenyl)-11,11-difluoro-9- | 0.001 |
| | hydroxy-3-methyl-3,4,8,9,10,11-hexahydro-1H- | |
| | pyrido[4′,3′:3,4]pyrazolo[1,5-a]azepine-2(7H)-carboxamide; | |
| 121 | (3R,9S*)-N-(2-(Difluoromethyl)-3-fluoropyridin-4-yl)-11,11- | 0.008 |
| | difluoro-9-hydroxy-3-methyl-3,4,8,9,10,11-hexahydro-1H- | |
| | pyrido[4′,3′:3,4]pyrazolo[1,5-a]azepine-2(7H)-carboxamide; | |
| 122 | (3R,9R*)-N-(2-(Difluoromethyl)-3-fluoropyridin-4-yl)-11,11- | 0.002 |
| | difluoro-9-hydroxy-3-methyl-3,4,8,9,10,11-hexahydro-1H- | |
| | pyrido[4′,3′:3,4]pyrazolo[1,5-a]azepine-2(7H)-carboxamide; | |
| 123 | (3R,9R*)-N-(2-Bromo-3-fluoropyridin-4-yl)-11-fluoro-9- | 0.018 |
| | hydroxy-3-methyl-3,4,8,9-tetrahydro-1H- | |
| | pyrido[4′,3′:3,4]pyrazolo[1,5-a]azepine-2(7H)-carboxamide; | |
| 124 | (3R,9S*)-N-(2-Bromo-3-fluoropyridin-4-yl)-11-fluoro-9- | 0.026 |
| | hydroxy-3-methyl-3,4,8,9-tetrahydro-1H- | |
| | pyrido[4′,3′:3,4]pyrazolo[1,5-a]azepine-2(7H)-carboxamide; | |
| 125 | (3R,9R*)-N-(3-Cyano-4-fluorophenyl)-11,11-difluoro-9- | 0.014 |
| | hydroxy-9-(hydroxymethyl)-3-methyl-3,4,8,9,10,11-hexahydro- | |
| | 1H-pyrido[4′,3′:3,4]pyrazolo[1,5-a]azepine-2(7H)-carboxamide; | |
| 126 | (3R,9S*)-N-(3-Cyano-4-fluorophenyl)-11,11-difluoro-9- | 0.005 |
| | hydroxy-9-(hydroxymethyl)-3-methyl-3,4,8,9,10,11-hexahydro- | |
| | 1H-pyrido[4′,3′:3,4]pyrazolo[1,5-a]azepine-2(7H)-carboxamide; | |
| 127 | (3R,9R*)-N-(2-Bromo-3-fluoropyridin-4-yl)-11,11-difluoro-9- | 0.005 |
| | (fluoromethyl)-9-hydroxy-3-methyl-3,4,8,9,10,11-hexahydro- | |
| | 1H-pyrido[4′,3′:3,4]pyrazolo[1,5-a]azepine-2(7H)-carboxamide; | |
| 128 | (3R,9R*)-N-(3-Cyano-4-fluorophenyl)-11,11-difluoro-9- | 0.001 |
| | (fluoromethyl)-9-hydroxy-3-methyl-3,4,8,9,10,11-hexahydro- | |
| | 1H-pyrido[4′,3′:3,4]pyrazolo[1,5-a]azepine-2(7H)-carboxamide; | |
| 129 | (3R,9S*)-N-(2-Bromo-3-fluoropyridin-4-yl)-11,11-difluoro-9- | 0.006 |
| | (fluoromethyl)-9-hydroxy-3-methyl-3,4,8,9,10,11-hexahydro- | |
| | 1H-pyrido[4′,3′:3,4]pyrazolo[1,5-a]azepine-2(7H)-carboxamide; | |
| 130 | (3R,9S*)-N-(3-Cyano-4-fluorophenyl)-11,11-difluoro-9- | 0.002 |
| | (fluoromethyl)-9-hydroxy-3-methyl-3,4,8,9,10,11-hexahydro- | |
| | 1H-pyrido[4′,3′:3,4]pyrazolo[1,5-a]azepine-2(7H)-carboxamide; | |
| 131 | (3R,9R)-N-(3-Cyano-4-fluorophenyl)-11,11-difluoro-9- | 0.003 |
| | (fluoromethyl)-3-methyl-3,4,8,9,10,11-hexahydro-1H- | |
| | pyrido[4′,3′:3,4]pyrazolo[1,5-a]azepine-2(7H)-carboxamide; | |
| 132 | (3R,9S*)-N-(3-Cyano-4-fluorophenyl)-11,11-difluoro-9- | 0.007 |
| | (fluoromethyl)-3-methyl-3,4,8,9,10,11-hexahydro-1H- | |
| | pyrido[4′,3′:3,4]pyrazolo[1,5-a]azepine-2(7H)-carboxamide; | |
| 133 | (S*)-N-(2-Bromo-3-fluoropyridin-4-yl)-11,11-difluoro-9- | 0.038 |
| | (hydroxymethyl)-3,4,8,9,10,11-hexahydro-1H- | |
| | pyrido[4′,3′:3,4]pyrazolo[1,5-a]azepine-2(7H)-carboxamide; | |
| 134 | (S*)-N-(3-Cyano-4-fluorophenyl)-11,11-difluoro-9- | 0.017 |
| | (hydroxymethyl)-3,4,8,9,10,11-hexahydro-1H- | |
| | pyrido[4′,3′:3,4]pyrazolo[1,5-a]azepine-2(7H)-carboxamide; | |
| 135 | (R*)-N-(2-Bromo-3-fluoropyridin-4-yl)-11,11-difluoro-9- | 0.019 |
| | (hydroxymethyl)-3,4,8,9,10,11-hexahydro-1H- | |
| | pyrido[4′,3′:3,4]pyrazolo[1,5-a]azepine-2(7H)-carboxamide; | |
| 136 | (R*)-N-(3-Cyano-4-fluorophenyl)-11,11-difluoro-9- | 0.005 |
| | (hydroxymethyl)-3,4,8,9,10,11-hexahydro-1H- | |
| | pyrido[4′,3′:3,4]pyrazolo[1,5-a]azepine-2(7H)-carboxamide; | |
| 137 | (R*)-N-(2-Bromo-3-fluoropyridin-4-yl)-11,11-difluoro-9- | 0.002 |
| | hydroxy-3,4,8,9,10,11-hexahydro-1H- | |
| | pyrido[4′,3′:3,4]pyrazolo[1,5-a]azepine-2(7H)-carboxamide; | |
| 138 | (R*)-N-(3-Cyano-4-fluorophenyl)-11,11-difluoro-9-hydroxy- | 0.001 |
| | 3,4,8,9,10,11-hexahydro-1H-pyrido[4′,3′:3,4]pyrazolo[1,5- | |
| | a]azepine-2(7H)-carboxamide; | |
| 139 | (R*)-N-(2-(Difluoromethyl)-3-fluoropyridin-4-yl)-11,11- | 0.021 |
| | difluoro-9-hydroxy-3,4,8,9,10,11-hexahydro-1H- | |
| | pyrido[4′,3′:3,4]pyrazolo[1,5-a]azepine-2(7H)-carboxamide; | |
| 140 | (S*)-N-(2-Bromo-3-fluoropyridin-4-yl)-11,11-difluoro-9- | 0.027 |
| | hydroxy-3,4,8,9,10,11-hexahydro-1H- | |
| | pyrido[4′,3′:3,4]pyrazolo[1,5-a]azepine-2(7H)-carboxamide; | |
| 141 | (S*)-N-(3-Cyano-4-fluorophenyl)-11,11-difluoro-9-hydroxy- | 0.016 |
| | 3,4,8,9,10,11-hexahydro-1H-pyrido[4′,3′:3,4]pyrazolo[1,5- | |
| | a]azepine-2(7H)-carboxamide; | |
| 142 | (S*)-N-(2-(Difluoromethyl)-3-fluoropyridin-4-yl)-11,11- | 0.046 |
| | difluoro-9-hydroxy-3,4,8,9,10,11-hexahydro-1H- | |
| | pyrido[4′,3′:3,4]pyrazolo[1,5-a]azepine-2(7H)-carboxamide; | |
| 143 | (3R)-N-(3-Cyano-4-fluorophenyl)-11,11-difluoro-10-hydroxy- | 0.154 |
| | 3-methyl-1,3,4,7,8,9,10,11-octahydro-2H- | |
| | pyrido[4′,3′:3,4]pyrazolo[1,5-a]azepine-2-carboxamide; | |

NT means not tested.

The disclosed subject matter is not to be limited in scope by the specific embodiments and examples described herein. Indeed, various modifications of the disclosure in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

1. A compound, or a pharmaceutically acceptable salt, solvate, stereoisomer, isotopic variant, or a N-oxide thereof, having the structure of Formula (I): 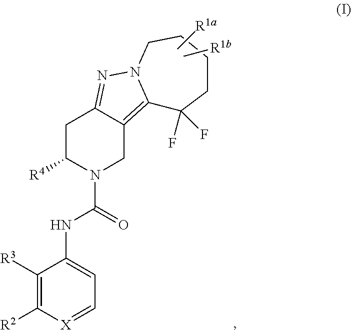
wherein
R^{1a} is H, F, or OH;
R^{1b} is selected from the group consisting of: F, OH, C₁₋₄alkyl, C₂₋₄alkenyl, C₂₋₄alkynyl, C₁₋₄haloalkyl, CH₂OH, C(CH₃)₂OH, CH₂CN, CH₂NH(C═O)CH₃, CH₂NH(C═O)OCH₃, CH₂OC₁₋₄haloalkyl, OC₁₋₄haloalkyl, CH₂NH(C═O)CF₃, and OC₁₋₄haloalkyl,
or R^{1a} and R^{1b} come together to form ═CH₂;
R² is selected from the group consisting of: Br, CN, and C₁₋₄haloalkyl;
R³ is H, or F;
R⁴ is H or C₁₋₄alkyl; and
X is selected from the group consisting of: CH, CF, and N.

2. The compound of claim 1, wherein R^{1a} is H.

3. The compound of claim 1, wherein R^{1a} is OH.

4. The compound of claim 1, wherein R^{1a} is F.

5. The compound of claim 1, wherein R^{1a} and R^{1b} come together to form ═CH₂.

6. The compound of claim 1, wherein R^{1a} is H and R^{1b} is selected from the group consisting of: F, OH, CH₂OH, C(CH₃)₂OH, CH₂NH(C═O)CH₃, CH₂NH(C═O)CF₃, CH₂OCH₂CHF₂, and OCH₂CHF₂.

7. The compound of claim 1, wherein R^{1a} is F and R^{1b} is CH₂OH.

8. The compound of claim 1, wherein R^{1a} is OH and R^{1b} is selected from the group consisting of: CH₃, CH₂CH₃, CH═CH₂, C≡CH, CH₂F, CH₂OH, CH₂CN, and CH₂OCH₂CHF₂.

9. The compound of claim 1, wherein R² is Br, CN, CHF₂ or CF₃.

10. The compound of claim 1, wherein R³ is H.

11. The compound of claim 1, wherein R³ is F.

12. The compound of claim 1, wherein R⁴ is H.

13. The compound of claim 1, wherein R⁴ is CH₃.

14. The compound of claim 1, wherein X is N.

15. The compound of claim 1, wherein X is CF.

16. The compound of claim 1, wherein X is CH.

17. The compound of claim 1, wherein 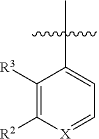
is 3-cyano-4-fluorophenyl, 4-fluoro-3-(trifluoromethyl)phenyl, 3-cyano-2,4-difluorophenyl, 3-bromo-2,4-difluorophenyl, 2-(difluoromethyl)-3-fluoropyridin-4-yl, or 2-bromo-3-fluoropyridin-4-yl.

18. The compound of claim 1, or a pharmaceutically acceptable salt, solvate, stereoisomer, isotopic variant, or a N-oxide thereof, having the structure of Formula (IA): 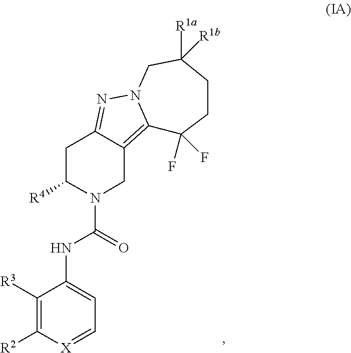
wherein
R^{1a} is H, or OH;
R^{1b} is selected from the group consisting of: F, OH, C₁₋₄alkyl, C₂₋₄alkenyl, C₂₋₄alkynyl, C₁₋₄haloalkyl, CH₂OH, C(CH₃)₂OH, CH₂CN, CH₂NH(C═O)CH₃, CH₂NH(C═O)OCH₃, CH₂OC₁₋₄haloalkyl, CH₂NH(C═O)CF₃, and OC₁₋₄haloalkyl,
or R^{1a} and R^{1b} come together to form ═CH₂;
R² is selected from the group consisting of: Br, CN, and C₁₋₄haloalkyl;
R³ is H, or F;
R⁴ is H or CH₃; and
X is selected from the group consisting of: CH, CF, and N.

19. The compound of claim 1, or a pharmaceutically acceptable salt, solvate, stereoisomer, isotopic variant, or a N-oxide thereof, having the structure of Formula (IB): 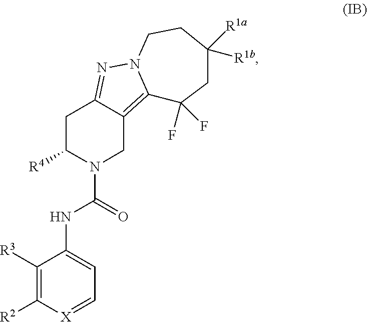
wherein,
R^{1a} is H, or OH;
R^{1b} is selected from the group consisting of: C₁₋₄haloalkyl, and CH₂OH;
R² is selected from the group consisting of: Br, CN, and C₁₋₄haloalkyl;
R³ is H, or F;
R⁴ is H or CH₃; and
X is selected from the group consisting of: CH, CF, and N.

20. A compound selected from the group consisting of:
N-(3-Cyano-4-fluorophenyl)-11,11-difluoro-8-methylene-3,4,8,9,10,11-hexahydro-1H-pyrido[4′,3′:3,4]pyrazolo[1,5-a]azepine-2(7H)-carboxamide;
11,11-Difluoro-N-(4-fluoro-3-(trifluoromethyl)phenyl)-8-methylene-3,4,8,9,10,11-hexahydro-1H-pyrido[4′,3′:3,4]pyrazolo[1,5-a]azepine-2(7H)-carboxamide;
(S)-N-(3-Cyano-4-fluorophenyl)-11,11-difluoro-8-hydroxy-3,4,8,9,10,11-hexahydro-1H-pyrido[4′,3′:3,4]pyrazolo[1,5-a]azepine-2(7H)-carboxamide;
(S)-11,11-Difluoro-N-(4-fluoro-3-(trifluoromethyl)phenyl)-8-hydroxy-3,4,8,9,10,11-hexahydro-1H-pyrido[4′,3′:3,4]pyrazolo[1,5-a]azepine-2(7H)-carboxamide;
(S)-N-(2-Bromo-3-fluoropyridin-4-yl)-11,11-difluoro-8-hydroxy-3,4,8,9,10,11-hexahydro-1H-pyrido[4′,3′:3,4]pyrazolo[1,5-a]azepine-2(7H)-carboxamide;
(S)-N-(3-Cyano-2,4-difluorophenyl)-11,11-difluoro-8-hydroxy-3,4,8,9,10,11-hexahydro-1H-pyrido[4′,3′:3,4]pyrazolo[1,5-a]azepine-2(7H)-carboxamide;
(S)-N-(3-Bromo-2,4-difluorophenyl)-11,11-difluoro-8-hydroxy-3,4,8,9,10,11-hexahydro-1H-pyrido[4′,3′:3,4]pyrazolo[1,5-a]azepine-2(7H)-carboxamide;
(R)-N-(3-Cyano-4-fluorophenyl)-11,11-difluoro-8-hydroxy-3,4,8,9,10,11-hexahydro-1H-pyrido[4′,3′:3,4]pyrazolo[1,5-a]azepine-2(7H)-carboxamide;
(R)-11,11-Difluoro-N-(4-fluoro-3-(trifluoromethyl)phenyl)-8-hydroxy-3,4,8,9,10,11-hexahydro-1H-pyrido[4′,3′:3,4]pyrazolo[1,5-a]azepine-2(7H)-carboxamide;
(R)-N-(2-Bromo-3-fluoropyridin-4-yl)-11,11-difluoro-8-hydroxy-3,4,8,9,10,11-hexahydro-1H-pyrido[4′,3′:3,4]pyrazolo[1,5-a]azepine-2(7H)-carboxamide;
(R)-N-(3-Cyano-2,4-difluorophenyl)-11,11-difluoro-8-hydroxy-3,4,8,9,10,11-hexahydro-1H-pyrido[4′,3′:3,4]pyrazolo[1,5-a]azepine-2(7H)-carboxamide;
(R)-N-(3-Bromo-2,4-difluorophenyl)-11,11-difluoro-8-hydroxy-3,4,8,9,10,11-hexahydro-1H-pyrido[4′,3′:3,4]pyrazolo[1,5-a]azepine-2(7H)-carboxamide;
(S)-N-(3-Cyano-4-fluorophenyl)-8-(2,2-difluoroethoxy)-11,11-difluoro-3,4,8,9,10,11-hexahydro-1H-pyrido[4′,3′:3,4]pyrazolo[1,5-a]azepine-2(7H)-carboxamide;
(R)-N-(3-Cyano-4-fluorophenyl)-8-(2,2-difluoroethoxy)-11,11-difluoro-3,4,8,9,10,11-hexahydro-1H-pyrido[4′,3′:3,4]pyrazolo[1,5-a]azepine-2(7H)-carboxamide;
(R)-N-(3-Cyano-4-fluorophenyl)-8,11,11-trifluoro-3,4,8,9,10,11-hexahydro-1H-pyrido[4′,3′:3,4]pyrazolo[1,5-a]azepine-2(7H)-carboxamide;
(R)-8,11,11-Trifluoro-N-(4-fluoro-3-(trifluoromethyl)phenyl)-3,4,8,9,10,11-hexahydro-1H-pyrido[4′,3′:3,4]pyrazolo[1,5-a]azepine-2(7H)-carboxamide;
(S)-N-(3-Cyano-4-fluorophenyl)-8,11,11-trifluoro-3,4,8,9,10,11-hexahydro-1H-pyrido[4′,3′:3,4]pyrazolo[1,5-a]azepine-2(7H)-carboxamide;
(S)-8,11,11-Trifluoro-N-(4-fluoro-3-(trifluoromethyl)phenyl)-3,4,8,9,10,11-hexahydro-1H-pyrido[4′,3′:3,4]pyrazolo[1,5-a]azepine-2(7H)-carboxamide;
(S*)-N-(3-Cyano-4-fluorophenyl)-11,11-difluoro-8-(hydroxymethyl)-3,4,8,9,10,11-hexahydro-1H-pyrido[4′,3′:3,4]pyrazolo[1,5-a]azepine-2(7H)-carboxamide;
(S*)-11,11-Difluoro-N-(4-fluoro-3-(trifluoromethyl)phenyl)-8-(hydroxymethyl)-3,4,8,9,10,11-hexahydro-1H-pyrido[4′,3′:3,4]pyrazolo[1,5-a]azepine-2(7H)-carboxamide;
(S*)-N-(3-Cyano-2,4-difluorophenyl)-11,11-difluoro-8-(hydroxymethyl)-3,4,8,9,10,11-hexahydro-1H-pyrido[4′,3′:3,4]pyrazolo[1,5-a]azepine-2(7H)-carboxamide;
(S*)-N-(2-Bromo-3-fluoropyridin-4-yl)-11,11-difluoro-8-(hydroxymethyl)-3,4,8,9,10,11-hexahydro-1H-pyrido[4′,3′:3,4]pyrazolo[1,5-a]azepine-2(7H)-carboxamide;
(R*)-N-(3-Cyano-4-fluorophenyl)-11,11-difluoro-8-(hydroxymethyl)-3,4,8,9,10,11-hexahydro-1H-pyrido[4′,3′:3,4]pyrazolo[1,5-a]azepine-2(7H)-carboxamide;
(R*)-11,11-Difluoro-N-(4-fluoro-3-(trifluoromethyl)phenyl)-8-(hydroxymethyl)-3,4,8,9,10,11-hexahydro-1H-pyrido[4′,3′:3,4]pyrazolo[1,5-a]azepine-2(7H)-carboxamide;
(R*)-N-(3-Cyano-2,4-difluorophenyl)-11,11-difluoro-8-(hydroxymethyl)-3,4,8,9,10,11-hexahydro-1H-pyrido[4′,3′:3,4]pyrazolo[1,5-a]azepine-2(7H)-carboxamide;
(R*)-N-(2-Bromo-3-fluoropyridin-4-yl)-11,11-difluoro-8-(hydroxymethyl)-3,4,8,9,10,11-hexahydro-1H-pyrido[4′,3′:3,4]pyrazolo[1,5-a]azepine-2(7H)-carboxamide;
(S*)-N-(3-Cyano-4-fluorophenyl)-842,2-difluoroethoxy)methyl)-11,11-difluoro-3,4,8,9,10,11-hexahydro-1H-pyrido[4′,3′:3,4]pyrazolo[1,5-a]azepine-2(7H)-carboxamide;
(S*)-8-((2,2-Difluoroethoxy)methyl)-11,11-difluoro-N-(4-fluoro-3-(trifluoromethyl)phenyl)-3,4,8,9,10,11-hexahydro-1H-pyrido[4′,3′:3,4]pyrazolo[1,5-a]azepine-2(7H)-carboxamide;
(R*)-N-(3-Cyano-4-fluorophenyl)-842,2-difluoroethoxy)methyl)-11,11-difluoro-3,4,8,9,10,11-hexahydro-1H-pyrido[4′,3′:3,4]pyrazolo[1,5-a]azepine-2(7H)-carboxamide;
(R*)-842,2-Difluoroethoxy)methyl)-11,11-difluoro-N-(4-fluoro-3-(trifluoromethyl)phenyl)-3,4,8,9,10,11-hexahydro-1H-pyrido[4′,3′:3,4]pyrazolo[1,5-a]azepine-2(7H)-carboxamide;
(R*)-N-(3-Cyano-4-fluorophenyl)-842,2-difluoroethoxy)methyl)-11,11-difluoro-8-hydroxy-3,4,8,9,10,11-hexahydro-1H-pyrido[4′,3′:3,4]pyrazolo[1,5-a]azepine-2(7H)-carboxamide;
(R*)-N-(2-Bromo-3-fluoropyridin-4-yl)-842,2-difluoroethoxy)methyl)-11,11-difluoro-8-hydroxy-3,4,8,9,10,11-hexahydro-1H-pyrido[4′,3′:3,4]pyrazolo[1,5-a]azepine-2(7H)-carboxamide;
(R*)-8((2,2-Difluoroethoxy)methyl)-N-(2-(difluoromethyl)-3-fluoropyridin-4-yl)-11,11-difluoro-8-hydroxy-3,4,8,9,10,11-hexahydro-1H-pyrido[4′,3′:3,4]pyrazolo[1,5-a]azepine-2(7H)-carboxamide;
(S*)-N-(3-Cyano-4-fluorophenyl)-842,2-difluoroethoxy)methyl)-11,11-difluoro-8-hydroxy-3,4,8,9,10,11-hexahydro-1H-pyrido[4′,3′:3,4]pyrazolo[1,5-a]azepine-2(7H)-carboxamide;
(S*)-N-(2-Bromo-3-fluoropyridin-4-yl)-842,2-difluoroethoxy)methyl)-11,11-difluoro-8-hydroxy-3,4,8,9,10,11-hexahydro-1H-pyrido[4′,3′:3,4]pyrazolo[1,5-a]azepine-2(7H)-carboxamide;
(S*)-8((2,2-Difluoroethoxy)methyl)-N-(2-(difluoromethyl)-3-fluoropyridin-4-yl)-11,11-difluoro-8-hydroxy-3,4,8,9,10,11-hexahydro-1H-pyrido[4′,3′:3,4]pyrazolo[1,5-a]azepine-2(7H)-carboxamide;
N-(3-Cyano-4-fluorophenyl)-11,11-difluoro-8-(fluoromethyl)-8-hydroxy-3,4,8,9,10,11-hexahydro-1H-pyrido[4′,3′:3,4]pyrazolo[1,5-a]azepine-2(7H)-carboxamide;
(S*)-N-(2-Bromo-3-fluoropyridin-4-yl)-11,11-difluoro-8-(fluoromethyl) -8-hydroxy-3,4,8,9,10,11-hexahydro-1H-pyrido[4′,3′:3,4]pyrazolo[1,5-a]azepine-2(7H)-carboxamide;
(R*)-N-(2-Bromo-3-fluoropyridin-4-yl)-11,11-difluoro-8-(fluoromethyl) -8-hydroxy-3,4,8,9,10,11-hexahydro-1H-pyrido[4′,3′:3,4]pyrazolo[1,5-a]azepine-2(7H)-carboxamide;
N-(3-Cyano-4-fluorophenyl)-8-(cyanomethyl)-11,11-difluoro-8-hydroxy-3,4,8,9,10,11-hexahydro-1H-pyrido[4′,3′:3,4]pyrazolo[1,5-a]azepine-2(7H)-carboxamide;
(R*)-N-(2-Bromo-3-fluoropyridin-4-yl)-8-(cyanomethyl)-11,11-difluoro-8-hydroxy-3,4,8,9,10,11-hexahydro-1H-pyrido[4′,3′:3,4]pyrazolo[1,5-a]azepine-2(7H)-carboxamide;
(R*)-8-(Cyanomethyl)-N-(2-(difluoromethyl)-3-fluoropyridin-4-yl)-11,11-difluoro-8-hydroxy-3,4,8,9,10,11-hexahydro-1H-pyrido[4′,3′:3,4]pyrazolo[1,5-a]azepine-2(7H)-carboxamide;
(S*)-N-(2-Bromo-3-fluoropyridin-4-yl)-8-(cyanomethyl)-11,11-difluoro-8-hydroxy-3,4,8,9,10,11-hexahydro-1H-pyrido[4′,3′:3,4]pyrazolo[1,5-a]azepine-2(7H)-carboxamide;
(S*)-8-(Cyanomethyl)-N-(2-(difluoromethyl)-3-fluoropyridin-4-yl)-11,11-difluoro-8-hydroxy-3,4,8,9,10,11-hexahydro-1H-pyrido[4′,3′:3,4]pyrazolo[1,5-a]azepine-2(7H)-carboxamide;
N-(3-Cyano-4-fluorophenyl)-8,11,11-trifluoro-8-(hydroxymethyl)-3,4,8,9,10,11-hexahydro-1H-pyrido[4′,3′:3,4]pyrazolo[1,5-a]azepine-2(7H)-carboxamide;
N-(2-Bromo-3-fluoropyridin-4-yl)-8,11,11-trifluoro-8-(hydroxymethyl)-3,4,8,9,10,11-hexahydro-1H-pyrido[4′,3′:3,4]pyrazolo[1,5-a]azepine-2(7H)-carboxamide;
(S*)-N-(2-Bromo-3-fluoropyridin-4-yl)-8,11,11-trifluoro-8-(hydroxymethyl)-3,4,8,9,10,11-hexahydro-1H-pyrido[4′,3′:3,4]pyrazolo[1,5-a]azepine-2(7H)-carboxamide;
(R*)-N-(2-Bromo-3-fluoropyridin-4-yl)-8,11,11-trifluoro-8-(hydroxymethyl)-3,4,8,9,10,11-hexahydro-1H-pyrido[4′,3′:3,4]pyrazolo[1,5-a]azepine-2(7H)-carboxamide;
8-Acetamidomethyl)-N-(3-cyano-4-fluorophenyl)-11,11-difluoro-3,4,8,9,10,11-hexahydro-1H-pyrido[4′,3′:3,4]pyrazolo[1,5-a]azepine-2(7H)-carboxamide;
8-(Acetamidomethyl)-N-(2-bromo-3-fluoropyridin-4-yl)-11,11-difluoro-3,4,8,9,10,11-hexahydro-1H-pyrido[4′,3′:3,4]pyrazolo[1,5-a]azepine-2(7H)-carboxamide;
N-(3-Cyano-4-fluorophenyl)-11,11-difluoro-842,2,2-trifluoroacetamido) methyl)-3,4,8,9,10,11-hexahydro-1H-pyrido[4′,3′:3,4]pyrazolo[1,5-a]azepine-2(7H)-carboxamide;
N-(2-Bromo-3-fluoropyridin-4-yl)-11,11-difluoro-842,2,2-trifluoroacetamido)methyl)-3,4,8,9,10,11-hexahydro-1H-pyrido[4′,3′:3,4]pyrazolo[1,5-a]azepine-2(7H)-carboxamide;
Methyl ((2-((3-cyano-4-fluorophenyl)carbamoyl)-11,11-difluoro-2,3,4,7,8,9,10,11-octahydro-1H-pyrido[4′,3′:3,4]pyrazolo[1,5-a]azepin-8-yl)methyl)carbamate;
Methyl ((2-((2-bromo-3-fluoropyridin-4-yl)carbamoyl)-11,11-difluoro-2,3,4,7,8,9,10,11-octahydro-1H-pyrido[4′,3′:3,4]pyrazolo[1,5-a]azepin-8-yl)methyl)carbamate;
N-(3-Cyano-4-fluorophenyl)-11,11-difluoro-8-hydroxy-8-methyl-3,4,8,9,10,11-hexahydro-1H-pyrido[4′,3′:3,4]pyrazolo[1,5-a]azepine-2(7H)-carboxamide;
(R*)-N-(3-Cyano-4-fluorophenyl)-11,11-difluoro-8-hydroxy-8-vinyl-3,4,8,9,10,11-hexahydro-1H-pyrido[4′,3′:3,4]pyrazolo[1,5-a]azepine-2(7H)-carboxamide;
(S*)-N-(3-Cyano-4-fluorophenyl)-11,11-difluoro-8-hydroxy-8-vinyl-3,4,8,9,10,11-hexahydro-1H-pyrido[4′,3′:3,4]pyrazolo[1,5-a]azepine-2(7H)-carboxamide;
N-(3-Cyano-4-fluorophenyl)-8-ethynyl-11,11-difluoro-8-hydroxy-3,4,8,9,10,11-hexahydro-1H-pyrido[4′,3′:3,4]pyrazolo[1,5-a]azepine-2(7H)-carboxamide;
(R*)-N-(3-Cyano-4-fluorophenyl)-8-ethynyl-11,11-difluoro-8-hydroxy-3,4,8,9,10,11-hexahydro-1H-pyrido[4′,3′:3,4]pyrazolo[1,5-a]azepine-2(7H)-carboxamide;
(S*)-N-(3-Cyano-4-fluorophenyl)-8-ethynyl-11,11-difluoro-8-hydroxy-3,4,8,9,10,11-hexahydro-1H-pyrido[4′,3′:3,4]pyrazolo[1,5-a]azepine-2(7H)-carboxamide;
(S*)-N-(3-Cyano-4-fluorophenyl)-8-ethyl-11,11-difluoro-8-hydroxy-3,4,8,9,10,11-hexahydro-1H-pyrido[4′,3′:3,4]pyrazolo[1,5-a]azepine-2(7H)-carboxamide;
(R*)-N-(3-Cyano-4-fluorophenyl)-8-ethyl-11,11-difluoro-8-hydroxy-3,4,8,9,10,11-hexahydro-1H-pyrido[4′,3′:3,4]pyrazolo[1,5-a]azepine-2(7H)-carboxamide;
(R*)-N-(2-Bromo-3-fluoropyridin-4-yl)-8-ethynyl-11,11-difluoro-8-hydroxy-3,4,8,9,10,11-hexahydro-1H-pyrido[4′,3′:3,4]pyrazolo[1,5-a]azepine-2(7H)-carboxamide;
(R*)-N-(2-(Difluoromethyl)-3-fluoropyridin-4-yl)-8-ethynyl-11,11-difluoro-8-hydroxy-3,4,8,9,10,11-hexahydro-1H-pyrido[4′,3′:3,4]pyrazolo[1,5-a]azepine-2(7H)-carboxamide;
(S*)-N-(2-Bromo-3-fluoropyridin-4-yl)-8-ethynyl-11,11-difluoro-8-hydroxy-3,4,8,9,10,11-hexahydro-1H-pyrido[4′,3′:3,4]pyrazolo[1,5-a]azepine-2(7H)-carboxamide;
(S*)-N-(2-(Difluoromethyl)-3-fluoropyridin-4-yl)-8-ethynyl-11,11-difluoro-8-hydroxy-3,4,8,9,10,11-hexahydro-1H-pyrido[4′,3′:3,4]pyrazolo[1,5-a]azepine-2(7H)-carboxamide;
(3R,8R)-N-(3-cyano-4-fluorophenyl)-11,11-difluoro-8-hydroxy-3-methyl-3,4,8,9,10,11-hexahydro-1H-pyrido[4′,3′:3,4]pyrazolo[1,5-a]azepine-2(7H)-carboxamide;
(3R,8R)-N-(2-Bromo-3-fluoropyridin-4-yl)-11,11-difluoro-8-hydroxy-3-methyl-3,4,8,9,10,11-hexahydro-1H-pyrido[4′,3′:3,4]pyrazolo[1,5-a]azepine-2(7H)-carboxamide;
(3R,8R)-11,11-Difluoro-N-(4-fluoro-3-(trifluoromethyl)phenyl)-8-hydroxy-3-methyl-3,4,8,9,10,11-hexahydro-1H-pyrido[4′,3′:3,4]pyrazolo[1,5-a]azepine-2(7H)-carboxamide;
(3R,8R)-N-(2-(Difluoromethyl)-3-fluoropyridin-4-yl)-11,11-difluoro-8-hydroxy-3-methyl-3,4,8,9,10,11-hexahydro-1H-pyrido[4′,3′:3,4]pyrazolo[1,5-a]azepine-2(7H)-carboxamide;
(3R,8S)-N-(3-Cyano-4-fluorophenyl)-11,11-difluoro-8-hydroxy-3-methyl-3,4,8,9,10,11-hexahydro-1H-pyrido[4′,3′:3,4]pyrazolo[1,5-a]azepine-2(7H)-carboxamide;
(3R,8S)-N-(2-Bromo-3-fluoropyridin-4-yl)-11,11-difluoro-8-hydroxy-3-methyl-3,4,8,9,10,11-hexahydro-1H-pyrido[4′,3′:3,4]pyrazolo[1,5-a]azepine-2(7H)-carboxamide;
(3R,8S)-11,11-Difluoro-N-(4-fluoro-3-(trifluoromethyl)phenyl)-8-hydroxy-3-methyl-3,4,8,9,10,11-hexahydro-1H-pyrido[4′,3′:3,4]pyrazolo[1,5-a]azepine-2(7H)-carboxamide;
(3R,8S)-N-(2-(Difluoromethyl)-3-fluoropyridin-4-yl)-11,11-difluoro-8-hydroxy-3-methyl-3,4,8,9,10,11-hexahydro-1H-pyrido[4′,3′:3,4]pyrazolo[1,5-a]azepine-2(7H)-carboxamide;
(3R,8S*)-N-(3-Cyano-4-fluorophenyl)-11,11-difluoro-8-(hydroxymethyl) -3-methyl-3,4,8,9,10,11-hexahydro-1H-pyrido[4′,3′:3,4]pyrazolo[1,5-a]azepine-2(7H)-carboxamide;
(3R,8S*)-11,11-Difluoro-N-(4-fluoro-3-(trifluoromethyl)phenyl)-8-(hydroxymethyl)-3-methyl-3,4,8,9,10,11-hexahydro-1H-pyrido[4′,3′:3,4]pyrazolo[1,5-a]azepine-2(7H)-carboxamide;
(3R,8S*)-N-(2-Bromo-3-fluoropyridin-4-yl)-11,11-difluoro-8-(hydroxymethyl)-3-methyl-3,4,8,9,10,11-hexahydro-1H-pyrido[4′,3′:3,4]pyrazolo[1,5-a]azepine-2(7H)-carboxamide;
(3R,8S*)-N-(2-(Difluoromethyl)-3-fluoropyridin-4-yl)-11,11-difluoro-8-(hydroxymethyl)-3-methyl-3,4,8,9,10,11-hexahydro-1H-pyrido[4′,3′:3,4]pyrazolo[1,5-a]azepine-2(7H)-carboxamide;
(3R,8R*)-N-(3-Cyano-4-fluorophenyl)-11,11-difluoro-8-(hydroxymethyl) -3-methyl-3,4,8,9,10,11-hexahydro-1H-pyrido[4′,3′:3,4]pyrazolo[1,5-a]azepine-2(7H)-carboxamide;
(3R,8R*)-11,11-Difluoro-N-(4-fluoro-3-(trifluoromethyl)phenyl)-8-(hydroxymethyl)-3-methyl-3,4,8,9,10,11-hexahydro-1H-pyrido[4′,3′:3,4]pyrazolo[1,5-a]azepine-2(7H)-carboxamide;
(3R,8R*)-N-(2-Bromo-3-fluoropyridin-4-yl)-11,11-difluoro-8-(hydroxymethyl)-3-methyl-3,4,8,9,10,11-hexahydro-1H-pyrido[4′,3′:3,4]pyrazolo[1,5-a]azepine-2(7H)-carboxamide;
(3R,8R*)-N-(2-(Difluoromethyl)-3-fluoropyridin-4-yl)-11,11-difluoro-8-(hydroxymethyl)-3-methyl-3,4,8,9,10,11-hexahydro-1H-pyrido[4′,3′:3,4]pyrazolo[1,5-a]azepine-2(7H)-carboxamide;
(3R,8R*)-N-(3-Cyano-4-fluorophenyl)-11,11-difluoro-8-(fluoromethyl)-8-hydroxy-3-methyl-3,4,8,9,10,11-hexahydro-1H-pyrido[4′,3′:3,4]pyrazolo[1,5-a]azepine-2(7H)-carboxamide;
(3R,8R*)-N-(2-Bromo-3-fluoropyridin-4-yl)-11,11-difluoro-8-(fluoromethyl)-8-hydroxy-3-methyl-3,4,8,9,10,11-hexahydro-1H-pyrido[4′,3′:3,4]pyrazolo[1,5-a]azepine-2(7H)-carboxamide;
(3R,8R*)-N-(2-(Difluoromethyl)-3-fluoropyridin-4-yl)-11,11-difluoro-8-(fluoromethyl)-8-hydroxy-3-methyl-3,4,8,9,10,11-hexahydro-1H-pyrido[4′,3′:3,4]pyrazolo[1,5-a]azepine-2(7H)-carboxamide;
(3R,8S*)-N-(3-Cyano-4-fluorophenyl)-11,11-difluoro-8-(fluoromethyl)-8-hydroxy-3-methyl-3,4,8,9,10,11-hexahydro-1H-pyrido[4′,3′:3,4]pyrazolo[1,5-a]azepine-2(7H)-carboxamide;
(3R,8S*)-N-(2-Bromo-3-fluoropyridin-4-yl)-11,11-difluoro-8-(fluoromethyl)-8-hydroxy-3-methyl-3,4,8,9,10,11-hexahydro-1H-pyrido[4′,3′:3,4]pyrazolo[1,5-a]azepine-2(7H)-carboxamide;
(3R,8S*)-N-(2-(Difluoromethyl)-3-fluoropyridin-4-yl)-11,11-difluoro-8-(fluoromethyl)-8-hydroxy-3-methyl-3,4,8,9,10,11-hexahydro-1H-pyrido[4′,3′:3,4]pyrazolo[1,5-a]azepine-2(7H)-carboxamide;
(3R,8S*)-N-(2-Bromo-3-fluoropyridin-4-yl)-8-(cyanomethyl)-11,11-difluoro-8-hydroxy-3-methyl-3,4,8,9,10,11-hexahydro-1H-pyrido[4′,3′:3,4]pyrazolo[1,5-a]azepine-2(7H)-carboxamide;
(3R,8S*)-8-(Cyanomethyl)-N-(2-(difluoromethyl)-3-fluoropyridin-4-yl)-11,11-difluoro-8-hydroxy-3-methyl-3,4,8,9,10,11-hexahydro-1H-pyrido[4′,3′:3,4]pyrazolo[1,5-a]azepine-2(7H)-carboxamide;
(3R,8R*)-N-(2-Bromo-3-fluoropyridin-4-yl)-8-(cyanomethyl)-11,11-difluoro-8-hydroxy-3-methyl-3,4,8,9,10,11-hexahydro-1H-pyrido[4′,3′:3,4]pyrazolo[1,5-a]azepine-2(7H)-carboxamide;
(3R,8R*)-8-(Cyanomethyl)-N-(2-(difluoromethyl)-3-fluoropyridin-4-yl) -11,11-difluoro-8-hydroxy-3-methyl-3,4,8,9,10,11-hexahydro-1H-pyrido[4′,3′:3,4]pyrazolo[1,5-a]azepine-2(7H)-carboxamide;
(3R,8R*)-N-(3-Cyano-4-fluorophenyl)-8-((2,2-difluoroethoxy)methyl)-11,11-difluoro-8-hydroxy-3-methyl-3,4,8,9,10,11-hexahydro-1H-pyrido[4′,3′:3,4]pyrazolo[1,5-a]azepine-2(7H)-carboxamide;
(3R,8S*)-N-(3-Cyano-4-fluorophenyl)-8-((2,2-difluoroethoxy)methyl)-11,11-difluoro-8-hydroxy-3-methyl-3,4,8,9,10,11-hexahydro-1H-pyrido[4′,3′:3,4]pyrazolo[1,5-a]azepine-2(7H)-carboxamide;
(3R,8R*)-N-(3-Cyano-4-fluorophenyl)-8-ethynyl-11,11-difluoro-8-hydroxy-3-methyl-3,4,8,9,10,11-hexahydro-1H-pyrido[4′,3′:3,4]pyrazolo[1,5-a]azepine-2(7H)-carboxamide;
(3R,8R*)-N-(2-Bromo-3-fluoropyridin-4-yl)-8-ethynyl-11,11-difluoro-8-hydroxy-3-methyl-3,4,8,9,10,11-hexahydro-1H-pyrido[4′,3′:3,4]pyrazolo[1,5-a]azepine-2(7H)-carboxamide;
(3R,8R*)-N-(2-(Difluoromethyl)-3-fluoropyridin-4-yl)-8-ethynyl-11,11-difluoro-8-hydroxy-3-methyl-3,4,8,9,10,11-hexahydro-1H-pyrido[4′,3′:3,4]pyrazolo[1,5-a]azepine-2(7H)-carboxamide;
(3R,8S*)-N-(3-Cyano-4-fluorophenyl)-8-ethynyl-11,11-difluoro-8-hydroxy-3-methyl-3,4,8,9,10,11-hexahydro-1H-pyrido[4′,3′:3,4]pyrazolo[1,5-a]azepine-2(7H)-carboxamide;
(3R,8S*)-N-(2-Bromo-3-fluoropyridin-4-yl)-8-ethynyl-11,11-difluoro-8-hydroxy-3-methyl-3,4,8,9,10,11-hexahydro-1H-pyrido[4′,3′:3,4]pyrazolo[1,5-a]azepine-2(7H)-carboxamide;
(3R,8S*)-N-(2-Difluoromethyl)-3-fluoropyridin-4-yl)-8-ethynyl-11,11-difluoro-8-hydroxy-3-methyl-3,4,8,9,10,11-hexahydro-1H-pyrido[4′,3′:3,4]pyrazolo[1,5-a]azepine-2(7H)-carboxamide;
(3R,8S*)-N-(3-Cyano-4-fluorophenyl)-11,11-difluoro-8-(2-hydroxypropan-2-yl)-3-methyl-3,4,8,9,10,11-hexahydro-1H-pyrido[4′,3′:3,4]pyrazolo[1,5-a]azepine-2(7H)-carboxamide;
(3R,8S*)-N-(2-Bromo-3-fluoropyridin-4-yl)-11,11-difluoro-8-(2-hydroxypropan-2-yl)-3-methyl-3,4,8,9,10,11-hexahydro-1H-pyrido[4′,3′:3,4]pyrazolo[1,5-a]azepine-2(7H)-carboxamide;
(3R,8R*)-N-(3-Cyano-4-fluorophenyl)-11,11-difluoro-8-(2-hydroxypropan-2-yl)-3-methyl-3,4,8,9,10,11-hexahydro-1H-pyrido[4′,3′:3,4]pyrazolo[1,5-a]azepine-2(7H)-carboxamide;
(3R,8R*)-N-(2-Bromo-3-fluoropyridin-4-yl)-11,11-difluoro-8-(2-hydroxypropan-2-yl)-3-methyl-3,4,8,9,10,11-hexahydro-1H-pyrido[4′,3′:3,4]pyrazolo[1,5-a]azepine-2(7H)-carboxamide;
methyl (((3R,8R*)-2-((3-cyano-4-fluorophenyl)carbamoyl)-11,11-difluoro-3-methyl-2,3,4,7,8,9,10,11-octahydro-1H-pyrido[4′,3′:3,4]pyrazolo[1,5-a]azepin-8-yl)methyl)carbamate;
Methyl (((3R,8R*)-2-((2-bromo-3-fluoropyridin-4-yl)carbamoyl)-11,11-difluoro-3-methyl-2,3,4,7,8,9,10,11-octahydro-1H-pyrido[4′,3′:3,4]pyrazolo[1,5-a]azepin-8-yl)methyl)carbamate;
Methyl (((3R,8R*)-2((2-(difluoromethyl)-3-fluoropyridin-4-yl) carbamoyl)-11,11-difluoro-3-methyl-2,3,4,7,8,9,10,11-octahydro-1H-pyrido[4′,3′:3,4]pyrazolo[1,5-a]azepin-8-yl)methyl)carbamate;
Methyl (((3R,8S*)-2-((3-cyano-4-fluorophenyl)carbamoyl)-11,11-difluoro-3-methyl-2,3,4,7,8,9,10,11-octahydro-1H-pyrido[4′,3′:3,4]pyrazolo[1,5-a]azepin-8-yl)methyl)carbamate;
Methyl (((3R,8S*)-2((2-bromo-3-fluoropyridin-4-yl)carbamoyl)-11,11-difluoro-3-methyl-2,3,4,7,8,9,10,11-octahydro-1H-pyrido[4′,3′:3,4]pyrazolo[1,5-a]azepin-8-yl)methyl)carbamate;
Methyl (((3R,8S*)-2((2-(difluoromethyl)-3-fluoropyridin-4-yl) carbamoyl)-11,11-difluoro-3-methyl-2,3,4,7,8,9,10,11-octahydro-1H-pyrido[4′,3′:3,4]pyrazolo[1,5-a]azepin-8-yl)methyl)carbamate;
(3R,8S*)-N-(3-Cyano-4-fluorophenyl)-8,11,11-trifluoro-8-(hydroxymethyl)-3-methyl-3,4,8,9,10,11-hexahydro-1H-pyrido[4′,3′:3,4]pyrazolo[1,5-a]azepine-2(7H)-carboxamide;
(3R,8S*)-N-(2-Bromo-3-fluoropyridin-4-yl)-8,11,11-trifluoro-8-(hydroxymethyl)-3-methyl-3,4,8,9,10,11-hexahydro-1H-pyrido[4′,3′:3,4]pyrazolo[1,5-a]azepine-2(7H)-carboxamide;
(3R,8S*)-N-(2-(Difluoromethyl)-3-fluoropyridin-4-yl)-8,11,11-trifluoro-8-(hydroxymethyl)-3-methyl-3,4,8,9,10,11-hexahydro-1H-pyrido[4′,3′:3,4]pyrazolo[1,5-a]azepine-2(7H)-carboxamide;
(3R,8R*)-N-(3-Cyano-4-fluorophenyl)-8,11,11-trifluoro-8-(hydroxyl methyl)-3-methyl-3,4,8,9,10,11-hexahydro-1H-pyrido[4′,3′:3,4]pyrazolo[1,5-a]azepine-2(7H)-carboxamide;
(3R,8R*)-N-(2-Bromo-3-fluoropyridin-4-yl)-8,11,11-trifluoro-8-(hydroxylmethyl)-3-methyl-3,4,8,9,10,11-hexahydro-1H-pyrido[4′,3′:3,4]pyrazolo[1,5-a]azepine-2(7H)-carboxamide;
(3R,8R*)-N-(2-(Difluoromethyl)-3-fluoropyridin-4-yl)-8,11,11-trifluoro-8-(hydroxymethyl)-3-methyl-3,4,8,9,10,11-hexahydro-1H-pyrido[4′,3′:3,4]pyrazolo[1,5-a]azepine-2(7H)-carboxamide;
(3R,9S*)-N-(2-Bromo-3-fluoropyridin-4-yl)-11,11-difluoro-9-hydroxyl-3-methyl-3,4,8,9,10,11-hexahydro-1H-pyrido[4′,3′:3,4]pyrazolo[1,5-a]azepine-2(7H)-carboxamide;
(3R,9R*)-N-(2-Bromo-3-fluoropyridin-4-yl)-11,11-difluoro-9-hydroxyl-3-methyl-3,4,8,9,10,11-hexahydro-1H-pyrido[4′,3′:3,4]pyrazolo[1,5-a]azepine-2(7H)-carboxamide;
(3R,9S*)-N-(3-Cyano-4-fluorophenyl)-11,11-difluoro-9-hydroxyl-3-methyl-3,4,8,9,10,11-hexahydro-1H-pyrido[4′,3′:3,4]pyrazolo[1,5-a]azepine-2(7H)-carboxamide;
(3R,9R*)-N-(3-Cyano-4-fluorophenyl)-11,11-difluoro-9-hydroxy-3-methyl-3,4,8,9,10,11-hexahydro-1H-pyrido[4′,3′:3,4]pyrazolo[1,5-a]azepine-2(7H)-carboxamide;
(3R,9S*)-N-(2-(Difluoromethyl)-3-fluoropyridin-4-yl)-11,11-difluoro-9-hydroxy-3-methyl-3,4,8,9,10,11-hexahydro-1H-pyrido[4′,3′:3,4]pyrazolo[1,5-a]azepine-2(7H)-carboxamide;
(3R,9R*)-N-(2-(Difluoromethyl)-3-fluoropyridin-4-yl)-11,11difluoro-9-hydroxy-3-methyl-3,4,8,9,10,11-hexahydro-1H-pyrido[4′,3′:3,4]pyrazolo[1,5-a]azepine-2(7H)-carboxamide;
(3R,9R*)-N-(2-Bromo-3-fluoropyridin-4-yl)-11-fluoro-9-hydroxy-3-methyl-3,4,8,9-tetrahydro-1H-pyrido[4′,3′:3,4]pyrazolo[1,5-a]azepine-2(7H)-carboxamide;
(3R,9S*)-N-(2-Bromo-3-fluoropyridin-4-yl)-11-fluoro-9-hydroxy-3-methyl-3,4,8,9-tetrahydro-1H-pyrido[4′,3′:3,4]pyrazolo[1,5-a]azepine-2(7H)-carboxamide;
(3R,9R*)-N-(3-Cyano-4-fluorophenyl)-11,11-difluoro-9-hydroxy-9-(hydroxymethyl)-3-methyl-3,4,8,9,10,11-hexahydro-1H-pyrido[4′,3′:3,4]pyrazolo[1,5-a]azepine-2(7H)-carboxamide;
(3R,9S*)-N-(3-Cyano-4-fluorophenyl)-11,11-difluoro-9-hydroxy-9-(hydroxymethyl)-3-methyl-3,4,8,9,10,11-hexahydro-1H-pyrido[4′,3′:3,4]pyrazolo[1,5-a]azepine-2(7H)-carboxamide;
(3R,9R*)-N-(2-Bromo-3-fluoropyridin-4-yl)-11,11-difluoro-9-(fluoromethyl)-9-hydroxy-3-methyl-3,4,8,9,10,11-hexahydro-1H-pyrido[4′,3′:3,4]pyrazolo[1,5-a]azepine-2(7H)-carboxamide;
(3R,9R*)-N-(3-Cyano-4-fluorophenyl)-11,11-difluoro-9-(fluoromethyl)-9-hydroxy-3-methyl-3,4,8,9,10,11-hexahydro-1H-pyrido[4′,3′:3,4]pyrazolo[1,5-a]azepine-2(7H)-carboxamide;
(3R,9S*)-N-(2-Bromo-3-fluoropyridin-4-yl)-11,11-difluoro-9-(fluoromethyl)-9-hydroxy-3-methyl-3,4,8,9,10,11-hexahydro-1H-pyrido[4′,3′:3,4]pyrazolo[1,5-a]azepine-2(7H)-carboxamide;
(3R,9S*)-N-(3-Cyano-4-fluorophenyl)-11,11-difluoro-9-(fluoromethyl) -9-hydroxy-3-methyl-3,4,8,9,10,11-hexahydro-1H-pyrido[4′,3′:3,4]pyrazolo[1,5-a]azepine-2(7H)-carboxamide;
(3R,9R)-N-(3-Cyano-4-fluorophenyl)-11,11-difluoro-9-(fluoromethyl) -3-methyl-3,4,8,9,10,11-hexahydro-1H-pyrido[4′,3′:3,4]pyrazolo[1,5-a]azepine-2(7H)-carboxamide;
(3R,9S*)-N-(3-Cyano-4-fluorophenyl)-11,11-difluoro-9-(fluoromethyl) -3-methyl-3,4,8,9,10,11-hexahydro-1H-pyrido[4′,3′:3,4]pyrazolo[1,5-a]azepine-2(7H)-carboxamide;
(S*)-N-(2-Bromo-3-fluoropyridin-4-yl)-11,11-difluoro-9-(hydroxymethyl)-3,4,8,9,10,11-hexahydro-1H-pyrido[4′,3′:3,4]pyrazolo[1,5-a]azepine-2(7H)-carboxamide;
(S*)-N-(3-Cyano-4-fluorophenyl)-11,11-difluoro-9-(hydroxymethyl) -3,4,8,9,10,11-hexahydro-1H-pyrido[4′,3′:3,4]pyrazolo[1,5-a]azepine-2(7H)-carboxamide;
(R*)-N-(2-Bromo-3-fluoropyridin-4-yl)-11,11-difluoro-9-(hydroxymethyl)-3,4,8,9,10,11-hexahydro-1H-pyrido[4′,3′:3,4]pyrazolo[1,5-a]azepine-2(7H)-carboxamide;
(R*)-N-(3-Cyano-4-fluorophenyl)-11,11-difluoro-9-(hydroxymethyl) -3,4,8,9,10,11-hexahydro-1H-pyrido[4′,3′:3,4]pyrazolo[1,5-a]azepine-2(7H)-carboxamide;
(R*)-N-(2-Bromo-3-fluoropyridin-4-yl)-11,11-difluoro-9-hydroxy-3,4,8,9,10,11-hexahydro-1H-pyrido[4′,3′:3,4]pyrazolo[1,5-a]azepine-2(7H)-carboxamide;
(R*)-N-(3-Cyano-4-fluorophenyl)-11,11-difluoro-9-hydroxy-3,4,8,9,10,11-hexahydro-1H-pyrido[4′,3′:3,4]pyrazolo[1,5-a]azepine-2(7H)-carboxamide;
(R*)-N-(2-(Difluoromethyl)-3-fluoropyridin-4-yl)-11,11-difluoro-9-hydroxy-3,4,8,9,10,11-hexahydro-1H-pyrido[4′,3′:3,4]pyrazolo[1,5-a]azepine-2(7H)-carboxamide;
(S*)-N-(2-Bromo-3-fluoropyridin-4-yl)-11,11-difluoro-9-hydroxy-3,4,8,9,10,11-hexahydro-1H-pyrido[4′,3′:3,4]pyrazolo[1,5-a]azepine-2(7H)-carboxamide;
(S*)-N-(3-Cyano-4-fluorophenyl)-11,11-difluoro-9-hydroxy-3,4,8,9,10,11-hexahydro-1H-pyrido[4′,3′:3,4]pyrazolo[1,5-a]azepine-2(7H)-carboxamide;
(S*)-N-(2-(Difluoromethyl)-3-fluoropyridin-4-yl)-11,11-difluoro-9-hydroxy-3,4,8,9,10,11-hexahydro-1H-pyrido[4′,3′:3,4]pyrazolo[1,5-a]azepine-2(7H)-carboxamide; and
(3R)-N-(3-Cyano-4-fluorophenyl)-11,11-difluoro-10-hydroxy-3-methyl-1,3,4,7,8,9,10,11-octahydro-2H-pyrido[4′,3′:3,4]pyrazolo[1,5-a]azepine-2-carboxamide;
or a pharmaceutically acceptable salts, solvates, or N-oxides or N-oxides thereof.

21. A pharmaceutical composition comprising:
(A) at least one compound selected from compounds of Formula (I): 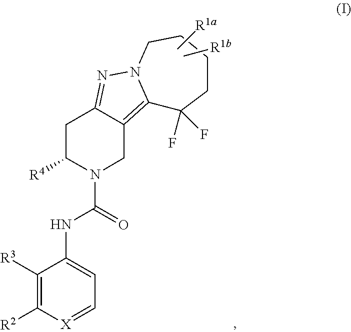
wherein
R^{1a} is H, or OH;
R^{1b} is selected from the group consisting of: F, OH, C₁₋₄alkyl, C₂₋₄alkenyl, C₂₋₄alkynyl, C₁₋₄haloalkyl, CH₂OH, C(CH₃)₂OH, CH₂CN, CH₂NH(C═O)CH₃, CH₂NH(C═O)OCH₃, CH₂OC₁₋₄haloalkyl, CH₂NH(C═O)CF₃, and OC₁₋₄haloalkyl,
or R^{1a} and R^{1b} come together to form ═CH₂;
R² is selected from the group consisting of: Br, CN, and C₁₋₄haloalkyl;
R³ is H, or F;
R⁴ is H or C₁₋₄alkyl; and
X is selected from the group consisting of: CH, CF, and N;
or a pharmaceutically acceptable salt, solvate, stereoisomer, isotopic variant, or a N-oxide of thereof; and
(B) at least one pharmaceutically acceptable excipient.

22. A pharmaceutical composition comprising at least one compound of claim 20 and at least one pharmaceutically acceptable excipient.

23. A method of treating an HBV infection in an individual in need thereof, comprising administering to the individual a therapeutically effective amount of at least one compound of claim 1.

24. A method of inhibiting or reducing the formation or presence of HBV DNA-containing particles or HBV RNA-containing particles in an individual in need thereof, comprising administering to the individual a therapeutically effective amount of a compound of claim 1.

25.-29. (canceled)
